(19) United States
(12) Patent Application Publication
(10) Pub. No.: US 2007/0122492 A1
(43) Pub. Date: May 31, 2007

(54) Plant extracts and dermatological uses thereof

(76) Inventors: Stephen Behr, Outremont (CA); Philippe Duret, Quebec (CA); Nathalie Gendron, Neuville (CA); Johane Guay, St-Augustin de Desmaures (CA); Bernard Lavallee, Ste. Foy (CA); Brigitte Page, Quebec (CA)

Correspondence Address: HELLER EHRMAN LLP, 275 MIDDLEFIELD ROAD, MENLO PARK, CA, 94025-3506, US

(21) Appl. No.: 10/533,025

(22) PCT Filed: Nov. 18, 2004
(86) PCT No.: PCT/CA04/02007

§ 371 Date: Nov. 20, 2006

(51) Int. Cl. A61K 36/45; A61K 36/53; A61K 36/899; A61K 36/8962; A61K 36/906; A61K 36/48; A61K 36/81; A61K 36/13; A61K 36/23

(52) U.S. Cl. 424/725; 424/750; 424/754; 424/732; 424/760; 424/770; 424/757; 424/756; 424/745

(57) ABSTRACT

The present invention provides for plant extracts and dermatological formulations comprising one or more plant extracts that are capable of inhibiting one or more extracellular proteases selected from the group of: matrix metalloprotease-1 (MMP-1), matrix metalloprotease-2 (MMP-2), matrix metalloprotease-3 (MMP-3), matrix metalloprotease-9 (MMP-9) and human leukocyte elastase (HLE). The present invention further provides for a rapid method for screening plant extracts to identify those having the above activity that are suitable for incorporation into the dermatological formulations of the invention. The invention also provides for the use of the plant extracts as dermatological agents suitable for the treatment or prevention of various dermatological conditions, including wrinkling or sagging of the skin, irradiation induced skin and/or hair damage, deepening of skin lines, elastotic changes in the skin, as well as for the routine care of the skin, hair and/or nails.

### FIELD OF INVENTION

The invention pertains to the field of dermatology, specifically within the field of dermatological preparations comprising plant extracts.

### BACKGROUND OF THE INVENTION

The skin is the most environmentally-stressed organ in mammals, particularly in humans. Not only is the skin subjected to germs, toxic chemicals and hostile environments, it is the only organ directly exposed to ultraviolet light (UV). In addition, the vitality of this organ is a consequence of genetic processes, which over time, lead to a decrease in the functionality of the skin. Hence, a variety of dermatological conditions may occur as a result of ongoing intrinsic factors (for example, chronological ageing, disease and allergies) and/or exposure to a number of extrinsic factors (such as infection, trauma, radiation, toxins and steroid use). Skin is a highly organized structure consisting of two principal parts. The outer thinner part, the epidermis or cuticle, is organised into four or five cell layers depending on its location. These layers are the stratum corneum, stratum lucidem (usually only present where the skin is thickened), stratum granulosum, stratum spinosum and stratum basale. The inner, thicker part of the skin, the dermis or true skin, is composed of a papillary layer above and a reticular layer below. The dermis also comprises blood vessels, nerves, hair follicles and sweat glands. The layer below the dermis, the hypodermis, comprises mainly loose connective tissue and adipose cells and may be considered part of the skin in that it functions to anchor the epidermis/dermis to the underlying bone and muscle. The hypodermis also supplies the dermis with blood vessels and nerves.

The cells of the skin, like many tissues, are generally in contact with a network of large extracellular macromolecules that occupy the spaces in a tissue between the component cells and between adjacent tissues. This extracellular matrix (ECM) functions as a scaffolding on which the cells and tissue are supported and is involved actively in regulating interaction of the cells that contact it. The principal macromolecules of the ECM include the collagens (the most abundant proteins in the body) and glycosaminoglycans (complex polysaccharides which are usually bonded to protein and then termed proteoglycans). Additional proteins that may be found in the ECM include elastin, fibronectin and laminin. The dermal layer of the skin is composed largely of ECM (or “connective tissue”) containing high proportions of collagen and elastin in which cells are embedded.

Components of the ECM are degraded by extracellular proteolytic enzymes that are secreted locally by cells. Extracellular proteases, in particular matrix metalloproteinases (MMPs), have been implicated in a number dermatological conditions, for example, in both chronological ageing and photo-ageing processes involve extracellular proteases (see, for example, U.S. Patent Application No. 200100513347). An age-related increase in levels of MMPs, in particular MMP-1, -2 and -9, in the skin has been demonstrated (see U.S. Patent Application No. 200100513347). An analogous increase in the level and/or activity of MMP-1, -2, -3 and -9 in the skin has also been shown to occur in response to extrinsic factors such as UV exposure (see U.S. Pat. No. 5,837,224). The ageing process (both chronological and photo-induced) involves the increased breakdown various components of the ECM in the skin, notably collagen, elastin and fibronectin. Enhanced expression of collagenase (MMP-1) and stromelysin-1 (MMP-3) has been described as playing a central role in connective tissue breakdown in the skin (Brenneisen, et al., (2002) Ann. N.Y. Acad. Sci., 973:31-43). Similarly, increased expression of serine elastase in hairless mouse models of chronological and photo-ageing was shown to result in increased fibronectin degradation (Labat-Robert, et al., (2000) J Photochem. Photobiol. B., 57:113-118).

Elastic fibers are essential extracellular matrix macromolecules comprising an elastin core surrounded by a mantle of fibrillin-rich microfibrils. These fibers endow connective tissues such as blood vessels, lungs and skin with the critical properties of elasticity and resilience (see review of elastic fibers by Kielty C M et al: J Cell Sci (2002) 115:2817-2828). Exposure to the sun is known to cause disorganization of elastin in the skin known as “elastosis,” which is also a hallmark of skin-ageing. Neutrophil elastase has been implicated in elastosis, for example, when compared to normal mice, mice that are deficient in neutrophil elastase are unaffected by exposure to UVB. In addition, an increase in elastase activity has been observed in the skin following chronic UVB irradiation (Tsukahara K et al Biol Pharm Bull 2001;24(9):998-1003). Both a synthetic inhibitor of fibroblast elastase and an extract of Sanguisorba officinalis L. inhibited wrinkle formation and maintained skin elasticity in the rat (Tsukahara K et al Biol Pharm Bull 2001;24(9):998-1003).

MMPs also play a role in the loss of elastic fibers in skin. Tissue loss during ageing and age-dependent pathologies are the result of a disturbed regulation of proteolytic activities in which elastase-type endopeptidases, especially MMP-2 and -9, are overactivated (Isnard N et al: Biomed Pharmacother. 2002 July;56(5):258-64). In addition, gelatinase B (MMP-9) has been shown to degrade fibrillin in human skin tissue sections (Berton A et al, Matrix Biol 2000;19(2):139-148).

In an effort to ameliorate the vast number of dermatological disorders, treatments spanning topical therapy (creams, oils, lotions, gels and sprays) to oral therapy, cosmetic procedures, injections and ultraviolet therapy have been developed. Topical skin applications, for example; are known in the art to help shield the skin from the vagaries of the environment. Conventional skin protections typically attempt to either protect the skin from UV light (see U.S. Pat. No. 5,141,741) or provide additional agents capable of neutralizing free radicals (U.S. Pat. No. 6,764,693). Methods of inhibiting either chronological or photo-ageing of the skin by application of UV blocking compounds in combination with compounds that inhibit MMPs have also been reported (U.S. Pat. Nos. 5,837,224; 6,130,254 and 6,365,630 and U.S. Patent Application No. 20010053347). Mercaptoketone and mercaptoalcohol compounds that inhibit the activity of MMPs and their use in treating or controlling disease states such as arthropathy, dermatological conditions, bone resorption, inflammatory diseases and tumor invasion have also been described (U.S. Pat. No. 6,307,101). Addition of certain plant extracts or phyto-compounds to preparations, such as lotions, creams and gels, to treat dermatological disorders has also been reported. These cosmetic compositions serve to shield the skin from UV light (U.S. Pat. Nos. 4,857,325; 5,141,741 and 6,342,208) and act as antioxidants in the neutralization of free radicals (U.S. Pat. No. 4,923,697). Some fruit extract-containing dermatological agents, capable of neutralizing free radicals, additionally moisturize and facilitate the hydration of the skin (see U.S. Pat. No. 6,800,292).

Other plant extracts useful in dermo-cosmetics have been described (see U.S. Pat. Nos. 6,682,763; 5,824,320 and 6,406,720). Here, external agents derived from olive plants are reported as having skin-beautifying effects, in particular, an anti-ageing effect related to the prevention and elimination of wrinkles and sags of the skin (U.S. Pat. No. 6,682,763). Furthermore, a whitening effect, which can lighten (U.S. Pat. No. 5,073,545) or prevent dark skin, melasma, ephelis and darkening or dullness of the skin has been reported (U.S. Pat. No. 6,682,763). Dermo-cosmetics containing plant extracts for application to the mucous membrane or exoskeleton, in addition to the skin, have also been considered (U.S. Pat. No. 6,406,720); the active ingredient of these cosmetics being derived from Spondias mom bin, Maprounea guianensis, Waltheria indica, Gouania blanchetiana, Cordia schomburgkii, Randia armata or Hibiscus furcellatus; Plant extracts useful in the treatment of eczema and/or psoriasis (U.S. Pat. Nos. 6,676,975 and 4,855,131), hemorrhoids (U.S. Pat. No. 5,627,216) and for maintaining general skin care (U.S. Pat. No. 6,193,975) have also been described.

A number of patents and publications report the inhibition of one or more extracellular proteases by compounds extracted from plants. For example, Sun et al., (1996) Phytotherapy Res., 10: 194-197, reports the inhibition in vitro of stromelysin (MMP-3) and collagenase by betulinic acid extracted from Doliocarpus verruculosis. Sazuka et al, (1997) Biosci. Biotechnol. Biochem., 61: 1504-1506, reports the inhibition of gelatinases (MMP-2 and MMP-9) and metastasis by compounds isolated from green and black teas. Kumagai et al, JP 08104628 A2, Apr. 1, 1996 (CA 125: 67741) reports the use of flavones and anthocyanines isolated from Scutellaris baicanlensis roots to inhibit collagenase. Gervasi et al., (1996) Biochem. Biophys. Res. Comm., 228: 530-538, reports the regulation of MMP-2 by some plant lectins and other saccharides. Dubois et al., (1998) FEBS Lett., 427: 275-278, reports the increased secretion of deleterious gelatinase-B (MMP-9) by some plant lectins. Nagase et al., (1998) Planta Med., 64: 216-219, reports the weak inhibition of collagenase by delphinidin, a flavonoid isolated from Solanum melongena.

Other reports include Asano et al. ((1998) Immunopharmacology, 39: 117-126), which describes the inhibition of TNF-α production using Tripterygium wilfordii Hook F. extracts; Maheu et al. ((1998) Arthritis Rheumatol, 41: 81-91), which reports the use of avocado/soy bean non-saponifiable extracts in the treatment of arthritis; Makimura et al. ((1993) J; Periodontol., 64: 630-636), which reports the use of green tea extracts to inhibit collagenases in vitro and Obayashi et al. ((1998) Nippon Keshonin Gijutsusha Kaishi, 32: 272-279 (CA 130: 92196)), which reports the inhibition of collagenase-I (MMP-1) from human fibroblast and neutrophil elastase by plant extract from Eucalyptus and Elder. Plant extracts derived from Capsicum Annuum L (U.S. Pat. No. 6,432,456) and from Brassica olearacea (U.S. Pat. No. 6,177,122) have also been described.

The effect of methanol extracts from medicinal plants on elastase activity has been reported by Lee and Kim (Inter. J. of Cosmetic Sci. 21:71-82 (1999)). Of approximately 150 extracts screened only the methanol extracts of A. catechu, C. cassia, M. fragrans, C. Ionga, A. katsumadia, and D. cassirrhizoma demonstrated good inhibition of elastase activity. Similarly, peptide-containing extracts of L. albus (PCT/FR00/01007, Publication No. WO 00/62789) have been shown to inhibit the activity of extracellular proteases including MMP-1, MMP-2 and MMP-9, using fibroblast models.

A process for obtaining plant extracts capable of inhibiting various extracellular proteases has been described in International Patent Application PCT/CA02/00285 (Publication No. WO 02/06992), in which the extracts were screened on the basis of their ability to inhibit extracellular proteases in in vitro assays. The ability of these extracts to inhibit extracellular proteases in vivo or to inhibit processes associated with the activity of such proteases, however, was not described or suggested.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the invention is to provide plant extracts and dermatological uses thereof. In accordance with one aspect of the present invention, there is provided a dermatological formulation comprising a physiologically acceptable carrier and an effective amount of one or more plant extracts having extracellular protease inhibiting activity, said plant extract derived from any one of the plants listed in Tables 1, 2, 3, 4 and 5 by solvent extraction, said extracellular protease selected from the group of: matrix metalloprotease-1 (MMP-1), matrix metalloprotease-2 (MMP-2), matrix metalloprotease-3 (MMP-3), matrix metalloprotease-9 (MMP-9) and human leukocyte elastase (HLE), wherein said extract affects one or more cellular activities in skin cells.

In accordance with another aspect of the present invention, there is provided a plant extract having extracellular protease inhibiting activity, said plant extract derived by solvent extraction from a plant selected from the group of: Aconitum napellus, Acorus calamus, Alchemilla mollis, Allium cepa, Allium sativum, Allium tuberosum, Ambrosia artemisuifolia, Anethum graveolens, Anthemis tinctoria, Aronia melanocarpa (Michx.) Ell., Arctostaphylos uva-ursi, Aronia×prunifolia, Artemisia dracunculus, Avena sativa, Beta vulgaris, Beta vulgaris L. subsp. Vulgaris, Borago officinalis, Brassica napus, Brassica oleracea, Brassica oleracea L. var. italica Plenck, Brassica rapa, Bromus inermis, Capsicum annuum L. var. annuum, Cerastium tomentosum, Chaerophyllum bulbosum, Chenopodium quinoa, Chenopodium quinoa subsp. Quinoa, Chenopodium quinoa Willd., Chichorium endivia, Chichorium endivia subsp. Endivia, Circium arvense, Citrullus lanatus, Cornus canadensis, Cornus sericea, Cynara cardunculus subsp. Cardunculus, Daucus carota, Daucus carota subsp carota L., Dolichos lablab, Euphorbia amygdaloides, Fagopyrum tataricum, Foeniculum vulgare, Frangula alnus, Galinsoga quadriradiata, Gentiana lutea, Geranium sanguineum, Geranium×cantabrigiense, Glycyrrhiza glabra, Hamamelis virginiana, Helianthus strumosus, Heliotropium arborescens, Hordeum vulgare subsp. Vulgare, Hypomyces lactifluorum, Juniperus communis L., Lentinus edodes, Lotus corniculatus, Manihot esculenta, Matricaria recutita, Melilotus albus, Melilotus alba Medik, Melissa officinalis, Mentha×piperita, Oenothera biennis, Pastinaca sativa L., Petroselinum crispum, Phaseolus vulgaris, Physalis philadelphica, Phytolacca decandra, Phytolacca decandra syn. P. americana, Pimpinella anisum, Pisum sativum, Potentilla anserina L., Potentilla fruticosa, Poterium sanguisorba, Pyrus communis, Raphanus raphanistrum, Rheum×hybridum, Rhus typhina L., Ribes nigrum L., Ribes sylvestre, Rodgersia spp., Rosmarinus officinalis, Rubus occidentalis, Rubus thibetanus, Rumex crispus, Rumex scutatus, Ruta graveolens, Salvia officinalis, Sambucus canadensis L., Setaria italica, Solanum melongena L., Sorghum dochna bicolor gr technicum, Stellaria media, Tanacetum cinerariifolium, Taraxacum officinale, Teucrium chamaedrys, Thymus fragantissimus, Thymus×citriodorus, Trifolium incarnatum, Triticosecale spp., Tropaeolum majus L., Tsuga canadensis, Tsuga diversifolia, Vaccinium angustifolium, Vaccinium angustifolium Ait., Vitia sp., ×Triticosecale spp., Zea mays L. and Zingiber officinale, and said extracellular protease selected from the group of: matrix metalloprotease-1 (MMP-1), matrix metalloprotease-2 (MMP-2), matrix metalloprotease-3 (MMP-3), matrix metalloprotease-9 (MMP-9) and human leukocyte elastase (HLE).

In accordance with another aspect of the present invention, the plant extract is derived from a plant selected from the group of: Beta vulgaris L., Brassica oleracea L., Capsicum annuum L, Chenopodium quinoa, Daucus carota L., Geranium×cantabrigiense, Juniperus communis L., Melilotus alba, Pastinaca sativa L., Potentilla anserina L., Rhus typhina L., Solanum melongena L., Tropaeolum majus L., Vaccinium angustifolium, ×Triticosecale spp. and Zea mays L.

In accordance with another aspect of the present invention, the plant extract is derived from the plant material by extraction with an alcohol, water, an aqueous buffer, or a combination thereof as solvent.

In accordance with another aspect, there is provided a use of a plant extract of the invention in the preparation of a dermatological formulation.

In accordance with another aspect, there is provided a use of a dermatological formulation of the present invention for the routine care of the skin, hair and/or nails.

In accordance with another aspect, there is provided a use of a dermatological formulation of the present invention to improve the health and/or appearance of the skin, hair and/or nails.

In accordance with another aspect, there is provided a use of a dermatological formulation of the present invention in the treatment or prevention of a dermatological condition.

In accordance with another aspect, there is provided a use of a dermatological formulation of the present invention to attenuate or prevent skin ageing.

In accordance with another aspect, there is provided a use of a plant extract of the present invention for the routine care of the skin, hair and/or nails.

In accordance with another aspect, there is provided a use of a plant extract of the present invention to improve the health and/or appearance of the skin, hair and/or nails.

In accordance with another aspect, there is provided a use of a plant extract of the present invention in the treatment or prevention of a dermatological condition.

In accordance with another aspect, there is provided a use of a plant extract of the present invention to attenuate or prevent skin ageing.

In accordance with another aspect of the present invention, there is provided a process for identifying a plant extract suitable for the preparation of a dermatological formulation, said process comprising the steps of: (a) generating a plurality of potential extracts by solvent extraction of plant material; (b) analysing the ability of each of said potential plant extracts to inhibit one or more extracellular protease selected from the group of: matrix metalloprotease-1 (MMP-1), matrix metalloprotease-2 (MMP-2), matrix metalloprotease-3 (MB-3), matrix metalloprotease-9 (MMP-9) and human leukocyte elastase (HLE); (c) selecting those potential extracts that are capable of inhibiting the activity of at least one of said extracellular proteases to provide a group of extracts; (d) analysing each extract in said group of extracts for the ability to affect one or more cellular activities in skin cells selected from the group of: attenuating the breakdown of collagen, fibronectin, fibrillin and/or elastin; attenuating endothelial cell migration; increasing collagen production; attenuating UV-induced extracellular protease activity and attenuating tractional forces generated by fibroblasts; and (e) selecting an extract that is capable of affecting one or more of said cellular activities to provide a plant extract suitable for the preparation of a dermatological formulation.

### BRIEF DESCRIPTION OF THE FIGURES

FIG. 1 presents an overview of a procedure that can be followed in accordance with one embodiment of the invention in order to generate plant extracts, each of which is derived from solid plant material;

FIG. 2 describes in further detail, the procedure of FIG. 1;

FIG. 3 presents an overview of a commercial procedure that can be followed to prepare plant extracts based on the procedure of FIG. 1;

FIG. 4 shows the effect of a plant extract of the invention derived from Rheum rhabarbarum on cord formation, (a) untreated cells; (b) cells treated with a positive control; (c) cells treated with an extract of the invention (1× concentration), and (d) cells treated with an extract of the invention (2× concentration);

FIG. 5 presents an overview of a procedure that can be followed in another embodiment of the invention in order to generate plant extracts, each of which is derived from solid plant material;

FIG. 6 describes in further detail, the procedure of FIG. 5;

FIG. 7 depicts the effect of plant extracts of the invention on the viability of human keratinocytes and fibroblasts;

FIG. 8 depicts the effect of plant extracts of the invention on the production of collagen in human dermal fibroblasts; and

FIG. 9 depicts the effect of plant extracts of the invention on the release of IL-8 from human skin keratinocytes.

### DETAILED DESCRIPTION OF THE INVENTION

The present invention provides for dermatological formulations comprising one or more plant extracts that are capable of inhibiting at least one skin extracellular protease (EP). In the context of the present invention, the terms “skin extracellular protease” and “skin EP” refer to the extracellular proteases: matrix metallpprotease-1 (MMP-1), matrix metalloprotease-2 (MMP-2), matrix metalloprotease-3 (MMP-3), matrix metalloprotease-9 (MMP-9) and human leukocyte elastase (HLE). The present invention further provides for a rapid method for screening plant extracts to identify those having the above activity that are suitable for incorporation into the dermatological formulations of the invention. The invention also provides for the use of plant extracts having the above activity as dermatological agents suitable for the treatment or prevention of various dermatological conditions, including wrinkling or sagging of the skin, irradiation-induced skin and/or hair damage, deepening of skin lines, elastotic changes in the skin, and the like, as well as for the routine care of the skin, hair and/or nails and to improve the health and/or appearance of the skin, hair and nails.

The present invention additionally provides for novel plant extracts identified by the methods described herein that inhibit one or more skin extracellular proteases, and which are suitable for use as dermatological agents. Semi-purified/purified active ingredients (i.e. molecules or compounds) isolated from a plant extract of the invention and the use of these active ingredients, alone or in combination with an extract, as dermatological agents are also contemplated.

The integumentary system of a mammal is made up of components (the skin, hair and nails) derived from the ectoderm and subjacent mesoderm. Mammalian skin is composed of a number of layers of cells embedded in an extracellular matrix (the ECM), which provides structure to the skin and comprises a number of polymeric structural components including collagen, elastin and fibronectin. Dispersed within the ECM are various types of cells, including fibroblasts and immune cells, which secrete EPs into the ECM. The ECM of the skin is in a constant state of flux, or turnover, which is tightly regulated and mediated in part by the secreted EPs, which are capable of degrading the structural components of the ECM. A shift in this turnover to an increased rate in the breakdown of one or more ECM structural components, such as collagen(s) or elastin, results in an increased degradation of the ECM and undesirable structural changes in the skin itself. Changes in the structure of the ECM can also affect the hair and nails, which are reliant on the skin for nourishment. Shifts in the balance of ECM turnover can occur as a consequence of a disease condition or of exposure of the skin to harmful elements (such as UV irradiation), or they can occur naturally, for example, as part of the ageing process.

Accordingly, inhibition of skin EPs can attenuate undesirable EP-mediated ECM degradation in the skin and structural changes associated therewith. One embodiment of the present invention provides for plant extracts that are capable of attenuating undesirable EP-mediated ECM degradation in the skin and structural changes associated therewith. EP-mediated ECM degradation refers to the breakdown of one or more component of the ECM surrounding the cells of mammalian skin including, for example, collagen, elastin, fibrillin and/or fibronectin. Undesirable skin structural changes include, for example, wrinkling and/or sagging of the skin, loss of elasticity, redness, inflammation, formation of lesions, thinning of the epithelium, abnormal migration of cells within the skin (such as that which occurs during angiogenesis or inflammation), or various combinations thereof.

Definitions

Unless defined otherwise, all technical and scientific terms used herein have the same meaning as commonly understood by one of ordinary skill in the art to which this invention belongs.

The term “potential plants,” as used herein, is intended to include all species of the Kingdom Plantae, including terrestrial, aquatic or other plants under the Division Chlorophyta, Division Rhodophora, Division Paeophyta, Division Bryophyta and Division Tracheophyta; Subdivision Lycopsida, Subdivision Sphenopsida, Subdivision Pteropsida and Subdivision Spermopsida; Class Gymnospermae, Class Angiospermae, Subclass Dicotyledonidae and Subclass Monocotyledonidae.

The term “plant material,” as used herein, refers to any part or parts of a plant taken either individually or in a group. Examples include, but are not limited to, leaves, flowers, roots, seeds, pods, stems, fruits, seed coats, buds, and other parts of a plant.

The term “potential extract,” refers to a composition prepared by contacting plant material with a solvent following the procedures described herein, which has not yet been determined to possess inhibitory activity against one or more extracellular protease. The potential extract can optionally be subjected to one or more separation 1 and/or purification step.

The term “plant extract of the invention,” as used herein, refers to a composition prepared by contacting plant material with a solvent following the procedures described herein, which demonstrates inhibitory activity against one or more extracellular protease selected from the group of: matrix metalloprotease-1 (MMP-1), matrix metalloprotease-2 (MMP-2), matrix metalloprotease-3 (MMP-3), matrix metalloprotease-9 (MMP-9) and human leukocyte elastase (HLE). The plant extract can be a primary extract or a substantially pure extract.

The terms “substantially purified” and “substantially pure” when used in reference to F a plant extract of the invention refer to an extract that has been subjected to at least one additional treatment subsequent to a first solvent extraction of plant material. Thus the present invention provides for primary extracts that result from a “one-step” solvent extraction of plant material followed optionally with a filtration or centrifugation step, and for substantially pure plant extracts that have been subjected to one or more additional steps, such as liquid-liquid extraction, solid-liquid extraction, chromatography, distillation, evaporation, filtration, and the like following the initial extraction process. Both primary extracts and substantially pure extracts are encompassed by the term “plant extracts of the present invention.”

The term “stressor,” as used herein, refers to a factor, such as a physical factor, a chemical compound, or a biological agent that is used to activate a defense response in a plant and thereby elicit production of various chemicals, including extracellular protease inhibitors. Elicitors and inducers are also considered to be stressors. The term “isolated” when used in reference to an active ingredient, such as a molecule or compound, refers to a form of the active ingredient that has been removed from the plant tissue from which it is derived. Typically, an isolated active ingredient is relatively free of proteins, nucleic acids, lipids, carbohydrates or other materials with which it is naturally associated in a plant. An isolated active ingredient may be further purified using routine and well-known methods such as those described herein. As such, an isolated active ingredient of the invention can constitute at least about one or a few percent of a sample, for example, at least about five percent. In one embodiment, the isolated active ingredient constitutes at least about twenty percent of a sample. In another embodiment, the isolated active ingredient is further purified to constitute at least about fifty percent of a sample. In other embodiments, the isolated active ingredient can be further purified to constitute at least about eighty percent, at least about ninety percent and at least about ninety-five percent or more of a sample.

The term “skin cell,” as used herein, refers to a cell normally present within the skin of a mammal. “Skin” refers to the epidermis (including the stratum germinativum, stratum spinosum, stratum granulosum, stratum lucidum and stratum corneum), the dermis (including the papillary dermis and the reticular dermis) and the hypodermis. The term “skin cells” thus includes, but is not limited to, keratinocytes, fibroblasts, endothelial cells (including vascular endothelial cells), basal cells, granular cells, Merkel cells, melanocytes, Langerhans cells, leukocytes, mastocytes, nerve cells, adipose cells and macrophages.

The term “attenuate,” as used herein, means to slow-down, inhibit or prevent.

The term “cell migration,” as used herein, refers to the movement, typically abnormal, of a cell or cells from one locus to another. Examples of cell migration include the movement of cells through the ECM or basal lamina during angiogenesis.

A “dermatological agent,” as used herein, refers to an extract, compound, composition or formulation intended for the routine care of the integumentary system, for improving the health and/or appearance of the integumentary system or for the treatment or prevention of a dermatological condition.

The term “dermatological condition,” as used herein, refers to a condition present on one or more of the components of the integumentary system of a subject, i.e. on the skin, hair or nails, caused by ageing or by intrinsic or extrinsic factors.

The term “treatment,” as used herein, refers to an intervention performed with the intention of improving a recipient's status. The improvement can be subjective or objective and is related to the amelioration of the symptoms associated with, preventing the development of, or altering the pathology of a condition being treated. Thus, the term treatment is used in the broadest sense, and includes the prevention (prophylaxis), moderation, reduction, and curing of a condition at various stages. Prevention of deterioration of a recipient's status is also encompassed by the term. Those in need of treatment include those already having the condition as well as those prone to, or at risk of developing, the condition and those in whom the condition is to be prevented.

The term “ameliorate” or “amelioration” includes the arrest, prevention, decrease, or improvement in one or more the symptoms, signs, and features of the condition being treated, both temporary and long-term.

The term “subject” or “patient,” as used herein, refers to a mammal in need of treatment or who would otherwise benefit from the use of a dermatological formulation of the invention.

As used herein, the term “about” refers to a +/−10% variation from the nominal value. It is to be understood that such a variation is always included in any given value provided herein, whether or not it is specifically referred to.

Other chemistry terms herein are used according to conventional usage in the art, as exemplified by The McGraw-Hill Dictionary of Chemical Terms (ed. Parker, S., 1985), McGraw-Hill, San Francisco).

Plant Extracts

The present invention provides for plant extracts suitable for use as dermatological agents. In accordance with the present invention, the plant extracts are capable of inhibiting one or more skin extracellular proteases selected from the group of: matrix metalloprotease-1 (MMP-1), matrix metalloprotease-2 (MMP-2), matrix metalloprotease-3 (MMP-3), matrix metalloprotease-9 (MMP-9) and human leukocyte elastase (HLE).

While some plant extracts have previously been identified that inhibit one or more extracellular proteases, the potential for plant extracts that inhibit any one of this particular group of proteases to be effective in various dermatological applications has not previously been established. The systematic evaluation of a large plant library to identify extracts from the plants in this library that inhibit one or more of this particular group of proteases and the subsequent evaluation of the extracts in cellular models as described herein has allowed this potential to be recognised. Accordingly, the plant extracts of the invention suitable for use as dermatological agents inhibit at least one of the above listed skin EPs. The present invention also contemplates plant extracts that inhibit two or more, three or more, four or more, or all five of MMP-1, MMP-2, MMP-3, MMP-9 and HLE.

In one embodiment of the present invention, the plant extract is capable of inhibiting at least P-1. In another embodiment, the plant extract is capable of inhibiting at least MMP-2. In a further embodiment, the plant extract is capable of inhibiting at least MMP-3; In another embodiment, the plant extract is capable of inhibiting at least MMP-9. In another embodiment, the plant extract is capable of inhibiting at least HLE.

In an alternative embodiment, the plant extract is capable of inhibiting at least two of MMP-1, MMP-2, MMP-3, MMP-9 and HLE. In a further embodiment, the plant extract is capable of inhibiting at least three of MMP-1, MMP-2, MMP-3, MMP-9 and HLE.

The plant extracts may be selected from extracts known in the art and subsequently tested for their ability to inhibit one or more of MMP-1, MMP-2, MMP-3, MMP-9 and/or HLE, or they may be identified using the process described herein. In one embodiment of the present invention, the plant extracts are derived from one of the plants listed in Tables 1 to 5. In another embodiment, the plant extracts are derived from one of the plants listed in Table 6.

In another embodiment, the plant extracts are derived from a: plant selected from the group of: Aconitum napellus, Acorus calamus, Alchemilla mollis, Allium cepa, Allium sativum, Allium tuberosum, Ambrosia artemisiifolia, Anethum graveolens, Anthemis tinctoria, Aronia melanocarpa (Michx) Ell., Arctostaphylos uva-ursi, Aronia×prunifolia, Artemisia dracunculus, Avena sativa, Beta vulgaris, Beta vulgaris L. subsp. Vulgaris, Borago officinalis, Brassica napus, Brassica oleracea, Brassica oleracea L. var. italica Plenck, Brassica rapa, Bromus inermis, Capsicum annuum L. var. annuum, Cerastium tomentosum, Chaerophyllum bulbosum, Chenopodium quinoa, Chenopodium quinoa subsp. Quinoa, Chenopodium quinoa Willd., Chichorium endivia, Chichorium endivia subsp. Endivia, Circium arvense, Citrullus lanatus, Cornus canadensis, Cornus sericea, Cynara cardunculus subsp. Cardunculus, Daucus carota, Daucus carota subsp carota L., Dolichos lablab, Euphorbia amygdaloides, Fagopyrum tataricum, Foeniculum vulgare, Frangula alnus, Galinsoga quadriradiata, Gentiana lutea, Geranium sanguineum, Geranium×cantabrigiense, Glycyrrhiza glabra, Hamamelis virginiana, Helianthus strumosus, Heliotropium arborescens, Hordeum vulgare subsp. Vulgare, Hypomyces lactifluorum, Juniperus communis L., Lentinus edodes, Lotus corniculatus, Manihot esculenta, Matricaria recutita, Melilotus albus, Melilotus alba Medik, Melissa officinalis, Mentha×piperita, Oenothera biennis, Pastinaca sativa L., Petroselinum crispum, Phaseolus vulgaris, Physalis philadelphica, Phytolacca decandra, Phytolacca decandra syn. P. americana, Pimpinella anisum, Pisum sativum, Potentilla anserina L., Potentilla fruticosa, Poterium sanguisorba, Pyrus communis, Raphanus raphanistrum, Rheum×hybridum, Rhus typhina L., Ribes nigrum L., Ribes sylvestre, Rodgersia spp., Rosmarinus officinalis, Rubus occidentalis, Rubus thibetanus, Rumex crispus, Rumex scutatus, Ruta graveolens, Salvia officinalis, Sambucus canadensis L., Setaria italica, Solanum melongena L., Sorghum dochna bicolor gr technicum, Stellaria media, Tanacetum cinerariifolium, Taraxacum officinale, Teucrium chamaedrys, Thymus fragantissimus, Thymus×citriodorus, Trifolium incarnatum, Triticosecale spp., Tropaeolum majus L., Tsuga canadensis, Tsuga diversifolia, Vaccinium angustifolium, Vaccinium angustifolium Ait., Vitia sp., ×Triticosecale spp., Zea mays L. and Zingiber officinale.

In another embodiment, the plant extracts are derived from a plant selected from the group of. Allium cepa, Allium sativum, Ambrosia artemisiifolia, Ambrosia artemisiifolia, Arctostaphylos uva-ursi, Aronia×prunifolia, Artemisia dracunculus, Avena sativa, Beta vulgaris, Beta vulgaris L. subsp. Vulgaris, Brassica napus, Brassica oleracea, Brassica oleracea L. var. italica Plenck, Brassica rapa, Bromus inermis, Capsicum annuum L. var. annuum, Chenopodium quinoa, Chenopodium quinoa subsp. Quinoa, Chenopodium quinoa Willd., Chichorium endivia, Chichorium endivia subsp. Endivia, Citrullus lanatus, Cornus sericea, Daucus carota, Daucus carota subsp carota L., Dolichos lablab, Euphorbia amygdaloides, Foeniculum vulgare, Galinsoga quadriradiata, Gentiana lutea, Geranium sanguineum, Geranium×cantabrigiense, Glycyrrhiza glabra, Helianthus strumosus, Hypomyces lactifluorum, Juniperus communis L., Lentinus edodes, Lotus corniculatus, Manihot esculenta, Matricaria recutita, Melilotus albus, Melilotus alba Medik., Melissa officinalis, Oenothera biennis, Pastinaca sativa L., Phaseolus vulgaris, Physalis philadelphica, Pimpinella anisum, Pisum sativum, Potentilla anserina L., Potentilla fruticosa, Raphanus raphanistrum, Rheum×hybridum, Rhus typhina L., Ribes sylvestre, Rodgersia spp., Rubus occidentalis, Rubus thibetanus, Rumex crispus, Rumex scutatus, Setaria italica, Solanum melongena L., Sorghum dochna bicolor gr technicum, Stellaria media, Tanacetum cinerariifolium, Taraxacum officinale, Thymus fragantissimus, Thymus×citriodorus, Trifolium incarnatum, Triticosecale spp., Tropaeolum majus L., Tsuga canadensis, Tsuga diversifolia, Vaccinium angustifolium, Vaccinium angustifolium Ait., Vitia sp., ×Triticosecale spp., Zea mays L. and Zingiber officinale

In another embodiment of the present invention, the plant extract is derived from a plant selected from the group of: Beta vulgaris L., Brassica oleracea L., Capsicum annuum L, Chenopodium quinoa, Daucus carota L., Geranium×cantabrigiense, Juniperus communis L., Melilotus alba, Pastinaca sativa L., Potentilla anserina L., Rhus typhina L., Solanum melongena L., Triticosecale spp., Tropaeolum majus L., Vaccinium angustifolium, and Zea mays L.

In accordance with the present invention, the plant extracts are solvent-based extracts obtained from the selected plant by solvent extraction. The solvent can be an aqueous solvent (such as water or a buffer), or it can be a liquid organic compound, or a combination of an aqueous solvent and a liquid organic compound. In one embodiment of the invention, the plant extract is an aqueous, alcoholic or aqueous alcoholic extract. In another embodiment, the plant extract is an aqueous, ethanolic, glycolic, aqueous-ethanolic or aqueous-glycolic extract. In a further embodiment, the glycol is butylene glycol.

Preparation of the Plant Extracts

The plant extracts are obtained by solvent extraction of plant material from a selected plant. The actual extraction process is not critical to the invention, but typically employs as solvent an aqueous solvent (such as water of a buffer), a liquid organic compound, or a combination thereof. Exemplary liquid organic compounds that can be used as solvents in the extraction process to prepare the plant extracts include, but are not limited to, primary alcohols such as methyl alcohol (methanol), ethyl alcohol (ethanol), 1-propanol and 1-butanol; secondary alcohols such as 2-propanol and 2-butanol; tertiary alcohols such as 2-methyl-2-propanol; liquid polyhydric alcohols such as glycerine and glycols; and other known organic solvents such as acetone, tetrahydrofuran, acetonitrile, 1,4-dioxane, pyridine, dimethylsulfoxide, N,N-dimethyl formamide, acetic acid, diethyl ether, hexane, heptane, dichloromethane and ethyl acetate. Suitable glycols include, for example, ethylene glycol, propylene glycol, diethylene glycol, dipropylene glycol and 1,3-butylene glycol.

When the extraction process is carried out using a solvent that comprises a mixture of an aqueous solvent and a liquid organic compound, the content of the liquid organic compound ranges from about 5% to about 95% by volume. In one embodiment, the content of the liquid organic compound in the solvent ranges from about 10% to about 90% by volume. In another embodiment, the content of the liquid organic compound in the solvent ranges from about 20% to about 90% by volume. In a further embodiment, the content of the liquid organic compound in the solvent ranges from about 20% to about 85% by volume. In another embodiment, the content of the liquid organic compound in the solvent ranges from about 20% to about 50% by volume. In an alternate embodiment, the content of the liquid organic compound in the solvent ranges from about 50% to about 85% by volume.

For dermatological applications wherein the plant extract will be formulated for topical use, a solvent that is compatible with mammalian skin can be selected. Examples of such solvents include, but are not limited to, water, an aqueous buffer, a combination of water/buffer and a lower alcohol or an anhydrous lower alcohol. In the context of the present invention, a lower alcohol refers to an alcohol having 1 to 4 carbon atoms, such as a primary, secondary, tertiary or liquid polyhydric alcohol. Accordingly, in one embodiment of the present invention, the solvent is selected from water, a lower alcohol or a combination thereof. In another embodiment, the lower alcohol is selected from the group of: methyl alcohol (methanol), ethyl alcohol (ethanol), 1-propanol, 1-butanol, 2-propanol, 2-butanol, 2-methyl-1-propanol, 2-methyl-2-propanol, glycerine, ethylene glycol, propylene glycol, diethylene glycol, dipropylene glycol and 1,3-butylene glycol.

When the extraction employs a combination of an aqueous solvent and a lower alcohol as solvent, the lower alcohol content of the solvent typically ranges from about 10% to about 95% by volume. In one embodiment of the present invention, the lower alcohol content of the solvent ranges from about 10% to about 90% by volume.

In another embodiment, the lower alcohol content of the solvent ranges from about 15% to about 90% by volume. In a further embodiment, the lower alcohol content of the solvent ranges from about 15% to about 50% by volume. In another embodiment, the lower alcohol content of the solvent ranges from about 50% to about 90% by volume. In an alternate embodiment, the solvent comprises a combination of an aqueous solvent and a lower alcohol, wherein the lower alcohol content is not less than 20% by volume.

A number of standard extraction techniques known in the art can be employed to prepare the plant extracts. In general, the extraction process entails contacting solid plant material with a solvent with adequate mixing and for a period of time sufficient to ensure adequate exposure of the solid plant material to the solvent such that inhibitory activity present in the plant material can be taken up by the solvent.

The plant material employed in the extraction process can be the entire plant, or it can be one or more distinct tissues from a plant, for example, leaves, flowers, roots, seeds, pods, stems, fruits, seed coats, buds, or various combinations thereof. The plant material can be fresh, dried or frozen. The plant material may be used immediately after harvesting or it can be stored for a period of time prior to being subjected to the extraction process. If the plant material is stored, it can be treated prior to storage, for example, by drying, freezing, lyophilising, or some combination thereof. The storage time may be of various durations, for example, the storage period may be between a few days and a few years. Typically storage times range between less than one week to about one year in duration.

If desired, the plant material can be derived from a plant that was subjected to a harvest stress treatment. A stress treatment comprises contacting or treating the plant, or material from the plant, with one or more stressor with the aim of inducing or eliciting increased production of one or more chemicals. The stressor can be a chemical compound or a physical treatment. Examples of chemical stressors include, but are not limited to, organic and inorganic acids including fatty acids, glycerides, phospholipids, glycolipids, organic solvents, amino acids, peptides, monosaccharides, oligosaccharides, polysaccharides, lipopolysaccharides, phenolics, alkaloids, terpenes, terpenoids, antibiotics, detergents, polyamines, peroxides, ionophores, and the like. Examples of physical stress treatments include, but are not limited to, ultraviolet radiation, sandblasting, low and high temperature stress, and osmotic stress induced by salt or sugars. Nutritional stress is defined as depriving the plant of essential nutrients (e.g. nitrogen, phosphorus or potassium) in order to induce or elicit increased production of one or more chemicals. The one or more stressor (i.e. chemical compound(s), physical treatment(s), or combination thereof) may be applied continuously or intermittently to the plant material. Various stressors and procedures for stressing plants prior to extract preparation have been described previously (see International Patent Application WO 02/06992) and are suitable for use in the present invention.

In one embodiment of the present invention, the plant extract is prepared from a plant that has been subjected to a stress treatment. In another embodiment, the extract is prepared from a plant that has been subjected to one or more chemical stressors. In a further embodiment, the extract is prepared from a plant that has been subjected to one or more chemical stressors selected from the group of: γ-linolenic acid, γ-linolenic acid lower alkyl esters, arachidonic acid and arachidonic acid lower alkyl esters. In a further embodiment, the extract is prepared from a plant that has been subjected to one or more physical stress. In yet another embodiment, the extract is derived from an unstressed plant.

If desired, the plant material can be treated prior to the extraction process in order to facilitate the extraction process. Typically such treatment results in the plant material being fragmented by some means such that a greater surface area is presented to the solvent. For example, the plant material can be crushed or sliced mechanically, using a grinder or other device to fragment the plant parts into small pieces or particles, or the plant material can be frozen in liquid nitrogen and then crushed or fragmented into smaller pieces.

The amount of the solvent used in the extraction can range from about 1× to about 100× (mass/mass) that of the solid plant material. In one embodiment of the present invention, the amount of solvent used in the extraction process ranges from about 1× to about 50× (mass/mass) that of the solid plant material.

A variety of conditions can be employed for the extraction process. Typically, the extraction procedures are conducted over a period of time between about 10 minutes and about 24 hours at a temperature between about 4° C. and about 50° C. However, temperatures between about 4° C. and about 90° C., for example between about 4° C. and about 70° C. can be employed. Similarly, extraction time may be varied depending on other extraction conditions, for example the extraction time can range from several minutes to several hours.

Adequate contact of the solvent with the plant material can be encouraged by shaking the suspension. Alternatively, an extraction device equipped with, for instance, a stirring machine, can be employed which may improve the extraction efficiency. The extraction can be carried out at ordinary pressure, under pressure or at reduced pressure established by, for example, aspiration. Appropriate extraction conditions can readily be determined or selected by one skilled in the art taking into consideration the production conditions such as production facilities and yields.

Following the extraction process, the liquid fraction (the primary plant extract) can be separated from the solid (insoluble) matter. Separation of the liquid and solid fractions can be achieved by one or more standard separation processes known to those skilled in the art, such as various centrifugation or filtration processes.

If desired, the primary extract can be subjected to one or more additional steps to further purify the extract. For example, the primary extract may be subjected to solid-liquid extraction, liquid-liquid extraction, solid-phase extraction (SPE), membrane filtration, ultrafiltration, dialysis, electrophoresis, solvent concentration, centrifugation, ultracentrifugation, liquid or gas phase chromatography (including size exclusion, affinity, etc.) with or without high pressure, lyophilisation, evaporation, precipitation with various “carriers” (including PVPP, carbon, antibodies, and the like), the use of supercritical fluids (such as CO₂), or various combinations thereof to provide a substantially pure extract.

Testing the Plant Extracts

Determination of Extracellular Protease Inhibitory Activity

Following the extraction process, the plant extract can be tested for its ability to inhibit one or more skin EPs selected from the group of: MMP-1, MMP-2, MMP-3, MMP-9 and HLE, using a variety of techniques known in the art including, but not limited to, those described herein. In the context of the present invention, a plant extract that decreases the activity of an EP by at least 20% is considered to be capable of inhibiting the activity of that protease. In one embodiment of the present invention, a plant extract that inhibits the activity of one or more of MMP-1, MMP-2, MM-3, MMP-9 and HLE by at least 20% is considered to be an extract of the invention. In another embodiment, the plant extract inhibits the activity of one or more of MMP-1, MMP-2, MMP-3, MMP-9 and HLE by at least 30%. In another embodiment, the plant extract inhibits the activity of one or more of MMP-1, MMP-2, MMP-3, MMP-9 and HLE by at least 40%. In a further embodiment, the plant extract inhibits the activity of one or more of MMP-1, MMP-2, MMP-3, MMP-9 and HLE by at least 45%. In another embodiment, the plant extract inhibits the activity of one or more of MMP-1, MMP-2, MMP-3, MMP-9 and HLE by at least 50%.

In order to determine whether the extracts inhibit a skin EP, the extract can be tested against an individual skin EP or against a panel comprising two or more of MMP-1, MMP-2, MMP-3, MMP-9 and HLE.

As indicated above, a variety of methods and techniques for measuring the ability of the extracts to inhibit the activity of a skin EP either qualitatively or quantitatively are known in the art. For example, there are currently several assays to measure the activity of MMPs and elastase (for a review of these methods, see Murphy and Crabbe, In Barrett (ed.) Methods in Enzymology. Proteolytic Enzymes: Aspartic Acid and Metallopeptidases, New York: Academic Press, 1995, 248: 470), including the gelatineolytic assay (which is based on the degradation of radio-labelled type I collagen), the zymography assay (which is based on the presence of negatively-stained bands following electrophoresis through substrate-impregnated SDS polyacrylamide gels) and a microtitre plate assay developed by Pacmen et al., (Biochem. Pharm. (1996) 52:105-111).

Other methods include those that employ auto-quenched fluorogenic substrates. Many fluorogenic substrates have been designed for quantification of the activity of MMPs and elastase, through fluorescent level variation measuring (reviewed by Nagase and Fields (1996) Biopolymers 40: 399-416). Another method of measuring EP activity makes use of the fluorescent activated substrate conversion (FASC) assay described in Canadian Patent No. 2,189,486 and in St-Pierre et al., ((1996) Cytometry 25: 374-380).

Various formats known in the art may be employed in the assays. For example, the extract may be tested against one or more EPs in a sequential fashion or it may be tested against a plurality, or array, of skin EPs simultaneously. The assays may be adapted to high throughput as is known in the art in order to facilitate simultaneous testing of an extract against a plurality of skin EPs.

The assays can be conducted using purified or semi-purified EPs. Methods of isolating and purifying EPs are well known in the art. In addition, many EPs are commercially available (for example, from Sigma-Aldrich, St. Louis, Mo. and Calbiochem, San. Diego, Calif.).

Alternatively, the ability of the extracts to inhibit the activity of skin EPs can be evaluated using cultures of cells that secrete one or more skin EPs. In this case a cell culture is contacted with an appropriate amount of the extract. After an appropriate period of time, the cells are extracted, centrifuged and the proteolytic activity in the supernatant is measured. This method is useful in determining the ability of an extract to inhibit a set of EPs secreted by a particular cell line or combination of cell lines. For example, assays can be conducted with cell lines derived from mammalian skin, such as keratinocytes or fibroblasts.

Inhibition of EP Activity in Skin Models

As an extension of the cell culture assays described above, the extracts may be tested in an appropriate skin model for their ability to inhibit one or more of MMP-1, MMP-2, MMP-3, MMP-9 and HLE. For example, an in vitro human skin model can be employed to test the extract(s). Such models are typically constructed from human fibroblasts and keratinocytes by first forming a gel comprising human dermal fibroblasts and collagen. Cell culture medium is added and the gel incubated for a sufficient number of days to allow for fibroblast proliferation, and for collagen and protease synthesis and secretion into the gel. Following this incubation period, donor-matched human epidermal keratinocytes in a biological medium are gently pipetted onto the gel and allowed to establish a confluent layer on its surface. The test plant extract is added and after a suitable incubation period (for example, between 6 and 24 hours), the gels are extracted and centrifuged and the proteolytic activity in the supernatant is assayed.

Immune cells can also be added to the above skin model in order to provide a source of elastase enzymes. Other examples of skin models are provided in the art, for example, see U.S. Pat. No. 6,079,415 and references therein.

In vivo Testing of EPe Inhibition

Alternatively, the ability of the extracts to inhibit skin EP activity may be assessed in vivo using various standard techniques. For example, the ability of the extracts to inhibit protease activity can be determined in animal models or human volunteers. An example of a suitable animal model would be a skh-1 mouse or nude mouse or rat that is treated with an extract of the invention and then exposed to UV radiation (see, Nishimori et al. (2001) J. Invest. Dernatol. 117:1458-1463). UV radiation is known to increase the level of activity of certain MMPs (see, for example, U.S. Pat. No. 6,130,254). Skin biopsies are taken from the animal and the amount of EP activity in the biopsied sample can be measured using standard techniques as an indication of the inhibitory activity of the test extract.

Human trials may also be used to evaluate the ability of an extract to inhibit EP activity in the skin. For example, skin biopsies can be taken from adult volunteers exposed to UV radiation and treated prior to or after UV exposure with an extract. The biopsy samples can be assessed for EP activity and compared to an appropriate control (for example, skin biopsies from individuals treated with a control compound or untreated individuals). Alternatively, as an age-related increase in the relative activities of MMP-1, MMP-2 and MMP-9 has been demonstrated (see, for example, U.S. Patent Application No. 20010053347), elderly individuals (for example, those over 80 years of age) could be used as volunteers for the trials without the requirement for UV exposure.

In order to assess the protease activity in skin biopsies, the samples are typically flash frozen, mechanically ground and/or homogenised. After centrifugation, the supernatants are isolated and used to assess EP activity in assays such as those outlined above.

In vitro Cellular Activity in Skin Cells

In order to be useful in dermatological applications, the selected plant extracts are capable of affecting one or more cellular activity of skin cells in a beneficial manner. The ability of plant extracts to affect one or more cellular activities in skin cells can be assessed in vitro using one, or a combination, of standard techniques known in the art. Cellular activities in skin cells that can be assessed in vitro include, but are not limited to, the breakdown of a structural component of the ECM, such as collagen, fibronectin, fibrillin and/or elastin; cell migration; collagen production; UV-induced extracellular protease activity and tractional forces generated by fibroblasts; response to oxidative stresses, inhibition of release of IL-8 or other cytokines, response to induced apoposis, wound healing.

For example, the ability of the extracts of the invention to attenuate the breakdown of one or more ECM component can be assessed in vitro using skin models such as those described above. After incubation with a plant extract, the gels can be extracted and assayed for the loss of one or more structural components of the ECM, such as elastin, collagen, fibronectin and/or fibrillin. Alternatively, the gels can be assayed for the presence of fragments of elastin, collagen, fibronectin and/or fibrillin using standard techniques as an indication of the breakdown of these components.

Elastin, for example, can be quantitated biochemically as desmosine or visualized histologically (Starcher B and Conrad M: Ciba Found Symp. (1995) 192:338-46). Alternatively, confocal microscopy can be used in visualize the dermal microfibrillar network (Watson R E et al: J Invest Dermatol. (1999) 112(5):782-7). Intact elastin and elastin fragments can also be measured by immunoblotting (Sakuraoka K et al: J Dermatol Sci (1996) 12(3):232-237).

Biochemical and/or immunochemistry methods can be used to assess changes in the amount of collagen in the gels. Ultrastructural methods can also be used to assess changes in the amount of collagen in the gels (Fligiel S E et al: J Invest Dermatol. (2003) 120(5):842-8). Type I collagen, the most abundant extracellular matrix protein deposited in cutaneous involvement, can be measured using the method described by Allanore Y et al (J Rheumatol. (2003) 30(1):68-73).

Quantitative reverse transcriptase-polymerase chain reaction analysis can be used to determine the presence of dermal elastosis, diminished fibrillin and type VII collagen expression (Bosset S et al: Br J Dermatol. (2003) 148(4):770-8).

Some of the more complex skin models allow for more sophisticated testing procedures such as those described by Roguet R (Skin Pharmacol Appl Skin Physiol. (2002) 15 Suppl 1:1-3), which can also be employed in testing the plant extracts.

In general, the ability of an extract to inhibit migration of cells can be assessed in vitro using standard cell migration assays. Typically, such assays are conducted in multi-well plates, the wells of the plate being separated by a suitable membrane into top and bottom sections. The membrane is coated with an appropriate compound, the selection of which is dependent on the type of cell being assessed and can be readily determined by one skilled in the art. Examples include collagen, gelatinee or Matrigel for endothelial cells. An appropriate chemo-attractant, such as EGM-2, IL-8, αFGF, βFGF and the like, is added to the bottom chamber as a chemo-attractant. An aliquot of the test cells together with the extract are added to the upper chamber. Typically various dilutions of the extract are tested. After a suitable incubation time, the membrane is rinsed, fixed and stained. The cells on the upper side of the membrane are wiped off, and then randomly selected fields on the bottom side are counted.

Inhibition of cell migration can also be assessed using the cord formation assay. In this assay endothelial cells with or without plant extract are plated onto Matrigel and incubated under appropriate conditions. After a suitable period of time (for example, between 18 and 24 hours), migration of cells is assessed by visual inspection to determine whether the cells have formed into cords.

Various cell lines can be used in cell migration assays. Examples of suitable endothelial cell lines include, but are not limited to, human umbilical vein endothelial cells (HUVECs), bovine aortic endothelial cells (BAECs), human coronary artery endothelial cells (HCAECs), bovine adrenal gland capillary endothelial cells (BCE) and vascular smooth muscle cells. HUVECs can be isolated from umbilical cords using standard methods (see, for example, Jaffe et al. (1973) J. Clin. Invest. 52: 2745), or they can be obtained from the ATCC or various commercial sources, as can other suitable endothelial cell lines.

The effect of the plant extracts on collagen I production in the skin cells can be assessed, for example, using immunochemical methods. One exemplary method involves measuring the release of the procollagen type I C-peptide (PIP) in skin cells treated with the extract and comparing this to the amount of PEP released by untreated controls and/or controls treated with a compound known to affect collagen production. ELISA kits suitable for assaying PIP are commercially available (for example from Takara Mirus Bio, Madison, Wis.). As PIP is cleaved off the procollagen molecule during formation of the collagen triple helix, the amount of this peptide released by the skin cells is stoichiometrically proportional to the amount of collagen synthesized.

UV-induced extracellular protease activity can be assessed by irradiating cultures of skin cells with UVA light and then treating the irradiated cells with the extract. Alternatively, the extract can be added to the cells prior to irradiation to assess the prophylactic effect of the extract. After a suitable period of incubation in an appropriate medium, supernatants can be removed from the cells and assayed for proteolytic activity as described above. Results can be compared to untreated cells and/or cells treated with a compound known to affect UV-induced protease activity.

Skin cells suitable for use in the above assays include human dermal fibroblasts, keratinocytes, melanocytes, Langerhans cells, cells of the hair follicle and cells of the immune system which produce proteases, including leukocytes, macrophages and lymphocytes.

As is known in the art, MMPs may act to extend anchoring of fibroblasts on the extracellular matrix, resulting in greater fibroblast tractional forces. Accordingly, the effect of the plant extracts on the tractional forces generated by fibroblasts can be assayed. This assay employs a model comprising fibroblasts embedded in a collagen matrix to create a derm-like environment. Such a model can be prepared by adding fibroblasts to a solution of collagen I in medium and then allowing the collagen to polymerize to form a gel. After an appropriate incubation period, the derm-like gel is treated with an extract and the amount of contraction measured over a period of time, for example, several days. The amount of contraction can be assessed for example, by digitally photographing the gel at various time points and calculating the gel area using appropriate software. The amount of contraction can be compared to untreated control gels and/or gels treated with a compound known to affect fibroblast tractional forces.

Additional Testing

The plant extracts may undergo additional testing if desired. For example, the ability of the plant extracts to affect one or more cellular activity of skin cells can be assessed in vivo and/or the plant extracts may be submitted to testing on human volunteers to assess their ability to exert the desired dermatological effect(s). The plant extracts may also undergo one or more safety, stability and/or bioavailability test prior to testing on human volunteers.

1. In vivo Testing

The ability of the extracts of the invention to affect one or more cellular activity of skin cells can be assessed in vivo using various standard techniques. For example, using appropriate animal models and/or human volunteers.

Degeneration of the ECM, in particular due to the breakdown of collagen and/or elastin, can be assessed in skin biopsies, for example, by histological examination of skin tissue after treatment with the extract. Methods described above for the determination of the breakdown of one or more structural components of the ECM can also be used on the biopsied samples. Histology can also be used to determine abnormal cell migration.

Skin changes, such as wrinkling and/or sagging, reddening, formation of lesions, abnormal pigmentation and the like, can be assessed by visual examination. For example, the effect of the plant extraction the skin can be evaluated by formulating the extract such that it is suitable for external application to the skin and susequently sensory tests can be conducted on the formulation using by a panel of human volunteers. A sensory test typically involves application of the formulation to the skin of the panelists on a regular basis, such as once or twice a day, over a period of several weeks. The effect of the formulation on the skin can be evaluated by inspecting the skin of the panelists and assessing visually the skin characteristic or characteristics being investigated, for example, the tenseness and gloss of the skin, a decrease of any wrinkles, sags, reddening, lesions and/or abnormal pigmentation.

Erythema in skin samples can be determined, for example, using commercially available chromameter. The ability of the plant extracts to reduce inflammation in the skin can also be assessed in human volunteers using standard techniques, including visual inspection.

The ability of the plant extract to inhibit endothelial cell migration can also be assessed in vivo, using standard techniques such as the CAM assay (see Brooks et al., in Methods in Molecular Biology, Vol. 129, pp. 257-269 (2000), ed. A. R. Howlett, Humana Press Inc., Totowa, N.J.; Ausprunk et al., (1975) Am. J. Pathol., 79:597-618; Ossonski et al., (1980) Cancer Res., 40:2300-2309), the Matrigel plug assay (see, for example, Passaniti, et al., (1992) Lab. Invest. 67:519-528) or the corneal micropocket assay (see D'Amato, et al., (1994) Proc. Natl, Acad. Sci. USA, 91:4082-4085; Koch et al., (1991) Agents Actions, 34:350-7; Kenyon, et al., (1996) Invest. Ophthalmol. Vis. Sci. 37:1625-1632).

The CAM assay measures neovascularization of whole tissue, wherein chick embryo blood vessels grow into the CAM or into the tissue transplanted on the CAM, and is a well-recognised assay model for in vivo angiogenesis. The Matrigel plug assay involves introducing an extract into cold liquid Matrigel which, after subcutaneous injection into a suitable animal model, solidifies and permits penetration by host cells and the formation of new blood vessels. After a suitable period of time, the animal is sacrificed, the Matrigel plug is recovered and angiogenesis is assessed in the Matrigel plug by measuring haemoglobin or by scoring selected regions of histological sections for vascular density. Modifications of this assay have also been described (see, for example, Akhtar et al., (2002) Angiogenesis 5:75-80; Kragh et al., (2003) Int J Oncol. 22:305-11). The corneal micropocket assay involves preparing pellets from a sterile hydron polymer containing a suitable amount of the extract. The pellets are surgically implanted into corneal stromal micropockets created at an appropriate distance medial to the lateral corneal limbus of a test animal. Angiogenesis can be quantitated at various times after pellet implantation through the use of stereomicroscopy. Typically, the length of neovessels generated from the limbal vessel ring toward the centre of the cornea and the width of the neovessels are measured.

2. Other Tests

In addition to the above tests, the plant extracts of the invention may be submitted to other standard tests to evaluate safety, cytotoxicity, stability, bioavailability and the like. Exemplary tests to determine the cytotoxicity of the extracts and their potential to induce cytokine release are described herein (see Examples X and XII).

The ability of an extract to penetrate the skin can be assessed, for example, by in vitro release tests (see, for example, the U.S. Center for Drug Evaluation and Research guidance document entitled “Guidance for Industry. Nonsterile Semisolid Dosage Forms. Scale-up and postapproval changes: in vitro release testing and in vivo bioequivalence documentation”). Typically, such testing is conducted using an open chamber diffusion cell, such as a Franz cell, fitted with an appropriate membrane. The test extract is placed on the upper side of the membrane and kept occluded to prevent solvent evaporation and compositional changes. A receptor fluid, such as aqueous buffer or hydro-alcoholic medium, is placed on the other side of the membrane in a receptor cell. Diffusion of the active component across the membrane is monitored by assay of sequentially collected samples of the receptor fluid. For the extracts of the invention, the assay could comprise, for example, testing the ability of the collected sample to inhibit EP activity. The membrane can be a synthetic membrane, for example polysulphone, cellulose acetate or nitrate, or polytetrafluoroethylene, or it can be a skin sample, such as a sample taken from a cadaver.

Other tests are known in the art (for example, see U.S. Pharmacopoeia XXII (1990)) and are suitable for testing the stability and/or safety of the extracts.

As will be readily apparent to one skilled in the art, a selected extract may need to meet certain criteria in order to meet regulatory requirements for human use. Conducting tests such as those described above, therefore, allows the suitability of an extract for human use to be assessed.

Isolation of Active Ingredients

The present invention also provides for active ingredients isolated from the plant extracts of the invention. In the context of the present invention an “active ingredient” is a compound or molecule that is capable of inhibiting one or more skin EPs selected from the group of: MMP-1, MMP-2, MMP-3, MMP-9 and HLE. The active ingredient may be proteinaceous or non-proteinaceous. Isolated active ingredients can be tested for their ability to inhibit one or more of MMP-1, MMP-2, MMP-3, MMP-9 and HLE using the procedures described above.

There are a number of techniques well known in the art for isolating active components from mixtures that may be employed to isolate the active ingredients from a plant extract of the invention. These techniques include, but are not limited to, solid-liquid extraction, liquid-liquid extraction, solid-phase extraction (SPE), membrane filtration, ultrafiltration, dialysis, electrophoresis, solvent concentration, centrifugation, ultracentrifugation, liquid or gas phase chromatography (including size exclusion, affinity, and the like) with or without high pressure, lyophilisation, evaporation, precipitation with various “carriers” (including PVPP, carbon, antibodies, and the like), or various combinations thereof. One skilled in the art, would appreciate how to use such options, in a sequential fashion, in order to enrich each successive fraction in the activity of interest by following its activity throughout the isolation procedure.

Solid-liquid extraction means include the use of soxhlet extractors, vortex shakers, ultrasounds and other means to enhance extraction, as well as recovery by filtration, centrifugation and related methods as described in the literature (see, for example, R. J. P. Cannell, Natural Products Isolation, Humana Press, 1998). Examples of solvents that may be used include, but are not limited to, hydrocarbon solvents, chlorinated solvents, organic esters, organic ethers, alcohols, water, and mixtures thereof. The use of supercritical solvents is also contemplated and includes the use of modifiers such as those described in V. H. Bright (Supercritical Fluid Technology, ACS Symp. Ser. Vol. 488, ch. 22, 1999).

Liquid-liquid extraction means include the use of various mixtures of solvents known in the art, including solvents under supercritical conditions. Typical solvents include, but are not limited to, hydrocarbon solvents, chlorinated solvents, organic esters, organic ethers, alcohols, water, various aqueous solutions, and mixtures thereof. The liquid-liquid extraction can be effected manually, or it can be semi-automated or completely automated, and the solvent can be removed or concentrated by standard techniques in the art (see, for example, S. Ahuja, Handbook of Bioseparations, Academic Press, 2000).

Solid-phase extraction (SPE) techniques include the use of cartridges, columns or other devices known in the art. The sorbents that may be used with such techniques include, but are not limited to, silica gel (normal phase), reverse-phase silica gel (modified silica gel), ion-exchange resins, and fluorisil. The invention also includes the use of scavenger resins or other trapping reagents attached to solid supports derived from organic or inorganic macromolecular materials to remove selectively active ingredients or other constituents from the extracts.

Membrane, reverse osmosis and ultrafiltration means include the use of various types of membranes known in the art, as well as the use of pressure, vacuum, centrifugal force, and/or other means that can be utilised in membrane and ultrafiltration processes (see, for example, S. Ahuja, Handbook of Bioseparations, Academic Press, 2000).

Dialysis means include membranes having a molecular weight cut-off varying from less than about 0.5 KDa to greater than about 50 KDa. The invention also covers the recovery of active ingredients from either the dialysate or the retentate by various means known in the art including, but not limited to, evaporation, reduced pressure evaporation, distillation, vacuum distillation, and lyophilization.

Chromatographic means include various means of carrying out chromatography known by those skilled in the art and described in the literature (see, for example, G. Sofer, L. Hagel, Handbook of Process Chromatography, Academic Press, 1997). Examples include, but are not limited to, regular column chromatography, flash chromatography, high performance liquid chromatography (HPLC), medium pressure liquid chromatography (MPLC), supercritical fluid chromatography (SFC), countercurrent chromatography (CCC), moving bed chromatography, simulated moving bed chromatography, expanded bed chromatography, and planar chromatography. With each chromatographic method, examples of sorbents that may be used include, but are not limited to, silica gel, alumina, fluorisil, cellulose and modified cellulose, various modified silica gels, ion-exchange resins, size exclusion gels and other sorbents known in the art (see, for example, T. Hanai, HPLC: A Practical Guide, RSC Press, UK 1999). The present invention also includes the use of two or more solvent gradients to effect the fractionation, partial purification, and/or purification of the active ingredients by chromatographic methods. Examples of solvents that may be utilised include, but are not limited to, hexanes, heptane, pentane, petroleum ethers, cyclohexane, heptane, diethyl ether, methanol, ethanol, isopropanol, propanol, butanol, isobutanol, tert-butanol, water, dichloromethane, dichloroethane, ethyl acetate, tetrahydrofuran, dioxane, tert-butyl methyl ether, acetone, and 2-butanone. When water or an aqueous phase is used, it may contain varying amounts of inorganic or organic salts, and/or the pH may be adjusted to different values with an acid or a base such that fractionation and/or purification is enhanced.

In the case of planar chromatography, the present invention includes the use of various forms of this type of chromatography including, but not limited to, one- and two dimension thin-layer chromatography (1D- and 2D-TLC), high performance thin-layer chromatography (HPTLC), and centrifugal thin-layer chromatography (centrifugal TLC).

In the case of countercurrent chromatography (CCC), the present invention includes the use of manual, semi-automated, and automated systems, and the use of various solvents and solvent combinations necessary to effect fractionation and/or purification of active ingredients (see, for example, W. D. Conway, R. J. Petroski, Modern Countercurrent Chromatography, ACS Symp. Ser. Vol. 593, 1995). Solvent removal and/or concentration can be effected by various means known in the art including, but not limited to, reduced pressure evaporation, evaporation, reduced pressure distillation, distillation, and lyophilization.

The present invention includes the isolation of active ingredients by expanded bed chromatography, moving and simulated moving bed chromatography, and other related methods known in the art (see, for example, G. Sofer, L. Hagel, Handbook of Process Chromatography, Academic Press, 1997 and S. Ahuja, Handbook of Bioseparations, Academic Press, 2000).

Selective precipitation means includes the use of various solvents and solvent combinations, the use of temperature changes, the addition of precipitant and/or modifiers, and/or modification of the pH by addition of base or acid to effect a selective precipitation of active ingredients or other constituents.

The invention also includes the isolation of active ingredients by steam distillation, hydrodistillation, or other related methods of distillation known in the art (see, for example, L. M. Harwood, C. J. Moody, Experimental Organic Chemistry, Blackwell Scientific Publications, UK, 1989).

Dermatological Formulations

The present invention further provides for formulations suitable for dermatological applications comprising one or more extract of the invention, one or more active ingredient, or a combination thereof. The formulations can optionally comprise other therapeutic or cosmetic agents.

The formulations are prepared by standard techniques such that they have acceptable toxicity and stability. In addition, if the formulation is to be administered by a route other than topical (e.g. systemic routes, such as oral, or via intraperitoneal, intravenous, subcutaneous and intramuscular injection), then the extract and/or active ingredient must demonstrate acceptable hepatotoxicity and must be sufficiently resistant to degradation to allow the site of action to be reached.

Testing for the above parameters and preparation of appropriate formulations can be readily achieved by one skilled in the art. Criteria which must be considered in the preparation of a formulation include, but are not limited to, the physicochemical and biochemical characteristics (bioavailability, toxicity, stability, etc.) of the extracts and/or active ingredients which make up the formulation. In particular, the formulation is prepared so as to preserve, as much as possible, the maximum inhibitory activity of the active components upon administration, without being harmful to the animal.

The formulations are prepared by mixing the extract(s) and/or active ingredients together with a physiologically acceptable carrier. Excipients, binders, diluents, and the like can also be included in the formulation. The extract(s) and/or active ingredients can be formulated independently if desired and the respective formulations subsequently combined using a diluent or the like and administered, or can be administered independently of each other, either concurrently or at staggered times to the subject.

The formulations according to the invention may be in solid, semisolid or liquid form and may be adapted for oral (capsules, tablets, phials, troches, and the like), parenteral, rectal, inhalation, or topical administration, and may be in unit dosage form. The formulation may be adapted for slow release in vivo as known in the art. The formulations of the invention may be used in conventional form including, but not limited to, solutions, syrups, troches, lozenges, aqueous or oily suspensions, dispersible powders or granules, emulsions, hard or soft capsules, elixirs, injectables, tablets, capsules, suppositories, hydrophobic and hydrophilic creams and lotions. The term parenteral as used herein includes subcutaneous injections, intravenous, intrathecal, intramuscular, intrasternal injection or infusion techniques.

Various physiologically acceptable carriers known in the art can be used in the dermatological formulations of the invention. Examples of suitable carriers include, but are not limited to, hydroxypropyl cellulose, starch (corn, potato, rice, wheat), pregelatinized starch, gelatine, sucrose, acacia, alginic acid, sodium alginate, guar gum, ethyl cellulose, carboxymethylcellulose sodium, carboxymethylcellulose calcium, polyvinylpyrrolidone, methylcellulose, hydroxypropyl methylcellulose, microcrystalline cellulose, polyethylene glycol, powdered cellulose, glucose, croscarmellose sodium, crospovidone, polacrilin potassium, sodium starch glycolate, tragacanth, calcium carbonate, dibasic calcium phosphate, tribasic calcium phosphate, kaolin, mannitol, talc, cellulose acetate phthalate, polyethylene phthalate, shellac, titanium dioxide, carnauba wax, microcrystalline wax, calcium stearate, magnesium stearate, castor oil, mineral oil, light mineral oil, glycerine, sorbitol, mannitol, stearic acid, sodium lauryl sulfate, hydrogenated vegetable oil (for example. peanut, cottonseed, sunflower, sesame, olive, corn, soybean), zinc stearate, ethyl oleate, ethyl laurate, agar, calcium silicate, magnesium silicate, silicon dioxide, colloidal silicon dioxide, calcium chloride, calcium sulfate, silica gel, castor oil, diethyl phthalate, glyercin, mono- and di-acetylated monoglycerides, propylene glycol, triacetin, alamic acid, aluminum monostearate, bentonite, bentonite magma, carbomer 934, carboxymethylcellulose sodium 12, carrageenan, hydroxyethyl cellulose, magnesium aluminum silicate, pectin, polyvinyl alcohol, povidine, sodium alginate, tragacanth, xanthan gum, and silicones.

Formulations intended for oral use may be prepared according to methods known in the art and may contain one or more agents such as sweetening agents, flavouring agents, colouring agents and preserving agents in order to provide elegant and palatable preparations. Tablets contain the extract(s) and/or active ingredients in admixture with non-toxic physiologically acceptable excipients that are suitable for the manufacture of tablets. These excipients may be, for example, inert diluents, such as calcium carbonate, sodium carbonate, lactose, calcium phosphate or sodium phosphate: granulating and disintegrating agents for example, corn starch, or alginic acid: binding agents, for example starch, gelatinee or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

Formulations for oral use may also be presented as hard gelatinee capsules wherein the extract(s) and/or active ingredients are mixed with an inert solid diluent, for example, calcium carbonate, calcium phosphate or kaolin, or as soft gelatinee capsules wherein the extract(s) and/or active ingredients are mixed with water or an oil medium, for example peanut oil, liquid paraffin or olive oil.

Aqueous suspensions contain extract(s) and/or active ingredients in admixture with excipients suitable for the manufacture of aqueous suspensions. Such excipients are suspending agents, for example, sodium carboxymethylcellulose, methyl cellulose, hydropropylmethylcellulose, sodium alginate, polyvinylpyrrolidone, gum tragacanth and gum acacia: dispersing or wetting agents may be a naturally-occurring phosphatide, for example, lecithin, or condensation products of an alkylene oxide with fatty acids, for example polyoxyethyene stearate, or condensation products of ethylene oxide with long chain aliphatic alcohols, for example hepta-decaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxy-benzoate, one or more colouring agents, one or more flavouring agents or one or more sweetening agents, such as sucrose or saccharin.

Oily suspensions may be formulated by suspending the extract(s) and/or active ingredients in a vegetable oil, for example, arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavouring agents may be added to provide palatable oral preparations. These formulations may be preserved by the addition of an anti-oxidant such as ascorbic acid.

Dispersible powders and granules suitable for preparation of an aqueous suspension by the addition of water provide the extract(s) and/or active ingredients in admixture with a dispersing or wetting agent, suspending agent and one or more preservatives. Suitable dispersing or wetting agents and suspending agents are exemplified by those described above. Additional excipients, for example, sweetening, flavouring and colouring agents, may also be present.

Formulations of the invention may also be in the form of oil-in-water emulsions. The oil phase may be a vegetable oil, for example, olive oil or arachis oil, or a mineral oil, for example liquid paraffin or mixtures of these. Suitable emulsifying agents may be naturally-occurring gums, for example, gum acacia or gum tragacanth, naturally-occurring phosphatides, for example soy bean, lecithin, and esters or partial esters derived from fatty acids and hexitol, anhydrides, for example sorbitan monoleate, and condensation products of the said partial esters with ethylene oxide, for example polyoxyethylene sorbitan monoleate. The emulsions may also contain sweetening and flavouring agents.

Syrups and elixirs may be formulated with sweetening agents, for example, glycerol, propylene glycol, sorbitol or sucrose. Such formulations may also contain a demulcent, a preservative and flavouring and colouring agents. The formulations can be in the form of a sterile injectable aqueous or oleaginous suspension. This suspension may be formulated according to methods known in the art using suitable dispersing or wetting agents and suspending agents such as those mentioned above. The sterile injectable preparation may also be sterile injectable solution or suspension in a non-toxic parentally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose various bland fixed oils may be employed including synthetic mono- or diglycerides. In addition, fatty acids such as oleic acid find use in the preparation of injectables.

In one embodiment of the present invention, the dermatological formulations are for oral administration. Such formulations can be presented as, for example, capsules, cachets, tablets, aerosol sprays, powders, granules, creams, pastes, gels, ointments, or as a solution or a suspension in an aqueous liquid, a non-aqueous liquid, an oil-in-water emulsion, or a water-in-oil liquid emulsion.

The formulations contemplated by the present invention include so-called herbal and nutraceutical formulations. For nutraceutical formulations comprising solid parts of plant(s), the plant(s) must be an edible plant. The extract(s) and/or active ingredients or plant parts can be used in these herbal remedies and nutraceutical formulations as solutions, purified solutions, or dry powders.

In another embodiment of the present invention, the dermatological formulations are for topical administration. Such formulations may be presented as, for example, aerosol sprays, powders, sticks, granules, creams, liquid creams, pastes, gels, lotions, syrups, ointments, on sponges or cotton applicators, or as a solution or a suspension in an aqueous liquid, a non-aqueous liquid, an oil-in-water emulsion, or a water-in-oil liquid emulsion.

Topical formulations intended for application to the skin, hair and/or nails can include one or more moisturizing agents, i.e. an agent that facilitates hydration of the skin by inhibiting or preventing loss of water from the skin, that absorbs water from the atmosphere and hydrates the skin, and/or that enhances the skin's ability to absorb water directly from the atmosphere. Moisturizing agents generally minimise or prevent the skin from drying and cracking. Moisturizers, when used, are typically present in an amount from about 0.01 to 20 weight percent of the formulation.

Suitable moisturizing agents include acidic components, hydrophobic agents, and hydrophilic agents, or combinations thereof. Examples of moisturizing agents that are acidic components include, but are not limited to, 2-hydroxyacetic acid (glycolic acid); 2-hydroxypropanoic acid (lactic acid); 2-methyl 2-hydroxypropanoic acid; 2-hydroxybutanoic acid; phenyl 2-hydroxyacetic acid; phenyl 2-methyl 2-hydroxyacetic acid; 3-phenyl 2-hydroxyacetic acid; 2,3-dihydroxypropanoic acid; 2,3,4-trihydroxybutanoic acid; 2,3,4,5,6-pentahydroxyhexanoic acid; 2-hydroxydodecanoic acid; 2,3,4,5-tetrahydroxypentanoic acid; 2,3,4,5,6,7-hexahydroxyheptanoic acid; diphenyl 2-hydroxyacetic acid; 4-hydroxymandelic acid; 4-chloromandelic acid; 3-hydroxybutanoic acid; 4-hydroxybutanoic acid; 2-bydroxyhexanoic acid; 5-hydroxydodecanoic acid; 12-hydroxydodecanoic acid; 10-hydroxydecanoic acid; 16-hydroxyhexadecanoic acid; 2-hydroxy-3-methylbutanoic acid; 2-hydroxy-4-methylpentanoic acid; 3-hydroxy-4-methoxymandelic acid; 4-hydroxy-3-methoxymandelic acid; 2-hydroxy-2-methylbutanoic acid; 3-(2-hydroxyphenyl) lactic acid; 3-(4-hydroxyphenyl) lactic acid; hexahydromandelic acid; 3-hydroxy-3-methylpentanoic acid; 4-hydroxydecanoic acid-5-hydroxydecanoic acid; aleuritic acid; 2-hydroxypropanedioic acid; 2-hydroxybutanedioic acid; tannic acid; salicylic acid; erythraric acid; threaric acid; arabiraric acid; ribaric acid; xylaric acid; lyxaric acid; glucaric acid; galactaric acid; mannaric acid; gularic acid; allaric acid; altraric acid; idaric acid; talaric acid; 2-hydroxy-2-methylbutanedioic acid; citric acid, isocitric acid, agaricic acid, quinic acid, glucoronic acid, glucoronolactone, galactoronic acid, galactoronolactone, uronic acids, uronolactones, ascorbic acid, dihydroascorbic acid, dihydroxytartaric acid, tropic acid, ribonolactone, gluconolactone, galactonolactone, gulonolactone, mannonolactone, citramalic acid; pyruvic acid, hydroxypyruvic acid, hydroxypyruvic acid phosphate and esters thereof; methylpyruvate, ethyl pyruvate, propyl pyruvate, isopropyl pyruvate; phenyl pyruvic acid and esters thereof; methyl phenyl pyruvate, ethyl phenyl pyruvate, propyl phenyl pyruvate; formyl formic acid and esters thereof; methyl formyl formate, ethyl formyl formate, propyl formyl formate; benzoyl formic acid and esters thereof; methyl benzoyl formate, ethyl benzoyl formate and propyl benzoyl formate; 4-hydroxybenzoyl formic acid and esters thereof; 4-hydroxyphenyl pyruvic acid and esters thereof; and 2-hydroxyphenyl pyruvic acid and esters thereof. It should be understood that one or more derivatives of the above acidic component, such as esters or lactones or pharmaceutically acceptable salts thereof, may also be used.

Examples of moisturizing agents that are hydrophobic agents include, but are not limited to, ceramide, borage oil (linoleic acid), tocopherol linoleate, dimethicone, glycerinee, and mixtures thereof. Examples of moisturizing agents that are hydrophilic agents include, but are not limited to, hyaluronic acid, sodium peroxylinecarbolic acid (sodium PCA), wheat protein (such as laurdimonium hydroxypropyl hydrolyzed wheat protein), hair keratin amino acids, and mixtures thereof. Sodium chloride may also be present, for example, when hair keratin amino acids are included as a moisturizer. Other moisturizing agents that may be included in the formulations include primrose oil and flax seed oil.

The formulation may further optionally include one or more of a cysteine component, magnesium component, manganese component, selenium component, and copper component. These components are known in the art to impart beneficial effects to the skin, hair and/or nails.

For example a cysteine component may assist in thickening the dermis and supplementing collagen and elastic tissue, which can lead to a reduction of wrinkles and other skin conditions. An example of a suitable cysteine component is N-acetyl cysteine, or a pharmaceutically acceptable salt thereof, which can be included in the formulation in an amount from about 1 to 10 weight percent. The copper component may contribute to the inhibition elastase activity. Various copper compounds, or pharmaceutically acceptable salts thereof, are suitable for inclusion in the formulations. For example, copper sebacate can be included in the formulation in an amount from about 5 to 20 weight percent.

The optional manganese component can be one of a variety of manganese compounds, or pharmaceutically acceptable salts thereof, for example, manganese ascorbate or a manganese ascorbic acid complex, which can be included in the formulation in an amount from about 0.5 to 10 weight percent. Suitable magnesium compounds include magnesium ascorbate or magnesium ascorbic acid complex. The magnesium component can be included in the formulation in an amount from about 1 to 10 weight percent. Suitable selenium compounds include selenium complexed with an amino acid, for example, L-selenomethionine. The selenium component can be included in the formulation in an amount from about 0.01 to 3 weight percent.

The dermatological formulation can also include one or more anti-inflammatory components which facilitate inhibition or suppression of inflammation on or in the skin or in adjacent bodily tissues and thereby helps to reduce redness and swelling of the skin. Examples of suitable anti-inflammatory components include vitamin E and derivatives thereof, zinc, allantoin, glycyrrhetic acid, azulene, mefenamic acid, phenylbutazone, indometacin, ibuprofen, ketoprofen, ε-aminocaproic acid, hydrocortisone, panthenol and derivatives and salts thereof, zinc oxide and diclofenac sodium. The anti-inflammatory component, when used, can be incorporated into the formulations of the present invention in an amount between about 0.001 to about 5 weight percent.

The formulation may also optionally comprise one or more anti-oxidants to help neutralize free radicals and minimise their effect on the skin. Anti-oxidants can be enzymatic or non-enzymatic type. Examples include the enzymatic anti-oxidants: superoxide dismutase (SOD), catalase, and glutathione peroxidase, and the non-enzymatic anti-oxidants: Vitamin E (for example, tocopherol) and derivatives thereof, Vitamin A (retinol), Vitamin C (ascorbic acid), carotenoids and derivatives thereof, echinacoside, caffeoyl derivatives, oligomeric proanthocyanidins or proanthanols (such as those obtained from grape seed extract), green tea polyphenols, dibutyl hydroxytoluene, butyl hydroxyanisole, tannin and derivatives thereof such as gallic acid and ellagic acid, flavonoids such as flavone, catechin, quercetin and leucoanthocyanidin, quinones such as ubiquinone and vitamin K, thiamines and salts thereof, riboflavins such as riboflavin and riboflavin acetate, pyridoxines such as pyridoxine hydrochloride and pyridoxine dioctanoate, nicotinic acids such as nicotinic acid anmide and benzyl nicotinate, bihirubin, mannitol, tryptophane, histidine and nordihydroguaiaretic acid.

When vitamin C is included in the formulation, it can be in the form of ascorbyl palmitate, dipalmitate L-ascorbate, sodium L-ascorbate-2-sulphate, or an ascorbic salt, such as sodium, potassium, and calcium, or mixtures thereof. Vitamin C can be included in the formulations in an amount from about 0.1 to 50 weight percent. Vitamin A, when included, is usually in the form of vitamin A palmitate. Vitamin A can be included in topical formulations in an amount from about 0.5 to 15 weight percent. Suitable carotenoids include, for example, beta-carotene, canthaxanthin, zeaxanthin, lycopen, lutein, crocetin, capsanthin, and mixtures thereof. Carotenoids can be included in the formulation in an amount from about 0.1 to 5 weight percent.

Other skin benefit ingredients can also be optionally included in the dermatological formulations of the present invention. Examples of skin benefit ingredients include, but are not limited to, sunscreens and sunblocks, essential fatty acids, retinoids, cell activators, blood-circulation promoters, tanning agents, alpha or beta hydroxy-acids, proteins, peptides and polysaccharides.

Sunscreens and sunblocks include those materials commonly employed to block ultraviolet light. Examples of suitable sunscreens and sunblocks include, but are not limited to, titanium dioxide, zinc oxide, talc, red veterinary petrolatum, a cinnamate (such as octyl methoxycinnamate), a benzone (such as oxybenzone or 2-hydroxy-4-methoxy benzophenone), a salicylate (such as homosalicylate or octyl salicylate), a benzoic acid (such as para-aminobenzoic acid), and a benzophenone (such as oxybenzophenone). Octyl methoxycinnamate and 2-hydroxy-4-methoxy benzophenone (also known as oxybenzone) are commercially available under the trademarks, Parsol MCX™ and Benzophenone-3™, respectively. The exact amount of sunscreen employed in the formulations will vary depending upon the degree of protection desired from the sun's UV radiation and can be readily determined by one skilled in the art.

Essential fatty acids (EFAs) are those fatty acids which are essential for the plasma membrane formation of all cells. In keratinocytes, EFA deficiency makes cells hyperproliferative. EFAs also enhance lipid biosynthesis in the epidermis and provide lipids used in barrier formation by the epidermis. Examples of essential fatty acids that may be included in the formulations include linoleic acid, γ-olinolenic acid, homo-γ-linolenic acid, columbinic acid, eicosa-(n-6,9,13)-trienoic acid, arachidonic acid, γ-linolenic acid, timnodonic acid, hexaenoic acid and mixtures thereof.

Azoles, such as climbazole, bifonazole, clotrimnazole, ketoconazole, miconazole, econazole, itraconazole, fluconazole, terconazole, butoconazole, sulconazole, lionazole and mixtures thereof, may also optionally be included in the formulations.

Cell activators include, for example, royal jelly, photosensitizers, cholesterol and derivatives thereof, fetal calf blood extract, vitamin A, retinols and retinoids, citric acid, lactic acid, tartaric acid, malic acid, glycolic acid, succinic acid, serine, glutamic acid, hydroxyproline, theanine, pyrrolidone carboxylic acid, yeast extract, Lactobacillus extract and Bifidobacterium bifidum extract. The cell activator(s) can be incorporated into the formulations in an amount between about 0.001 and 5 weight percent.

Examples of blood circulation promoters are cepharanthine, tocopherol and derivatives thereof, nicotinic acid and derivatives thereof; nonanoic acid vanillylamide, capsaicine, zingerone, cantharides tincture, ichthammol, caffeine, tannic acid, α-borneol, cyclandelate, cinnarizine, tolazoline, acetylcholine, verapamil, γ-oryzanol, camphor, hinokitiol, and enzymes such as lipases and papain. The blood circulation promoter(s) can be incorporated into the formulations in an amount between about 0.01 to 20 weight percent.

The formulations of the present invention can further optionally comprise one or more thickener. A thickener will usually be present in amounts from 0.1 to 20% by weight of the formulation. Exemplary thickeners are cross-linked polyacrylate materials available under the trademark Carbopol™ (B.F. Goodrich Company), xanthan gum, carrageenan, gelatinee, karaya, pectin and locust bean gum. Under certain circumstances the thickening function may be accomplished by a moisturizer component of the formulation. For instance, silicone gums and esters such as glycerol stearate have dual functionality.

Other adjunct minor components can also optionally be incorporated into the dermatological formulations, for example, colouring agents, opacifiers, perfumes and preservatives (for example, imidazolidinyl urea, dimethyl imidazolidinone or diazolidinyl urea). Amounts of these materials can range from 0.001% up to 20% by weight of the formulation.

The dermatological formulations intended for topical application can be packaged in a suitable container to suit the viscosity and intended use. For example, a lotion or fluid cream can be packaged in a bottle or a roll-ball applicator, a capsule, a propellant-driven aerosol device or a container fitted with a pump suitable for finger operation. When the composition is a cream or paste, it can simply be stored in a non-deformable bottle or squeeze container, such as a tube or a lidded jar.

USE

The plant extracts of the invention and/or active ingredients derived from the extracts, and formulations comprising the extracts and/or active ingredients are suitable for use for the routine care of the skin, hair and/or nails, to improve the health and/or appearance of the skin, hair and/or nails and in the treatment or prevention of a variety of dermatological conditions.

In the context of the present invention, a dermatological condition is a condition present on one or more of the components of the integumentary system of a subject, such as the skin, hair or nails, that is caused by ageing or by intrinsic or extrinsic factors. Intrinsic factors include, for example, the genetic make up of an individual as well as pathological conditions that cause undesirable effects on the skin, hair or nails. Extrinsic factors include, but are not limited to, sunlight, radiation, air pollution, wind, cold, dampness, heat, chemicals, smoke, and smoking.

Thus, an effective amount of one or more plant extracts and/or active ingredients of the invention, or a dermatological formulation comprising an effective amount of one or more plant extracts and/or active ingredients can be administered to a mammal as part of routine skin/hair/nail maintenance, in order to improve the health and/or appearance of the skin, hair and/or nails or in order to treat or prevent a dermatological condition. In one embodiment of the present invention, the plant extracts, active ingredients or formulations are administered topically to a mammal.

Examples of dermatological conditions contemplated by the present invention include, but are not limited to, dry skin; dandruff; acne; keratosis; psoriasis; eczema; pruritus; age spots; reduced skin moisture; spider veins; senile purpura; lentigines; melasmas; deepening of skin lines; blotches; wrinkles; blemished skin; nodules; atrophy; rosacea; impetigo; elastotic changes characterized by leathery, course, rough, dry and yellowish skin; telangiecatic skin; hyperpigmented skin; hyperkeratotic skin; inflammatory dermatoses; “bullous” diseases, such as epidermolysis bullosa; hair breakage; hair loss; weathering damage; thinning of the hair; brittle nails; thinning nails; flaking nails and ridged nails.

Improving the health and/or appearance of the skin, hair and nails, includes, for example, eliminating or preventing the dark skin, melasma or ephelis generated or formed due to a variety of causes such as exposure to ultraviolet rays, changes in the hormone balance and genetic programs; lightening the dullness of the skin; improving the gloss and/or firmness of the skin; inhibiting or preventing the progress of the skin-ageing phenomenon; reducing minor blemishes; controlling dandruff; reducing redness or inflammation of the scalp, and the like. The dermatological formulations of the present invention can also be used to promote wound healing and/or decrease the risk of scarring.

In another embodiment, an effective amount one or more plant extracts and/or active ingredients of the invention, or a dermatological formulation comprising an effective amount of one or more plant extracts and/or active ingredients is administered to a mammal in order to attenuate one or more undesirable structural changes in the skin, such as wrinkling and/or sagging of the skin, loss of skin elasticity, redness, inflammation, formation of lesions, thinning of the epithelium, abnormal migration of cells within the skin (such as that which occurs during angiogenesis or inflammation), or various combinations thereof.

One embodiment of the present invention provides for the use of an effective amount of one or more plant extracts and/or active ingredients of the invention, or a dermatological formulation comprising an effective amount of one or more plant extracts and/or active ingredients as a skin care product. In the context of the present invention a “skin care product” refers to a product intended for use in the maintenance and optimization of skin health and preservation, from the standpoint of appearance and function. In another embodiment of the present invention, the skin care product is an anti-ageing product. An anti-ageing product is a product intended to use in attenuating or preventing skin ageing due to intrinsic or extrinsic factors. Skin ageing phenomena include, for example, skin thinning, fine and coarse skin wrinkling, sagging, loss of elasticity, and the like. Accordingly, the present invention provides for the administration of an effective amount one or more plant extracts and/or active ingredients of the invention, or a dermatological formulation comprising an effective amount of one or more plant extracts and/or active ingredients to a mammal in order to produce an anti-ageing effect.

By “effective amount” it is meant an amount of the plant extract or active ingredient that provides a beneficial effect in the treatment of a dermatological condition or a desired skin improvement effect. It should be understood by one of ordinary skill in the art that this amount will vary depending on the application and on the individual subject and will be readily determinable by one of skill in the art.

Appropriate doses of a formulation comprising the plant extract(s) and/or active ingredient will also vary according to the age, body weight, and response of the individual patient. In general, the total daily dose range, is from about 0.01 mg to about 2,000 mg of the plant extract(s) and/or active ingredient administered in about one to ten doses or applications.

Commercial Processes For Preparing Plant Extracts of the Invention

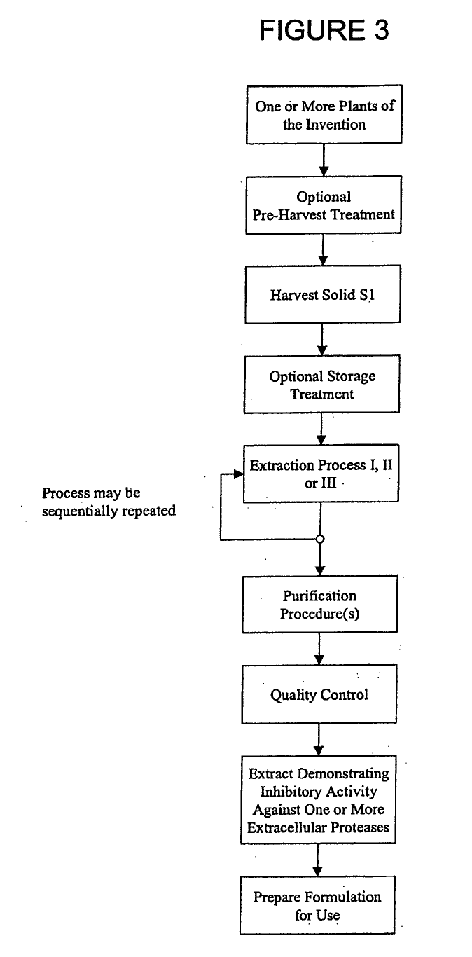

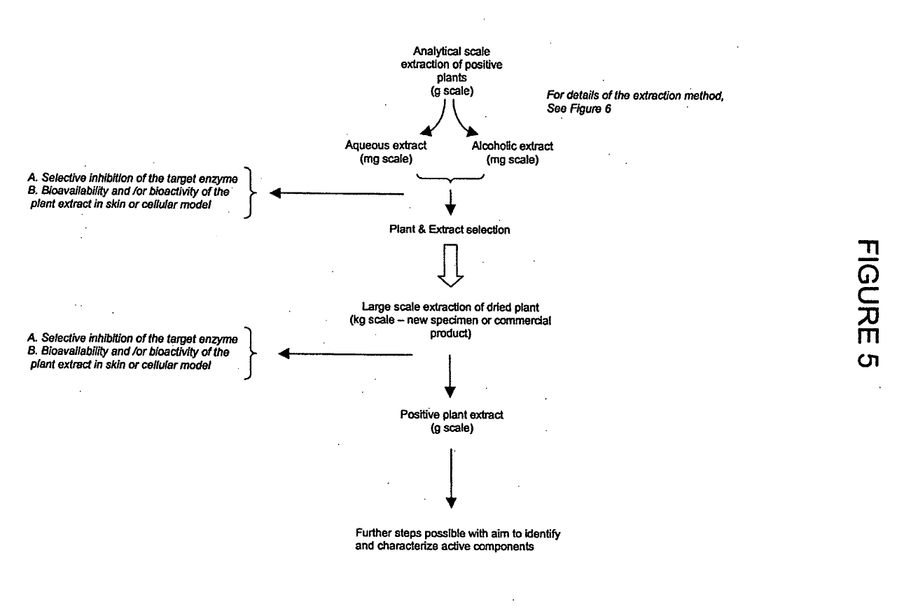

The present invention contemplates the large-scale preparation of the plant extracts of the invention. The extracts can be prepared on a commercial scale using the extraction process employed in the analytical scale preparation the extract of interest. One embodiment of this aspect of the invention is presented in FIG. 3. In this embodiment, the small-scale extraction procedure is simply scaled-up and additional steps of quality control are included to ensure reproducible results. Similarly the process outlined in FIG. 5 can be adapted for scale-up for commercial purposes.

Also contemplated by the present invention are modifications to the small-scale procedure that may be required during scale-up for industrial level production of the extract. Such modifications include, for example, alterations to the solvent being used or to the extraction procedure employed in order to compensate for variations that occur during scale-up and render the overall procedure more amenable to industrial scale production, or more cost effective. Modifications of this type are standard in the industry and would be readily apparent to those skilled in the art.

Process for Identifying Additional Plant Extracts

The present invention further provides for a rapid method for screening plant extracts to identify those capable of inhibiting one or more of MMP-1, MMP-2, MMP-3, MMP-9 and HLE, which are suitable for incorporation into the dermatological formulations of the invention.

The process comprises the following general steps: (a) generating a plurality of extracts from plant material by solvent extraction; (d) analysing the ability of each plant extract to inhibit one or more of MMP-1, MMP-2, MMP-3, MMP-9 and HLE; and selecting those extracts that are capable of inhibiting the activity of at least one of the listed EPs. The extracts exhibiting inhibitory activity can then be screened for their ability to affect one or more cellular activities in skin cells, such as attenuating the breakdown of a structural component of the ECM (i.e. collagen, fibronectin, fibrillin and/or elastin); attenuating endothelial cell migration; increasing collagen production; attenuating UV-induced extracellular protease activity and attenuating tractional forces generated by fibroblasts. Those extracts that are effective in the cellular screen are considered to be suitable candidates for inclusion in the dermatological formulations provided that they exhibit suitable stability and toxicity profiles.

The plurality of extracts in step-(a) above can be generated from plant-material from a single plant source using different solvents or the plurality of extracts can be generated by first selecting a group of plants of interest, harvesting plant material from each plant in the group, then extracting the plant material with a solvent or solvents to generate a plurality of extracts.

The extracts to be screened are prepared from plant material derived from a plant or plants of interest, i.e. “potential plants.” Potential plants include all species of the Kingdom Plantae, including terrestrial, aquatic or other plants that can be subjected to the methodology described herein in order to generate an extract that can be tested for its ability to inhibited at least one of the above-listed skin EPs.

Examples of potential plants include, but are not limited to, those belonging to the following classifications: Superdivision Spermatophyta—Seed plants; Division Coniferophyta—Conifers; Class Pinopsida, Order Pinales; Family Araucariaceae—Araucaria family; Family Cephalotaxaceae—Plum Yew family; Family Cupressaceae—Cypress family; Family Pinaceae—Pine family; Family Podocarpaceae—Podocarpus family; Family Taxodiaceae—Redwood family; Order Taxales, Family Taxaceae—Yew family; Division Cycadophyta—Cycads, Class Cycadopsida, Order Cycadales, Family Cycadaceae—Cycad family; Family Zamiaceae—Sago-palm family; Division Ginkgophyta—Ginkgo, Class Ginkgoopsida, Order Ginkgoales, Family Ginkgoaceae—Ginkgo family; Division Gnetophyta—Mormon tea and other gnetophytes, Class Gnetopsida, Order Ephedrales, Family Ephedraceae—Mormon-tea family; Order Gnetales, Family Gnetaceae—Gnetum family; Division Magnoliophyta—Flowering plants, Class Liliopsida—Monocotyledons, Subclass Alismatidae, Order Alismatales, Family Alismataceae—Water-plantain family, Family Butomaceae—Flowering Rush family, Family Limnocharitaceae—Water-poppy family; Order Hydrocharitales, Family Hydrocharitaceae—Tape-grass family; Order Najadales, Family Appnogetonaceae—Cape-pondweed family, Family Cymodoceaceae—Manatee-grass family, Family Juncaginaceae—Arrow-grass family, Family Najadaceae—Water-nymph family, Family Posidoniaceae—Posidonia family, Family Potamogetonaceae—Pondweed family, Family Ruppiaceae—Ditch-grass family, Family Scheuchzeriaceae—Scheuchzeria family, Family Zannichelliaceae—Homed pondweed family, Family Zosteraceae—Eel-grass family; Subclass Arecidae, Order Arales, Family Acoraceae—Calamus family, Family Araceae—Arum family, Family Lemnaceae—Duckweed family; Order Arecales, Family Arecaceae—Palm family; Order Cyclanthales, Family Cyclanthaceae—Panama Hat family; Order Pandanales, Family Pandanaceae—Screw-pine family; Subclass Commelinidae, Order Commelinales, Family Commelinaceae—Spiderwort family, Family Mayacaceae—Mayaca family, Family Xyridaceae—Yellow-eyed Grass family; Order Cyperales, Family Cyperaceae—Sedge family, Family Poaceae—Grass family; Order Eriocaulales, Family Eriocaulaceae—Pipewort family; Order Juncales, Family Juncaceae—Rush family; Order Restionales, Family Joinvilleaceae—Joinvillea family; Order Typhales, Family Sparganiaceae—Bur-reed family, Family Typhaceae—Cat-tail family; Subclass Liliidae, Order Liliales, Family Agavaceae—Century-plant family, Family Aloeaceae—Aloe family, Family Dioscoreaceae—Yam family, Family Haemodoraceae—Bloodwort family, Family Hanguanaceae—Hanguana family, Family Iridaceae—Iris family, Family Liliaceae—Lily family, Family Philydraceae—Philydraceae family, Family Pontederiaceae—Water-Hyacinth family, Family Smilacaceae—Catbrier family, Family Stemonaceae—Stemona family, Family Taccaceae—Tacca family; Order Orchidales, Family Burmanniaceae—Burmannia family, Family Orchidaceae—Orchid family; Subclass Zingiberidae, Order Bromeliales, Family Bromeliaceae—Bromeliad family; Order Zingiberales, Family Cannaceae—Canna family, Family Costaceae—Costus family, Family Heliconiaceae—Heliconia family, Family Marantaceae—Prayer-Plant family, Family Musaceae—Banana family, Family Zingiberaceae—Ginger family; Class Magnoliopsida—Dicotyledons, Subclass Asteridae, Order Asterales, Family Asteraceae—Aster family; Order Callitrichales, Family Callitrichaceae—Water-starwort family, Family Hippuridaceae—Mare's-tail family; Order Calycerales, Family Calyceraceae—Calycera family; Order Campanulales, Family Camnpanulaceae F—Bellflower family, Family Goodeniaceae—Goodenia family, Family Sphenocleaceae—Spenoclea family; Order Dipsacales, Family Adoxaceae—Moschatel family, Family Caprifoliaceae—Honeysuckle family, Family Dipsacaceae—Teasel family, Family Valerianaceae—Valerian family; Order Gentianales, Family Apocynaceae—Dogbane family, Family Asclepiadaceae—Milkweed family, Family Gentianaceae—Gentian family, Family Loganiaceae—Logania family; Order Lamiales, Family Boraginaceae—Borage family, Family Lamiaceae—Mint family, Family Lennoaceae—Lennoa family, Family Verbenaceae—Verbena family, Order Plantaginales, Famnily Plantaginaceae—Plantain family; Order Rubiales, Family Rubiaceae—Madder family; Order Scrophulariales, Family Acanthaceae—Acanthus family, Family Bignoniaceae—Trumpet-creeper family, Family Buddlejaceae—Butterfly-bush family, Family Gesneriaceae—Gesneriad family, Family Lentibulariaceae—Bladderwort family, Family Myoporaceae—Myoporum family, Family Oleaceae—Olive family, Family Orobanchaceae—Broom-rape family, Family Pedaliaceae—Sesame family, Family Scrophulariaceae—Figwort family; Order Solanales, Family Convolvulaceae—Morning-glory family, Family Cuscutaceae—Dodder family, Family Fouquieriaceae—Ocohillo family, Family Hydrophyllaceae—Waterleaf family, Family Menyanthaceae—Buckbean family, Family Polemoniaceae—Phlox family, Family Solanaceae—Potato family; Subclass Caryophyllidae, Order Caryophyllales, Family Achatocarpaceae—Achatocarpus family, Family Aizoaceae—Fig-marigold family, Family Amaranthaceae—Amaranth family, Family Basellaceae—Basella family, Family Cactaceae—Cactus family, Family Caryophyllaceae—Pink family, Family Chenopodiaceae—Goosefoot family, Family Molluginaceae—Carpet-weed family, Family Nyctaginaceae—Four o'clock family, Family Phytolaccaceac—Pokeweed family, Family Portulacaceae—Purslane family; Order Plumbaginales, Family Plumbaginaceae—Leadwort family; Order Polygonales, Family Polygonaceae—Buckwheat family; Subclass Dilleniidae, Order Batales, Family Bataceae—Saltwort family; Order Capparales, Family Brassicaceae—Mustard family, Family Capparaceae—Caper family, Family Moringaceae—Horse-radish tree family, Family Resedaceae—Mignonette family; Order Diapensiales, Family Diapensiaceae—Diapensia family; Order Dilleniales, Family Dilleniaceae—Dillenia family, Family Paeoniaceae—Peony family; Order Ebenales, Family Ebenaceae—Ebony family, F Family Sapotaceae—Sapodilla family, Family Styracaceae—Storax family, Family Symplocaceae—Sweetleaf family; Order Ericales, Family Clethraceae—Clethra family, Family Cyrillaceae—Cyrilla family, Family Empetraceae—Crowberry family, Family Epacridaceae—Epacris family, Family Ericaceae—Heath family, Family Monotropaceae—Indian Pipe family, Family Pyrolaceae—Shinleaf family; Order Lecythidales, Family Lecythidaceae—Brazil-nut family; Order Malvales, Family Bombacaceae—Kapok-tree family, Family Elaeocarpaceae—Elaeocarpus family, Family Malvaceae—Mallow family, Family Sterculiaceae—Cacao family, Family Tiliaceae—Linden family; Order Nepenthales, Family Droseraceae—Sundew family, Family Nepenthaceae—East Indian Pitcher-plant family, Family Sarraceniaceae—Pitcher-plant family; Order Primulales, Family Myrsinaceae—Myrsine family, Family Primnulaceae—Primrose family, Family Theophrastaceae—Theophrasta family; Order Salicales, Family Salicaceae—Willow family; Order Theales, Family Actinidiaceae—Chinese Gooseberry family, Family Caryocaraceae—Souari family, Family Clusiaceae—Mangosteen family, Family Dipterocarpaceae—Meranti family, Family Elatinaceae—Waterwort family, Family Marcgraviaceae—Shingle Plant family, Family Ochnaceae—Ochna family, Family Theaceae—Tea family; Order Violales, Family Begoniaceae—Begonia family, Family Bixaceae—Lipstick-tree family, Family Caricaceae—Papaya family, Famnily Cistaceae—Rock-rose family, Family Cucurbitaceae—Cucumber family, Family Datiscaceae—Datisca family, Family Flacourtiaceae—Flacourtia family, Family Frankeniaceae—Frankenia family, Family Loasaceae—Loasa family, Family Passifloraceae—Passion-flower family, Family Tamaricaceae—Tamarix family, Family Turneraceae—Turnera family, Family Violaceae—Violet family; Subclass Hamamelidae, Order Casuarinales, Family Casuarinaceae—She-oak family; Order Fagales, Family Betulaceae—Birch family, Family Fagaceae—Beech family; Order Hamamelidales, Family Cercidiphyllaceae—Katsura-tree family, Family Hamamelidaceae—Witch-hazel family, Family Platanaceae—Plane-tree family; Order Juglandales, Family. Juglandaceae—Walnut family; Order Leitneriales, Family Leitneriaceae—Corkwood family; Order Myricales, Family Myricaceae—Bayberry family; Order Urticales, Family Cannabaceae—Hemp family, Family Cecropiaceae—Cecropia family, Family Moraceae—Mulberry family, Family Uhlaceae—Elm family, Family Urticaceae—Nettle family; Subclass Magnoliidae, Order Aristolochiales, Family Aristolochiaceae—Birthwort family; Order Illiciales, Family Illiciaceae—Star-anise family, Family Schisandraceae—Schisandra family; Order Laurales, Family Calycanthaceae—Strawberry-shrub family, Family Hemandiaceae—Hemandia family, Family Lauraceae—Laurel famnily, Family Monimiaceae—Monimia family; Order Magnoliales, Family Annonaceae—Custard-apple family, Family Canellaceae—Canella family, Family Magnoliaceae—Magnolia family, Family Myristicaceae—Nutmeg family, Family Sonneratiaceae—Sonneratia family, Family Winteraceae—Wintera family; Order Nymphaeales, Family Cabombaceae—Water-shield family, Family Ceratophyllaceae—Hornwort family, Family Nelumbonaceae—Lotus-lily family, Family Nymphaeaceae—Water-lily family; Order Papaverales, Family Fumariaceae—Fumitory family, Family Papaveraceae—Poppy family; Order Piperales, Family Chloranthaceae—Chloranthus family, Family Piperaceae—Pepper family, Family Saururaceae—Lizard's-tail family; Order Ranunculales, Family Berberidaceae—Barberry family, Family Lardizabalaceae—Lardizabala family, Family Menispermaceae—Moonseed family, Family Ranunculaceae—Buttercup family, Family Sabiaceae—Sabia family; Subclass Rosidae, Order Apiales, Family Apiaceae—Carrot family, Family Araliaceae—Ginseng family; Order Celastrales, Family Aquifoliaceae—Holly family, Family Celastraceae—Bittersweet family, Family Corynocarpaceae—Karaka family, Family Hippocrateaceae—Hippocratea family, Family Icacinaceae—Icacina family, Family Stackhousiaceae—Stackhousia family; Order Comales, Family Comaceae—Dogwood family, Family Garryaceae—Silk Tassel family, Family Nyssaceae—Sour Gum family; Order Euphorbiales, Family Buxaceae—Boxwood family, Family Euphorbiaceae—Spurge family, Family Simnmondsiaceae—Jojoba family; Order Fabales, Family Fabaceae—Pea family; Order Geraniales, Family Balsaminaceae—Touch-me-not family, Family Geraniaceae—Geranium family, Family Limnanthaceae—Meadow-Foam family, Family Oxalidaceae—Wood-Sorrel family, Family Tropaeolaceae—Nasturtium family; Order Haloragales, Family Gunneraceae—Gunnera family, Family Haloragaceae—Water Milfoil family; Order Linales Family Erythroxylaceae—Coca family, Family Linaceae—Flax family; Order Myrtales, Family Combretaceae—Indian Almond family, Family Lythraceae—Loosestrife family, Family Melastomataceae—Melastome family, Family Myrtaceae—Myrtle family, Family Onagraceae—Evening Primrose family, Family Punicaceae—Pomegranate family, Family Thymelaeaceae—Mezereum family, Family Trapaceae—Water Chestnut family; Order Podostemales, Family Podosteimaceae—River-weed family; Order Polygalales, Family Krameriaceae—Krameria family, Family Malpighiaceae—Barbados Chemy family, Family Polygalaceae—Milkwort family; Order Proteales, Family Proteaceae—Protea family; Order Rafflesiales, Family Rafflesiaceae—Rafflesia family; Order Rhamnales, Family Elaeagnaceae—Oleaster family, Family Rhamnaceae—Buckthorn family, Family Vitaceae—Grape family; Order Rhizophorales, Family Rhizophoraceae—Red Mangrove family; Order Rosales, Family Brunelliaceae—Brunellia family, Family Chrysobalanaceae—Cocoa-plum family, Family Connaraceae—Cannarus family, Family Crassulaceae—Stonecrop family, Family Crossosomataceae—Crossosoma family, Family Cunoniaceae—Cunonia family, Family Grossulariaceae—Currant family, Family Hydrangeaceae—Hydrangea family, Family Pittosporaceae—Pittosporum family Family Rosaceae—Rose family, Family Saxifragaceae—Saxifrage family, Family Surianaceae—Suriana family; Order Santalales, Family Balanophoraceae—Balanophora family, Family Eremolepidaceae—Catkin-mistletoe family, Family Loranthaceae—Showy Mistletoe family, Family Olacaceae—Olax family, Family Santalaceae—Sandalwood family, Family Viscaceae—Christmas Mistletoe family; Order Sapindales, Family Aceraceae—Maple family, Family Anacardiaceae—Sumac family, Family Burseraceae—Frankincense family, Family Hippocastanaceae—Horse-chestnut family, Family Meliaceae—Mahogany family, Family Rutaceae—Rue family, Family Sapirdaceae—Soapberry family, Family Simaroubaceae—Quassia family, Family Staphyleaceae—Bladderniut family, Family Zygophyllaceae—Creosote-bush family.

In one embodiment, potential plants comprise: Abelmoschus esculentus, Abies balsamea, Abies cephalonica, Abies firma, Abies lasiocarpa, Acer campestre, Acer mandshurica, Acer palmaturn “burgundy,” Acer tataricum, Acer truncatum, Achillea millefolium, Achillea ptarmica, Achillea tomentosa, Acolypha hispida, Aconitum napellus, Aconitum spp., Acorus calamus, Actaea racemosa, Actinidi colonicta, Actinidia arguta, Actinidia chinensis, Actinidia colomicta, Adansonia digitata, Adianthum radiatum, Adianthum trapezieformis, Adiantum pedatum, Adiantum tenerum, Aechmea luddemoniana, Aesculus hypocastanum, Aesculus waertilensis, Aesculus woerlitzenis, Aessopteria crasifolia, Aframomum melegueta, Agaricus bisporus, Agastache foeniculum, Agastache mexuicana, Agatis robusta, Ageratum conizoides, Aglaonema commutatus, Agrimonia eupatora, Agropyron cristatum, Agropyron repens, Agrostis alba, Agrostis stolonifera, Ailantus altissima, Ajuga reptans, Alcea rosea, Alchemilla mollis, Alchemilla sp., Alium cermum, Alkanna tinctoria, Allium ampeloprasum, Allium cepa, Allium fistulosum, Allium grande, Allium nutans, Allium porrum, Alium sativum, Allum schoenoprasum, Albium sp., Allum tuberosum, Allium victorialis, Aloe vera, Alpinia officinarum, Althaea officinalis, Alum japonica, Amaranthus caudatus, Amaranthus retroflexus, Amaranthus tricolor, Ambrosia artemisiifolia, Amelanchier alnifolia, Amelanchier canadensis, Amelanchier sanguinea, Amelanchier sanguinea×A. laevis, Amelanchier spicata, Amigdalus nana, Amsonia tabemaemontana, Ananas comosus, Anaphalis margaritacea, Anemona japonica, Anethum graveolens, Angelica archangelica, Angelica dahurica, Angelica sinensis, Antericum ramosum, Anthemis tinctoria, Anthoxanthum odoratum, Anthriscus cerefolium, Anthurium altersianum, Anthurium andreanum, Anthurium elegans, Anthurium guildingii, Anthurium hookeri, Anthurium magnificum, Anthyrium filis-femina, Anthyrium nopponicum, Apium graveolens, Apocynum cannabinum, Arachis hypogaea, Aralia cordata, Aralia nudicaulis, Aralis mandshurica, Archirantus bidentata, Arctium lappa, Arctium minus, Arctostaphylos uva-ursi, Armoracea rusticana, Armoraica ristica, Aronia melanocarpa, Aronia×prunifolia, Arrhenatherum elatius, Artemisia abrotanum, Artemisia absinthium, Artemisia dracunculus, Artemisia ludoviciana, Artemisia vulgaris, Asarum europaeum, Asclepias incamata, Asclepias tuberosa, Asimina triloba, Asorum canadensis, Asparagus officinalis, Asplenium australasicum, Aster spp, Aster-Nova anglicae, Astilbe chinensis, Astilbe×arendsii, Astilboides tabularis, Astragulus sinicus, Athyrium asperum, Atriplex hortensis, Atropa belladonna, Austolachia australis, Avena sativa, Averrhoa carambola, Bactisia australis, Baptisia tinctoria, Barbaric sp., Beckmannia eruciformis, Begonia convolvulacea, Begonia eminii, Begonia glabra, Begonia mannii, Begonia polygonoides, Bellis perennis, Berberis thungergi, Berberis vulgaris, Bergenia crassifolia, Bergenia×schmidtii, Beta vulgaris, Betula alba, Betula alleghaniensis, Betula daurica, Betula glandulosa, Betula nigra, Betula pendula, Bocconia cordata, Boechimeria boloba, Boesenbergia rotunda, Boletus edulis, Borago officinalis, Boxus sempervirens, Brassica cepticepa, Brassica chinensis, Brassica juncea, Brassica napa, Brassica napus, Brassica nigra, Brassica oleracea, Brassica rapa, Bromelia balansae, Bromus inermis, Brugmansi graveolens (ralf), Brugmansia suaveolens, Bruginansia suaveolens, Buddleja davidii, Bupleurum falcatum, Butomus umbellatus, Buxus microphilla “japonica”, Buxus microphylla, Cachris alpina, Cactus officinalis, Caladium spp., Calamagrostis arundiflora, Calamintha nepeta, Calathea zebrina, Calendula officinalis, Calicatus floridus, Camellia sinensis, Campanula carpatica, Campanula rapunculus, Canna indica, Cantharellus cibarius, Capparis spinosa inemis, Capsella bursa-pastoris, Capsicum annuum, Capsicum frutescens, Carex morrowii, Carica papaya, Carlina acaulis, Carpinus caroliniana, Carthamus tinctorius, Carum capsicum, Carum carvi, Carya cordiformis, Caryota ureus, Casia hebecarpa, Castanea sativa, Castanea spp., Celosia cristata, Celtis occidentalis, Centaurea dealbata, Centaurea soistitialis, Centauria maculata, Cerastium tomentosum, Cerasus japonica, Cerasus maghabab, Ceratoramia mexicana, Chaenomeles×superba, Chaernomelis superba, Chaerophyllum bulbosum, Chamaemelum nobile, Charnaechrista fasciculata, Charnaeciparis pisifera, Chelidonium majus, Chenopodium album, Chenopodium bonus-henricus, Chenopodium quinoa, Chrysanthemum coronarium, Cicer arietinum, Cichorium endivia subsp. endivia, Cichorium intybus, Cinnamomum verum, Cirsium arvense, Cissus discolor, Cistus incanus, Citinis coggriaria, Citrullus colocynthis, Citrullus lanatus, Citrus limettoides, Citrus limon, Citrus reticulata, Citrus sinensis, Citrus×paradisi, Clematis alpina, Clematis armandii, Clematis chiisanensis, Clematis rectae, Clerodendrurn speciossicum, Cobiaeum varilartum, Coccoloba caracasana, Cocculus laurifolius, Cocos nucifera, Coix lacryma-jobi, Colocasia spp., Comus mass, Convalaria majalis, Conyza-canadensis, Corchorus olitorius, Coreopsis verticillata, Coriandruim sativum, Cornus alba, Cornus canadensis, Cornus mas, Cornus sericea, Coronolla varia, Coryllus avelana, Corylus maxima, Cosmos sulphureus, Cotinus coggygria, Cotoneaster fangianus, Cotoneaster horisontalis, Cotynus cogygria, Crambe cordifolia, Cramble cardifolia, Crataegus praegophyrum, Crataegus sanguinea, Crataegus spp., Crataegus submollis, Crategus macrophyllum, Crithmum maritimum, Cryptotaenia canadensis, Crytomium fortunei, Cucumis anguria, Cucumis melo, Cucumis metuliferus, Cucumis sativus, Cucurbita maxima, Cucurbita moschata, Cucurbita pepo, Cullen corylifolium, Cuminum cyminum, Cupress lusitanica, Cupressus sempervirens, Curcuma longa, Curcuma zedoaria, Cycas cirinalis, Cyclonia oblonga, Cymbopogon citratus, Cymbopogon martinii, Cynara cardunculus subsp. cardunculus, Cynnamonum zeylonicum, Cyperus alternifolius, Cyperus esculentus, Dactylis glomerata, Dahlia spp., Darura stramonium, Datisca cannabina, Datura metel, Datura stramonium, Daucus carota, Deutria scabra, Dieffenbachia leopoldii, Dieffenbachia segiunae, Digitalis lutea, Digitalis purpurea, Dimocarpus longan, Diopiros kaka, Dioscorea batatas, Diospyros kaki, Dipsacus sativus, Dirca palustris, Dolichos lablab, Dracaena fragrans, Dracaena sp., Dryopteris filis-max, Dryopteris filix-mas, Echinacea purpurea, Echinochloa frumentacea, Echinops sphae, Eleagnus angustifolia, Eleagnus cemutata, Eleusine coracana, Encephalaris horridum, Epilobium augustifolium, Equisetum hyemale, Equisetum variegatum, Erigeron speciosus, Eriobotria japonica, Eriobotrya japonica, Eruca vesicaria, Erungiurm campestre, Erysimumw perofskianum, Erythrinia caffra, Erythrinia crista, Erythrinia glabeliferus, Eschscholzia californica, Eucaliptus rudis, Eucomia ulurifolia, Euonimus elata, Euonomus europea, Euonomus verrucosa, Euphorbia amygdaloides, Fagopyrum esculentum, Fagopyrum suffruticosum, Fagopyrum tataricum, Fagus silvatica, Fautenousus qualiqualia, Festuca rubra, Feucrium hamedris, Ficus benjaminii, Ficus elastica, Ficus purnila, Ficus religiosa, Ficus sp., Ficus triangularis, Filipendula rubra, Filipendula ulmaria, Filipendula vulgaris, Foeniculum vulgare, Foenix zeulonica, Forsithsia suspensa, Forsitsia europea, Forsythia×intermedia, Fortunella spp., Fragaria×ananassa, Frangula alnus, Fraxinus exelsior, Fuchsia magellanica, Fuchsia spp., Fucus vesiculosus, Fumaria officinalis, Galinsoga quadriradiata, Galium aparine, Galium odoratum, Gallium sporium, Gardenia jasminoides, Gaultheria hispidula, Gaultheria procumbens, Genista multibracteata, Gentiana cruciata, Gentiana littorala, Gentiana lutea, Gentiana macrophylla, Gentiana tibetica, Geranium maculata, Geranium phaeum, Geranium pratense, Geranium sanguineum, Geranium×cantabrigiense, Geum fanieri, Geum macrophyllum, Geum rivale, Gingko biloba, Glaux maritima, Glechoma hederacea, Glyceria maxima, Glycine max, Glycyrrhiza glabra, Gnetum guemon, Gossypium herbaceum, Gratiola officinalis, Gravilea robusta, Guizotia abyssinica, Haemanthus katharina, Hamamelis mollis, Hamamelis virginiana, Haser trilobum, Hedeoma pulegioides, Hedychium coronarium, Hedychium spp., Helenium spp., Helianthus annus, Helianthus stumosus, Helianthus tuberosus, Helichrysum angustifolium, Helichrysum thianschanicum, Heliotropium arborescens, Helleborus niger, Heraclelum pubescens, Herba schizonepetae, Hemerocalis spp., Hibiscus cannabinus, Hissopus zeraucharicus, Hiuga reptans, Hordeum hexastichon, Hordeum vulgare, Hordeum vulgare subsp. vulgare, Hosta fortuna, Hosta fortunaea, Hosta lancefolia, Hosta sieboldiana, Hosta zibalda, Houttuynia cordata, Humulus lupulus, Hydrangea quercifolia, Hydrastis canadensis, Hydrocotile asiatica, Hylotelephium spp., Hymenoxys hoopesii, Hyoscyamus niger, Hypericum henryi, Hypericum perforatum, Hypericum spp., Hypomyces lactifluorum, Hyppoach rhamnoides, Hyssopus officinalis, Iberis amara, Iberis sempervirens, Ilex agnifolium, Ilex comuta, Inula helenium, Ipomea tricolor, Ipomoea aquatica, Ipomoea batatas, Iris alida, Iris pseudocarpus, Iris versicolor, Isatis tinctoria, Jacobinia sp., Jasminum frutocarus, Jeffersonia diphylla, Juca sp., Juglands regia, Juglans nigra, Juniperus “blue pacific”, Juniperus communis, Keyleiteria paniculata, Kochia scoparia, Koeleria glauca, Kolkwitzia amabilis, Korria japonica, Krameria lappacea, Lactuca sativa, Lactuca serriola, Lal lab purpurea, Lamiastrum galeobdolon, Lapia dulcis, Laportea canadensis, Larix dedidua, Laserpitium latifolium, Lathyrus sativus, Lathyrus sylvestris, Laurus nobilis, Lavandula angustifolia, Lavandula latifolia, Lavandula officinalis, Ledum groenlandicum, Lens culinaris subsp. culinaris, Lentinus edodes, Leontopodium alpinum, Leonurus cardiaca, Lepidium sativum, Leucanthemum vulgare, Levisticurn officinale, Liatris spinata, Liclum barbatum, Ligularia dentata, Ligustrum vulgare, Linaria vulgaris, Lindera benzoin, Linium hirsutum, Linum usitatissimum, Lippa dulcis, Litchi chinensis, Livistona fragrans, Lobelia siphitica, Lolium multiflorum, Lolium perenne, Lonicera ramosissima, Lonicera syringantha, Lotus cornicuiatus, Lotus tetragonolobus, Luglands nigra, Lunaria annua, Lupinus luteaus, Lupinus polyphyllus, Luzula sylvatica, Lychnis chalcedonica, Lycodium japonicum, Lycopersicon esculentum, Lycopersicon pimpinellifolium, Lysimachia clethroides, Lythrum salicaria, Madia sativa, Magnolia agrifolia, Magnolia cobus, Magnolia loebheril, Magnolia stellata, Magnolia×loebneri, Malus hupehensis, Malus prunifolia, Malus spp., Malva moschata, Malva sylvestris, Malva verticillata, Mangifera indica, Manihot esculenta, Marrubium vulgare, Matricaria recutita, Matricaria spp., Matteuccia pensylvanica, Matteucia strutioptoris, Medicago sativa, Melaleuca altemifolia, Melilotus albus, Melilotus officinalis, Melissa officinalis, Mentha arvensis, Mentha pulegium, Mentha spicata, Mentha suaveolens, Mentha×piperita, Menyanthes trifoliata, Mespilus germanica, Metasequoia glyptotrobioldes, Metrosideros excelsa, Microbiata decussata, Microlepia platphylla, Microlepia platyphylla, Microsorium punctatum, Minispermum dauricum, Mirica certifera, Miscanthus sacchariflorus, Miscanthus sinensis, Momordica charantia, Monarda didyma, Monarda fistulosa, Monarda spp., Monstera deliciosa, Monstera pertusa, Montia perfoliata, Morms alba, Murraya exotica, Musa textilis, Musa×paradisiaca, Myrica pensylvanica, Myrthus communis, Nasturtium officinale, Nepeta cataria, Nicodernia diversifolia, Nicotiana rustica, Nicbtiana tabacum, Nigella sativa, Ocimrnum Basilicum, Ocirnum tenuiflorum, Oenothera biennis, Oenothera fruticosa subsp fruticosa, Olea europaea, Olea olcaster, Onobrychis viciifolia, Onoclea sensibilis, Ophiopogon japonicus, Opuntia spp., Oreopanax capitata, Origanum majorana, Origanum vulgare, Oryza sativa, Osmanthus spp., Osmunda regalis, Osmundastrum claytonionum, Ostrea carpinifolia, Ostrea connote, Oxalis deppei, Oxobachus nictogenea, Oxyria digyna, Pachyra affinis, Paeonia daurica, Paeonia lactiflora, Paeonia rubra, Paeonia spp., Paeonua suffructicisa, Panax quinquefolius, Panicum miliaceum, Parrotia persica, Parthenosicus tricuspidata, Passiflora caerulea, Passiflora spp., Pastinaca sativa, Pegamun hamalis, Pelargonium zonale, Pennisetum alopecuroides, Penstemon digitalis, Pentaphylloides fruticosa, Perilla frutescens, Persea americana, Petasites japonicus, Petroselinum crispum, Peucedanum cervaria, Peucedanum oreaselinum, Pfaffia paniculata, Phacelia tanacetifolia, Phalaris arundinacea, Phalaris canariensis, Phaseolus acutifolius, Phaseolus coccineus, Phaseolus vulgaris, Phebodium aureum, Philadelphus coronarius, Philodendron amurense, Phleum pratense, Phlox paniculata, Phoenix dactylifera, Phylidendron speciosus, Phyllanthus grandifolium, Phyllitis scolopendrium, Phyrnatosorus scolopendria, Physalis alkekengi, Physalis creticola, Physalis grisea, Physalis philadelphica, Physalis spp., Physostegia virginiana, Phytolacca americana, Picea schrenkiana, Pieras japonica, Pigelia pennata, Pimpinella anisum, Pinus bungiana, Pinus cembra, Pinus mugo, Pinus pinea, Pinus pumila, Pinus salinifolia, Pinus silvestris, Pinus sirtrobus, Pinus strobus, Piper chaba, Piper nigrum, Pisum sativum, Pithecelobium unguis, Pittisporum tibica, Plantago coronopus, Plantago major, Plantago minor, Platanus acidentalis, Platicada grandiflora, Plectranthus fruticosus, Plectranthus spp., Pleurotus spp., Plumbago zeylanica, Poa compressa, Poa pratensis, Podocarpus spinulosus, Podophyllum amodii, Podophyllum peltatum, Poligonum aviculare, Poligomun latifolia, Polygonium odoratum, Polygonum aviculare, Polygonum chinense, Polygonum cuspidatum, Polygonum pensylvanicum, Polygonum persicaria, Polymonium ceruleum, Polyschium braunii, Pongamia pinnata, Pontederia cordata, Populus incrassata, Populus tremula, Populus×petrowskyana, Portulaca oleacea, Potentilla alba, Potentilla anserina, Potentilla fruticosa, Poterium sangiusorba, Primula veris, Princepia sp., Prunella vulgaris, Prunus armeniaca, Prunus cerasifera, Prunus cerasus, Prunus persica, Prunus serotica, Prunus spp., Prunus tomentosa, Prunus xocane, Psathyrostachys juncea, Pseudotsuga menzisia, Psidium guajava, Psidium spp., Psychotria metbacteriodomasica, Psychotria nigropunctata, Pteridium aquilinum, Pterigota alata, Puansetia sp., Pulmonaria molissima, Pulmonaria officinalis, Pulmonaria saccharata, Punica granatum, Pyrus communis, Pyrus pyrifolia, Quercus castanufolia, Quercus imbricaria, Quercus nigra, Quercus robur “fastigiata,” Quercus rubra, Quercus trojana, Raphanus raphanistrum, Raphanus sativus, Ratibiunda columnus-Fera, Rauwolfia tetraphylla, Rehmannia glutinosa, Reseda luteola, Reseda odorata, Rheum officinale, Rheum palmatum, Rheum×hybridum, Rhododendron spp., Rhus aromatica, Rhus toxicodenta, Rhus trilobata, Ribes americanum, Ribes grossularia, Ribes nigrum, Ribes sylvestre, Ribes uva-crispa, Ribes×nidigrolaria, Ricinus communis, Rimula japonica, Rodgersia podophylla, Rodgersia spp., Rosa cocanica, Rosa multiflora, Rosa rugosa, Rosmriarinus officinalis, Rubus allegheniensis, Rubus arcticus, Rubus canadensis, Rubus idaeus, Rubus occidentalis, Rubus phoenicolasius, Rubus pubescens, Rubus thibetanus, Rudbeckia maxima, Rumex acetosa, Rumex acetosella, Rumex crispus, Rumex patientia, Rumex scutatus, Ruschia indurata, Ruta graveolens, Saccharum officinarum, Salis babilonics, Salix purpurea, Salix tamarisifolia, Salvia elegans, Salvia officinalis, Salvia sclarea, Salvia sylvestris, Sambucus canadensis, Sambucus ebulus, Sambucus nigra, Sanchezia nobilis, Sanguisorba minor, Sanguisorba officinalis, Santolina chamnaecyparissus, Saponaria officinalis, Satureja hortensis, Satureja montana, Satureja repandra, Schisandra chinensis, Scolymus hispanicus, Scorzonera hispanica, Scotch pine, Scrophularia nodosa, Scutellaria certicola, Scutellaria lateriflora, Scutellarian altissirna, Secale cereale, Sechium edule, Sedum alburn, Sedum telchium, Sempervivum tectorum, Senecio platifilla, Senecio vuigaris, Senseviera sp., Serenoa repens, Seringa josiceae, Serratula tinctoria, Seruginea uffruticisa, Sesamum indicum, Sesbania exaltata, Sesbania speciosa, Setaria italica, ibirea altaiensis, Sidalcea spp., Silene vulgaris, Silybum marianum, Sinapis alba subsp. alba, Siringa vulgaris, Sium sisarum, Sluffera sp., Solanum duicamara, Solanum melongena, Solanum scabrum, Solanum tuberosum, Soleirolia soleirolii, Solidago caesia, Solidago canadensis, Solidago spp., Solidago virgaurea, Solidago×hybrida, Sonchus oleraceus, Sorbocotoneaster sp., Sorbus aucuparia, Sorbus cominicta, Sorghum bicolor, Sorghum×drummondii, Spartina potentiflora, Spathiphyllum cochlearispaturn, Spathiphyllum grandiflorum, Spinacia oleracea, Stachis lanata, Stachys affinis, Stachys byzantina, Stachys macrantha, Staphylea trifolia, Stellaria graminea, Stellaria media, Stephanandra incisa, Stepochlaena tenuifolia, Sterulia elata, Stevartia coreana, Stewartia pseudocamellia, Stipa capillata, Strelitzia reginae, Sulda sanganea, Sundapsis spp., Symphitium officinalis, Symphoricarpbs albus, Symphoricarpos orbiculatus, Symphytum officinale, Syngonium aurutum, Syngonium podophyllum, Taccus bacata, Tagetes minuta, Talictrum minus, Talictrum sp., Tamarindus india, Tamarindus indica, Tanacetum balsamita, Tanacetum balsamita subsp. balsamita, Tanacetum cinerariifolium, Tanacetum parthenium, Tanacetum vulgare, Tapeinochilos spectabilis, Taraxacum officinale, Taraxacum officinalis, Taxodium dixticum, Taxus cuspidata, Taxus hiksii, Taxus media, Taxus×media, Tetraclinis articulata hinensis, Tetradenia riparia, Teucrium chamaedrys, Thalictrum aquilegiifolium, Thalictumi flavum, Thlaspi arvense, Thuja occidentalis, Thymus camosus, Thymus cretaceus, Thymus cytridorus “aureus, Thymus fragantissimus, Thymus herba-barona, Thymus lemabarona, Thymus portugalense, Thymus praecox, Thymus praecox subsp. arcticus, Thymus pseudolamginosus, Thymus pseudolanuginosus, Thymus puleglodes “lemons”, Thymus puliglodes, Thymus serphylum, Thymus speciosa, Thymus thrasicus, Thymus vulgaris, Thymus vulgaris “argenteus,” Thymus vulgaris “oregano,” Thymus wooly, Thymus×citriodorus, Tiarella cordifolia, Tiarella spp., Tragopogon porrifolius, Tragopogon spp., Trambe pontica, Trevesia sungaica, Trichosanthes kirilowii, Trifolium hybridum, Trifolium incaamatum, Trifolium pannomncum, Trifolium pratense, Trifolium repens, Trigonella foenum-graecum, Triticum aestivum, Triticum aestivum subsp. spelta, Triticum turgidum, Trollius×cultorum, Tropaeolum majus, Tsuga canadensis, Tsuga canadensis “penola”, Tsuga diversifolia, Tsuga mertensiana, Tuja orientalis “eligantissima”, Tula ocidentalis “columbia,” Tulip tree, Tumera ulmifolia, Tussilago farfara, Typha latifolia, Ulmus americana, Ulmnus pumila, Urtica dioica, Uschusa sp., Uvwlaria perfoliata, Vaccinium angustifolium, Vaccinium corymbosum, Vaccinium macrocarpon, Valeriana officinalis, Valerianella locusta, Veratrum nigrum, Veratnim viride, Verbascum thapsus, Verbena officinalis, Verium oleander, Vemonia gigantea, Veronica austriaca ssp teucrium, Veronica beccabunga, Veronica officinalis, Viburnum opulus, Viburnum plicatum, Vicia faba, Vicia sativa, Vicia villosa, Vigna angularis, Vigna mungo, Vigna unguiculata, Vinca minor, Vincetocsicum officinalis, Vitis labrissa, Vitis spp., Weigela coraeensis, Weigela hortensis, Withania somnifera, ×Triticosecale spp., Xanthium sibiricum, Xanthium strumarium, Xanthosoma sagittifolium, Xeupressocyparis deylandii, Yucca elephantipes, Yucca filamentosa, Zea mays, Zelcova, Zingiber officinalis and Zingiber officinale.

Groups of potential plants may also be selected based on their indigenous geographical regions. For example, one group of potential plants could comprise plants that are indigenous to arid regions, for example, those located between 35° north latitude and 35° south latitude. In accordance with another embodiment of the present invention, therefore, potential plants comprise: the agave, Agavaceae, family including such members as: Yucca elata, Y. breviflora, Agave deserti, A. chrysantha, Dasylirion wheeleri; the buckwheat, Polygonaceae, family, such as Eriogonum fasciculatum; the crowfoot, Ranunculaceae, family, such as Delphinium scaposum, Anemone tuberosa and D. parishii; the poppy, Papaveraceae, family, including Platystemon californicus, Argemone pleiacantha, Corydalis aurea, Eschschoizia californica and Ar. corymbosa; members of the mustard, Cruciferae, family, such as Dithyrea californica, Streptanthus carinatus and Lesquerella gordoni; members of the legume, Leguminosae, family, such as Acacia greggii, Prosopis velutina, A. constrica, Senna covesii, Cercidium floridum, C. microphyllum, Lotus huminstratus, Krameria parvifolia, Parkinsonia aculeata, Calliendia eriophylla, Lupinus arizonicus, Olyneya tesota, Astragalus lentiginosus, Psorothamunus spinosus and Lupinus sparsiflorus; members of the loasa family, Loasaceae, including Mentzelia involucrata, M. pumila and Mohavea Confertiflora; members of the cactus, Cactaceae, family, such as Carnegiea gigantia, Opuntia leptocaulis, Ferocactus wislizenii, O. bigelovii, O. pheacantha, O. versicolor, O. fulgida, Echinocereus engelmannii, Manmmillaria microcarpa, O. basilaris, Stenocereins thurberi, O. violacea, M. tetrancistra, O. ramosissima, O. acanthocarpa, E. pectinatins and O. arbuscula; members of the evening primrose, Onagraceae, family, such as Oenothera deltoides, Camissonia claviformis and Oe. primiveris; members of the milkweed, Asclepiadaceae, family, including Asclepias erosa, A. sublata and Sarcostemma cynanchoides; members of the borage, Boraginaceae, family, such as Cryptantha augusti folia and Amsinckia intermedia; members of the sunflower, Compositae, family, including Baccharis sarothroides, Monoptiilon belloides, Erieron divergens, Zinnia acerosa, Melampodium leucanthan, Chaenactis fremontii, Calycoseris wrightii, Malacothrix californica, Helianthus annus, H. niveus, Geraea canescens; Hymenothrix wislizenii, Encelia farinosa, Psilostrophe cooperi, Baileya multiradiata, Bebbia juncea, Senecio douglasii, Trixis californica, Machaeranthera tephrodes, Xylorhiza tortifolia, Cirsiinm neomexicanum, Antennaria parviflora and Ch. douglasii; members of the caltrop, Zygophyllaceae, family, including Larrea tridentata and Kallstroemia grandiflora; members of the mallow, Malvaceae, family, including Hibiscus coulteri, H. denudatus and Sphaeralcea ambigua; members of the phlox, Polemoniaceae, family, such as Luanthus aureus; members of the unicorn plant, Martyniaceae, family, such as Proboscidiea altheaefolia; members of the gourd, Cucurbitaceae, family, such as Cucurbita digitata; members of the lily, Lilaceae, family, including Calochortus kennedyi, Dichelostemma pulchellum, Allium macropetalum and Hesperocallis indulata; members of the ocotillo, Fouquieriaceae, family, including Fouquieria splendens; members of the figwort, Scrophulariaceae, family, such as Castilleja sp., Penstemon parryi and Orthocarpus purpurascens; members of the acanthus, Acanthaceae, family, including Anisacanthus thurberi, Justicia californica and Ruellia nudiflora; members of the four o'clock, Nyctaginaceae, family, such as Allionia incamata, Abronia villosa and Mirabilis multiflora; members of the geranium, Geraniaceae, family, including Erodium cicutarium; members of the waterleaf, Hydrophyllaceae, family, such as Nama demissum, Phacelia bombycina and Ph. distans; members of the bignonia, Bignoniaceae, family, such as Chilopsis linearis; members of the vervain, Verbenaceae, family, including Glandularia gooddugii and Verbena neomexicana; members of the mint, Labiatae, family, such as Hyptis emoryi and Salvia columbariae; members of the broomrape, Orobanchaceae, family, such as Orobanche cooperi; members of the portulaca, Portulaceae, family, such as Talinum auriantiacum; members of the carpet-weed, Aizoaceae, family, such as Sesuvium verrucosum; members of the flax, Linaceae, family, such as Linum lewisii; members of the potato, Solanaceae, family, including Nicotiana trigonophylla and Physalis lobata; and members of the cochlospermum, Cochlospermaceae, family, such as Amoreuxia palmatifida.

If desired, the potential plant(s) can be subjected to a harvest stress treatment. A stress treatment comprises contacting or treating the potential plant(s), or material from the potential plant(s), with one or more stressor. The stressor can be a chemical compound or a physical treatment. Examples of suitable stressors are provided above. Various combinations of stressors and treatment regimes can also be employed as would be apparent to one skilled in the art.

The plant material may be used immediately after harvest, or it can be stored for a period of time prior to performing the extraction procedure(s). If desired, the plant material can be treated prior to storage, for example, by drying, freezing, lyophilising, or some combination thereof. Following treatment to prepare the plant material for storage, the plant material may be stored for a period of time prior to preparation of the extract. The storage time may be of various duration, for example, the storage period may be between a few days and a few years. In one embodiment of the invention, the plant material is stored for a period of less than one week. In another embodiment, the plant material is stored for a period between one week to one month. In a further embodiment, the plant material is stored for a period of between one month to six months. In other embodiments, the plant material is stored for periods of between four months to one year and for a period over one year in duration.

The Extraction Process

Various extraction processes are known in the art and can be employed in the process of the present invention (see, for example, International Patent Application WO 02/06992).

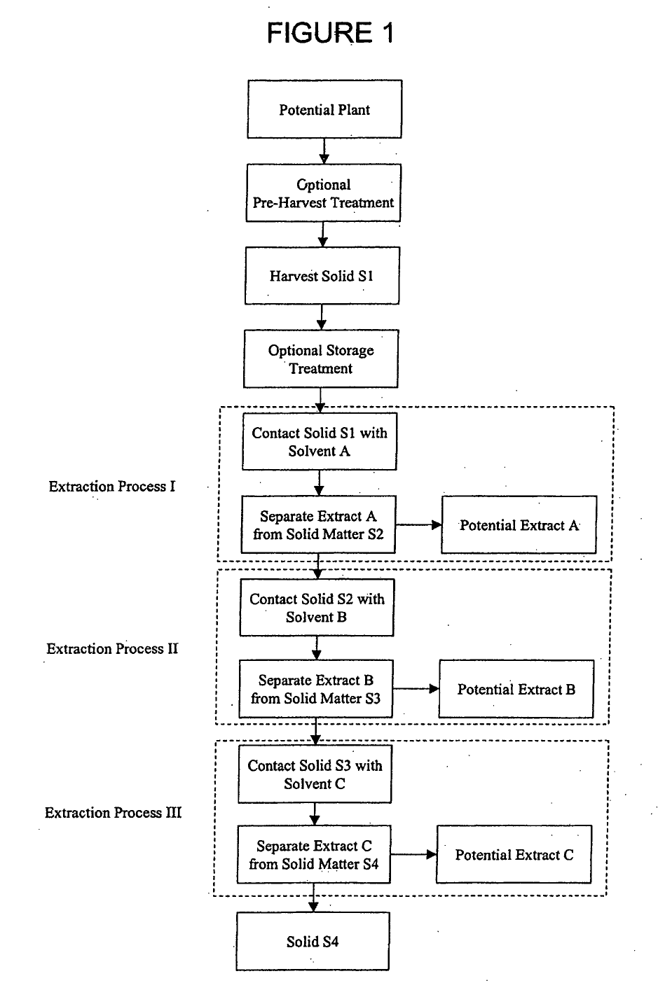

In one embodiment of the present invention the plant material is subjected to an extraction process as depicted in FIG. 1. In accordance with this embodiment, three basic extraction processes are performed in sequence to generate potential extracts A, B and C.

In other embodiments of the present invention, greater or fewer extraction processes are contemplated. For example, in an alternative embodiment, the plant material is subjected to an extraction process as depicted in FIG. 5. In accordance with this embodiment, the plant material is, subjected to two separate extraction processes concurrently resulting in two separate potential extract As.

Regardless of the number of extraction processes, the procedure for each extraction process entails contacting the solid plant material with a solvent with adequate mixing and for a period of time sufficient to ensure adequate exposure of the solid plant material to the solvent such that inhibitory activity present in the plant material can be taken up by the solvent. Typically, the extraction procedures are conducted over a period of time between about 10 minutes and about 24 hours at a temperature between about 4° C. and about 50° C. Other times and temperatures may be employed in the extraction process as described above. Adequate contact of the solvent with the plant material can be encouraged by shaking the suspension. The liquid fraction is then separated from the solid (insoluble) matter resulting in the generation of two fractions: a liquid fraction, which is a potential extract, and a solid fraction. Separation of the liquid and solid fractions can be achieved by one or more standard processes known to those skilled in the art.

In accordance with the embodiment depicted in FIG. 1, the extraction process is then repeated with a second and a third solvent. Solvents A, B and C in FIG. 1 generally represent separate classes of solvents, for example, aqueous, alcoholic and F organic. The solvents can be applied in specific order, for example, a polar to non-polar order or in a non-polar to polar order. Alternatively, the solvents can be applied in a random sequence. In all cases, however, the solid matter should be dried prior to contact with the subsequent solvent.

The plant material employed in the extraction process can be the entire potential plant, or it can be one or more distinct tissues from a plant, for example, leaves, seeds, roots, stems, flowers, and the like, or various combinations thereof. The plant material can be fresh, dried or frozen. If desired, the plant material can be treated prior to the extraction process in order to facilitate the extraction process. Typically such treatment results in the plant material being fragmented by some means such that a greater surface area is presented to the solvent. For example, the plant material can be crushed or sliced mechanically, using a grinder or other device to fragment the plant parts into small pieces or particles, or the plant material can be frozen liquid nitrogen and then crushed or fragmented into smaller pieces.

The solvent used for each extraction process can be aqueous, alcoholic or organic, or a combination thereof. In one embodiment of the present invention, plant material is extracted with an aqueous solvent. In another embodiment, an aqueous solvent comprising an aqueous buffer at pH 6-8 for a period of between 30 minutes to 8 hours at a temperature between about 4 to about 50° C. is used for the extraction.

In an alternate embodiment of the invention, plant material is extracted with an alcoholic solvent, such as ethanol, methanol, 1-propanol, 1-butanol, 2-propanol, 2-butanol, 2-methyl-1-propanol, 2-methyl-2-propanol, glycerine, ethylene glycol, propylene glycol, diethylene glycol, dipropylene glycol or 1,3-butylene glycol or a combination of alcoholic solvents. In one embodiment, a combination of ethanol and methanol is used as the alcoholic solvent, wherein the range of ethanol:methanol is between about 50:50 and about 85:15. In another embodiment, a glycol is used as the alcoholic solvent. In a further embodiment, the plant material is contacted with an alcoholic solvent for a time period between about 10 minutes to one hour at a temperature between about 4 to about 25° C.

In an alternate embodiment, plant material is extracted with an alcoholic solvent in combination with a co-solvent, which may be aqueous or organic. In one embodiment, a combination of ethanol and water is used as the solvent, wherein the range of ethanol:water is between about 50:50 and about 85:15. In another embodiment, a combination of a glycol and water is used as the solvent, wherein the range of glycol:water is between about 95:5 and about 50:50.

In an alternate embodiment, plant material is extracted with an organic solvent, such as diethylether, hexane, heptane, dichloromethane, or ethylacetate. In one embodiment, dichloromethane is used as the solvent and the plant material is shaken for one to twenty-four hours with the solvent.

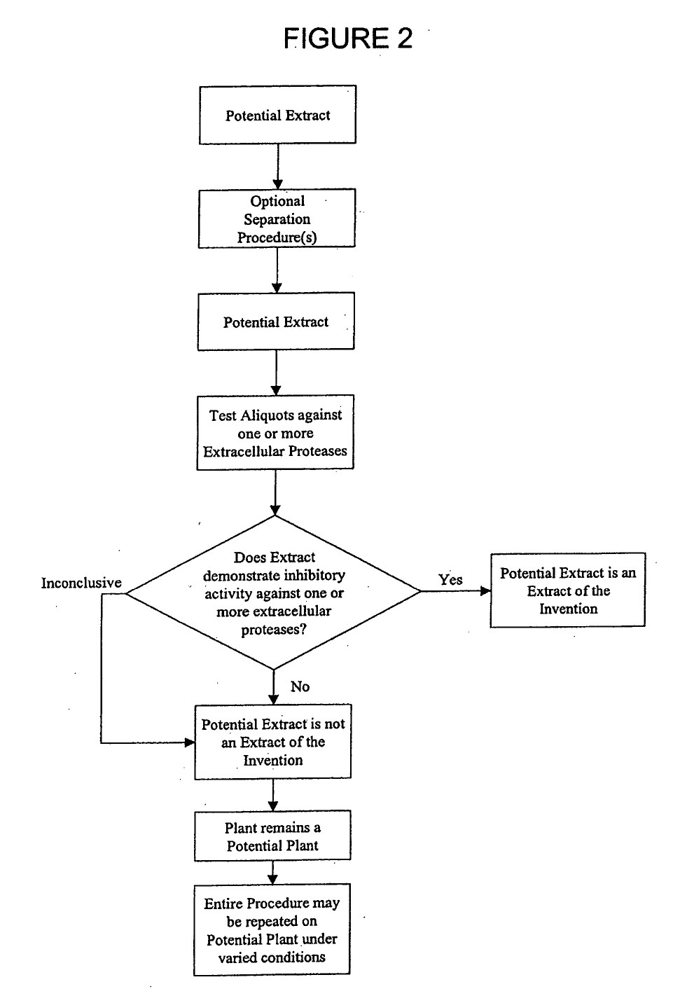

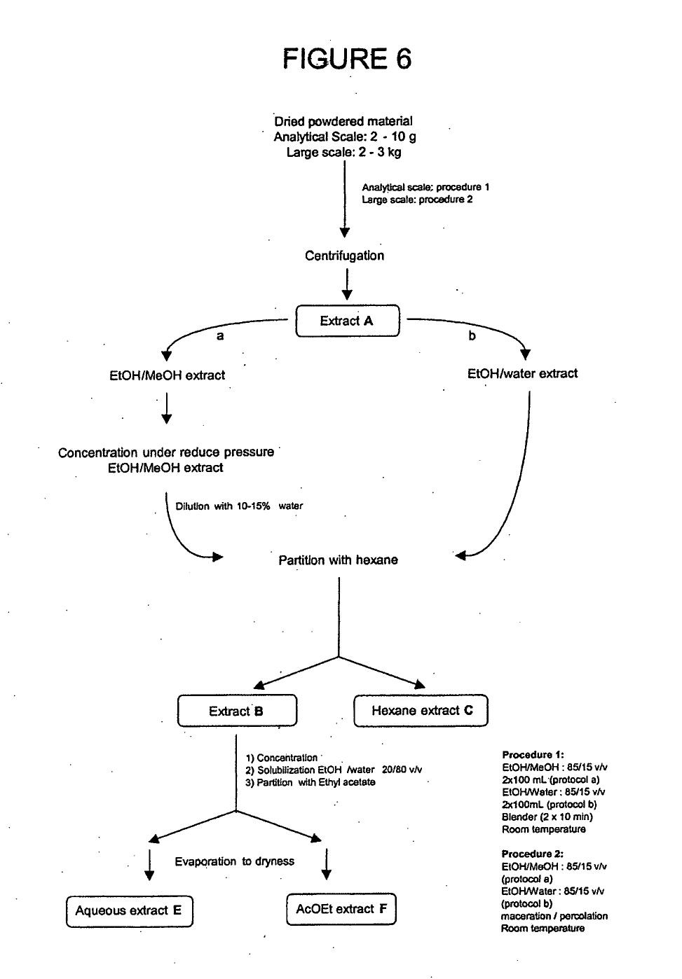

Once the potential extracts have been isolated, they can be tested directly (after being dissolved or dispersed in a suitable solvent) for their ability to inhibit skin EP activity, or they may be subjected to further procedures as described below and outlined in FIGS. 2 and 6. For example, the potential extracts can be subjected to procedures to remove fatty acids or chlorophyll components that may interfere with the protease activity or other assays. Various procedures known in the art may be employed. In one embodiment, one or more additional partitioning step using an organic solvent, such as hexane, heptane or ethyl acetate, is included. The liquid potential extract can be concentrated and solubilised in an appropriate solvent prior to the one or more partitioning step, if desired.

The present invention contemplates that the extraction process may be carried out on various scales including known large, medium and small-scale methods of preparing extracts.

Determination of Skin Extracellular Protease Inhibiting Activity

Following the extraction process, the potential extracts are tested for their ability to inhibit one or more skin EPs selected from the group of: MMP-1, MMP-2, MMP-3, MMP-9 and HLE, using one of a variety of techniques known in the art including, but not limited to, those described herein. Those plant extracts that decrease the activity of at least one skin EP by at least 20% are selected for further testing. In one embodiment of the present invention, plant extracts that inhibit the activity of one or more of MMP-1, MMP-2, MMP-3, MMP-9 and HLE by at least 30% are selected. In another embodiment, plant extracts that inhibit the activity of one or more of MMP-1, MMP-2, MMP-3, MMP-9 and HLE by at least 40% are selected. In another embodiment, plant extracts that inhibit the activity of one or more of MMP-1, MMP-2, MMP-3, MMP-9 and HLE by at least 50% are selected.

In order to determine whether the potential extracts inhibit a skin EP, the extracts can be tested against an individual skin EP or against a panel comprising two or more of MMP-1, MMP-2, MMP-3, MMP-9 and HLE. Similarly, the extracts can be tested individually or a plurality of extracts can be tested simultaneously using high-throughput assays, as known in the art. Simultaneous testing of a plurality of extracts maximizes the number of extracts that can be tested in a set period of time and thus decreases the overall time for the screening process.

Cellular Screening of Extracts

Those extracts identified as being capable of inhibiting one or more of MMP-1, MMP-2, MMP-3, MMP-9 and HLE are subsequently screened for their ability to affect one or more cellular activities in skin cells. Such cellular activities include, for example, attenuating the breakdown of a structural component of the ECM (i.e. collagen, fibronectin, fibrillin and/or elastin); attenuating endothelial cell migration; increasing collagen production; attenuating UV-induced extracellular protease activity and/or attenuating tractional forces generated by fibroblasts. The extracts can be tested using standard methods such as those described above.

Further Testing

The extracts identified by the above process may be submitted to other standard tests, such as cytotoxicity tests, stability tests, bioavailability tests and the like, to determine their suitability for inclusion in a dermatological formulation of the invention. Exemplary tests are described above.

To gain a better understanding of the invention described herein, the following examples are set forth. It should be understood that these examples are for illustrative purposes only. Therefore, they should not limit the scope of this invention in any way.

### EXAMPLES

### Example I

### Preparation of Stressed and Non-stressed Plant Extracts (Method A)

Optional Pre-Harvest Treatment: Aerial parts of a living plant were sprayed with an aqueous solution of gamma linolenic acid (6,9,12-Octadecatrienoic acid, Sigma L-2378) (stress G) or arachidonic acid-(5,8,11,14-Eicosatetraenoic acid, Sigma A-3925) (stress A) (400 μM in water with 0.125% (v/v) Triton X-100) to completely cover the leaves. Twenty to twenty-four hours after the stress, plants were harvested.

Harvest Solid S1 and Optional Storage Treatment: More than 4 grams of leaves, stems, fruit, flowers, seeds or other plant parts were harvested from stressed or non-stressed plants and frozen immediately in dry ice, then transferred as soon as possible to a −20° C. freezer until use. Plant materials may be stored at −20° C. for than a year without losing inhibitory activity. Temperature was monitored to ensure a constant condition.

Stressed and non-stressed plant specimens were collected as wet samples and stored at −20° C. for various periods of time, and were submitted to a process which generates 3 subfractions: aqueous, ethanolic and organic fractions. The complete extraction process was performed in a continuous cycle using the following steps. An initial 5 g of plant specimen was homogenized in liquid nitrogen with a blender. The resulting powder was weighed.

Extraction Process I—Aqueous Extraction: To each 4.5 grams of plant powder, 12 ml of a cold solution of 100 mM Tris, pH 7.0 was added. The mixture was thoroughly vortexed for 2 minutes. The mixture was kept on ice for 30 minutes and vortexed after each 10 minute period of time. The sample was centrifuged in a Corex™ 30 ml tube for 5 minutes at 4500 rpm. The resulting supernatant was decanted in a 15 ml tube after filtration with a Miracloth™ filter. This extract represents Potential Extract A in FIG. 1. The pellet, referred to as Solid S2, was kept for ethanolic extraction.

The aqueous extract (Potential Extract A) was further purified in order to determine its EP inhibition capability. The Potential Extract A was purified by size-exclusion chromatography, wherein the aqueous extract was chromatographed on a calibrated Sephadex G-25 column (1×10 cm) using a 20 mM Tris-HCl, 150 mM NaCl, pH 7.5 buffer as eluant. Fractions corresponding to compounds that appeared to have a molecular weight (NW) less than 1500 daltons (D) were pooled to constitute the purified aqueous extract.

Prior to analysis of the aqueous extract for inhibitory activity as described in Example II, the extract was treated with 10% gelatine-Sepharose (Pharmacia Biotech, Uppsala, Sw.) in order to remove unspecific enzyme ligands. To 1 mL of extract, 100 μL of gelatine-Sepharose resin was added in a microassay tube, the solution in the tube was mixed, kept on ice for 30 minutes, and then centrifuged 5 minutes at 5,000 rpm. The supernatant was removed and used directly for assays.

Extraction Process II—Alcoholic Extraction: To the pellet, Solid S2, collected from the previous aqueous extraction, 12 ml of cold ethanol:methanol (85:15) was added and the mixture was thoroughly vortexed for 2 minutes. The mixture was kept on ice for 30 minutes and vortexed every 10 minutes. The sample was centrifuged in a Core x™ 30 ml tube for 5 minutes at 4,500 rpm. The resulting supernatant was decanted in a 15 ml tube after filtration with a Miracloth™ filter. The pellet, referred to as Solid S3, was kept for the subsequent organic extraction. This extract represents Potential Extract B.

The ethanolic extract, Potential Extract B, was purified by liquid/liquid extraction prior to analysis by enzymatic assay. For this purpose, 1 ml of ethanolic extract was evaporated under vacuum, dissolved in 150 μl of dimethylsulfoxide (DMSO), and completed to a final volume of 1.5 ml with Tris buffer (final concentration: Tris-HCl mM; pH 7.5). Four ml of hexane was added to the Tris phase in a glass tube and the tube was thoroughly vortexed, then allowed to form a biphasic liquid. The organic phase was removed and the extract was submitted to a second round of liquid/liquid extraction. The aqueous phase was removed and treated with 10% gelatine-Sepharose (Pharmacia Biotech, Uppsala, Sw) to remove non-specific enzyme ligands prior to conducting subsequent assays. To 1 ml of extract, 100 μL of gelatine-Sepharose resin was added in a microassay tube, the tube was mixed, kept on ice for 30 minutes, and then centrifuged 5 minutes at 5,000 rpm. Supernatant was removed and used directly for assays as described in Example II.

Extraction Process III—Organic Extraction: To the pellet, Solid S3, collected from the previous ethanolic extraction, 12 ml of cold dichloromethane was added and the mixture was thoroughly vortexed for 2 minutes. The mixture was kept on ice for 30 minutes and vortexed after each 10 minutes period. The sample was centrifuged in a Corex™ 30 ml tube for 5 minutes at 4,500 rpm. The resulting supernatant was decanted in a 15 ml glass tube after filtration with a Miracloth™ filter. The final pellet was discarded. The organic solvent was evaporated under vacuum and the phase was dissolved with dimethylsulfoxide (DMSO). This extract represents Potential Extract C, which was further purified by solid phase extraction prior to analysis by enzymatic assay.

In order to assay the Potential Extract C, the organic extract was diluted 1:10 in a solution of DMSO:Methanol:Tris (20 mM, pH 7.5) (10:50:40) (Solution A), i.e., 220 μl of extract was added to 2.0 ml of solution A. After 10 seconds of vigorous vortex, the mix was sonicated for 10 seconds. Dissolved extracts were subsequently applied to a solid phase extraction plate (Discovery SPE-96, Sigma Chemical Co, St-Louis, Mo.). After initial conditioning of the columns with 1 ml of methanol, columns were equilibrated with solution A, and extract samples were deposited on the columns. Elution was completed with solution A (final volume of 2 ml) and this fraction was used directly in assays as described in Example II.

### Example II

### In vitro Enzyme Inhibition Assays

The inhibitory activity of sample compositions towards human MMP-1, human MMP-2, human MMP-3, human MMP-9 and/or human leukocyte elastase (HLE) were determined using either fluorogenic substrates or the FASC assay.

Measurement of Human MMP-1, -2, -3 and -9 Activity with Fluorogenic Peptidic Substrates

MMP-1, -2, -9 were purified from natural sources (human immortalized cell lines: 8505C (Deutsche Sammlung von Mikroorganismen und Zellkulturen GmbH) for MMP-1, HT-1080 (ATCC, Manassas, Va.) for MMP-2 and THP-1 (ATCC, Manassas, Va.) for MMP-9) as described in literature and based on protocols found in I. M. Clark: <<Matrix metalloproteinases protocols>>, Humana Press (2001). Recombinant human MMP-3 was overexpressed in E. coli and purified according to Windsor L J, Steele D L (2001), Methods Mol Biol 151:191-205. Proteolytic activity of these proteases was evaluated with the assay based on the cleavage of auto-quenched peptide substrate: (MCA-Pro-Leu-Gly-Leu-Dpa-Ala-Arg-NH₂ TFA [Dpa=N-3-(2,4-dinitrophenyl)-L-2,3-diaminopropionyl]) for MMP-1, -2, and -9; and, MCA-Arg-Pro-Lys-Pro-Val-Glu-Nva-Trp-Arg-Lys(DNP)—NH₂ (DNP=2,4-dinitrophenyl; Nva=L-norvaline) for MMP-3 (Calbiochem, San Diego, Calif.). In the intact peptide, Dpa or DNP quenches the MCA fluorescence. Cleavage of the peptide causes release of the fluorescent MCA group which was then quantitated on a fluorometer (Gemini XS, Molecular Devices, Sunnyvale, Calif.). The assay was performed in TNCZ assay buffer (20 mM Tris-HCl; NaCl 150 mM; CaCL₂ 5 mM; ZnCl₂ 0.5 mM; pH 7.5) with human purified proteases (I. M. Clark: Matrix metalloproteinases protocols, Humana Press (2001)). The substrate, primarily dissolved in DMSO was then redissolved in TNCZ buffer for the assay. In a typical assay, 10 μl of purified enzyme (1-50 ng) and 5 μl of dissolved substrate (final concentration of 10 μM) was mixed in a final volume of 75 μl (completed with TNCZ). All assays were performed in 96 well plate and the reaction was started by the addition of substrate. Assays were measured (excitation 325 nm, emission 392 nm) for 20, 40 and 60 minutes.

Measurement of Human MMP-9 or Human Leukocyte Elastase (HLE) Activity Using the FASC Assay

Human leukocyte elastase was obtained from Calbiochem (San Diego, Calif.). Human MMP-9 was purified as previously described. The assay was based on the method described in Canadian Patent No. 2,189,486 (1996) and by St-Pierre et al., (Cytometry (1996) 25:374-380. For the assay, 5 μl of the purified enzyme (1-100 ng), 5 μl of concentrated buffer solution (20 mM Tris-HCl; NaCl 150 mM; CaCL₂ 5 mM; ZnCl₂ 0.5 mM; pH 7.5), and 5 μl of gelatine-FITC beads were typically used in a final volume of 100 μl. The assay was performed by incubation of the reaction mixture for 90 minutes at 37° C. The reaction was stopped by the transfer of the mix in 0.5 ml of 20 mM Tris, 150 mM NaCl; pH 9.5 buffer. This tube was analyzed in a flow cytometer (Epics MCL, Beckman Coulter, Mississauga, Ontario) as described in Canadian Patent No. 2,189,486.

Measurement of HLE Activity with a Fluorogenic Proteic Substrate

HLE was obtained from Calbiochem (San Diego, Calif.). The activity of HLE was measured by an assay based on the increase of fluorescence of a proteic substrate (beta-casein) heavily labelled with Alexa-488 dye (Molecular Probes, Eugene, Or). The substrate, when highly labelled with the dye, will almost quench the dye fluorescence. Cleavage of the substrate will result in an increase of the fluorescence which can be measured with a spectrofluorometer, and which was proportional to protease activity. Typically, 10 μl of purified HLE (10-50 ng) and 10 μL of beta-casein-Alexa488 (100 ng) were assayed in final volume of 75 μl adjusted with 20 mM TNCZ buffer. The reaction was performed as already described except that the fluorescence was read at excitation 488 nm/emission 525 nm wavelengths.

Inhibition Assay for Plant Extracts

Before a typical assay, aqueous extracts prepared as described in Example I were preincubated with 1:10 of gelatine-Sepharose 4B™ for 30 minutes to remove fluorescence quenching. For the ethanolic extract, an initial hexane extraction was performed and samples were treated with 1:10 of gelatine-Sepharose 4B™ to remove quenching.

In a typical fluorescent assay, 10 μl of purified enzyme at concentrations previously mentioned for the enzymatic assay, 5 μl of dissolved fluorogenic peptide or 10 μl of dissolved fluorescent proteic substrate (final concentration of 10 μM) and 40 μL of the aqueous, ethanolic or organic extract to be tested were mixed in a final volume of 75 μl (completed with TNCZ for fluorogenic peptide substrate assay or 20 mM citrate pH 3.3 buffer for fluorescent protein substrate assay). All assays were performed in 96 well plate and the reaction was started by the addition of substrate. Assays were measured (excitation 325 nm, emission 392 nm for peptide and excitation 488 nm/emission 525 nm wavelengths for protein) for 20, 40 and 60 minutes. Activity and inhibition values were determined from the increase in fluorescence

For the FASC assay, 35 μl of the treated extract prepared as described in Example I, 5 μl of the purified enzyme prepared as described previously, 5 μl of concentrated buffer solution (TNCZ), and 5 μl of gelatine-FITC beads were typically used. The initial step of the assay was the incubation of the reaction without beads for a 30 minutes period on ice to allow the binding of inhibitors to enzyme. Fluorescent beads were added and the reaction mix was incubated for 90 minutes at 37° C. The reaction was stopped by transfer of the mix in 0.5 ml of 20 mM Tris, 150 mM NaCl; pH 9.5 buffer. This tube was analyzed in the flow cytometer (Epics MCL, Beckman Coulter, Mississauga, Ontario) as described in Canadian Patent Application No. 2,189,486 (1996).

Results of the inhibition studies are shown in Tables 1-5 for aqueous (O), ethanolic (R) and organic (S) extracts from exemplary stressed (A: Arachidonic acid and G: Gamma-linolenic acid) and non-stressed (T) plant sources. The inhibition is reported as percentage (%) of inhibition of substrate degradation as compared with substrate degradation in the absence of the extract. Percentage inhibition was calculated according to the formula:
Percentage (%) inhibition=[E_{A}−E_{B}/E_{A}]×100

wherein E_{A} is the protease activity in the absence of the plant extract and E_{B} is the protease activity in the presence of the extract.

TABLE 1
Inhibition of MMP-1 by Plant Extracts

| | | | Inhibition |
|---|---|---|---|
| Latin Name | Stress | Extract | (%) |
| Achillea millefolium | A | O | 22.2 |
| Acorus calamus | A | O | 100.0 |
| Actinidia arguta | A | O | 56.4 |
| Agastache foeniculum | A | S | 30.4 |
| Alchemilla mollis | A | 4 | 36.4 |
| Allium cepa | A | O | 61.4 |
| Allium grande | A | R | 46.5 |
| Allium porrum | A | R | 25.0 |
| Allium porrum | A | O | 98.9 |
| Allium sativum | A | O | 42.5 |
| Allium sativum | A | R | 98.7 |
| Allium schoenoprasum | A | R | 22.3 |
| Allium Tuberosum | A | R | 29.9 |
| Allium Tuberosum | A | O | 100.0 |
| Althaea officinalis | A | S | 21.6 |
| Angelica archangelica | A | S | 45.9 |
| Anthemis nobilis | A | R | 34.5 |
| Aralia nudicaulis | A | O | 100.0 |
| Armoracia rusticana | A | O | 31.2 |
| Armoracia rusticana | A | S | 39.7 |
| Aronia melanocarpa | A | R | 39.8 |
| Aster sp | A | O | 67.6 |
| Beckmannia eruciformis | A | O | 24.1 |
| Beta vulgaris | A | R | 41.2 |
| Beta vulgaris spp. Maritima | A | O | 44.1 |
| Brassica napus | A | O | 26.3 |
| Brassica oleracea | A | S | 28.6 |
| Brassica oleracea | A | R | 33.8 |
| Brassica oleracea | A | O | 100.0 |
| Brassica rapa | A | R | 61.4 |
| Calamintha nepeta | A | R | 40.2 |
| Camellia sinensis | A | O | 39.3 |
| capsicum annuum | A | R | 34.3 |
| capsicum annuum | A | O | 88.3 |
| Capsicum frutescens | A | R | 39.4 |
| Chenopodium bonus-henricus | A | O | 100.0 |
| Chenopodium bonus-henricus | A | R | 37.3 |
| Chenopodium quinoa | A | O | 66.3 |
| Chrysanthenum coronarium | A | R | 37.4 |
| Cichorium intybus | A | R | 22.0 |
| Cichorium intybus | A | S | 66.9 |
| Citrullus lanatus | A | O | 41.9 |
| Cornus canadensis | A | S | 73.0 |
| Crataegus sp | A | O | 100.0 |
| Cucumis Anguria | A | S | 34.2 |
| Cucurbita moschata | A | O | 27.3 |
| Cucurbita pepo | A | O | 84.9 |
| Cymbopogn citratus | A | O | 100.0 |
| Cymbopogon citratus | A | R | 22.1 |
| Cyperus esculentus | A | R | 25.8 |
| Cyperus esculentus | A | O | 28.1 |
| Dactylis glomerata | A | O | 25.5 |
| Daucus carota | A | O | 43.4 |
| Daucus carota | A | R | 100.0 |
| Dipsacus sativus | A | O | 35.3 |
| Dirca palustris | A | S | 47.9 |
| Eruca vesicaria | A | R | 33.7 |
| Eschscholzia californica | A | O | 61.1 |
| Eschscholzia californica | A | R | 74.1 |
| Filipendula rubra | A | O | 51.7 |
| Foeniculum vulgare | A | O | 86.2 |
| Fragaria x ananassa | A | O | 23.7 |
| Fragaria Xananassa | A | S | 40.6 |
| Fragariax ananassa | A | R | 28.3 |
| Galinsoga ciliata | A | R | 29.7 |
| Gallium odoratum | A | 6 | 48.8 |
| Gaultheria hispidula | A | R | 23.9 |
| Glycine max | A | R | 24.7 |
| Glycine max | A | S | 29.6 |
| Glycine max | A | O | 100.0 |
| Guizotia abyssinica | A | S | 39.4 |
| Hamamelis virginiana | A | R | 49.1 |
| Helianthus Tuberosus | A | O | 95.9 |
| Heliotropium arborescens | A | R | 25.0 |
| Hordeum hexastichon | A | O | 100.0 |
| Hordeum vulgare | A | O | 46.2 |
| Hordeum vulgare subsp. Vulgare | A | O | 43.8 |
| Inula helenium | A | O | 25.8 |
| Lathyrus sativus | A | 0 | 27.1 |
| Leonurus cardiaca | A | O | 34.4 |
| Levisticum officinale | A | R | 31.7 |
| Lolium multiflorum | A | O | 39.0 |
| Lotus corniculatus | A | O | 100.0 |
| Malva sylvestris | A | R | 22.8 |
| Matricaria recutita | A | O | 25.1 |
| Matteucia pensylvanica | A | R | 48.1 |
| Medicago sativa | A | R | 25.1 |
| Melissa officinalis | A | O | 100.0 |
| Mentha piperita | A | O | 60.1 |
| Mentha suaveolens | A | O | 35.1 |
| Nepeta cataria | A | O | 100.0 |
| Nicotiana rustica | A | R | 20.7 |
| Origanum vulgare | A | R | 60.5 |
| Origanum vulgare | A | O | 73.2 |
| Perilla frutescens | A | R | 74.4 |
| Perilla frutescens | A | O | 92.4 |
| Petroselinum crispum | A | R | 77.4 |
| Phacelia tanacetifolia | A | R | 52.8 |
| Phaseolus coccineus | A | R | 20.9 |
| Phaseolus coccineus | A | S | 34.2 |
| Phaseolus Vulgaris | A | S | 29.2 |
| Phaseolus vulgaris | A | R | 56.1 |
| Phaseolus Vulgaris | A | R | 60.0 |
| Phaseolus Vulgaris | A | O | 100.0 |
| Phlox paniculata | A | O | 100.0 |
| Pimpinella anisum | A | S | 100.0 |
| Pimpinella anisum | A | R | 72.2 |
| Plantago coronopus | A | R | 23.7 |
| Plectranthus sp. | A | O | 25.0 |
| Poa compressa | A | O | 31.5 |
| Potentilla anserina | A | R | 71.2 |
| Pysalis ixocarpa | A | R | 32.1 |
| Raphanus raphanistrum | A | O | 31.5 |
| Raphanus sativus | A | O | 100.0 |
| Raphanus sativus | A | O | 30.2 |
| Rheum officinale | A | O | 79.1 |
| Rheum rhabarbarum | A | R | 22.9 |
| Rheum rhabarbarum | A | R | 32.8 |
| Ribes nigrum | A | O | 100.0 |
| Ribes nigrum | A | R | 100.0 |
| Ribes salivum | A | R | 48.6 |
| Ribes sylvestre | A | S | 26.5 |
| Ribes uva-crispa | A | R | 100.0 |
| Rubus canadensis | A | R | 46.1 |
| Rubus canadensis | A | R | 53.1 |
| Rubus idaeus | A | R | 100.0 |
| Salvia officianalis | A | O | 100.0 |
| Salvia sclarea | A | S | 43.8 |
| Satureja montana | A | R | 100.0 |
| Solanum dulcamara | A | S | 43.8 |
| Solanum melanocerasum | A | R | 37.2 |
| Solanum tuberosum | A | R | 100.0 |
| Sorghum dochna | A | O | 100.0 |
| Stachys byzantina | A | S | 28.9 |
| Stellaria media | A | S | 33.1 |
| Tanacetum parthenium | A | O | 28.9 |
| Tanacetum vulgare | A | R | 76.0 |
| Taraxacum officinale | A | O | 65.7 |
| Thymus praecox subsp arcticus | A | O | 64.2 |
| Thymus praecox subsp arcticus | A | R | 88.2 |
| Thymus vulgaris | A | R | 42.7 |
| Thymus x citriodorus | A | O | 34.7 |
| Trichosanthes kirilowii | A | R | 31.8 |
| Trifolium hybridum | A | R | 96.0 |
| Trifolium incarnatum | A | R | 100.0 |
| Trifolium pannonicum | A | R | 27.7 |
| Trifolium repens | A | R | 79.5 |
| Vaccinum augustifolium | A | R | 52.5 |
| Vaccinum macrocarpon | A | O | 64.5 |
| Vicia sativa | A | O | 60.8 |
| Vicia sativa | A | R | 28.6 |
| Vicia villosa | A | R | 64.7 |
| Vicia villosa | A | O | 57.3 |
| Vigna sesquipedalis | A | O | 33.0 |
| Vigna sesquipedalis | A | R | 24.4 |
| Vigna unguiculata | A | R | 20.6 |
| Vitia spp | A | R | 72.6 |
| Vitia spp | A | O | 100.0 |
| Zea Mays | A | R | 99.2 |
| Zea Mays | A | O | 100.0 |
| Abelmochus esculentus | G | R | 37.6 |
| Aconitum napellus | G | O | 100.0 |
| Allium ampeloprasum | G | R | 33.4 |
| Allium ascalonicum | G | R | 31.5 |
| Allium cepa | G | O | 34.4 |
| Allium cepa | G | R | 36.4 |
| AAllium sativum | G | R | 53.2 |
| Allium Tuberosum | G | R | 68.3 |
| Althaea officianalis | G | O | 47.7 |
| Althaea officinalis | G | S | 30.7 |
| Althaea officinalis | G | S | 44.3 |
| Althea officinalis | G | R | 83.6 |
| Anethum graveolens | G | S | 44.3 |
| Apium graveolens | G | R | 27.7 |
| Armoracia rusticana | G | O | 51.8 |
| Armoracia rusticana | G | S | 47.1 |
| Aronia melanocarpa | G | S | 66.5 |
| Artemisia dracunculus | G | S | 79.0 |
| Artemisia dracunculus | G | R | 50.3 |
| Asparagus officinalis | G | O | 96.4 |
| Bellis perennis | G | R | 44.1 |
| Beta vulgaris spp. Maritima | G | R | 43.7 |
| Beta vulgaris spp. Maritima | G | O | 34.9 |
| Betula glandulosa | G | S | 40.8 |
| Borago officinalis | G | O | 30.3 |
| Borago officinalis | G | R | 29.7 |
| Brassica cepticepa | G | R | 21.9 |
| Brassica oleracea | G | O | 33.6 |
| Brassica oleracea | G | O | 100.0 |
| Brassica rapa | G | O | 42.5 |
| Brassica rapa | G | R | 40.2 |
| Calamintha nepeta | G | O | 28.7 |
| Calendula officinalis L. | G | O | 100.0 |
| Camellia sinensis | G | O | 46.4 |
| Campanula rapunculus | G | R | 27.2 |
| Capsella bursa-pastoris | G | R | 24.1 |
| Capsicum annum | G | O | 36.0 |
| Chaerophyllum bulbosum | G | R | 38.9 |
| Chenopodium quinoa | G | O | 100.0 |
| Cichorium intybus | G | S | 44.6 |
| Circium arvense | G | R | 30.3 |
| Citrullus lanatus | G | R | 21.2 |
| Cucurbita pepo | G | O | 59.5 |
| Cucurbita Pepo | G | O | 40.2 |
| Cuminum cyminum | G | R | 25.5 |
| Cymbopogon citratus | G | R | 33.7 |
| Datura stramonium | G | O | 73.5 |
| Daucus carota | G | O | 86.0 |
| Daucus carota | G | O | 27.9 |
| Dryopteris filix-mas | G | O | 21.9 |
| Erysimum perofskianum | G | O | 24.4 |
| Fagopyrum esculentum | G | O | 100.0 |
| Foeniculum vulgare | G | O | 28.0 |
| Foeniculum vulgare | G | R | 57.3 |
| Gaultheria hispidula | G | O | 44.2 |
| Gaultheria procumbens | G | R | 94.8 |
| Glechoma hederacea | G | O | 25.5 |
| Glycine max | G | S | 100.0 |
| Glycyrrhiza glabra | G | O | 24.9 |
| Guizotia abyssinica | G | R | 30.3 |
| Helenium hoopesii | G | 0 | 28.6 |
| Helianthus annuus | G | O | 33.6 |
| Helianthus tuberosus | G | O | 54.4 |
| Hordeum vulgare | G | O | 28.8 |
| Hordeum vulgare subsp. Vulgare | G | R | 28.1 |
| Hypericum henryi | G | R | 80.0 |
| Iberis amara | G | O | 44.6 |
| Lactuca sativa | G | R | 25.3 |
| Lathyrus sylvestris | G | O | 90.2 |
| Lavandula angustifolia | G | R | 22.5 |
| Lepidium Sativum | G | S | 29.5 |
| Levisticum officinale | G | O | 100.0 |
| Lolium multiflorum | G | O | 24.9 |
| Lolium multiflorum | G | R | 27.1 |
| Lotus corniculatus | G | O | 52.2 |
| Lycopersicon esculentum | G | R | 24.4 |
| Lycopersicon pimpinellifolium | G | R | 30.3 |
| Malus hupehensis | G | R | 65.8 |
| Malva verticillata | G | R | 43.1 |
| Matricaria recutita | G | S | 100.0 |
| Matteucia pensylvanica | G | R | 57.5 |
| Melissa officinalis | G | O | 28.5 |
| Mentha piperita | G | O | 36.0 |
| Mentha spicata | G | S | 20.3 |
| Mentha spicata | G | S | 26.0 |
| Mentha suaveolens | G | O | 60.5 |
| Nepeta cataria | G | O | 24.1 |
| Nicotiana rustica | G | R | 28.1 |
| Nicotiana tabacum | G | R | 40.6 |
| Oenothera biennis | G | R | 28.4 |
| Oenothera biennis | G | O | 100.0 |
| Origanum vulgare | G | S | 100.0 |
| Origanum vulgare | G | O | 20.1 |
| Origanum vulgare | G | O | 85.4 |
| Oryza Sativa | G | R | 53.3 |
| Panax quinquefolius | G | S | 100.0 |
| Panicum miliaceum | G | S | 100.0 |
| Passiflora caerula | G | O | 20.9 |
| Pastinaca sativa | G | R | 68.4 |
| Pastinaca sativa | G | O | 100.0 |
| Pennisetum alopecuroides | G | R | 100.0 |
| Petroselinum crispum | G | R | 73.0 |
| Phalaris canariensis | G | O | 100.0 |
| Phaseolus coccineus | G | R | 29.9 |
| Phaseolus coccineus | G | R | 67.6 |
| Phaseolus coccineus | G | O | 32.4 |
| Phaseolus vulgaris | G | R | 33.4 |
| Phaseolus vulgaris | G | R | 60.2 |
| Phaseolus vulgaris | G | R | 22.3 |
| Phaseolus vulgaris | G | O | 87.7 |
| Phlox paniculata | G | O | 89.3 |
| Physalis pruinosa | G | O | 37.0 |
| Plantago coronopus | G | R | 48.1 |
| Plantago major | G | O | 47.0 |
| Plectranthus sp. | G | O | 97.2 |
| Potentilla anserina | G | R | 22.0 |
| Prunella vulgaris | G | O | 21.2 |
| Raphanus Raphanistrum | G | O | 95.9 |
| Raphanus sativus | G | O | 67.7 |
| Reseda odorata | G | O | 40.6 |
| Rheum officinale | G | O | 82.1 |
| Rheum rhabarbarum | G | R | 48.1 |
| Ribes Nigrum | G | R | 100.0 |
| Ribes Sylvestre | G | O | 42.9 |
| Ricinus communis | G | O | 73.5 |
| Rubus Phoenicalasius | G | R | 31.4 |
| Ruta graveolens | G | R | 100.0 |
| Salvia officinalis | G | R | 100.0 |
| Santolina | G | R | 28.1 |
| Satureja hortensis | G | R | 100.0 |
| Satureja repandra | G | O | 57.1 |
| Scrophularia nodosa | G | R | 41.6 |
| Scutelaria lateriflora | G | S | 72.1 |
| Sium sisarum | G | O | 99.7 |
| Solanum dulcamara | G | R | 65.4 |
| Solanum melanocerasum | G | R | 32.4 |
| Solanum melorgena | G | O | 100.0 |
| Solanum tuberosum | G | S | 46.4 |
| Sorghum caffrorum | G | R | 100.0 |
| Sorghum dochna | G | R | 51.4 |
| Sorghum dochna | G | R | 39.6 |
| Sorghum sudanense | G | O | 97.4 |
| Stachys byzantina | G | O | 41.4 |
| Stellaria media | G | O | 33.8 |
| Symphytum officinale | G | O | 52.0 |
| Tanacetum parthenium | G | O | 79.1 |
| Tanacetum vulgare | G | O | 100.0 |
| Taraxacum officinale | G | S | 25.9 |
| Teucrium chamaedrys | G | O | 100.0 |
| Teucrium chamaedrys | G | R | 48.0 |
| Thymus praecox subsp arcticus | G | R | 73.1 |
| Thymus x citriodorus | G | O | 52.2 |
| Trichosanthes kirilowii | G | O | 35.9 |
| Trifolium hybridum | G | R | 76.0 |
| Trifolium incarnatum | G | R | 73.4 |
| Trifolium pannonicum | G | R | 24.8 |
| Trifolium repens | G | R | 48.5 |
| Triticosecale spp. | G | R | 48.5 |
| Triticum spelta | G | R | 22.9 |
| Tropaeolum majus | G | S | 23.4 |
| Urtica dioica | G | O | 96.4 |
| Vaccinium corymbosum | G | S | 60.7 |
| Vaccinium corymbosum | G | R | 61.4 |
| Vaccinum angustifolium | G | R | 54.7 |
| Vicia sativa | G | R | 68.8 |
| Vicia sativa | G | O | 31.5 |
| Vicia villosa | G | O | 100.0 |
| Vicia villosa | G | R | 35.5 |
| Vigna sesquipedalis | G | R | 23.0 |
| Vitia spp | G | R | 36.9 |
| Withania somnifera | G | O | 44.0 |
| Xanthium strumarium | G | R | 37.6 |
| Zea mays | G | O | 100.0 |
| Aconitum napellus | T | R | 100.0 |
| Agaricus bisporus | T | R | 58.9 |
| Agaricus bisporus | T | O | 100.0 |
| Allium ampeloprasum | T | R | 43.3 |
| Allium ascalonicum | T | R | 34.5 |
| Allium cepa | T | R | 53.5 |
| Allium cepa | T | O | 45.8 |
| Allium grande | T | R | 43.2 |
| Allium schoenoprasum | T | R | 47.1 |
| Allium Tuberosum | T | R | 74.6 |
| Allium Tuberosum | T | O | 33.6 |
| Aloe vera | T | R | 34.1 |
| Althaea officinalis | T | S | 47.8 |
| Amelanchier alnitolia | T | R | 59.1 |
| Ananas comosus | T | O | 100.0 |
| Anthemis nobilis | T | O | 22.7 |
| Anthriscus cerefolium | T | O | 56.8 |
| Apium graveolens | T | R | 29.8 |
| Aralia nudicaulis | T | O | 100.0 |
| Armoracia rusticana | T | O | 58.9 |
| Artemisia dracunculus | T | O | 100.0 |
| Asparagus officinalis | T | R | 25.2 |
| Atriplex hortensis | T | R | 44.7 |
| Bellis perennis | T | R | 58.1 |
| Beta vulgaris | T | R | 37.3 |
| Betula glandulosa | T | O | 23.5 |
| Boletus edulis | T | S | 64.2 |
| Brassica juncea | T | R | 35.6 |
| Brassica napus | T | O | 100.0 |
| Brassica oleracea | T | R | 33.2 |
| Brassica oleracea | T | O | 49.7 |
| Camellia sinensis | T | O | 24.7 |
| Camellia sinensis | T | R | 45.7 |
| Canna edulis | T | R | 26.2 |
| Carum carvi | T | O | 100.0 |
| Chaerophyllum bulbosum | T | R | 40.9 |
| Chrysanthemun coronarium (Chp suey) | T | R | 48.1 |
| Chrysanthenum coronarium | T | R | 29.9 |
| Chrysanthenum coronarium | T | R | 100.0 |
| Cichorium endivia | T | R | 20.5 |
| Cichorium endivia | T | R | 21.9 |
| Cichorium intybus | T | S | 50.6 |
| Cichorium intybus | T | R | 31.7 |
| Cichorium intybus | T | R | 52.9 |
| Citrullus lanatus | T | O | 100.0 |
| Citrus paradisi | T | O | 40.6 |
| Cocos nucifera | T | O | 27.2 |
| Cornus canadensis | T | S | 44.9 |
| Crithmum maritimum | T | R | 32.3 |
| Cucumis anguria | T | O | 22.6 |
| Cucurbita moschata | T | O | 33.5 |
| Cucurbita moschata (Early Butternut) | T | R | 32.3 |
| Cucurbita pepo | T | O | 89.0 |
| Cuminum cyminum | T | R | 54.3 |
| Curcuma zedoaria | T | S | 100.0 |
| Cymbopogon citratus | T | O | 42.6 |
| Datura metel | T | O | 24.8 |
| Datura metel | T | R | 25.5 |
| Dioscorea batatas | T | R | 100.0 |
| Dipsacus sativus | T | O | 85.0 |
| Dryopteris filix-mas | T | O | 46.4 |
| Erigeron canadensis | T | O | 100.0 |
| Eruca vesicaria | T | R | 30.9 |
| Erysimum perofskianum | T | O | 23.0 |
| Eschscholzia californica | T | O | 37.8 |
| Eschscholzia californica | T | R | 20.8 |
| Fagopyrum esculentum | T | O | 100.0 |
| Fagopyrum tartaricum | T | R | 78.5 |
| Foeniculum vulgare | T | O | 63.4 |
| Foeniculum vulgare | T | O | 27.2 |
| Forsythia x intermedia | T | S | 32.0 |
| Fragaria x ananassa | T | S | 33.0 |
| Galinsoga ciliata | T | R | 25.8 |
| Gaultheria procumbens | T | O | 46.8 |
| Hedeoma pulegioides | T | O | 73.6 |
| Helianthus tuberosus | T | O | 39.3 |
| Hordeum vulgare | T | O | 32.4 |
| Humulus lupulus | T | O | 21.1 |
| Hypericum henryi | T | R | 29.3 |
| Hypericum perforatum | T | R | 42.7 |
| Iberis amara | T | O | 29.5 |
| Ipomea aquatica | T | R | 22.9 |
| Lathyrus Sativus | T | R | 69.4 |
| Laurus nobilis | T | O | 70.2 |
| Lavandula latifolia | T | O | 100.0 |
| Lens culinaris subsp. Culinaris | T | O | 70.2 |
| Lepidium sativum | T | O | 100.0 |
| Levisticum officinale | T | O | 100.0 |
| Lolium multiflorum | T | O | 35.1 |
| Lunaria annua | T | O | 100.0 |
| Lycopersicon pimpinellifolium | T | R | 24.4 |
| Malus hupehensis | T | R | 73.1 |
| Malus sp. | T | R | 80.9 |
| Malva sylvestris | T | R | 34.7 |
| Malva sylvestris | T | O | 100.0 |
| Manihot esculenta | T | R | 33.0 |
| Melissa officinalis | T | O | 100.0 |
| Melissa officinalis | T | O | 100.0 |
| Mentha suaveolens | T | S | 39.7 |
| Nigella sativa | T | R | 58.9 |
| Nigella sativa | T | R | 100.0 |
| Ocimum Basilicum | T | R | 100.0 |
| Origanum majorana | T | O | 41.5 |
| Origanum vulgare | T | O | 29.8 |
| Origanum vulgare | T | R | 33.1 |
| Panax quinquefolius | T | R | 75.2 |
| Passiflora spp. | T | S | 32.0 |
| Pastinaca sativa | T | R | 20.8 |
| Perroselinum crispum | T | R | 55.4 |
| Petroselinum crispum | T | R | 76.1 |
| Petroselinum crispum | T | O | 24.1 |
| Peucedanum oreaselinum | T | O | 21.0 |
| Phacelia tanacetifolia | T | R | 48.6 |
| Phalaris canariensis | T | O | 56.4 |
| Phaseolus coccineus | T | R | 22.7 |
| Phaseolus mungo | T | R | 47.4 |
| Phaseolus vulgaris | T | R | 40.0 |
| Phaseolus vulgaris | T | O | 29.4 |
| Phoenix dactylifera | T | R | 46.3 |
| Physalis ixocarpa goldie ou pourpre | T | R | 28.9 |
| Phytolacca americana | T | O | 100.0 |
| Plectranthus sp. | T | O | 73.8 |
| Pleurotus spp. | T | O | 100.0 |
| Poa compressa | T | O | 22.3 |
| Poa pratensis | T | O | 73.1 |
| Populus Tremula | T | O | 100.0 |
| Prunella vulgaris | T | O | 38.0 |
| Psoralea corylifolia | T | S | 96.4 |
| Pteridium aquilinum | T | R | 100.0 |
| Raphanus raphanistrum | T | O | 100.0 |
| Raphanus sativus | T | R | 33.7 |
| Raphanus sativus | T | R | 28.0 |
| Raphanus sativus | T | O | 100.0 |
| Reseda luteola | T | S | 69.6 |
| Reseda odorata | T | O | 51.8 |
| Rheum officinale | T | O | 46.7 |
| Rheum officinale | T | S | 100.0 |
| Ribes nigrum | T | R | 30.0 |
| Ribes Sativum | T | R | 61.7 |
| Ribes Sylvestre | T | R | 75.4 |
| Ricinus communis | T | S | 100.0 |
| Rosmarinus officinalis | T | R | 29.0 |
| Rubus canadensis | T | R | 86.1 |
| Sabal serrulata | T | R | 100.0 |
| Salvia officinalis | T | O | 100.0 |
| Sambucus canadensis | T | O | 24.8 |
| Satureja montana | T | R | 100.0 |
| Satureja repandra | T | S | 27.2 |
| Satureja repandra | T | O | 36.4 |
| Satureja repandra | T | R | 42.0 |
| Scrophularia nodosa | T | R | 68.8 |
| Secale cereale | T | O | 100.0 |
| Setaria italica | T | R | 23.2 |
| Silybum marianum | T | O | 73.5 |
| Solanum melongena | T | R | 20.1 |
| Solanum tuberosum | T | S | 24.4 |
| Solidago virgaurea | T | R | 71.4 |
| Sorghum dochna | T | O | 22.5 |
| Stachys byzantina | T | O | 39.2 |
| Stellaria media | T | O | 43.3 |
| Symphytum officinale | T | O | 58.7 |
| Tanacetum parthenium | T | O | 100.0 |
| Tanacetum vulgare | T | O | 32.5 |
| Taraxacum officinale | T | S | 27.8 |
| Teucrium chamaedrys | T | R | 62.9 |
| Teucrium chamaedrys | T | O | 100.0 |
| Thalpsi arvense | T | O | 21.2 |
| Thymus praecox subsp arcticus | T | R | 60.9 |
| Tragopogon porrifolium | T | R | 24.6 |
| Trifolium incarnatum | T | R | 33.7 |
| Trifolium pannonicum | T | R | 72.4 |
| Trifolium repens | T | R | 72.4 |
| Triticosecale spp. | T | R | 33.7 |
| Tropaeolum majus | T | R | 100.0 |
| Tropaeolum majus | T | O | 31.5 |
| Vaccinium angustifolium | T | O | 100.0 |
| Vaccinium angustifolium | T | S | 42.1 |
| Vaccinium macrocarpon | T | S | 30.9 |
| Vicia villosa | T | R | 35.5 |
| Vigna sesquipedalis | T | R | 24.0 |
| Vigna unguiculata | T | R | 31.6 |
| Vinca minor | T | O | 28.7 |
| Withania somnifera | T | O | 26.9 |
| Xanthium strumarium | T | O | 30.9 |
| Zea mays | T | R | 20.1 |
| Zea mays | T | O | 32.2 |

TABLE 2
inhibition of MMP-2 by Plant Extracts

| | | | Inhibition |
|---|---|---|---|
| Latin name | Stress | Extract | (%) |
| Achillea millefolium | A | S | 21.9 |
| Achillea millefolium | A | O | 63.0 |
| Achillea millefolium | A | R | 100.0 |
| Aconitum napellus | A | R | 71.0 |
| Alcea rosea | A | R | 67.9 |
| Alchemilla mollis | A | O | 64.4 |
| Allium ascalonicum | A | R | 20.9 |
| Allium cepa | A | R | 84.3 |
| Allium grande | A | R | 36.7 |
| Allium porrum | A | O | 100.0 |
| Allium porum | A | S | 51.9 |
| Allium porum | A | R | 66.7 |
| Allium sativum | A | R | 100.0 |
| Allium schoenoprasum | A | R | 73.5 |
| Allium Tuberosum | A | S | 24.3 |
| Allium Tuberosum | A | O | 83.6 |
| Allium Tuberosum | A | R | 89.3 |
| Aloe vera | A | R | 69.7 |
| Althaea officinalis | A | S | 27.6 |
| Althaea officinalis | A | R | 64.7 |
| Amaranthus gangeticus | A | S | 29.4 |
| Anethum graveolens | A | O | 100.0 |
| Apium graveolens | A | S | 25.1 |
| Apium graveolens | A | R | 52.1 |
| Aralia cordata | A | S | 66.4 |
| Aralia cordata | A | R | 92.2 |
| Aralia nudicaulis | A | O | 29.4 |
| Arctium minus | A | S | 28.4 |
| Armoracia rusticana | A | S | 20.2 |
| Armoracia rusticana | A | O | 55.0 |
| Arrhenatherum elatius | A | S | 40.2 |
| Artemisia dracunculus | A | S | 39.7 |
| Asparagus officinalis | A | S | 29.3 |
| Atriplex hortensis | A | R | 33.6 |
| Avena sativa | A | R | 37.2 |
| Beta vulgaris | A | S | 45.4 |
| Beta vulgaris | A | R | 95.9 |
| Beta vulgaris spp. Maritima | A | R | 100.0 |
| Brassica chinensis | A | R | 49.6 |
| Brassica napus | A | O | 28.5 |
| Brassica napus | A | S | 52.4 |
| Brassica napus | A | R | 82.4 |
| Brassica nigra | A | O | 29.2 |
| Brassica oleracea | A | R | 31.2 |
| Brassica oleracea | A | R | 31.4 |
| Brassica oleracea | A | R | 64.0 |
| Brassica oleracea | A | S | 68.7 |
| Brassica oleracea | A | R | 75.3 |
| Brassica oleracea | A | O | 100.0 |
| Brassica rapa | A | S | 27.6 |
| Brassica rapa | A | R | 33.4 |
| Brassica rapa | A | O | 57.6 |
| Brassica rapa | A | R | 58.1 |
| Brassica rapa | A | R | 84.5 |
| Calamintha nepeta | A | O | 65.0 |
| Camellia sinensis | A | S | 21.9 |
| Camellia sinensis | A | R | 26.5 |
| Camellia sinensis | A | O | 79.0 |
| Cana edulis | A | R | 45.5 |
| Canna edulis | A | S | 20.2 |
| Capsella bursa-pastoris | A | S | 35.5 |
| capsicum annuum | A | S | 61.5 |
| capsicum annuum | A | O | 89.8 |
| capsicum annuum | A | R | 100.0 |
| Capsicum frutescens | A | S | 66.6 |
| Capsicum frutescens | A | R | 100.0 |
| Carthamus tinctorius | A | R | 21.3 |
| Carthamus tinctorius | A | R | 21.5 |
| Chaerophyllum bulbosom | A | R | 57.2 |
| Chelidonium majus | A | S | 34.4 |
| Chenopodium bonus-henricus | A | R | 43.5 |
| Chenopodium bonus-henricus | A | O | 100.0 |
| Chenopodium bonus-henricus | A | R | 76.4 |
| Chenopodium quinoa | A | O | 92.0 |
| Chrysanthemum coronarium | A | R | 48.6 |
| Chrysanthemum coronarium | A | O | 49.7 |
| Chrysanthemun coronarium | A | R | 47.3 |
| Chrysanthenum coronarium | A | R | 26.7 |
| Cicer arietinum | A | S | 22.0 |
| Ciccr arietinum | A | O | 23.6 |
| Cichorium intybus | A | S | 21.1 |
| Cichorium intybus | A | R | 100.0 |
| Citrullus lanatus | A | S | 65.5 |
| Citrullus lanatus | A | R | 96.3 |
| Citrullus lanatus | A | O | 100.0 |
| Coix Lacryma-Jobi | A | O | 32.2 |
| Cornus canadensis | A | S | 52.8 |
| Cosmos sulphureus | A | R | 72.5 |
| Crataegus spp | A | O | 100.0 |
| Cryptotaenia canadensis | A | R | 50.6 |
| Cryptotaenia canadensis | A | O | 51.3 |
| Cucumis anguria | A | S | 53.4 |
| Cucumis Anguria | A | R | 84.9 |
| Cucumis melo | A | R | 91.7 |
| Cucurbita Maxima | A | S | 34.9 |
| Cucurbita Maxima | A | R | 41.7 |
| Cucurbita moschata | A | R | 36.8 |
| Cucurbita moschata | A | S | 37.4 |
| Cucurbita pepo | A | S | 48.1 |
| Cucurbita pepo | A | R | 85.7 |
| Curcuma zedoaria | A | S | 21.0 |
| Curcuma zedoaria | A | R | 32.1 |
| Curcurbita maxima | A | S | 27.0 |
| Cymbopogon citratus | A | R | 34.5 |
| Cymbopogon citratus | A | O | 100.0 |
| Cymbopogon martinii | A | S | 47.4 |
| Dactylis glomerata | A | S | 20.6 |
| Dactylis glomerata | A | O | 75.0 |
| Daucus carota | A | S | 44.5 |
| Daucus carota | A | R | 70.5 |
| Dipsacus sativus | A | O | 40.4 |
| Dirca palustris | A | S | 27.2 |
| Dolichos Lablab | A | S | 54.2 |
| Dryopteris filix-mas | A | R | 76.3 |
| Echinacea purpurea | A | R | 42.9 |
| Eleusine coracana | A | S | 37.5 |
| Eleusine coracana | A | O | 100.0 |
| Erigeron canadensis | A | O | 45.7 |
| Eruca vesicaria | A | R | 80.2 |
| Eschscholzia californica | A | S | 42.4 |
| Eschscholzia californica | A | O | 75.0 |
| Eschscholzia californica | A | R | 88.8 |
| Fagopyrum esculentum | A | O | 100.0 |
| Fagopyrum tartaricum | A | R | 38.6 |
| Fagopyrum tartaricum | A | S | 40.3 |
| Fagopyrum tartaricum | A | O | 71.0 |
| Filipendula rubra | A | R | 36.3 |
| Foeniculum vulgare | A | R | 41.6 |
| Foeniculum vulgare | A | S | 84.4 |
| Foeniculum vulgare | A | O | 100.0 |
| Forsythia intermedia | A | R | 35.8 |
| Fragaria x ananassa | A | R | 97.2 |
| Galinsoga ciliata | A | R | 54.0 |
| Galium odoratum | A | O | 34.3 |
| Galium odoratum | A | O | 100.0 |
| Gaultheria hispidula | A | S | 35.8 |
| Gaultheria hispidula | A | R | 100.0 |
| Glaux maritima | A | R | 46.5 |
| Glycine max | A | S | 27.0 |
| Glycine Max | A | R | 43.1 |
| Glycine max | A | O | 100.0 |
| Guizotia abyssinica | A | S | 29.8 |
| Guizotia abyssinica | A | R | 32.5 |
| Hamamelis virginiana | A | R | 75.7 |
| Helianthus annuus | A | R | 69.0 |
| Helianthus Tuberosus | A | R | 22.2 |
| Helianthus tuberosus | A | R | 69.7 |
| Helianthus Tuberosus | A | O | 100.0 |
| Hordeum hexastichon | A | R | 22.3 |
| Hordeum hexastichon | A | R | 34.9 |
| Hordeum hexastichon | A | O | 86.9 |
| Hordeum vulgare | A | O | 74.8 |
| Hordeum vulgare subsp. Vulgare | A | S | 34.5 |
| Hordeum vulgare subsp. Vulgare | A | O | 74.2 |
| Hyssopus officinalis | A | O | 57.5 |
| Inula helenium | A | S | 26.8 |
| Ipomoea Batatas | A | S | 20.1 |
| Lathyrus sativus | A | S | 28.7 |
| Lathyrus sativus | A | 0 | 100.0 |
| Lathyrus sylvestris | A | R | 42.4 |
| Lavandula latifolia | A | O | 39.1 |
| Lepidium sativum | A | O | 20.1 |
| Lepidium sativum | A | S | 49.0 |
| Levisticum officinale | A | S | 23.0 |
| Levisticum officinale | A | O | 29.8 |
| Linum usitatissimum | A | R | 56.9 |
| Lolium multiflorum | A | S | 41.5 |
| Lolium multiflorum | A | O | 92.3 |
| Lotus corniculatus | A | O | 95.5 |
| Lotus tetragonolobus | A | R | 76.7 |
| Lycopersicon esculentum | A | S | 35.3 |
| Lycopersicon esculentum | A | R | 78.1 |
| Lycopersicon esculentum | A | R | 85.6 |
| Lycopersicon pimpinollifolium | A | R | 74.9 |
| Malva moschata | A | S | 21.5 |
| Malva moschata | A | O | 44.5 |
| Malva verticillata | A | R | 22.0 |
| Matricaria recutita | A | S | 40.9 |
| Matricaria recutita | A | O | 67.3 |
| Melaleuca alternifolia | A | O | 65.0 |
| Melilotus albus | A | S | 50.7 |
| Melilotus albus | A | O | 100.0 |
| Melissa officinalis | A | O | 42.4 |
| Mentha pulegium | A | O | 88.3 |
| Mentha spicata | A | O | 94.8 |
| Mentha suaveolens | A | O | 82.9 |
| Nepeta cataria | A | O | 100.0 |
| Nicotiana rustica | A | S | 24.0 |
| Nicotiana rustica | A | R | 100.0 |
| Nicotiana tabacum | A | S | 42.5 |
| Nicotiana tabacum | A | R | 61.1 |
| Nigella sativa | A | R | 81.7 |
| Ocimum tenuiflorum | A | R | 23.1 |
| Oenothera biennis | A | R | 28.6 |
| Origanum majorana | A | O | 52.9 |
| Origanum majorana | A | R | 100.0 |
| Origanum vulgare | A | O | 66.8 |
| Panax quinquefolius | A | S | 31.8 |
| Pastinaca sativa | A | S | 27.7 |
| Pastinaca sativa | A | R | 33.8 |
| Petasites japonicus | A | S | 26.2 |
| Petroselinum crispum | A | R | 69.1 |
| Phalaris canariensis | A | S | 28.4 |
| Phalaris canariensis | A | R | 29.7 |
| Phalaris canariensis | A | O | 94.3 |
| Phaseolus coccineus | A | S | 30.8 |
| Phaseolus coccineus | A | R | 79.5 |
| Phaseolus coccineus | A | O | 80.9 |
| Phaseolus mungo | A | R | 59.8 |
| Phaseolus vulgaris | A | S | 47.3 |
| Phaseolus Vulgaris | A | R | 74.4 |
| Phaseolus vulgaris | A | R | 83.2 |
| Phaseolus Vulgaris | A | O | 100.0 |
| Phlox paniculata | A | O | 23.7 |
| Phlox paniculata | A | R | 81.7 |
| Physalis alkekengi | A | R | 23.5 |
| Physalis Ixocarpa | A | O | 85.8 |
| Physalis ixocarpa | A | R | 91.5 |
| Physalis Pruinosa | A | R | 25.7 |
| Physalis Pruinosa | A | O | 83.5 |
| Phytolacca decandra | A | O | 31.5 |
| Phytolacca decandra | A | S | 38.5 |
| Pimpinella anisum | A | S | 100.0 |
| Pimpinella anisum | A | R | 100.0 |
| Plantago coronopus | A | R | 36.0 |
| Plantago coronopus | A | R | 38.4 |
| Plantago coronopus | A | O | 53.6 |
| Plantago major | A | R | 65.3 |
| Plectranthus sp. | A | O | 74.2 |
| Poa compressa | A | S | 37.3 |
| Poa compressa | A | R | 49.8 |
| Poa compressa | A | O | 100.0 |
| Polygonum pensylvanicum | A | R | 63.5 |
| Polygonum pensylvanicum | A | O | 72.9 |
| Polygonum persicaria | A | S | 27.5 |
| Polygonum persicaria | A | O | 43.0 |
| Poterium sanguisorba | A | R | 100.0 |
| Poterium Sanquisorba | A | O | 84.2 |
| Pteridium aquilinum | A | O | 45.1 |
| Pteridium aquilinum | A | R | 100.0 |
| Pysalis ixocarpa | A | R | 87.3 |
| Raphanus raphanistrum | A | S | 32.2 |
| Raphanus sativus | A | R | 25.3 |
| Raphanus sativus | A | S | 47.5 |
| Raphanus sativus | A | R | 83.5 |
| Raphanus sativus | A | R | 84.7 |
| Raphanus Sativus | A | O | 100.0 |
| Rheum officinale | A | O | 44.0 |
| Ribes nigrum | A | O | 100.0 |
| Ribes nigrum | A | R | 100.0 |
| Ricinus communis | A | O | 100.0 |
| Rosa rugosa | A | R | 25.2 |
| Rosa rugosa | A | S | 26.6 |
| Rosa rugosa | A | O | 83.2 |
| Rosmarinus officinalis | A | R | 68.2 |
| Rubus idaeus | A | O | 81.9 |
| Rubus ideaus | A | R | 73.4 |
| Rumex Acetosa | A | S | 24.2 |
| Rumex Acetosa | A | R | 85.5 |
| Rumex Acetosa | A | O | 100.0 |
| Rumex crispus | A | 0 | 46.7 |
| Rumex crispus | A | R | 100.0 |
| Ruta graveolens | A | O | 100.0 |
| Saccharum officinarum | A | R | 80.8 |
| Salix purpurea | A | S | 56.7 |
| Salvia officinalis | A | S | 24.1 |
| Salvia officinalis | A | O | 91.8 |
| Salvia sclarea | A | O | 99.7 |
| Santolina chamaecyparissus | A | O | 83.8 |
| Satureja hortensis | A | O | 79.1 |
| Satureja hortensis | A | R | 100.0 |
| Satureja montana | A | R | 60.4 |
| Satureja montana | A | O | 76.1 |
| Scorzonera hispanica | A | S | 22.1 |
| Secale cereale | A | R | 47.2 |
| Secale cereale | A | O | 67.2 |
| Senecio vulgaris | A | S | 23.2 |
| Senecio vulgaris | A | R | 76.6 |
| Sesamum indicum | A | R | 100.0 |
| Sesamum indicum | A | S | 100.0 |
| Solanum dulcamara | A | R | 54.5 |
| Solanum melanocerasum | A | S | 45.4 |
| Solanum melanocerasum | A | R | 85.2 |
| Solanum melanocerasum | A | O | 88.7 |
| Solanum melongena | A | S | 42.5 |
| Solanum melongena | A | R | 85.9 |
| Sonchus oleraceus | A | R | 25.6 |
| Sorghum caffrorum | A | R | 39.6 |
| Sorghum dochna | A | S | 30.0 |
| Sorghum dochna | A | R | 48.0 |
| Sorghum dochna | A | O | 62.0 |
| Sorghum durra | A | R | 72.1 |
| Sorghum durra | A | O | 94.6 |
| Sorghum sudanense | A | O | 100.0 |
| Spinacia oleracea | A | S | 23.6 |
| Stachys affinis | A | R | 74.4 |
| Stachys byzantina | A | R | 48.4 |
| Stachys byzantina | A | O | 100.0 |
| Stellaria graminea | A | S | 20.8 |
| Stellaria graminea | A | R | 37.5 |
| Stellaria media | A | R | 49.0 |
| Stellaria media | A | S | 50.7 |
| Symphytum officinale | A | R | 44.2 |
| Tanacetum cinerariifolium | A | R | 100.0 |
| Tanacetum parthenium | A | S | 30.4 |
| Tanacetum vulgare | A | S | 28.6 |
| Tanacetum vulgare | A | R | 100.0 |
| Taraxacum officinale | A | R | 59.1 |
| Thymus praecox subsp arcticus | A | R | 43.5 |
| Thymus vulgaris | A | S | 30.1 |
| Thymus x citriodorus | A | R | 100.0 |
| Trichosanthes kirilowii | A | S | 29.2 |
| Trichosanthes kirilowii | A | O | 42.1 |
| Trigonella foenumgraecum | A | O | 53.4 |
| Triticosecal spp. | A | R | 44.8 |
| Triticum aestivum | A | R | 65.5 |
| Triticum durum | A | O | 53.9 |
| Triticum spelta | A | R | 26.4 |
| Triticum spelta | A | S | 36.7 |
| Triticum spelta | A | O | 51.9 |
| Tropaeolum majus | A | R | 25.8 |
| Urtica dioica | A | O | 22.9 |
| Urtica dioica | A | S | 30.6 |
| Vaccinium Corymbosum | A | R | 100.0 |
| Veratrum viride | A | R | 33.2 |
| Verbascum thapsus | A | S | 22.9 |
| Veronica beccabunga | A | R | 52.8 |
| Veronica officinalis | A | R | 84.2 |
| Vicia sativa | A | R | 100.0 |
| Vicia villosa | A | S | 32.9 |
| Vicia villosa | A | R | 100.0 |
| Vigna angularis | A | R | 54.0 |
| Vigna sesquipedalis | A | S | 48.3 |
| Vigna sesquipedalis | A | R | 73.0 |
| Vigna sesquipedalis | A | O | 96.6 |
| Vigna unguiculata | A | R | 70.7 |
| Vinca minor | A | S | 22.1 |
| Vinca minor | A | R | 88.4 |
| Vitis sp. | A | S | 20.9 |
| Vitis sp. | A | R | 30.4 |
| Xanthium sibiricum | A | S | 39.2 |
| Xanthium sibiricum | A | R | 47.8 |
| Xanthium sibiricum | A | O | 70.1 |
| Zea mays | A | R | 100.0 |
| Zea Mays | A | O | 100.0 |
| Abelmochus esculentus | G | S | 21.6 |
| Abelmochus esculentus | G | R | 79.3 |
| Achillea millefolium | G | O | 62.7 |
| Aconitum napellus | G | O | 82.0 |
| Acorus calamus | G | S | 100.0 |
| Ageratum conyzoides | G | S | 49.3 |
| Alcea rosea | G | R | 64.4 |
| Alchemilla mollis | G | S | 21.5 |
| Alchemilla mollis | G | R | 30.2 |
| Alchemilla mollis | G | O | 55.7 |
| Allium ampeloprasum | G | O | 36.1 |
| Allium ampeloprasum | G | R | 52.8 |
| Allium ascalonicum | G | O | 68.9 |
| Allium cepa | G | S | 40.2 |
| Allium cepa | G | R | 66.4 |
| Allium cepa | G | O | 100.0 |
| Allium grande | G | R | 36.4 |
| AAllium sativum | G | S | 29.5 |
| AAllium sativum | G | R | 68.4 |
| AAllium sativum | G | O | 100.0 |
| Allium schoenoprasum | G | S | 47.1 |
| Allium schoenoprasum | G | R | 61.7 |
| Allium Tuberosum | G | S | 23.8 |
| Allium Tuberosum | G | O | 54.5 |
| Allium Tuberosum | G | R | 85.9 |
| Aloe vera | G | R | 53.6 |
| Althaea officinalis | G | S | 37.4 |
| Altheaa officinalis | G | S | 42.4 |
| Amaranthus caudathus | G | S | 30.9 |
| Amaranthus caudathus | G | O | 56.7 |
| Amaranthus gangeticus | G | S | 23.1 |
| Anethum graveolens | G | S | 23.9 |
| Angelica archangelica | G | S | 22.0 |
| Angelica archangelica | G | S | 24.9 |
| Apium graveolens | G | O | 33.0 |
| Apium graveolens | G | R | 44.8 |
| Apium graveolens | G | S | 54.1 |
| Apium graveolens | G | R | 84.1 |
| Aralia nudicaulis | G | R | 51.8 |
| Arctium minus | G | S | 25.4 |
| Armoracia rusticana | G | O | 52.1 |
| Aronia melanocarpa | G | S | 22.5 |
| Aronia melanocarpa | G | R | 82.3 |
| Artemisia dracunculus | G | R | 53.6 |
| Artemisia dracunculus | G | R | 58.8 |
| Artemisia dracunculus | G | S | 100.0 |
| Artemisia dracunculus | G | O | 100.0 |
| Asclepias incarnata | G | S | 26.9 |
| Asparagus officinalis | G | S | 24.0 |
| Asparagus officinalis | G | R | 65.9 |
| Asparagus officinalis | G | O | 95.0 |
| Aster spp | G | O | 48.4 |
| Beckmannia eruciformis | G | O | 24.8 |
| Bellis perennis | G | O | 52.6 |
| Beta vulgaris | G | S | 45.3 |
| Beta vulgaris | G | R | 100.0 |
| Beta vulgaris spp. Maritima | G | R | 100.0 |
| Brassica cepticepa | G | R | 52.9 |
| Brassica chinensis | G | R | 41.9 |
| Brassica juncea | G | R | 22.8 |
| Brassica napus | G | S | 22.9 |
| Brassica oleracea | G | R | 45.5 |
| Brassica oleracea | G | R | 47.1 |
| Brassica oleracea | G | S | 62.9 |
| Brassica oleracea | G | R | 77.9 |
| Brassica oleracea | G | O | 100.0 |
| Brassica rapa | G | S | 26.5 |
| Brassica rapa | G | R | 38.9 |
| Brassica rapa | G | R | 53.6 |
| Calamintha nepeta | G | S | 20.4 |
| Calamintha nepeta | G | O | 78.0 |
| Camellia sinensis | G | O | 100.0 |
| Campanula rapunculus | G | R | 60.6 |
| Canna edulis | G | O | 78.1 |
| Capsella bursa-pastoris | G | S | 30.7 |
| Capsella bursa-pastoris | G | R | 60.6 |
| capsicum annuum | G | S | 70.8 |
| capsicum annuum | G | O | 80.0 |
| capsicum annuum | G | R | 100.0 |
| Capsicum frutescens | G | S | 63.2 |
| Capsicum frutescens | G | R | 100.0 |
| Carthamus tinctorius | G | R | 100.0 |
| Centaurea solstitialis | G | S | 46.4 |
| Cerastium tomentosum | G | R | 52.3 |
| Chenopodium bonus-henricus | G | S | 22.0 |
| Chenopodium quinoa | G | S | 31.0 |
| Chenopodium quinoa | G | O | 53.4 |
| Chrysanthemun coronarium | G | R | 76.2 |
| Chrysanthenum coronarium | G | R | 54.2 |
| Cicer arietinum | G | S | 23.1 |
| Cichorium endivia subsp endivia | G | S | 28.7 |
| Cichorium endivia subsp endivia | G | O | 68.7 |
| Cichorium intybus | G | S | 41.4 |
| Cichorium intybus | G | O | 62.1 |
| Circium arvense | G | S | 25.3 |
| Circium arvense | G | R | 59.3 |
| Citrullus lanatus | G | S | 24.8 |
| Citrullus lanatus | G | R | 41.1 |
| Citrullus lanatus | G | R | 100.0 |
| Cosmos sulphureus | G | R | 77.9 |
| Cosmos sulphureus | G | S | 79.4 |
| Cucumis sativus | G | S | 39.9 |
| Cucumis sativus | G | S | 39.9 |
| Cucurbita maxima | G | S | 33.9 |
| Cucurbita maxima | G | R | 43.4 |
| Cucurbita maxima | G | O | 100.0 |
| Cucurbita moschata | G | S | 41.3 |
| Cucurbita pepo | G | S | 42.8 |
| Cucurbita pepo | G | S | 45.4 |
| Cucurbita Pepo | G | R | 83.0 |
| Cuminum cyminum | G | O | 66.2 |
| Curcuma zedoaria | G | R | 33.9 |
| Cymbopogon citratus | G | R | 65.8 |
| Cymbopogon martinii motia | G | S | 41.4 |
| Cymbopogon martinii motia | G | O | 60.5 |
| Dactylis glomerata | G | S | 21.9 |
| Dactylis glomerata | G | O | 61.2 |
| Datura stramonium | G | S | 27.0 |
| Daucus carota | G | O | 21.3 |
| Daucus carota | G | S | 31.0 |
| Daucus carota | G | R | 100.0 |
| Digitalis purpurea | G | S | 30.9 |
| Dipsacus sativus | G | O | 63.6 |
| Dirca palustris | G | O | 23.1 |
| Dolichos Lablab | G | S | 33.0 |
| Dryopteris filix-mas | G | R | 100.0 |
| Echinacea purpurea | G | R | 93.4 |
| Eleusine coracana | G | S | 30.0 |
| Erigeron speciosus | G | S | 28.9 |
| Errhenatherum elatius | G | S | 55.6 |
| Eruca vesicaria | G | R | 54.7 |
| Eschscholzia californica | G | S | 47.9 |
| Eschscholzia californica | G | O | 75.9 |
| Fagopyrum tartaricum | G | O | 41.1 |
| Filipendula rubra | G | R | 38.5 |
| Foeniculum vulgare | G | R | 70.0 |
| Foeniculum Vulgare | G | S | 100.0 |
| Galinsoga ciliata | G | S | 34.6 |
| Galinsoga ciliata | G | R | 48.2 |
| Gaultheria hispidula | G | R | 60.5 |
| Gaultheria hispidula | G | O | 100.0 |
| Gaultheria hispidula | G | S | 100.0 |
| Glaux maritima | G | R | 59.3 |
| Glycine max | G | R | 21.1 |
| Glycine max | G | S | 24.4 |
| Glycine max | G | O | 28.1 |
| Guizotia abyssinica | G | S | 26.0 |
| Guizotia abyssinica | G | R | 36.8 |
| Guizotia abyssinica | G | O | 100.0 |
| Hedeoma pulegioides | G | O | 94.6 |
| Helianthus annuus | G | S | 35.5 |
| Helianthus annuus | G | O | 75.0 |
| Helianthus annuus | G | R | 79.9 |
| Helianthus strumosus | G | O | 100.0 |
| Helianthus tuberosus | G | R | 64.2 |
| Helichrysum thianschanicum | G | O | 61.1 |
| Helleborus niger | G | R | 48.0 |
| Hordeum hexastichon | G | S | 26.8 |
| Hordeum vulgare | G | O | 65.4 |
| Hordeum vulgare subsp. Vulgare | G | O | 75.8 |
| Humulus lupulus | G | S | 26.0 |
| Hypericum henryi | G | R | 20.2 |
| Hypericum henryi | G | O | 71.1 |
| Hyssopus officinalis | G | O | 100.0 |
| Iberis amara | G | S | 21.2 |
| Inula helenium | G | S | 24.3 |
| Lactuca sativa | G | R | 100.0 |
| Lactuca serriola | G | R | 69.3 |
| Laportea canadensis | G | R | 100.0 |
| Lathyrus sylvestris | G | O | 39.6 |
| Lavandula angustifolia | G | O | 70.0 |
| Lavandula latifolia | G | S | 22.7 |
| Lepidium Sativum | G | R | 30.6 |
| Lepidium sativum | G | S | 53.3 |
| Levisticum officinale | G | O | 80.7 |
| Lolium multiflorum | G | O | 34.5 |
| Lotus corniculatus | G | S | 32.9 |
| Lotus corniculatus | G | O | 100.0 |
| Lotus tetragonolobus | G | R | 79.9 |
| Lycopersicon esculentum | G | S | 28.2 |
| Lycopersicon esculentum | G | R | 75.4 |
| Lycopersicon pimpinellifolium | G | R | 81.4 |
| Malus hupehensis | G | R | 32.5 |
| Malus hupehensis | G | S | 41.2 |
| Malva moschata | G | O | 47.1 |
| Malva sylvestris | G | S | 23.1 |
| Malva verticillata | G | R | 39.9 |
| Matricaria recutita | G | O | 30.0 |
| Matricaria recutita | G | S | 71.3 |
| Melaleuca alternifolia | G | O | 58.3 |
| Melilotus alba | G | S | 41.1 |
| Melilotus albus | G | O | 88.8 |
| Melilotus albus | G | R | 100.0 |
| Melissa officinalis | G | O | 47.8 |
| Mentha arvensis | G | R | 33.9 |
| Mentha arvensis | G | O | 63.3 |
| Mentha piperita | G | S | 32.3 |
| Mentha piperita | G | O | 85.9 |
| Mentha piperita | G | R | 100.0 |
| Mentha spicata | G | S | 28.9 |
| Mentha spicata | G | R | 37.5 |
| Mentha suaveolens | G | R | 25.6 |
| Mentha suaveolens | G | O | 70.3 |
| Momordica charantia | G | R | 52.9 |
| Monarda didyma | G | S | 22.0 |
| Monarda didyma | G | O | 100.0 |
| Monarda fistulosa | G | O | 26.0 |
| Nepeta cataria | G | S | 23.4 |
| Nicotiana tabacum | G | S | 45.2 |
| Nigella sativa | G | R | 94.7 |
| Ocimum basilicum | G | S | 23.0 |
| Ocimum basilicum | G | O | 100.0 |
| Ocimum tenuiflorum | G | R | 45.3 |
| Oerothera biennis | G | R | 54.3 |
| Origanum majorana | G | O | 100.0 |
| Origanum majorana | G | R | 100.0 |
| Origanum vulgare | G | R | 93.3 |
| Origanum vulgare | G | O | 93.5 |
| Origanum vulgare | G | S | 97.4 |
| Oxalis Deppei | G | S | 28.7 |
| Oxalis Deppei | G | R | 87.2 |
| Oxalis Deppei | G | O | 100.0 |
| Oxyria digyna | G | R | 54.5 |
| Panicum miliaceum | G | O | 71.1 |
| Panicum miliaceum | G | R | 100.0 |
| Panicum miliaceum | G | S | 100.0 |
| Passiflora caerula | G | S | 26.3 |
| Passiflora caerula | G | R | 72.1 |
| Pastinaca sativa | G | S | 24.3 |
| Pastinaca sativa | G | R | 90.2 |
| Petroselinum crispum | G | R | 87.6 |
| Petroselinum crispum | G | O | 100.0 |
| Phalaris canariensis | G | R | 100.0 |
| Phalaris canariensis | G | O | 100.0 |
| Phaseolus acutifolius | G | R | 79.6 |
| Phaseolus coccineus | G | S | 28.3 |
| Phaseolus coccineus | G | R | 80.4 |
| Phaseolus mungo | G | R | 37.2 |
| Phaseolus vulgaris | G | R | 54.3 |
| Phaseolus vulgaris | G | S | 59.0 |
| Phaseolus vulgaris | G | O | 73.7 |
| Phaseolus vulgaris | G | R | 100.0 |
| Phlox paniculata | G | R | 37.7 |
| Phlox paniculata | G | O | 77.0 |
| Phlox paniculata | G | R | 80.8 |
| Physalis ixocarpa | G | S | 30.5 |
| Physalis ixocarpa | G | R | 78.3 |
| Physalis ixocarpa | G | R | 80.9 |
| Physalis pruinosa | G | O | 63.2 |
| Phytolacca americana | G | S | 36.1 |
| Phytolacca americana | G | O | 100.0 |
| Pimpinella anisum | G | S | 26.1 |
| Pimpinella anisum | G | R | 30.0 |
| Pisum sativum | G | S | 28.4 |
| Plantago coronopus | G | R | 27.8 |
| Plantago coronopus | G | O | 51.1 |
| Plantago coronopus | G | R | 67.5 |
| Plantago major | G | S | 30.3 |
| Plantago major | G | O | 64.6 |
| Poa compressa | G | O | 63.0 |
| Poa compressa | G | S | 67.4 |
| Poa compressa | G | R | 89.0 |
| Poa pratensis | G | S | 28.2 |
| Polygonum aviculare | G | R | 100.0 |
| Polygonum pensylvanicum | G | S | 27.7 |
| Polygonum pensylvanicum | G | O | 54.1 |
| Polygonum persicaria | G | S | 32.0 |
| Polygonum persicaria | G | O | 35.7 |
| Polygonum persicaria | G | R | 100.0 |
| Portulaca oleracera | G | R | 51.5 |
| Poterium sanguisorba | G | O | 89.9 |
| Poterium sanguisorba | G | R | 100.0 |
| Poterium sanquisorba | G | S | 23.7 |
| Prunella vulgaris | G | S | 26.7 |
| Prunus cerasifera | G | R | 95.3 |
| Raphanus Raphanistrum | G | R | 41.7 |
| Raphanus Raphanistrum | G | S | 43.5 |
| Raphanus sativus | G | R | 41.0 |
| Raphanus sativus | G | S | 44.6 |
| Raphanus sativus | G | R | 50.5 |
| Raphanus sativus | G | R | 86.1 |
| Raphanus sativus | G | O | 100.0 |
| Reseda odorata | G | O | 58.3 |
| Rheum officinale | G | O | 30.7 |
| Ribes nigrum | G | O | 54.3 |
| Ribes nigrum | G | R | 63.8 |
| Ribes Sylvestre | G | R | 100.0 |
| Ricinus communis | G | R | 41.5 |
| Ricinus communis | G | O | 100.0 |
| Rosmarinus officinalis | G | R | 90.0 |
| Rubus idaeus | G | S | 37.1 |
| Rubus ideaus | G | R | 26.6 |
| Rubus occidentalis | G | R | 35.1 |
| Rumex crispus | G | R | 30.3 |
| Rumex crispus | G | S | 100.0 |
| Rumex patientia | G | R | 41.0 |
| Rumex patientia | G | S | 41.9 |
| Ruta graveolens | G | S | 47.9 |
| Ruta graveolens | G | R | 82.1 |
| Saccharum officinarum | G | R | 100.0 |
| Salvia elegens | G | O | 100.0 |
| Salvia officinalis | G | S | 35.3 |
| Salvia officinalis | G | O | 100.0 |
| Salvia officinalis | G | R | 100.0 |
| Sambucus ebulus | G | R | 53.9 |
| Santolina chamaecyparissus | G | S | 36.4 |
| Santolina chamaecyparissus | G | 0 | 69.5 |
| Santolina chamaecyparissus | G | R | 100.0 |
| Saponaria officinalis | G | S | 29.8 |
| Satureja hortensis | G | O | 97.4 |
| Satureja hortensis | G | R | 100.0 |
| Satureja montana | G | O | 59.2 |
| Satureja repandra | G | S | 35.3 |
| Satureja repandra | G | O | 66.2 |
| Scorzonera hispanica | G | S | 24.5 |
| Scrophularia nodosa | G | S | 24.5 |
| Scrophularia nodosa | G | O | 30.0 |
| Scrophularia nodosa | G | R | 55.6 |
| Scutellaria lateriflora | G | S | 20.3 |
| Scutellaria lateriflora | G | R | 83.1 |
| Secale cereale | G | O | 51.1 |
| Senecio vulgaris | G | R | 42.5 |
| Sesamum indicum | G | S | 34.3 |
| Sesamum indicum | G | R | 44.5 |
| Silene vulgaris | G | S | 34.1 |
| Sium sisarum | G | O | 100.0 |
| Solanum melanocerasum | G | S | 40.6 |
| Solanum melanocerasum | G | R | 85.4 |
| solanum melongena | G | S | 58.2 |
| solanum melongena | G | O | 83.0 |
| solanum melongena | G | R | 85.6 |
| Solanum tuberosum | G | O | 40.2 |
| Sonchus oleraceus | G | R | 41.1 |
| Sorghum dochna | G | S | 25.0 |
| Sorghum dochna | G | O | 64.3 |
| Sorghum dochna | G | R | 100.0 |
| sorghum durra | G | R | 60.1 |
| Sorghum durra | G | O | 100.0 |
| Sorghum sudanense | G | O | 98.0 |
| Spinacia oleracea | G | S | 24.9 |
| Spinacia oleracea | G | O | 100.0 |
| Stachys byzantina | G | R | 78.8 |
| Stellaria graminea | G | S | 29.3 |
| Stellaria media | G | S | 33.4 |
| Stellaria media | G | R | 45.4 |
| Symphytum officinale | G | O | 57.5 |
| Tanacetum cinerariifolium | G | R | 100.0 |
| Tanacetum parthenium | G | R | 28.2 |
| Tanacetum vulgare | G | S | 25.2 |
| Tanacetum vulgare | G | R | 39.3 |
| Tanacetum vulgare | G | O | 81.2 |
| Taraxacum officinale | G | R | 51.1 |
| Thymus fragantissimus | G | S | 29.9 |
| Thymus fragantissimus | G | O | 55.3 |
| Thymus praecox subsp arcticus | G | S | 27.7 |
| Thymus serpyllum | G | R | 74.9 |
| Thymus vulgaris | G | S | 23.3 |
| Thymus vulgaris | G | R | 86.4 |
| Thymus x citriodorus | G | R | 97.6 |
| Tragopogon porrifolius | G | R | 76.2 |
| Trichosanthes kirilowii | G | O | 87.7 |
| Trigonella foenumgraecum | G | S | 31.0 |
| Trigonella foenumgraecum | G | O | 84.0 |
| Triticosecale spp | G | S | 26.5 |
| Triticosecale spp | G | O | 73.5 |
| Triticum aestivum | G | R | 62.4 |
| Triticum durum | G | O | 51.9 |
| Triticum spelta | G | S | 24.5 |
| Triticum spelta | G | O | 32.9 |
| Triticum turgidum | G | O | 25.1 |
| Tropaeolum majus | G | S | 21.3 |
| Tropaeolum majus | G | R | 45.6 |
| Urtica dioica | G | S | 21.3 |
| Urtica dioica | G | O | 100.0 |
| Valerianella locusta | G | O | 32.2 |
| Veratrum viride | G | R | 77.7 |
| Verbascum thapsus | G | S | 34.0 |
| Veronica beccabunga | G | R | 44.1 |
| Veronica officinalis | G | S | 38.8 |
| Veronica officinalis | G | R | 87.5 |
| Viburnum trilobum | G | O | 62.6 |
| Vicia faba | G | S | 22.2 |
| Vicia sativa | G | 0 | 74.8 |
| Vicia sativa | G | R | 100.0 |
| Vicia villosa | G | R | 100.0 |
| Vigna angularis | G | R | 65.2 |
| Vigna sesquipedalis | G | S | 35.1 |
| Vigna sesquipedalis | G | R | 73.8 |
| Vigna sesquipedalis | G | O | 100.0 |
| Vigna unguiculata | G | S | 65.9 |
| Vigna unguiculata | G | R | 84.5 |
| Vinca minor | G | S | 22.1 |
| Vitis sp. | G | R | 40.1 |
| Vitis sp. | G | O | 74.7 |
| Withania somnifera | G | S | 37.3 |
| Withania somnifera | G | O | 91.0 |
| Xanthium sibiricum | G | S | 38.4 |
| Xanthium sibiricum | G | O | 100.0 |
| Xanthium strumarium | G | S | 37.7 |
| Xanthium strumarium | G | O | 39.6 |
| Xanthium strumarium | G | R | 40.0 |
| Zea mays | G | S | 43.3 |
| Zea mays | G | O | 64.4 |
| Zea mays | G | R | 68.3 |
| Perilla frutescens | T | R | 100.0 |
| Abies lasiocarpa | T | S | 20.2 |
| Abies lasiocarpa | T | R | 59.1 |
| Achillea millefolium | T | O | 84.7 |
| Aconitum napellus | T | O | 22.0 |
| Aconitum napellus | T | R | 100.0 |
| Adiantum pedatum | T | R | 100.0 |
| Agaricus bisporus | T | R | 52.1 |
| Agaricus bisporus | T | R | 65.6 |
| Ageratum conyzoides | T | S | 26.7 |
| Agropyron repens | T | S | 30.2 |
| Agrostis Stolonifera | T | O | 100.0 |
| Alcea rosea | T | R | 63.7 |
| Alchemilla mollis | T | R | 28.6 |
| Allium ampeloprasum | T | R | 55.9 |
| Allium ampeloprasum | T | O | 60.4 |
| Allium ascalonicum | T | S | 20.4 |
| Allium ascalonicum | T | O | 73.4 |
| Allium cepa | T | S | 33.8 |
| Allium cepa | T | S | 35.6 |
| Allium cepa | T | R | 48.0 |
| Allium cepa | T | R | 78.6 |
| Allium grande | T | R | 32.4 |
| Allium schoenoprasum | T | R | 67.7 |
| Allium Tuberosum | T | S | 38.8 |
| Allium Tuberosum | T | O | 82.5 |
| Allium Tuberosum | T | R | 85.2 |
| Aloe vera | T | R | 74.6 |
| Althaea officianalis | T | S | 37.7 |
| Althaea officinalis | T | O | 55.3 |
| Althaea officinalis | T | R | 72.3 |
| Amaranthus caudathus | T | O | 53.5 |
| Amaranthus gangeticus | T | S | 28.1 |
| Ananas comosus | T | R | 37.9 |
| Ananas comosus | T | O | 100.0 |
| Angelica archangelica | T | R | 41.3 |
| Anthemis nobilis | T | O | 100.0 |
| Anthemis nobilis | T | R | 100.0 |
| Anthriscus cerefolium | T | S | 21.9 |
| Anthriscus cerefolium | T | O | 67.1 |
| Apium graveolens | T | R | 35.5 |
| Apium graveolens | T | R | 52.1 |
| Aralia cordata | T | R | 100.0 |
| Aralia nudicaulis | T | R | 31.2 |
| Arctium minus | T | S | 31.3 |
| Arctium minus | T | O | 73.7 |
| Armoracia rusticana | T | O | 49.9 |
| Arrhenatherum elatius | T | O | 100.0 |
| Artemisia dracunlus | T | S | 100.0 |
| Asclepias incarnata | T | S | 32.3 |
| Asparagus officinalis | T | S | 48.2 |
| Atriplex hortensis | T | R | 28.4 |
| Avena sativa | T | R | 31.3 |
| Avena sativa | T | O | 70.6 |
| Avena sativa | T | R | 100.0 |
| Averrhoa carambola | T | R | 44.0 |
| Bellis perennis | T | R | 82.0 |
| Beta vulgaris | T | S | 33.7 |
| Beta vulgaris | T | R | 100.0 |
| Betula glandulosa | T | O | 53.5 |
| Boletus edulis | T | S | 21.8 |
| Borago officinalis | T | S | 42.3 |
| Borago officinalis | T | R | 78.5 |
| Brassica hirta | T | R | 53.1 |
| Brassica hirta | T | O | 68.9 |
| Brassica napus | T | S | 45.1 |
| Brassica napus | T | R | 82.9 |
| Brassica oleracea | T | R | 38.8 |
| Brassica oleracea | T | R | 49.7 |
| Brassica oleracea | T | O | 75.5 |
| Brassica oleracea | T | R | 77.0 |
| Brassica oleracea | T | S | 77.2 |
| Brassica rapa | T | R | 25.4 |
| Brassica rapa | T | O | 37.9 |
| Brassica rapa | T | S | 47.7 |
| Brassica rapa | T | R | 64.7 |
| Brassica rapa | T | R | 81.8 |
| Calamintha nepeta | T | O | 57.6 |
| Calendula officinalis | T | S | 32.6 |
| Camellia sinensis | T | S | 21.0 |
| Camellia sinensis | T | R | 43.8 |
| Camellia sinensis | T | O | 66.2 |
| Canna edulis | T | O | 100.0 |
| Cantharellus cibarias | T | S | 26.0 |
| capsicum annuum | T | S | 54.6 |
| capsicum annuum | T | R | 100.0 |
| Capsicum frutescens | T | S | 60.9 |
| Capsicum frutescens | T | R | 100.0 |
| Carex morrowii | T | R | 24.4 |
| Carica papaya | T | S | 20.8 |
| Carthamus tinctorius | T | R | 39.6 |
| Carya cordiformis | T | R | 100.0 |
| Cerastium tomentosum | T | R | 54.8 |
| Chaerophyllum bulbosum | T | S | 42.2 |
| Chaerophyllum bulbosum | T | R | 74.3 |
| Chelidonium majus | T | S | 20.3 |
| Chenopodium quinoa | T | O | 76.0 |
| Chrysanthemum coronarium | T | S | 30.6 |
| Chrysanthemum parthenium | T | R | 57.2 |
| chrysanthemun coronarium | T | R | 56.5 |
| Chrysanthenum coronarium | T | R | 81.6 |
| Cicer arietinum | T | O | 32.2 |
| Cichorium endivia subsp endivia | T | R | 27.1 |
| Cichorium endivia subsp. Endivia | T | S | 26.9 |
| Cichorium endivia subsp. Endivia | T | O | 64.5 |
| Cichorium intybus | T | S | 22.7 |
| Cichorium intybus | T | R | 53.5 |
| Cimicifuga racemosa | T | S | 41.1 |
| Cimicifuga racemosa | T | R | 68.4 |
| Circium arvense | T | S | 42.5 |
| Circium arvense | T | R | 64.5 |
| Citrullus lanatus | T | S | 72.4 |
| Citrullus lanatus | T | O | 92.2 |
| Citrullus lanatus | T | R | 100.0 |
| Citrus limettoides | T | O | 77.1 |
| Citrus limon | T | R | 43.6 |
| Citrus paradisi | T | S | 21.8 |
| Citrus paradisi | T | R | 90.9 |
| Citrus sinensis | T | R | 46.7 |
| Colocasia sp | T | R | 43.4 |
| Colocasia sp | T | O | 84.3 |
| Corchorus olitorius | T | R | 22.7 |
| Coriandrum sativum | T | S | 20.4 |
| Cornus canadensis | T | S | 66.0 |
| Cosmos sulphureus | T | R | 47.1 |
| Crataegus submollis | T | S | 21.2 |
| Crataegus submollis | T | O | 94.3 |
| Cucumis anguria | T | S | 49.4 |
| Cucumis anguria | T | R | 84.1 |
| Cucumis melo | T | S | 56.6 |
| Cucumis melo | T | R | 92.4 |
| Cucumis melo | T | O | 100.0 |
| Cucumis metuliferus | T | S | 29.5 |
| Cucumis sativus | T | S | 28.3 |
| Cucurbita maxima | T | S | 26.7 |
| Cucurbita maxima | T | O | 34.7 |
| Cucurbita maxima | T | R | 62.1 |
| Cucurbita moschata | T | R | 30.7 |
| Cucurbita moschata | T | S | 33.4 |
| Cucurbita moschata | T | S | 48.3 |
| Cucurbita moschata | T | R | 98.8 |
| Cucurbita moschata | T | O | 100.0 |
| Cucurbita pepo | T | S | 45.8 |
| Cucurbita pepo | T | R | 80.2 |
| Cucurbita pepo | T | O | 98.9 |
| Cuminum cyminum | T | O | 54.0 |
| Curcuma zedoaria | T | S | 100.0 |
| Cymbopogon citratus | T | S | 21.0 |
| Cymbopogon martinii motia | T | S | 27.5 |
| Cynara scolymus | T | S | 23.1 |
| Cynara scolymus | T | O | 83.4 |
| Cyperus esculentus | T | R | 100.0 |
| Dactilis Glomerata | T | S | 30.8 |
| Dactilis Glomerata | T | O | 34.5 |
| Daucus carota | T | S | 27.1 |
| Daucus carota | T | R | 56.8 |
| Daucus Carota | T | O | 100.0 |
| Digitalis purpurea | T | S | 38.4 |
| Dirca palustris | T | S | 45.9 |
| Dolichos lablab | T | S | 46.6 |
| Dryopteris filix-mas | T | O | 29.5 |
| Dryopteris filix-mas | T | R | 100.0 |
| Echinacea purpurea | T | R | 59.3 |
| Echinacea purpurea | T | O | 87.8 |
| Eleusine coracana | T | S | 28.6 |
| Eleusine coracana | T | R | 80.0 |
| Erigeron canadensis | T | O | 100.0 |
| Eruca vesicaria | T | R | 60.5 |
| Erysimum perofskianum | T | S | 28.2 |
| Erysimum perofskianum | T | R | 85.2 |
| Eschscholzia californica | T | S | 49.9 |
| Eschscholzia californica | T | O | 74.5 |
| Fagopyrum esculentum | T | O | 52.9 |
| Fagopyrum tartaricum | T | S | 25.6 |
| Fagopyrum tartaricum | T | R | 68.4 |
| Fagopyrum tartaricum | T | O | 100.0 |
| Festuca rubra | T | O | 51.6 |
| Festuca rubra | T | S | 56.6 |
| Festuca rubra | T | R | 71.7 |
| Foeniculum vulgare | T | S | 36.5 |
| Foeniculum vulgare | T | R | 41.4 |
| Foericulum vulgare | T | O | 100.0 |
| Fortunella spp | T | R | 53.9 |
| Fragaria x ananassa | T | R | 28.1 |
| Galinsoga ciliata | T | S | 43.2 |
| Galinsoga ciliata | T | R | 73.3 |
| Galium odoratum | T | S | 42.0 |
| Galium odoratum | T | O | 94.2 |
| Glaux Maritima | T | R | 24.8 |
| Glycine max | T | R | 37.2 |
| Glycine max | T | O | 100.0 |
| Glycine max | T | R | 100.0 |
| Glycine max | T | S | 100.0 |
| Gossypium herbaceum | T | R | 48.7 |
| Guizotia abyssinica | T | S | 26.8 |
| Guizotia abyssinica | T | R | 100.0 |
| Hedeoma pulegioides | T | R | 20.3 |
| Hedeoma pulegioides | T | O | 72.7 |
| Helianthus annuus | T | R | 56.1 |
| Helianthus strumosus | T | O | 100.0 |
| Helianthus tuberosus | T | S | 25.3 |
| Helianthus tuberosus | T | R | 28.1 |
| Helianthus tuberosus | T | O | 78.6 |
| Helianthus tuberosus | T | R | 91.5 |
| Helichrysum angustifolium | T | R | 83.4 |
| Helichrysum angustifolium | T | S | 88.3 |
| Helichrysum thianschanicum | T | O | 26.0 |
| Heliotropium arborescens | T | R | 100.0 |
| Helleborus niger | T | R | 23.0 |
| Hibiscus cannabinus | T | R | 37.9 |
| Hordeum vulgare | T | O | 75.9 |
| Hordeum vulgare supsp vulgare | T | S | 20.5 |
| Hordeum vulgare supsp vulgare | T | O | 62.3 |
| Humulus lupulus | T | S | 44.7 |
| Humulus lupulus | T | O | 70.6 |
| Hypericum henryi | T | O | 76.8 |
| Hypericum henryi | T | R | 99.8 |
| Hypericum perforatum | T | R | 38.8 |
| Hyssopus officinalis | T | O | 100.0 |
| Iberis amara | T | O | 100.0 |
| Juniperus communis | T | S | 100.0 |
| Kochia scoparia | T | S | 25.2 |
| Koeleria glauca | T | S | 23.1 |
| Lactuca sativa | T | R | 70.5 |
| Lactuca serriola | T | R | 34.1 |
| Laportea canadensis | T | R | 61.3 |
| Lathyrus sylvestris | T | R | 48.6 |
| Laurus nobilis | T | O | 73.6 |
| Lavandula angustifolia | T | R | 35.0 |
| Lavandula angustifolia | T | O | 100.0 |
| Lavandula latifolia | T | O | 77.1 |
| Lepidium sativum | T | S | 35.2 |
| Lepidium sativum | T | R | 48.1 |
| Lepidium sativum | T | O | 72.9 |
| Levisticum officinale | T | S | 38.7 |
| Levisticum officinale | T | O | 60.3 |
| Linum usitatissimum | T | R | 24.7 |
| Lolium multiflorum | T | S | 39.8 |
| Lolium multiflorum | T | O | 74.1 |
| Lonicera ramosissima | T | S | 34.4 |
| Lonicera ramosissima | T | O | 80.5 |
| Lonicera syringantha | T | R | 58.4 |
| Lotus corniculatus | T | S | 36.0 |
| Lotus corniculatus | T | O | 100.0 |
| Lotus tetragonolobus | T | R | 76.1 |
| Lunaria annua | T | R | 47.4 |
| Lycopersicon esculentum | T | R | 69.7 |
| Lycopersicon pimpinellifolium | T | R | 58.7 |
| Malus hupehensis | T | R | 53.1 |
| Malus hupehensis | T | S | 100.0 |
| Malus sp. | T | R | 72.6 |
| Malva moschata | T | O | 96.7 |
| Malva verticillata | T | R | 35.8 |
| Manihot esculenta | T | R | 53.7 |
| Melaleuca alternifolia | T | S | 21.5 |
| Melaleuca alternifolia | T | O | 78.7 |
| Melilotus albus | T | R | 79.7 |
| Melilotus officinalis | T | S | 34.6 |
| Melilotus officinalis | T | R | 100.0 |
| Melissa officinalis | T | O | 100.0 |
| Mentha piperita | T | S | 24.5 |
| Mentha pulegium | T | O | 100.0 |
| Mentha suaveolens | T | O | 20.9 |
| Miscanthus sinensis Andress | T | S | 69.1 |
| Momordica charantia | T | R | 54.9 |
| Monarda didyma | T | S | 31.3 |
| Monarda fistulosa | T | S | 21.3 |
| Monarda fistulosa | T | O | 100.0 |
| Montia perfoliata | T | R | 67.2 |
| Musa paradisiaca | T | R | 47.3 |
| nasturtium officinale | T | S | 55.7 |
| Nepeta cataria | T | S | 20.7 |
| Nepeta cataria | T | S | 69.0 |
| Nepeta cataria | T | O | 100.0 |
| Nicotiana rustica | T | S | 52.8 |
| Nicotiana rustica | T | R | 88.1 |
| Nicotiana tabacum | T | S | 50.3 |
| Nicotiana tabacum | T | R | 91.5 |
| Nigella sativa | T | R | 34.2 |
| Nigella sativa | T | R | 90.3 |
| Nigella sativa | T | R | 100.0 |
| Ocimum Basilicum | T | S | 21.6 |
| Ocimum Basilicum | T | O | 100.0 |
| Ocimum tenuiflorum | T | R | 44.5 |
| Oenothera biennis | T | R | 48.2 |
| Onobrychis viciifolia | T | S | 34.4 |
| Onobrychis viciifolia | T | O | 35.6 |
| Opuntia sp. | T | S | 23.5 |
| Origanum vulgare | T | S | 20.7 |
| Origanum vulgare | T | R | 76.7 |
| Origanum vulgare | T | O | 100.0 |
| Oryza sativa | T | R | 60.8 |
| Oxalis Deppei | T | S | 22.2 |
| Oxalis Deppei | T | R | 81.4 |
| Passiflora caerulea | T | S | 36.9 |
| Passiflora caerulea | T | R | 87.0 |
| Passiflora spp | T | R | 54.6 |
| Pastinaca sativa | T | S | 24.8 |
| Pastinaca sativa | T | R | 74.7 |
| Perroselinum crispum | T | R | 85.2 |
| Perroselinum crispum | T | O | 100.0 |
| Persea americana | T | R | 43.1 |
| Petasites Japonicus | T | S | 21.9 |
| Petroselinum crispum | T | R | 52.8 |
| Peucedanum oreaselinum | T | R | 41.9 |
| Phalaris canariensis | T | R | 41.1 |
| Phalaris canariensis | T | O | 100.0 |
| Phaseolus acutifolius | T | R | 88.2 |
| Phaseolus coccineus | T | S | 22.2 |
| Phaseolus coccineus | T | R | 36.4 |
| Phaseolus coccineus | T | R | 86.7 |
| Phaseolus coccineus | T | O | 100.0 |
| Phaseolus mungo | T | S | 43.0 |
| Phaseolus vulgaris | T | S | 62.9 |
| Phaseolus vulgaris | T | R | 71.9 |
| Phaseolus vulgaris | T | R | 73.0 |
| Phaseolus vulgaris | T | O | 100.0 |
| Phlox paniculata | T | R | 23.1 |
| Phlox paniculata | T | R | 92.8 |
| Physalis alkekengi | T | R | 39.5 |
| Physalis ixocarpa | T | R | 36.7 |
| Physalis ixocarpa | T | R | 75.9 |
| Physalis pruinosa | T | R | 65.6 |
| Physalis pruinosa | T | R | 71.0 |
| Physalis pruinosa | T | O | 100.0 |
| Physalis pruinosa | T | O | 100.0 |
| Phytolacca decandra | T | S | 39.3 |
| Phytolacca decandra | T | O | 42.0 |
| Pimpinella anisum | T | S | 27.9 |
| Pimpinella anisum | T | R | 35.8 |
| Pimpinella anisum | T | O | 49.9 |
| Pimpinella anisum | T | R | 55.5 |
| Pisum sativum | T | S | 22.3 |
| Plantago coronopus | T | R | 35.2 |
| Plantago coronopus | T | R | 46.0 |
| Plantago coronopus | T | O | 73.5 |
| Plantago major | T | S | 22.3 |
| Plectranthus sp. | T | S | 59.2 |
| Pleurotus spp | T | R | 26.6 |
| Poa compressa | T | S | 33.4 |
| Poa compressa | T | R | 75.7 |
| Poa compressa | T | O | 100.0 |
| Poa pratensis | T | S | 25.4 |
| Polygonum pensylvanicum | T | O | 66.8 |
| Polygonum pensylvanicum | T | R | 73.3 |
| Polygonum persicaria | T | S | 27.1 |
| Polygonum persicaria | T | O | 50.8 |
| Populus incrassata | T | O | 74.3 |
| Populus incrassata | T | S | 100.0 |
| Prunus armeniaca | T | R | 55.0 |
| Prunus cerasus | T | O | 100.0 |
| Prunus persica | T | S | 26.0 |
| Prunus persica | T | R | 46.2 |
| Psoralea corylifolia | T | S | 47.4 |
| Pteridium aquilinum | T | R | 100.0 |
| Pyrus communis | T | R | 42.9 |
| Raphanus raphanistrum | T | S | 24.4 |
| Raphanus raphanistrum | T | R | 56.9 |
| Raphanus raphanistrum | T | O | 62.1 |
| Raphanus raphanistrum | T | O | 100.0 |
| Raphanus sativus | T | R | 48.9 |
| Raphanus sativus | T | S | 59.8 |
| Raphanus sativus | T | R | 81.6 |
| Reseda odorata | T | O | 71.3 |
| Rhamnus frangula | T | O | 44.6 |
| Rhamnus frangula | T | R | 74.4 |
| Rheum officinale | T | O | 73.9 |
| Rheum officinale | T | S | 100.0 |
| Ricinus communis | T | O | 100.0 |
| Rosmarinus officinalis | T | O | 100.0 |
| Rosmarinus officinalis | T | R | 100.0 |
| Rubus ideaus | T | R | 78.1 |
| Rumex acetosella | T | R | 42.2 |
| Rumex crispus | T | 0 | 73.1 |
| Rumex patientia | T | S | 52.0 |
| Ruta graveolens | T | S | 34.7 |
| Ruta graveolens | T | O | 100.0 |
| Saccharum officinarum | T | S | 59.6 |
| Saccharum officinarum | T | R | 66.1 |
| Saliva elegans | T | S | 36.3 |
| Saliva elegans | T | O | 44.3 |
| Salvia officinalis | T | S | 28.2 |
| Salvia officinalis | T | O | 100.0 |
| Salvia sclarea | T | R | 38.6 |
| Sambucus canadensis | T | S | 36.3 |
| Sambucus canadensis | T | R | 64.5 |
| Sambucus canadensis | T | O | 100.0 |
| Sanguisorba minor | T | O | 73.1 |
| Sanguisorba minor | T | R | 100.0 |
| Santolina chamaecyparissus | T | 0 | 27.7 |
| Santolina chamaecyparissus | T | R | 100.0 |
| Saponaria officinalis | T | R | 100.0 |
| Satureja hortensis | T | O | 62.2 |
| Satureja hortensis | T | R | 100.0 |
| Satureja montana | T | S | 34.7 |
| Satureja montana | T | O | 36.3 |
| Satureja montana | T | R | 100.0 |
| Satureja repandra | T | O | 47.0 |
| Satureja repandra | T | S | 47.6 |
| Satureja repandra | T | R | 84.6 |
| Scolymus hispanicus | T | R | 35.8 |
| Scorzorera hipanica | T | R | 99.4 |
| Scrophularia nodosa | T | S | 29.1 |
| Scrophularia nodosa | T | R | 90.1 |
| Scrophularia nodosa | T | O | 100.0 |
| Scutellaria lateriflora | T | S | 30.9 |
| Scutellaria lateriflora | T | R | 63.9 |
| Secale cereale | T | O | 100.0 |
| Senecio vulgaris | T | S | 24.7 |
| Senecio vulgaris | T | R | 32.2 |
| Sesamum indicum | T | R | 100.0 |
| Silene vulgaris | T | S | 25.6 |
| Sium sisarum | T | O | 81.4 |
| Sium sisarum | T | O | 100.0 |
| Solanum melanocerasum | T | S | 28.0 |
| Solanum melanocerasum | T | R | 78.8 |
| Solanum melanocerasum | T | R | 99.6 |
| Solanum melongena | T | S | 70.5 |
| Sorghum caffrorum | T | S | 28.1 |
| Sorghum dochna | T | R | 40.6 |
| Sorghum dochna | T | O | 100.0 |
| Sorghum durra | T | R | 29.7 |
| Sorghum durra | T | O | 78.9 |
| Sorghum sudanense | T | R | 74.6 |
| Sorghum sudanense | T | O | 100.0 |
| Spinacia oleracea | T | S | 28.5 |
| Spinacia oleracea | T | O | 62.7 |
| Stachys byzantina | T | R | 66.9 |
| Stachys byzantina | T | O | 100.0 |
| Stellaria media | T | S | 21.4 |
| Stellaria media | T | R | 87.1 |
| Stipa capillata | T | R | 37.5 |
| Symphytum officinale | T | O | 58.5 |
| Tanacetum cinerariifolium | T | O | 100.0 |
| Tanacetum cinerariifolium | T | R | 100.0 |
| Tanacetum parthenium | T | R | 100.0 |
| Tanacetum vulgare | T | R | 20.8 |
| Taraxacum officinale | T | R | 76.3 |
| Teucrium chamaedrys | T | O | 75.6 |
| Thalpsi arvense | T | O | 64.1 |
| Thymus fragantissimus | T | S | 21.4 |
| Thymus praecox subsp arcticus | T | S | 36.4 |
| Thymus pseudolanuginosus | T | S | 21.1 |
| Thymus pseudolanuginosus | T | O | 75.4 |
| Thymus serpyllum | T | O | 64.2 |
| Thymus vulgaris | T | R | 71.5 |
| Thymus x citriodorus | T | S | 27.6 |
| Tragopogon porrifolium | T | S | 44.8 |
| Tragopogon porrifolius | T | O | 39.1 |
| Tragopogon porrifolius | T | R | 57.9 |
| Tragopogon sp. | T | R | 20.0 |
| Trifolium repens | T | R | 79.7 |
| Trigonella foenum graecum | T | O | 28.4 |
| Trigonella foenum graecum | T | S | 34.8 |
| Triticosecale spp | T | S | 28.5 |
| Triticosecale spp | T | O | 100.0 |
| Triticum aestivum | T | R | 32.9 |
| Triticum aestivum | T | O | 67.7 |
| Triticum durum | T | O | 47.7 |
| Triticum spelta | T | O | 37.1 |
| Triticum turgidumm | T | O | 41.2 |
| Tropaeolum majus | T | S | 42.7 |
| Tropaeolum majus | T | R | 77.6 |
| Tsuga diversifolia | T | R | 53.4 |
| Typha latifolia | T | S | 29.2 |
| Urtica dioica | T | S | 29.5 |
| Vaccinium angustifolium | T | R | 59.4 |
| Vaccinium angustifolium | T | R | 100.0 |
| Vaccinium macrocarpon | T | S | 51.1 |
| Vaccinium macrocarpon | T | O | 64.7 |
| Valerianella locusta | T | S | 22.7 |
| Valerianella locusta | T | O | 24.8 |
| Veronica beccabunga | T | R | 33.3 |
| Veronica officinalis | T | R | 59.2 |
| Veronica officinalis | T | O | 100.0 |
| Viburnum trilobum | T | O | 71.2 |
| Vicia faba | T | S | 25.5 |
| Vicia faba | T | R | 27.0 |
| Vicia sativa | T | O | 56.6 |
| Vicia villosa | T | R | 100.0 |
| Vigna angularis | T | R | 49.2 |
| Vigna sesquipedalis | T | R | 77.4 |
| Vigna sesquipedalis | T | O | 100.0 |
| Vigna unguiculata | T | S | 27.2 |
| Vigna unguiculata | T | R | 59.0 |
| Vinca minor | T | R | 39.2 |
| Vitis sp. | T | R | 31.9 |
| Vitis sp. | T | S | 36.3 |
| Vitis sp. | T | O | 72.2 |
| Weigela coraeensis | T | S | 32.9 |
| Weigela coraeensis | T | R | 61.5 |
| Withania somnifera | T | S | 36.1 |
| Withania somnifera | T | O | 83.3 |
| Xanthium sibiricum | T | S | 32.1 |
| Xanthium sibiricum | T | R | 33.2 |
| Xanthium sibiricum | T | O | 62.4 |
| Xanthium strumarium | T | S | 47.2 |
| Xanthium strumarium | T | O | 74.3 |
| Zea mays | T | R | 55.7 |
| Zea mays | T | O | 100.0 |
| Zingiber officinale | T | R | 79.0 |

TABLE 3
Inhibition of MMP-3 by Plant Extracts

| | | | Inhibition |
|---|---|---|---|
| Latin name | Stress | Extract | (%) |
| Achillea millefolium | A | O | 21.4 |
| Allium Tuberosum | A | S | 32.5 |
| Anethum graveolens | A | S | 26.0 |
| Anthemis nobilis | A | R | 20.3 |
| Anthemis tinctoria | A | R | 58.0 |
| Apium graveolens | A | R | 34.1 |
| Arctium minus | A | R | 53.9 |
| Arctium minus | A | O | 100.0 |
| Arctostaphylos uva-ursi | A | S | 58.6 |
| Aronia melanocarpa | A | R | 32.2 |
| Artemisia Absinthium | A | O | 100.0 |
| Artemisia dracunculus | A | R | 23.4 |
| Artemisia dracunculus | A | S | 63.0 |
| Aster sp | A | O | 42.4 |
| Atropa belladonna | A | O | 23.8 |
| Beta vulgaris | A | S | 24.1 |
| Beta vulgaris | A | O | 42.9 |
| Beta vulgaris | A | O | 94.3 |
| Beta vulgaris | A | R | 97.9 |
| Beta vulgaris var. condivata | A | O | 21.2 |
| Brassica napus | A | S | 25.0 |
| Brassica napus | A | O | 100.0 |
| Brassica oleracea | A | S | 39.9 |
| Canna edulis | A | S | 39.6 |
| Capsicum annuum | A | S | 35.4 |
| Capsicum frutescens | A | S | 27.2 |
| Cichorium intybus | A | O | 20.2 |
| Cichorium intybus | A | R | 26.5 |
| Cichorium intybus | A | S | 28.2 |
| Citrullus lanatus | A | S | 21.7 |
| Citrullus lanatus | A | O | 27.8 |
| Citrullus lanatus | A | R | 34.4 |
| Coix Lacryma-Jobi | A | S | 37.3 |
| Coix Lacryma-Jobi | A | O | 78.1 |
| Cosmos sulphureus | A | R | 26.8 |
| Crataegus submollis | A | S | 22.3 |
| Crataegus submollis | A | R | 61.6 |
| Cucumis anguria | A | S | 27.8 |
| Cucurbita Maxima | A | S | 28.9 |
| Cucurbita moschata | A | S | 32.9 |
| Cucurbita pepo | A | S | 50.9 |
| Datisca cannabina | A | R | 43.3 |
| Datisca cannabina | A | S | 100.0 |
| Digitalis purpurea | A | R | 20.0 |
| Dipsacus sativus | A | R | 64.8 |
| Dirca palustris | A | S | 29.6 |
| Dryopteris filix-mas | A | R | 22.0 |
| Dryopteris filix-mas | A | O | 32.8 |
| Echinacea purpurea | A | O | 100.0 |
| Fagopyrum tataricum | A | R | 28.3 |
| Fagopyrum tataricum | A | O | 29.7 |
| Filipendula rubra | A | S | 43.7 |
| Filipendula rubra | A | R | 63.2 |
| Fragaria x ananassa | A | R | 41.5 |
| Fragaria x ananassa | A | S | 67.1 |
| Fragaria x ananassa | A | O | 99.6 |
| Fragaria x ananassa | A | R | 31.7 |
| Gaultheria hispidula | A | R | 50.5 |
| Glycyrrhiza glabra | A | R | 56.2 |
| Hedeoma pulegioides | A | O | 51.7 |
| Helianthus tuberosus | A | O | 22.9 |
| Hordeum vulgare subsp vulgare | A | S | 36.0 |
| Hypericum henryi | A | R | 67.2 |
| Hypericum perforatum | A | R | 31.7 |
| Hyssopus officinalis | A | R | 21.6 |
| Iris versicolor | A | R | 53.6 |
| Isatis tinctoria | A | S | 32.9 |
| Levisticum officinale | A | O | 46.7 |
| Lotus tetragonolobus | A | R | 26.2 |
| Matricaria recutita | A | S | 43.5 |
| Matteucia pensylvanica | A | R | 24.7 |
| Melissa officinalis | A | S | 30.3 |
| Mentha suaveolens | A | R | 91.7 |
| Nepeta cataria | A | S | 30.3 |
| Nigella sativa | A | O | 26.0 |
| Ocinum tenuiflorum | A | O | 33.0 |
| Ocinum tenuiflorum | A | R | 49.8 |
| Perilla frutescens | A | R | 34.8 |
| Petasites japonicus | A | R | 38.0 |
| Phaseolus mungo | A | O | 62.6 |
| Phaseolus vulgaris | A | S | 21.2 |
| Phaseolus vulgaris | A | O | 50.6 |
| Phaseolus Vulgaris | A | R | 100.0 |
| Phlox paniculata | A | S | 46.4 |
| Physalis alkekengi | A | O | 37.5 |
| Plantago major | A | O | 27.3 |
| Polygonum aviculare linné | A | S | 24.8 |
| Polygonum persicaria | A | S | 59.1 |
| Potentilla anserina | A | R | 40.1 |
| Poterium sanguisorba | A | R | 75.7 |
| Prunus cerasifera | A | R | 80.0 |
| Ptaridium aquilinus | A | R | 39.6 |
| Raphanus raphanistrum | A | S | 28.2 |
| Raphanus sativus | A | S | 64.4 |
| Ribes nigrum | A | O | 47.6 |
| ribes uva-crispa | A | R | 21.0 |
| ribes uva-crispa | A | O | 100.0 |
| Rosa rugosa | A | S | 21.4 |
| Rosmarinus officinalis | A | R | 27.3 |
| Rubus allegheniensis | A | R | 81.0 |
| Rubus arcticus | A | R | 51.0 |
| Rubus canadensis | A | R | 48.8 |
| Rubus idaeus | A | S | 28.5 |
| Rubus idaeus | A | R | 35.1 |
| Rubus pubescens | A | O | 50.4 |
| Rubus thibetanus | A | O | 39.1 |
| Rumex patientia | A | S | 24.8 |
| Ruta graveolens | A | O | 56.1 |
| Salvia officinalis | A | R | 43.2 |
| Santolina chamaecyparissus | A | R | 27.0 |
| Scutellaria lateriflora | A | R | 53.5 |
| Solanum melongena | A | S | 21.8 |
| Solidago canadensis | A | S | 27.4 |
| Stachys affinis | A | S | 100.0 |
| Stellaria media | A | O | 24.4 |
| Tanacetum vulgare | A | R | 62.1 |
| Thymus praecox subsp arcticus | A | S | 28.4 |
| Thymus praecox subsp arcticus | A | O | 31.8 |
| Trichosanthes kirilowii | A | S | 23.2 |
| Vaccinium Corymbosum | A | R | 100.0 |
| Vaccinium macrocarpon | A | S | 48.6 |
| Vaccinum augustifolium | A | R | 56.6 |
| Vigna angularia | A | O | 23.1 |
| Vigna sesquipedalis | A | O | 37.8 |
| Vigna unguiculata | A | S | 52.5 |
| Vinca minor | A | O | 23.2 |
| Vitis sp. | A | S | 20.8 |
| Vitis sp. | A | O | 21.5 |
| Vitis sp. | A | R | 33.6 |
| Xanthium sibiricum | A | S | 27.3 |
| Aconitum napellus | G | O | 59.0 |
| Agropyron repens | G | O | 69.4 |
| Alchemilla mollis | G | S | 30.6 |
| Alchemilla mollis | G | O | 73.3 |
| Allium grande | G | O | 33.4 |
| Anethum graveolens | G | S | 40.5 |
| Aronia melanocarpa | G | O | 100.0 |
| Artemisia absinthium | G | S | 31.3 |
| Artemisia absinthium | G | O | 67.9 |
| Artemisia dracunculus | G | S | 100.0 |
| Atropa belladonna | G | S | 41.2 |
| Bellis perennis | G | S | 48.4 |
| Brassica oleracea | G | S | 26.4 |
| Brassica oleracea | G | O | 40.6 |
| Brassica rapa | G | S | 21.4 |
| Capsicum annuum | G | S | 35.0 |
| Capsicum annuum | G | S | 35.7 |
| Capsicum frutescens | G | S | 27.5 |
| Chelidonium majus | G | O | 34.7 |
| Cichorium intybus | G | R | 34.4 |
| Coix Lacryma-Jobi | G | S | 20.2 |
| Cosmos sulphureus | G | O | 32.9 |
| Crataegus submollis | G | S | 25.6 |
| Crataegus submollis | G | R | 28.6 |
| Cucumis anguria | G | S | 33.6 |
| Cucurbita maxima | G | S | 44.6 |
| Cucurbita moschata | G | S | 33.4 |
| Cucurbita pepo | G | S | 25.3 |
| Cymbopogon citratus | G | S | 30.3 |
| Cymbopogon martinii | G | S | 61.1 |
| Daucus carota | G | O | 30.0 |
| Dryopteris filix-mas | G | S | 26.0 |
| Dryopteris filix-mas | G | R | 45.3 |
| Echinacea purpurea | G | O | 51.8 |
| Echinochloa frumentacea | G | S | 30.3 |
| Fagopyrum esculentum | G | R | 50.9 |
| Fagopyrum tartaricum | G | O | 44.0 |
| Fagopyrum tartaricum | G | R | 46.0 |
| Filipendula rubra | G | S | 53.1 |
| Filipendula rubra | G | R | 58.7 |
| Forsythia intermedia | G | O | 52.9 |
| Fragaria x ananassa | G | R | 40.7 |
| Fragaria x ananassa | G | R | 28.1 |
| Gaultheria hispidula | G | R | 72.8 |
| Gaultheria hispidula | G | O | 100.0 |
| Gaultheria procumbens | G | R | 24.1 |
| Glycine max | G | S | 31.2 |
| Glycyrrhiza glabra | G | R | 37.1 |
| Guizotia abyssinica | G | R | 35.4 |
| Hamamelis virginiana | G | S | 29.1 |
| Hamamelis virginiana | G | R | 67.1 |
| Helenium hoopesii | G | R | 39.8 |
| Helianthus tuberosus | G | O | 32.8 |
| Hordeum hexastichon | G | S | 60.9 |
| Humulus lupulus | G | R | 61.2 |
| Humulus lupulus | G | S | 90.5 |
| Hypericum henryi | G | R | 100.0 |
| Hypericum perforatum | G | R | 43.4 |
| Hyssopus officinalis | G | S | 25.1 |
| Hyssopus officinalis | G | O | 48.2 |
| Iris versicolor | G | R | 47.0 |
| Isatis tinctoria | G | S | 32.1 |
| Lavandula angustifolia | G | S | 43.9 |
| Levisticum officinale | G | O | 51.4 |
| Malus hupehensis | G | S | 24.2 |
| Malus hupehensis | G | R | 37.2 |
| Malva sylvestris | G | O | 73.7 |
| Matricaria recutita | G | S | 31.5 |
| Melaleuca alternifolia | G | S | 21.5 |
| Melissa officinalis | G | S | 32.8 |
| Melissa officinalis | G | R | 44.8 |
| Melissa officinalis | G | O | 82.4 |
| Mentha piperita | G | R | 77.3 |
| Mentha pulegium | G | R | 41.1 |
| Monarda didyma | G | S | 31.8 |
| Nepeta cataria | G | R | 25.8 |
| Nepeta cataria | G | O | 84.9 |
| Nigella sativa | G | O | 44.9 |
| Ocinum tenuiflorum | G | R | 23.7 |
| Oenothera biennis | G | S | 25.6 |
| Origanum vulgare | G | S | 28.6 |
| Origanum vulgare | G | R | 31.2 |
| Pennisetum alopecuroides | G | S | 49.9 |
| Petroselinum crispum | G | S | 31.5 |
| Peucedanum oreaselinum | G | R | 68.3 |
| Phaseolus acutifolius | G | R | 25.4 |
| Phaseolus acutifolius | G | O | 61.8 |
| Phaseolus vulgaris | G | O | 24.4 |
| Phaseolus vulgaris | G | S | 35.6 |
| Phlox paniculata | G | S | 27.2 |
| Physalis alkekengi | G | R | 26.1 |
| Physalis alkekengi | G | O | 54.9 |
| Plantago major | G | O | 55.9 |
| Plectranthus sp. | G | R | 23.0 |
| Polygonum persicaria | G | S | 41.1 |
| Potentilla anserina | G | R | 55.4 |
| Poterium sanguisorba | G | R | 76.4 |
| Prunus cerasifera | G | R | 55.3 |
| Ptaridium aquilinus | G | R | 44.5 |
| Rhaphanus sativus | G | O | 98.1 |
| Rheum x cultorum | G | R | 27.0 |
| Ribes nidigrolaria | G | R | 22.0 |
| Ribes Silvestris | G | R | 88.8 |
| Rosmarinus officinalis | G | R | 39.4 |
| Rubus idaeus | G | S | 100.0 |
| Rubus ideaus | G | O | 37.0 |
| Rubus Phoenicalasius | G | R | 24.9 |
| Rubus pubescens | G | O | 23.0 |
| Rubus thibetanus | G | O | 41.2 |
| Rumex patientia | G | S | 36.2 |
| Salvia officinalis | G | O | 34.5 |
| Salvia officinalis | G | R | 89.5 |
| Sanguisorba officinalis | G | S | 46.8 |
| Santolina chamaecyparissus | G | R | 33.7 |
| Secale cereale | G | S | 24.4 |
| Senecio vulgaris | G | R | 37.6 |
| Solanum melongena | G | S | 21.1 |
| Solanum tuberosum | G | S | 27.6 |
| Sorghum dochna | G | S | 23.7 |
| Sorghum dochna | G | R | 56.3 |
| Symphytum officinale | G | S | 25.2 |
| Teucrium chamaedrys | G | S | 75.4 |
| Thymus praecox subsp arcticus | G | S | 28.4 |
| Thymus praecox subsp arcticus | G | O | 52.1 |
| Thymus x citriodorus | G | R | 25.3 |
| Triticum durum | G | S | 21.9 |
| Triticum turgidum | G | O | 80.2 |
| Vaccinium angustifolium | G | R | 47.6 |
| Vaccinium angustifolium | G | R | 48.1 |
| Vaccinium angustifolium | G | R | 71.0 |
| Vaccinium corymbosum | G | R | 60.6 |
| Vaccinium corymbosum | G | R | 61.7 |
| Vaccinium corymbosum | G | O | 99.4 |
| Vaccinium macrocarpon | G | R | 100.0 |
| Vaccinum angustifolium | G | O | 24.4 |
| Vaccinum angustifolium | G | R | 41.5 |
| Valeriana officinalis | G | R | 33.5 |
| Veronica officinalis | G | S | 27.0 |
| Vicia faba | G | O | 31.2 |
| Vicia faba | G | R | 44.7 |
| Vigna angularia | G | O | 40.8 |
| Vigna angularis | G | S | 39.4 |
| Vigna unguiculata | G | O | 26.1 |
| Vitis sp. | G | R | 62.4 |
| Vitis sp. | G | S | 63.3 |
| Vitis sp. | G | O | 82.0 |
| Withania somnifera | G | S | 22.4 |
| Xanthium strumarium | G | S | 20.7 |
| Zea mays | G | S | 26.1 |
| Zea mays | G | R | 67.5 |
| Abies lasiocarpa | T | R | 46.2 |
| Acorus calamus | T | R | 21.8 |
| Actinidia arguta | T | R | 64.6 |
| Agropyron repens | T | O | 48.3 |
| Alchemilla mollis | T | R | 100.0 |
| Alchemilla mollis | T | O | 100.0 |
| Allium cepa | T | R | 39.8 |
| Allium cepa | T | O | 45.2 |
| Allium tuberosum | T | R | 28.2 |
| Allium tuberosum | T | S | 28.8 |
| Alpinia officinarum | T | S | 26.4 |
| Amelanchier alnitolia | T | R | 78.3 |
| Amelanchier sanguinea x A. laevis | T | R | 66.5 |
| angelica archangelica | T | S | 25.2 |
| Apium graveolens | T | R | 43.3 |
| Aralia cordata | T | S | 31.5 |
| Aralia nudicaulis | T | S | 37.7 |
| Aralia nudicaulis | T | R | 48.5 |
| Aronia melanocarpa | T | S | 26.0 |
| Aronia melanocarpa | T | O | 53.3 |
| Aronia prunifolia | T | R | 79.2 |
| Artemisia absinthium | T | O | 100.0 |
| Artemisia dracunlus | T | S | 42.0 |
| Ayperus esculentus | T | O | 67.8 |
| Beta vulgaris | T | R | 27.9 |
| Beta vulgaris | T | S | 33.2 |
| Beta vulgaris | T | O | 53.0 |
| Borago officinalis | T | O | 55.7 |
| Brassica Napus | T | O | 71.9 |
| Brassica oleracea | T | O | 37.0 |
| Brassica oleracea | T | S | 46.9 |
| Brassica rapa | T | S | 36.7 |
| Bromus inermis | T | R | 42.8 |
| Calendula officinalis L. | T | S | 28.4 |
| Camellia sinensis syn. Thea sinensis | T | R | 86.4 |
| Capsicum annus | T | S | 29.7 |
| Capsicum annus | T | R | 43.7 |
| Capsicum frutescens (tabasco) | T | S | 22.0 |
| Carya cordiformis | T | R | 27.5 |
| Chaerophyllum bulbosum | T | S | 27.1 |
| Chaerophyllum bulbosum | T | O | 100.0 |
| Chelidonium majus | T | O | 54.0 |
| Chrysanthemum parthenium | T | S | 50.4 |
| Chrysanthenum coronarium | T | S | 25.8 |
| Cichorium intybus | T | R | 23.9 |
| Citrullus lanatus | T | S | 33.2 |
| Citrullus lanatus (Garden baby) | T | S | 21.4 |
| Citrus limettoides | T | O | 39.2 |
| Citrus limon | T | O | 60.4 |
| Corchorus olitorius | T | S | 28.6 |
| Cornus canadensis L. | T | O | 50.0 |
| Cornus canadensis L. | T | R | 80.6 |
| Cosmos sulphureus | T | R | 20.5 |
| Cosmos sulphureus | T | S | 27.0 |
| Crataegus sp | T | S | 43.9 |
| Crataegus submollis | T | O | 24.2 |
| Crataegus submollis | T | R | 55.1 |
| Cucumis anguria | T | S | 33.2 |
| Cucumis sativus Fanfare | T | S | 35.4 |
| Cucurbita moschata | T | S | 30.4 |
| Cucurbita pepo | T | R | 23.8 |
| Cucurbita pepo | T | S | 46.6 |
| Cuminum cyminum | T | S | 23.1 |
| Curcuma zedoaria | T | S | 20.8 |
| Cymbopogon citratus | T | S | 39.7 |
| Dolichus lablab | T | S | 25.8 |
| Dryopteris filix-mas | T | O | 54.0 |
| Echinacea purpurea | T | S | 20.4 |
| Eriobotrya japonica | T | O | 34.8 |
| Eriobotrya japonica | T | S | 42.9 |
| Foericulum vulgare | T | O | 33.1 |
| Fragaria x ananassa | T | S | 20.3 |
| Fragaria x ananassa | T | R | 42.8 |
| Glycine max | T | O | 26.3 |
| Glycine max | T | O | 30.5 |
| Gossypium herbaceum | T | R | 22.5 |
| Guizotia abyssinica | T | R | 46.6 |
| Hamamelis virginiana | T | S | 33.1 |
| Hamamelis virginiana | T | S | 33.1 |
| Hamamelis virginiana | T | R | 44.8 |
| Hedeoma pulegiodes | T | O | 46.8 |
| Helenium hoopesii | T | R | 27.9 |
| Helianthus annus | T | S | 22.7 |
| Helianthus strumosus | T | O | 30.0 |
| Heliotropium arborescens | T | O | 53.7 |
| Helleborus niger | T | S | 40.5 |
| Hibiscus cannabinus | T | O | 34.0 |
| Hordeum vulgare subsp. Vulgare | T | O | 100.0 |
| Humulus lupulus | T | S | 24.9 |
| Humulus lupulus | T | R | 55.1 |
| Humulus lupulus | T | R | 77.6 |
| Humulus lupulus | T | S | 79.1 |
| Humulus lupulus | T | S | 100.0 |
| Humulus lupulus | T | R | 100.0 |
| Humulus lupulus | T | S | 100.0 |
| Hypericum henryi | T | R | 100.0 |
| Hypericum perforatum | T | O | 99.3 |
| Hypomyces lactiflorum | T | O | 20.5 |
| Iris versicolor | T | R | 48.5 |
| Juniperus communis | T | R | 33.8 |
| Lactuca serriola | T | R | 21.5 |
| Laportea canadensis | T | S | 37.7 |
| Lavendula angustifolia | T | S | 91.7 |
| Lepidium sativum | T | R | 24.7 |
| Levisticum officinale | T | O | 24.9 |
| Lolium perenne | T | S | 22.3 |
| Lonicera ramosissima | T | R | 42.5 |
| Lonicera syringantha | T | R | 21.1 |
| Malus | T | O | 53.1 |
| Malus hupehensis (Pamp.) Rehd. | T | R | 76.5 |
| Malus sp. | T | R | 39.8 |
| Malus sp. | T | R | 45.7 |
| Malva moschata | T | S | 22.8 |
| Malva sylvestris | T | O | 57.6 |
| Matteucia pensylvanica | T | R | 20.1 |
| Melissa officinalis | T | O | 55.0 |
| Mentha piperita | T | R | 35.5 |
| Mentha piperita | T | O | 43.9 |
| Mentha piperita | T | R | 56.6 |
| Mentha pulegium | T | O | 33.3 |
| Mentha pulegium | T | R | 56.2 |
| Mentha spicata | T | O | 43.4 |
| Mentha spicata | T | O | 58.0 |
| Nicotiana tabacum | T | R | 27.3 |
| Nigella sativa | T | R | 25.1 |
| Ocimum Basilicum | T | R | 20.2 |
| Ocnothera bienris | T | S | 37.8 |
| Origanum marjonara | T | R | 45.2 |
| Origanum vulgare | T | S | 21.3 |
| Origanum vulgare | T | O | 23.3 |
| Origanum vulgare | T | R | 23.6 |
| Origanum vulgare | T | O | 37.2 |
| Panicum miliaceum | T | S | 20.6 |
| Panicum miliaceum | T | S | 30.7 |
| Pastinaca saliva | T | R | 26.1 |
| Pastinaca sativa | T | O | 100.0 |
| Peucedanum oreaselinum | T | S | 39.6 |
| Peucedanum oreaselinum | T | R | 53.4 |
| Phaseolus vulgaris | T | S | 21.8 |
| Phaseolus vulgaris | T | O | 23.6 |
| Phaseolus vulgaris | T | O | 59.8 |
| Physalis alkekengi | T | O | 55.5 |
| Physalis pruinosa | T | S | 24.8 |
| Plantago major | T | O | 77.1 |
| Poa compressa | T | R | 54.4 |
| Polygonium chinense | T | O | 36.3 |
| Polygonium chinense | T | R | 61.4 |
| Polygonum persicaria | T | S | 21.3 |
| Populus incrassata | T | S | 50.7 |
| Populus incrassata | T | S | 50.7 |
| Populus x petrowskyana | T | R | 66.7 |
| Prunus cerasifera | T | O | 26.1 |
| Prunus cerasifera | T | R | 64.2 |
| Psidium guajaba | T | S | 22.9 |
| Ptaridium aquilinus | T | R | 43.0 |
| Pyrus pyrifolia | T | S | 28.2 |
| Rahmnus frangula | T | R | 25.9 |
| Raphanus sativus | T | R | 21.4 |
| Raphanus sativus | T | O | 36.9 |
| Rhamnus frangula | T | O | 43.2 |
| Rheum rhabarbarum | T | O | 28.5 |
| Rheum x cultorum | T | R | 28.2 |
| Rianus communis | T | S | 32.4 |
| Ribes nidigrolaria | T | S | 28.5 |
| Ribes nigrum | T | R | 49.9 |
| Rosa rugosa | T | S | 29.1 |
| Rosmarinum officinalis | T | R | 48.2 |
| Rubus arcticus | T | R | 59.1 |
| Rubus ideaus | T | O | 21.5 |
| Rubus pubescens | T | O | 51.8 |
| Rubus thibetanus | T | O | 33.7 |
| Rumex patientia | T | S | 34.4 |
| Ruta graveolens | T | O | 24.3 |
| Salvia (elegens) | T | O | 37.2 |
| Salvia (elegens) | T | R | 42.9 |
| Salvia officinalis | T | R | 67.3 |
| Sambucus canadensis | T | S | 30.2 |
| Sanguisorba minor | T | R | 21.0 |
| Sanguisorba minor | T | R | 29.9 |
| Sanguisorba minor | T | R | 30.8 |
| Sanguisorba minor | T | R | 44.5 |
| Santolina | T | R | 43.8 |
| Sarratula tinctoria | T | S | 37.7 |
| Satureja montana | T | R | 45.0 |
| Satureja repandra | T | S | 46.3 |
| Scorzorera hipanica | T | R | 25.7 |
| Scuttellaria lateriflora | T | S | 41.2 |
| Setaria italica | T | S | 33.4 |
| Solidago canadensis | T | S | 78.5 |
| Stachys affinis | T | S | 100.0 |
| Stachys byzantina | T | O | 100.0 |
| Stellaria media (linné) Cyrillo | T | O | 51.2 |
| Tanacetum vulgare | T | R | 30.5 |
| Tepary | T | R | 31.7 |
| Tepary | T | O | 39.7 |
| Thymus serpyllum | T | O | 29.9 |
| Thymus serpyllum | T | R | 32.8 |
| Thymus x citriodorus | T | S | 22.1 |
| Tiarella cordifolia | T | R | 46.8 |
| Tragopogon porrifolium | T | R | 26.3 |
| Tragopogon porrifolium | T | R | 29.8 |
| Tragopogon porrifolium | T | O | 58.0 |
| Triticale sp. | T | O | 25.3 |
| Tropaeolum majus | T | O | 46.9 |
| Tropaeolum majus | T | O | 55.8 |
| Tropaeolum majus | T | R | 64.7 |
| Tsuga can0adensis | T | R | 39.2 |
| Vaccinium angustifolium | T | R | 28.0 |
| Vaccinium angustifolium | T | S | 29.6 |
| Vaccinium angustifolium | T | R | 33.3 |
| Vaccinium angustifolium Ait. | T | R | 100.0 |
| Vaccinium macrocarpon | T | S | 25.1 |
| Vaccinium macrocarpon | T | R | 27.4 |
| Vaccinium macrocarpon | T | O | 35.4 |
| Vaccinium macrocarpon | T | R | 80.5 |
| Vaccinium macrocarpon | T | O | 90.5 |
| Valeriana officinalis | T | O | 33.0 |
| Veratrum viride | T | S | 46.8 |
| Verbascum thapsus | T | O | 33.4 |
| Vicia faba | T | R | 26.6 |
| Vicia faba | T | O | 35.8 |
| Vigna angularia | T | S | 29.3 |
| Vigna angularia | T | O | 54.0 |
| Vigna sesquipedalis | T | O | 100.0 |
| Vigna unguiculata | T | S | 49.5 |
| Vitia sp. | T | O | 99.6 |
| Vitis sp | T | R | 50.9 |
| Vitis sp. | T | R | 75.8 |
| Weigela coracensis | T | S | 22.8 |
| Weigela coracensis | T | S | 22.8 |
| Weigela hortensis | T | R | 54.9 |
| Zea mays | T | O | 74.3 |

TABLE 4
Inhibition of MMP-9 by Plant Extracts

| | | | Inhibition |
|---|---|---|---|
| Latin name | Stress | Extract | (%) |
| Abelmochus esculentus | A | S | 26.8 |
| Achillea millefolium | A | S | 41.6 |
| Aconitum napellus | A | O | 47.7 |
| Acorus calamus | A | O | 83.2 |
| Actinidia arguta | A | S | 26.8 |
| Adiantum pedatum | A | O | 20.7 |
| Agastache foeniculum | A | S | 100.0 |
| Agrimonia eupatoria | A | W | 21.4 |
| Agropyron cristatum | A | R | 51.4 |
| Agropyron repens | A | S | 27.3 |
| Agrostis alba | A | R | 40.6 |
| Agrostis Stofonifera | A | R | 35.4 |
| Alcea rosea | A | S | 45.8 |
| Alkanna tinctoria | A | S | 42.5 |
| Allium cepa | A | O | 49.7 |
| Allium grande | A | R | 71.4 |
| Allium porrum | A | S | 28.0 |
| Allium porrum | A | O | 82.0 |
| Allium sativum | A | S | 23.7 |
| Allium schoenoprasum | A | O | 45.5 |
| Allium tuberosum | A | V | 20.1 |
| Allium Tuberosum | A | O | 91.5 |
| Althaea officinalis | A | S | 29.6 |
| Amaranthus gangeticus | A | O | 25.1 |
| Amaranthus gangeticus | A | R | 31.1 |
| Amaranthus gangeticus | A | S | 73.2 |
| Amaranthus retroflexus | A | S | 20.4 |
| Ambrosia artemisiifolia | A | R | 50.1 |
| Amelanchier sanguinea | A | W | 37.6 |
| Anthemis nobilis | A | O | 40.4 |
| Anthemis nobilis | A | R | 66.7 |
| Anthemis tinctorium | A | S | 30.3 |
| Apium graveolens | A | R | 71.2 |
| Arachis hypogaea | A | V | 23.5 |
| Aralia cordata | A | S | 21.2 |
| Aralia cordata | A | S | 56.3 |
| Arctium minus | A | R | 31.1 |
| Arctostaphylos uva-ursi | A | S | 31.2 |
| Arctostaphylos uva-ursi | A | O | 31.2 |
| Arctostaphylos uva-ursi | A | R | 59.7 |
| Armoracia rusticana | A | W | 25.1 |
| Armoracia rusticana | A | S | 56.2 |
| Aronia melanocarpa | A | S | 26.8 |
| Aronia melanocarpa | A | S | 41.3 |
| Aronia melanocarpa | A | O | 44.8 |
| Aronia melanocarpa | A | W | 47.7 |
| Aronia melanocarpa | A | R | 55.7 |
| Aronia melanocarpa | A | V | 100.0 |
| Arrhenatherum elatius | A | R | 40.4 |
| Artemisia dracunculus | A | S | 51.1 |
| Asparagus officinalis | A | S | 20.9 |
| Asparagus officinalis | A | S | 32.6 |
| Aster sp | A | O | 29.5 |
| Aster sp | A | R | 80.0 |
| Atropa belladonna | A | S | 47.4 |
| Beta vulgaris | A | S | 25.3 |
| Beta vulgaris | A | R | 26.6 |
| Beta vulgaris | A | W | 34.0 |
| Beta vulgaris | A | O | 42.0 |
| Beta vulgaris | A | V | 44.0 |
| Beta vulgaris spp. Maritima | A | R | 44.0 |
| Beta vulgaris var. condivata | A | R | 35.4 |
| Brassica napus | A | S | 24.6 |
| Brassica napus | A | R | 53.1 |
| Brassica napus | A | O | 100.0 |
| Brassica nigra | A | S | 24.2 |
| Brassica oleracea | A | R | 33.0 |
| Brassica oleracea | A | R | 36.0 |
| Brassica oleracea | A | W | 36.2 |
| Brassica oleracea | A | S | 73.1 |
| Brassica Oleracea | A | O | 100.0 |
| Brassica rapa | A | R | 31.0 |
| Brassica rapa | A | W | 38.6 |
| Brassica rapa | A | V | 42.8 |
| Brassica rapa | A | R | 48.8 |
| Brassica rapa | A | S | 68.2 |
| Brassica rapa | A | O | 89.2 |
| Bromus inermis | A | R | 51.4 |
| Campanula rapunculus | A | O | 25.1 |
| Canna edulis | A | S | 31.1 |
| Canna edulis | A | O | 47.6 |
| Canna edulis | A | R | 68.9 |
| Capsella bursa-pastoris | A | R | 32.5 |
| Capsicum annuum | A | O | 22.0 |
| Capsicum annuum | A | R | 24.0 |
| capsicum annuum | A | S | 55.7 |
| Capsicum frutescens | A | S | 30.3 |
| Capsicum frutescens | A | O | 34.7 |
| Carthamus tinctorius | A | R | 28.5 |
| Carum carvi | A | S | 38.6 |
| Chelidonium majus | A | O | 27.9 |
| Chenopodium bonus-henricus | A | R | 47.4 |
| Chenopodium bonus-henricus | A | O | 20.7 |
| Chenopodium bonus-henricus | A | W | 23.2 |
| chenopodium bonus-henricus | A | S | 62.8 |
| Chenopodium quinoa | A | V | 23.1 |
| Chenopodium quinoa | A | W | 34.7 |
| Chrysanthemum leucanthemum | A | O | 20.6 |
| Chrysanthemum leucanthemum | A | R | 30.9 |
| Chrysanthemun coronarium (Chp | A | R | 26.4 |
| Suey) | | | |
| Chrysanthenum coronarium | A | S | 66.6 |
| Cichorium intybus | A | S | 44.7 |
| Citrullus lanatus | A | S | 62.1 |
| Citrullus lanatus | A | O | 70.6 |
| Coronus canadensis | A | S | 48.5 |
| Cosmos sulphureus | A | S | 23.4 |
| Cosmos sulphureus | A | O | 37.0 |
| Crataegus sp | A | V | 32.4 |
| Crataegus sp | A | S | 45.5 |
| Crataegus sp | A | R | 100.0 |
| Crataegus submollis | A | S | 45.5 |
| Cryptotaenia canadensis | A | W | 26.4 |
| Cucumis Anguria | A | R | 27.2 |
| Cucumis anguria | A | S | 36.6 |
| Cucumis anguria | A | O | 38.5 |
| Cucumis melo | A | O | 59.2 |
| Cucumis sativus | A | R | 39.8 |
| Cucumis sativus | A | O | 49.4 |
| Cucumis sativus | A | S | 54.4 |
| Cucurbita Maxima | A | O | 46.7 |
| Cucurbita moschata | A | S | 32.1 |
| Cucurbita pepo | A | O | 37.0 |
| Curburbita pepo | A | R | 41.0 |
| Curburbita pepo | A | S | 43.9 |
| Curcuma zedoaria | A | S | 67.6 |
| Curcurbita maxima | A | S | 25.8 |
| Cymbopogon citratus | A | O | 26.7 |
| Dactylis glomerata | A | R | 27.2 |
| Datisca cannabina | A | S | 26.9 |
| Datisca cannabina | A | O | 38.0 |
| Daucus carota | A | R | 30.8 |
| Daucus carota | A | O | 31.9 |
| Dirca palustris | A | O | 27.3 |
| Dirca palustris | A | S | 34.2 |
| Dolicos Lablab | A | S | 22.0 |
| Dolicos Lablab | A | R | 25.3 |
| Dryopteris filix-mas | A | S | 24.9 |
| Dryopteris filix-mas | A | R | 40.6 |
| Eleusine coracana | A | S | 20.2 |
| Eleusine coracana | A | R | 20.9 |
| Eleusine coracana | A | O | 71.1 |
| Elymus junceus | A | R | 45.4 |
| Erigeron canadensis | A | S | 35.7 |
| Eruca vesicaria | A | R | 59.9 |
| Fagopyrum esculentum | A | V | 20.7 |
| Fagopyrum tartaricum | A | W | 30.3 |
| Fagopyrum tartaricum | A | O | 33.2 |
| Festuca rubra | A | R | 31.8 |
| Foeniculum Vulgare | A | W | 27.4 |
| Foeniculum vulgare | A | O | 50.6 |
| Forsythia intermedia | A | O | 100.0 |
| Fragaria x ananassa | A | V | 30.0 |
| Fragaria x ananassa | A | S | 36.3 |
| Galium odoratum | A | R | 26.9 |
| Gaultheria hispidula | A | R | 28.4 |
| Gaultheria hispidula | A | S | 40.7 |
| Gentiana lutea | A | R | 34.7 |
| Glechoma hederacea | A | S | 37.6 |
| Glycine max | A | R | 38.1 |
| Glycine Max | A | O | 56.4 |
| Glycine max | A | S | 71.4 |
| Glycyrrhiza glabra | A | S | 62.6 |
| Glycyrrhiza glabra | A | W | 100.0 |
| Guizotia abyssinica | A | R | 91.9 |
| Hamamelis virginiana | A | S | 41.0 |
| Hamamelis virginiana | A | R | 74.6 |
| Hedeoma pulegioides | A | O | 22.0 |
| Helianthus tuberosus | A | W | 21.2 |
| Helianthus tuberosus | A | W | 51.5 |
| Helichrysum angustifolium | A | V | 21.0 |
| Heliotropium arborescens | A | S | 54.1 |
| Helleborus niger | A | S | 37.8 |
| Hordeum hexastichon | A | W | 38.0 |
| Hyssopus officinalis | A | O | 25.1 |
| Inula helenium | A | S | 29.7 |
| Isatis tinctoria | A | S | 41.5 |
| Lactuca serrila | A | R | 41.3 |
| Lactuca serriola | A | S | 46.6 |
| Laportea canadensis | A | S | 26.3 |
| Lathyrus sativus | A | O | 22.2 |
| Lathyrus sativus | A | R | 50.2 |
| Lathyrus sylvestris | A | V | 31.3 |
| Lathyrus sylvestris | A | W | 31.8 |
| Laurus nobilis | A | S | 25.7 |
| Laurus nobilis | A | V | 30.0 |
| Lavandula latifolia | A | S | 40.3 |
| Leonurus cardiaca | A | R | 27.0 |
| Lepidium sativum | A | S | 41.8 |
| Levisticum officinale | A | S | 29.0 |
| Levisticum officinale | A | O | 44.9 |
| Linaria vulgaris miller | A | O | 23.6 |
| Linum usitatissimum | A | R | 33.3 |
| Lolium multiflorum | A | S | 29.0 |
| Lolium perenne | A | R | 52.0 |
| Lotus corniculatus | A | R | 62.9 |
| Lotus tetragonolobus | A | S | 62.9 |
| Lycopersicon esculentum | A | S | 26.1 |
| Lycopersicon esculentum | A | W | 33.0 |
| Malva moschata | A | S | 31.8 |
| Malva sylvestris | A | S | 21.4 |
| Malva verticillata | A | R | 43.4 |
| Matteucia pensylvanica | A | R | 26.9 |
| Medicago sativa | A | V | 20.4 |
| Melilotus albus | A | R | 53.9 |
| Melissa officinalis | A | S | 21.4 |
| Melissa officinalis | A | O | 36.8 |
| Melissa officinalis | A | R | 53.7 |
| Mentha piperita | A | S | 57.7 |
| Mentha pulegium | A | S | 66.1 |
| Mentha spicata | A | S | 67.7 |
| Mentha suaveolens | A | S | 51.8 |
| Momordica charantia | A | R | 29.7 |
| Momordica charantia | A | S | 72.1 |
| Nicotiana rustica | A | O | 30.3 |
| Nicotiana rustica | A | S | 59.1 |
| Nicotiana tabacum | A | S | 39.0 |
| Nicotiana tabacum | A | W | 47.6 |
| Nicotiana tabacum | A | O | 100.0 |
| Nigella sativa | A | R | 59.4 |
| Oenothera biennis | A | O | 21.3 |
| Oenothera biennis | A | O | 36.7 |
| Origanum vulgare | A | W | 21.3 |
| Origanum vulgare | A | V | 42.7 |
| Oryza sativa | A | W | 56.5 |
| Oxyria digyna | A | W | 35.1 |
| Oxyria digyna | A | V | 76.4 |
| Pastinaca sativa | A | V | 20.3 |
| Pastinaca sativa | A | W | 23.2 |
| Pastinaca sativa | A | O | 42.1 |
| Pastinaca sativa | A | R | 46.9 |
| Phalaris canariensis | A | R | 20.3 |
| Phalaris canariensis | A | O | 80.5 |
| Phaseolus mungo | A | O | 51.3 |
| Phaseolus mungo | A | S | 74.1 |
| Phaseolus vulgaris | A | V | 23.0 |
| Phaseolus vulgaris | A | O | 51.4 |
| Phaseolus vulgaris | A | S | 62.6 |
| Phlox paniculata | A | O | 41.0 |
| Physalis alkekengi | A | R | 31.6 |
| Physalis ixocarpa | A | S | 45.2 |
| Physalis Ixocarpa | A | O | 65.3 |
| Physalis Pruinosa | A | O | 87.3 |
| Phytolacca americana | A | S | 49.6 |
| Phytolacca americana | A | O | 89.8 |
| Pimpinella anisum | A | S | 100.0 |
| Plantago coronopus | A | S | 48.3 |
| Plantago coronopus | A | O | 89.3 |
| Plantago major | A | S | 21.8 |
| Poa compressa | A | R | 22.4 |
| Poa compressa | A | S | 49.3 |
| Poa pratensis | A | R | 22.4 |
| Polygonum pensylvanicum | A | S | 43.3 |
| Polygonum persicaria | A | O | 21.6 |
| Polygonum persicaria | A | S | 38.5 |
| Potentilla anserina | A | S | 26.3 |
| Potentilla anserina | A | O | 31.2 |
| Poterium Sanquisorba | A | S | 29.2 |
| Pteridium aquilinum | A | S | 27.3 |
| Raphanus sativus | A | W | 22.7 |
| Raphanus sativus | A | R | 30.8 |
| Raphanus sativus | A | R | 40.2 |
| Raphanus sativus | A | S | 71.5 |
| Raphanus sativus | A | O | 100.0 |
| Rheum rhabarbarum | A | S | 21.3 |
| Rheum rhabarbarum | A | V | 67.9 |
| Rheum rhabarbarum | A | W | 72.4 |
| Ribes nidigrolaria | A | W | 32.6 |
| Ribes nidigrolaria | A | V | 64.6 |
| Ribes nigrum | A | W | 23.6 |
| Ribes nigrum | A | V | 27.2 |
| Ribes nigrum | A | S | 41.0 |
| Ribes nigrum | A | O | 65.8 |
| Ribes Nigrum | A | W | 100.0 |
| Ribes Salivum | A | R | 75.4 |
| Ribes Sylvestre | A | V | 27.7 |
| Ribes Sylvestre | A | W | 100.0 |
| ribes uva-crispa | A | S | 24.4 |
| Ribes Uva-crispa | A | W | 36.6 |
| Ricinus communis | A | R | 21.6 |
| Rosa rugosa | A | V | 30.6 |
| Rosa rugosa | A | S | 36.2 |
| Rosa rugosa | A | W | 39.3 |
| Rosmarinus officinalis | A | W | 27.2 |
| Rosmarinus officinalis | A | R | 45.7 |
| Rubus allegheniensis | A | S | 53.7 |
| Rubus canadensis | A | V | 27.0 |
| Rubus canadensis | A | S | 41.0 |
| Rubus canadensis | A | W | 41.2 |
| Rubus canadensis | A | S | 45.1 |
| Rubus idaeus | A | V | 24.3 |
| Rubus idaeus | A | S | 39.7 |
| Rubus idaeus | A | W | 62.2 |
| Rubus idaeus | A | R | 37.0 |
| Rumex acetosella | A | V | 75.8 |
| Rumex acotosa | A | W | 25.5 |
| Rumex crispus | A | R | 73.3 |
| Rumex crispus | A | O | 60.5 |
| Rumex patientia | A | O | 49.4 |
| Rumex patientia | A | S | 65.8 |
| Rumex Scutatus | A | W | 25.5 |
| Rumex Scutatus | A | V | 61.9 |
| Rumex Scutatus | A | O | 93.8 |
| Ruta graveolens | A | S | 25.8 |
| Ruta graveolens | A | W | 27.1 |
| Salix purpurea | A | S | 22.1 |
| Salix purpurea | A | R | 33.8 |
| Salvia elegans | A | W | 23.7 |
| Salvia officinalis | A | V | 20.8 |
| Salvia officinalis | A | S | 31.4 |
| Salvia sclarea | A | S | 28.0 |
| Satureja montana | A | W | 21.7 |
| Scuttellaria latenflora | A | S | 54.1 |
| Secale cereale | A | V | 22.6 |
| Secale cereale | A | S | 22.9 |
| Secale cereale | A | W | 26.9 |
| Sesamum indicum | A | O | 21.2 |
| Setaria italica | A | O | 27.0 |
| Sium Sisarum | A | R | 32.6 |
| Sium Sisarum | A | O | 42.7 |
| Solanum dulcamara | A | S | 43.3 |
| Solanum dulcamara | A | O | 48.6 |
| Solanum melanocerasum | A | O | 21.3 |
| Solanum melongena | A | R | 20.5 |
| Solanum melongena | A | V | 35.6 |
| Solanum melongena | A | O | 49.4 |
| Solanum melongena | A | S | 65.2 |
| Solidago sp | A | R | 32.7 |
| Spinacia oleracea | A | S | 41.0 |
| Stachys affinis | A | R | 22.5 |
| Stachys affinis | A | S | 43.9 |
| Stachys affinis | A | O | 92.0 |
| Symphytum officinale | A | S | 28.0 |
| Tanacetum cinerariifolium | A | O | 20.3 |
| Tanacetum cinerariifolium | A | R | 69.7 |
| Tanacetum vulgare | A | O | 20.2 |
| Tanacetum vulgare | A | S | 84.2 |
| Teucrium chamaedrys | A | O | 20.4 |
| Teucrium chamaedrys | A | R | 20.4 |
| Thymus serpyllum | A | W | 24.3 |
| Thymus vulgaris | A | S | 42.5 |
| Thymus x citriodorus | A | W | 27.4 |
| Tragopogon porrifolius | A | W | 21.9 |
| Tragopogon porrifolius | A | V | 26.2 |
| Trifolium hybridum | A | R | 30.9 |
| Trifolium pannonicum | A | R | 41.0 |
| Trifolium repens | A | R | 51.3 |
| Trigonella foenum graecum | A | S | 44.2 |
| Triticum spelta | A | S | 30.0 |
| Triticum turgidum | A | S | 31.3 |
| Typha latifolia | A | S | 57.7 |
| Urtica dioica | A | O | 26.5 |
| Urtica dioica | A | S | 50.2 |
| Vaccinium Corymbosum | A | W | 39.9 |
| Vaccinium Corymbosum | A | S | 64.8 |
| Vaccinum augustifolium | A | R | 44.8 |
| Vaccinum macrocarpon | A | S | 100.0 |
| Veratrum viride | A | S | 29.1 |
| Veratrum viride | A | O | 31.8 |
| Verbascum thapsus | A | S | 42.6 |
| Verbascum thapsus | A | O | 75.2 |
| Viburnum trilobum | A | V | 97.4 |
| Vicia sativa | A | R | 53.3 |
| Vicia villosa | A | R | 48.9 |
| Vigna unguiculata | A | R | 27.0 |
| Vigna unguiculata | A | O | 44.8 |
| Vigna unguiculata | A | S | 55.5 |
| Vinca minor | A | S | 35.1 |
| Vitis sp. | A | V | 52.2 |
| Vitis sp. | A | S | 59.6 |
| Vitis sp. | A | R | 87.8 |
| Xanthium sibiricum | A | S | 57.1 |
| Zea mays | A | V | 26.1 |
| Zea mays | A | W | 32.1 |
| Zea Mays | A | O | 38.7 |
| Achillea millefolium | G | S | 45.5 |
| Aconitum napellus | G | S | 24.0 |
| Aconitum napellus | G | O | 53.9 |
| Acorus calamus | G | O | 87.6 |
| Acorus calamus | G | S | 100.0 |
| Actinidia arguta | G | S | 33.8 |
| Adiantum pedatum | G | R | 31.6 |
| Adiantum pedatum | G | S | 31.7 |
| Ageratum conyzoides | G | S | 23.1 |
| Agropyron cristatum | G | R | 64.1 |
| Agropyron repens | G | S | 29.2 |
| Agropyron repens | G | O | 32.6 |
| Agrostis Stolonifera | G | R | 34.4 |
| Alcea rosea | G | S | 22.7 |
| Alchemilla mollis | G | S | 30.5 |
| Alchemilla mollis | G | W | 33.2 |
| Allium ampeloprasum | G | O | 53.4 |
| Allium cepa | G | S | 22.5 |
| Allium cepa | G | O | 60.7 |
| Allium schoenoprasum | G | S | 21.1 |
| Allium schoenoprasum | G | O | 60.4 |
| Allium tuberosum | G | S | 38.8 |
| Allium tuberosum | G | O | 74.4 |
| Althaea officianalis | G | S | 54.9 |
| Amaranthus candathus | G | O | 42.6 |
| Amaranthus caudathus | G | W | 27.1 |
| Amaranthus gangeticus | G | S | 56.8 |
| Amaranthus gangeticus | G | S | 74.4 |
| Ambrosia artemisiifolia | G | R | 49.0 |
| Amelanchier sanguinea | G | W | 45.2 |
| Angelica archangelica | G | S | 20.9 |
| Anthemis nobilis | G | R | 58.9 |
| Apium graveolens | G | O | 30.4 |
| Apium graveolens | G | S | 36.4 |
| Apium graveolens | G | R | 60.6 |
| Arachis hypogaea | G | W | 26.0 |
| Aralia cordata | G | S | 66.0 |
| Arctium minus | G | O | 26.6 |
| Arctium minus | G | R | 30.8 |
| Arctostaphylos uva-ursi | G | S | 29.3 |
| Arctostaphylos uva-ursi | G | O | 38.8 |
| Arctostaphylos uva-ursi | G | R | 80.2 |
| Armoracia rusticana | G | S | 62.7 |
| Aronia melanocarpa | G | O | 26.7 |
| Aronia melanocarpa | G | V | 100.0 |
| Aronia melanocarpa | G | R | 100.0 |
| Aronia melanocarpa (Michx.) Ell. | G | W | 39.1 |
| Artemisia dracunculus | G | O | 44.3 |
| Artemisia dracunculus | G | S | 65.4 |
| Asclepias incarnata | G | R | 20.3 |
| Asparagus officinalis | G | O | 22.3 |
| Asparagus officinalis | G | S | 26.6 |
| Asparagus officinalis | G | W | 28.7 |
| Aster sp | G | O | 34.3 |
| Aster sp | G | R | 62.6 |
| Atropa belladonna | G | S | 34.9 |
| Beta vulgaris | G | R | 28.3 |
| Beta vulgaris | G | R | 42.2 |
| Beta vulgaris | G | O | 47.0 |
| Beta vulgaris spp. Maritima | G | O | 46.7 |
| Brassica cepticepa | G | R | 26.7 |
| Brassica cepticepa | G | S | 68.3 |
| Brassica juncea | G | O | 45.0 |
| Brassica juncea | G | S | 66.1 |
| Brassica Napus | G | S | 27.5 |
| Brassica Napus | G | R | 37.6 |
| Brassica napus | G | O | 94.8 |
| Brassica nigra | G | S | 36.4 |
| Brassica oleracea | G | R | 38.7 |
| Brassica oleracea | G | W | 39.0 |
| Brassica oleracea | G | R | 49.4 |
| Brassica oleracea | G | S | 76.1 |
| Brassica oleracea | G | O | 100.0 |
| Brassica rapa | G | R | 21.1 |
| Brassica rapa | G | S | 64.0 |
| Brassica rapa | G | O | 100.0 |
| Bromus inermis | G | R | 36.7 |
| Campanula rapunculus | G | O | 59.9 |
| Canna edulis | G | O | 20.8 |
| Canna edulis | G | O | 83.1 |
| Capsicum annuum | G | R | 20.2 |
| Capsicum annuum | G | S | 29.6 |
| Capsicum annuum | G | O | 51.5 |
| Capsicum annuum | G | S | 60.8 |
| Capsicum frutescens | G | S | 32.8 |
| Carthamus tinctorius | G | R | 29.8 |
| Carum carvi | G | S | 30.4 |
| Chelidonium majus | G | O | 39.9 |
| Chenopodium bonus-henricus | G | O | 63.0 |
| Chenopodium quinoa | G | O | 34.1 |
| Chenopodium quinoa | G | W | 42.8 |
| Chenopodium quinoa | G | V | 46.1 |
| Chichorium endivia subsp endivia | G | W | 22.0 |
| Chichorium endivia subsp endivia | G | S | 22.9 |
| Chrysanthemum coronarium | G | R | 23.2 |
| Chrysanthemum coronarium | G | S | 68.4 |
| Chrysanthemum leucanthemum | G | R | 20.5 |
| Cicer arietinum | G | S | 25.7 |
| Cichorium intybus | G | W | 51.1 |
| Cichorium intybus | G | S | 53.4 |
| Citrullus lanatus | G | S | 36.5 |
| Citrullus lanatus | G | O | 71.5 |
| Coix Lacryma-Jobi | G | O | 21.0 |
| Cornus canadensis | G | S | 34.8 |
| Crataegus sp | G | W | 54.0 |
| Crataegus submollis | G | S | 31.3 |
| Cryptotaenia canadensis | G | W | 32.1 |
| Cucumis anguria | G | S | 27.3 |
| Cucumis anguria | G | O | 32.5 |
| Cucumis sativus | G | O | 39.4 |
| Cucumis sativus | G | S | 69.4 |
| Cucurbita maxima | G | O | 34.1 |
| Cucurbita maxima | G | S | 42.6 |
| Cucurbita moschata | G | S | 32.0 |
| Cucurbita moschata | G | O | 39.2 |
| Cucurbita pepo | G | S | 28.8 |
| Cucurbita pepo | G | O | 32.6 |
| Curcuma zedoaria | G | O | 23.3 |
| Curcuma zedoaria | G | S | 57.6 |
| Cymbopogon citratus | G | O | 70.1 |
| Cynara scolymus | G | S | 20.2 |
| Cynara scolymus | G | O | 37.5 |
| Cynara scolymus | G | R | 88.7 |
| Cyperus esculentus | G | S | 66.7 |
| Datura metel | G | S | 29.2 |
| Datura stramonium | G | O | 27.6 |
| Daucus carota | G | O | 24.2 |
| Daucus carota | G | R | 29.3 |
| Dipsacus sativus | G | S | 48.7 |
| Dirca palustris | G | O | 29.9 |
| Dirca palustris | G | S | 36.4 |
| Dolichos Lablab | G | S | 35.8 |
| Dolichos Lablab | G | R | 74.5 |
| Dryopteris filix-mas | G | S | 27.9 |
| Dryopteris filix-mas | G | R | 42.6 |
| Echinochloa frumentacea | G | O | 68.4 |
| Eleusine coracana | G | O | 47.8 |
| Elymus junceus | G | R | 42.7 |
| Erigeron canadensis | G | S | 37.8 |
| Erigeron speciosus | G | R | 34.6 |
| Errhenatherum elatius | G | R | 34.4 |
| Fagopyrum tartaricum | G | W | 31.4 |
| Foeniculum vulgare | G | W | 28.0 |
| Foeniculum vulgare | G | S | 44.6 |
| Foeniculum vulgare | G | O | 68.9 |
| Foeniculum Vulgare | G | R | 100.0 |
| Forsythia intermedia | G | O | 100.0 |
| Forsythia x intermedia | G | O | 79.5 |
| Galium odoratum | G | S | 32.4 |
| Galium odoratum | G | R | 100.0 |
| Gaultheria hispidula | G | R | 48.4 |
| Gaultheria hispidula | G | S | 80.4 |
| Gaultheria hispidula | G | O | 100.0 |
| Gaultheria procumbens | G | S | 26.9 |
| Gaultheria procumbens | G | W | 54.3 |
| Glechoma hederacea | G | S | 26.6 |
| Glycine max | G | R | 52.5 |
| Glycine max | G | O | 67.9 |
| Glycine max | G | O | 75.8 |
| Glycyrrhiza glabra | G | R | 21.4 |
| Glycyrrhiza glabra | G | V | 21.6 |
| Glycyrrhiza glabra | G | W | 100.0 |
| Guizotia abyssinica | G | R | 91.4 |
| Hamamelis virginiana | G | O | 39.8 |
| Hamamelis virginiana | G | R | 78.8 |
| Hamamelis virginiana | G | S | 96.6 |
| Hedeoma pulegioides | G | S | 45.4 |
| Helenium hoopesii | G | S | 22.6 |
| Helenium hoopesii | G | O | 52.8 |
| Helianthus annuus | G | R | 22.0 |
| Helianthus annuus | G | S | 31.6 |
| Helianthus strumosus | G | R | 30.5 |
| Helianthus strumosus | G | O | 71.7 |
| Helianthus tuberosus | G | W | 21.2 |
| Helianthus tuberosus | G | S | 50.7 |
| Helianthus tuberosus L. | G | R | 24.9 |
| Heliotropium arborescens | G | S | 40.0 |
| Heliotropium arborescens | G | O | 45.6 |
| Helleborus niger | G | S | 38.0 |
| Hordeum vulgare | G | S | 21.5 |
| Humulus lupulus | G | O | 35.1 |
| Hypericum sp | G | W | 26.1 |
| Hyssopus officinalis | G | S | 74.5 |
| Iberis amara | G | O | 20.9 |
| Iberis amara | G | S | 21.7 |
| Inula helenium | G | S | 27.6 |
| Ipomoea batatas | G | S | 37.5 |
| Isatis tinctoria | G | S | 48.0 |
| Lachica serrola | G | R | 53.0 |
| Lactuca sativa | G | W | 24.5 |
| Laportea canadensis | G | S | 36.0 |
| Laportea canadensis | G | O | 81.7 |
| Lathyrus sativus | G | W | 37.8 |
| Lathyrus sylvestris | G | R | 40.7 |
| Lathyrus sylvestris | G | O | 79.1 |
| Laurus nobilis | G | S | 22.7 |
| Lavandula angustifolia | G | S | 31.7 |
| Lavandula latifolia | G | O | 27.2 |
| Ledum groenlandicum | G | S | 61.1 |
| Leonurus cardiaca | G | O | 22.6 |
| Lepidium sativum | G | S | 23.3 |
| Levisticum officinale | G | S | 23.1 |
| Levisticum officinale | G | W | 27.5 |
| Levisticum officinale | G | O | 41.3 |
| Linum usitatissimum | G | R | 21.4 |
| Lolium perenne | G | R | 32.7 |
| Lotus corniculatus | G | R | 54.2 |
| Malus hupehensis | G | R | 26.4 |
| Malva verticillata | G | R | 37.9 |
| Matricaria recutita | G | O | 50.3 |
| Medicago sativa | G | W | 29.1 |
| Melilotus albus | G | R | 52.1 |
| Melissa officinalis | G | O | 22.7 |
| Melissa officinalis | G | S | 35.9 |
| Melissa officinalis | G | R | 38.6 |
| Mentha piperita | G | S | 64.4 |
| Mentha suaveolens | G | W | 22.5 |
| Momordica charantia | G | R | 29.3 |
| Momordica charantia | G | S | 90.6 |
| Nepeta cataria | G | R | 50.5 |
| Nicotiana rustica | G | O | 35.3 |
| Nicotiana rustica | G | S | 100.0 |
| Nicotiana tabacum | G | S | 31.6 |
| Nicotiana tabacum | G | O | 100.0 |
| Nigella sativa | G | R | 24.2 |
| Ocimum basilicum | G | S | 30.6 |
| Oenothera biennis | G | O | 48.0 |
| Oenothera biennis | G | R | 76.6 |
| Origanum vulgare | G | V | 41.3 |
| Oryza Saliva | G | O | 22.1 |
| Oxyria digyna | G | O | 26.5 |
| Oxyria digyna | G | V | 70.3 |
| Panicum miliaceum | G | O | 94.4 |
| Pastinaca sativa | G | R | 29.4 |
| Pastinaca sativa | G | S | 79.2 |
| Pennisetum alopecuroides | G | O | 22.0 |
| Petasites japonicus | G | S | 29.2 |
| Peucedanum oreaselinum | G | O | 21.3 |
| Phacelia tanacetifolia | G | R | 23.5 |
| Phalaris arundinacea | G | R | 47.5 |
| Phalaris canariensis | G | R | 23.1 |
| Phalaris canariensis | G | O | 100.0 |
| Phaseolus coccineus | G | O | 37.0 |
| Phaseolus coccineus | G | R | 74.1 |
| Phaseolus mungo | G | O | 42.2 |
| Phaseolus mungo | G | S | 52.2 |
| Phaseolus vulgaris | G | V | 35.5 |
| Phaseolus vulgaris | G | S | 48.0 |
| Phaseolus vulgaris | G | O | 58.1 |
| Phlox paniculata | G | S | 32.2 |
| Phlox paniculata | G | O | 40.1 |
| Physalis ixocarpa | G | O | 20.6 |
| Physalis pruinosa | G | O | 80.0 |
| Phytolacca americana | G | S | 62.0 |
| Phytolacca americana | G | O | 100.0 |
| Pimpinella anisum | G | S | 37.3 |
| Pisum sativum | G | W | 34.4 |
| Pisum sativum | G | O | 63.3 |
| Plantago coronopus | G | O | 42.7 |
| Plantago coronopus | G | S | 46.4 |
| Plantago major | G | O | 28.3 |
| Plantago major | G | S | 41.4 |
| Plectranthus sp. | G | S | 29.3 |
| Poa compressa | G | R | 22.1 |
| Poa compressa | G | S | 45.5 |
| Poa pratensis | G | R | 35.7 |
| Polygonum pensylvanicum | G | S | 38.3 |
| Polygonum persicaria | G | S | 31.0 |
| Potentilla anserina | G | O | 46.8 |
| Poterium sanquisorba | G | S | 24.7 |
| Poterium sanquisorba | G | W | 30.6 |
| Prunus cerasifera | G | R | 45.9 |
| Pteridium aquilinum | G | S | 22.4 |
| Raphanus Raphanistrum | G | S | 36.5 |
| Raphanus Raphanistrum | G | O | 75.0 |
| Raphanus sativus | G | R | 20.8 |
| Raphanus sativus | G | R | 27.5 |
| Raphanus sativus | G | S | 35.4 |
| Rheum rhabarbarum | G | S | 27.0 |
| Ribes Grossularia | G | W | 33.7 |
| Ribes nidigrolaria | G | S | 30.7 |
| Ribes nidigrolaria | G | V | 40.5 |
| Ribes nigrum | G | V | 35.9 |
| Ribes nigrum | G | W | 58.6 |
| Ribes Silvestris | G | V | 26.9 |
| Ribes Silvestris | G | W | 100.0 |
| Ricinus communis | G | R | 21.8 |
| Rosmarinus officinalis | G | S | 24.7 |
| Rosmarinus officinalis | G | W | 30.9 |
| Rosmarinus officinalis | G | R | 60.3 |
| Rubus ideaus | G | O | 32.5 |
| Rubus ideaus | G | S | 47.0 |
| Rubus occidentalis | G | S | 39.4 |
| Rubus occidentalis | G | R | 74.1 |
| Rumex acetosa | G | W | 45.6 |
| Rumex acetosella | G | W | 22.8 |
| Rumex acetosella | G | V | 31.5 |
| Rumex crispus | G | O | 25.9 |
| Rumex crispus | G | R | 70.3 |
| Rumex patientia | G | O | 39.8 |
| Rumex patientia | G | S | 54.2 |
| Rumex scutatus | G | W | 23.8 |
| Rumex scutatus | G | V | 69.9 |
| Rumex scutatus | G | O | 78.8 |
| Ruta graveolens | G | R | 30.7 |
| Ruta graveolens | G | S | 61.5 |
| Salvia elagens | G | W | 25.4 |
| Salvia elegans | G | S | 31.1 |
| Sambucus canadensis | G | W | 80.6 |
| Sambucus ebulus | G | W | 26.1 |
| Sambucus ebulus | G | V | 34.4 |
| Sambucus ebulus | G | S | 37.8 |
| Sanguisorba officinalis | G | R | 100.0 |
| Santolina chamaecyparissus | G | R | 21.7 |
| Santolina chamaecyparissus | G | S | 25.2 |
| Satureja montana | G | O | 21.2 |
| Scuttellaria lateriflora | G | S | 37.0 |
| Secale cereale | G | S | 26.7 |
| Secale cereale | G | W | 27.3 |
| Serratula tinctoria | G | S | 36.2 |
| Serratula tinctoria | G | O | 70.3 |
| Sesamum indicum | G | O | 27.6 |
| Sesamum indicum | G | S | 44.3 |
| Silybum marianum | G | S | 34.7 |
| Sium sisarum | G | O | 79.0 |
| Solanum dulcamara | G | R | 25.2 |
| Solanum dulcamara | G | S | 64.6 |
| solanum melongena | G | S | 36.6 |
| solanum melongena | G | O | 40.1 |
| solanum melongena | G | V | 50.0 |
| solanum melongena | G | S | 74.9 |
| Solanum tuberosum | G | S | 39.1 |
| Solanum tuberosum | G | O | 39.2 |
| Solidago sp | G | R | 30.7 |
| Sorghum caffrorum | G | O | 87.9 |
| Sorghum dochna | G | W | 20.6 |
| Sorghum dochna | G | O | 20.6 |
| Sorghum dochna | G | S | 34.1 |
| Sorghum dochna | G | O | 97.0 |
| Sorghum durra | G | O | 30.6 |
| sorghum durra | G | S | 30.6 |
| sorghum durra | G | O | 48.6 |
| Sorghum sudanense | G | S | 21.7 |
| Sorghum sudanense | G | O | 24.6 |
| Sorghum sudanense | G | V | 32.1 |
| Spinacia oleracea | G | S | 53.2 |
| Stachys Affinis | G | S | 25.0 |
| Stachys Affinis | G | R | 27.8 |
| Stachys Affinis | G | O | 100.0 |
| Symphytum officinale | G | W | 21.7 |
| Symphytum officinale | G | O | 25.2 |
| Symphytum officinale | G | S | 34.6 |
| Tanacetum cinerariifolium | G | R | 52.4 |
| Tanacetum vulgare | G | R | 27.1 |
| Tanacetum vulgare | G | S | 72.7 |
| Teucrium chamaedrys | G | R | 24.6 |
| Teucrium chamaedrys | G | O | 52.8 |
| Thymus fragantissumus | G | R | 100.0 |
| Thymus vulgaris | G | V | 24.2 |
| Thymus x citriodorus | G | S | 23.7 |
| Tiarella cordifolia | G | S | 20.8 |
| Tiarella cordifolia | G | O | 30.8 |
| Tragopogon porrifolius | G | O | 22.8 |
| Trifolium hybridum | G | R | 24.7 |
| Trifolium pannonicum | G | R | 65.5 |
| Trifolium repens | G | R | 57.5 |
| Trigonella foenumgraecum | G | S | 37.6 |
| Triticum furgidum | G | S | 56.5 |
| Triticum spelta | G | S | 40.8 |
| Tropaeolum majus | G | O | 76.1 |
| Typha latifolia | G | S | 43.3 |
| Urtica dioica | G | S | 40.3 |
| Vaccinium angustifolium | G | S | 42.4 |
| Vaccinium corymbosum | G | S | 61.5 |
| Vaccinium macrocarpon | G | S | 43.7 |
| Vaccinum angustifolium | G | R | 23.1 |
| Veratrum viride | G | S | 43.6 |
| Verbascum thapsus | G | S | 37.8 |
| Verbascum thapsus | G | O | 87.0 |
| Veronica officinalis | G | S | 30.5 |
| Viburnum trilobum | G | S | 49.4 |
| Viburnum trilobum | G | R | 100.0 |
| Viburnum trilobum | G | V | 100.0 |
| Vicia faba | G | R | 50.5 |
| Vicia sativa | G | R | 42.4 |
| Vicia villosa | G | R | 89.2 |
| Vigna angularia | G | R | 28.1 |
| Vigna angularia | G | S | 71.5 |
| Vigna unguiculata | G | R | 21.0 |
| Vigna unguiculata | G | O | 38.7 |
| Vigna unguiculata | G | S | 61.1 |
| Vinca minor | G | O | 33.6 |
| Vinca minor | G | S | 34.3 |
| Vitis sp. | G | O | 29.0 |
| Vitis sp. | G | W | 50.2 |
| Vitis sp. | G | S | 53.3 |
| Vitis sp. | G | V | 63.0 |
| Vitis sp. | G | R | 86.6 |
| Withania somnifera | G | S | 20.3 |
| Xanthium sibiricum | G | S | 34.7 |
| Xanthium strumarium | G | S | 23.2 |
| Zea mays | G | V | 20.1 |
| Zea mays | G | S | 45.9 |
| Zea mays | G | O | 97.5 |
| Abelmochus esculentus | T | S | 24.8 |
| Abies lasiocarpa | T | W | 44.7 |
| Achillea millefolium | T | O | 24.1 |
| Achillea millefolium | T | S | 59.2 |
| Aconitum napellus | T | S | 40.6 |
| Aconitum napellus | T | O | 41.6 |
| Acorus calamus | T | O | 47.1 |
| Actinidia arguta | T | S | 21.8 |
| Adiantum pedatum | T | S | 26.8 |
| Adiantum pedatum | T | O | 45.8 |
| Adiantum pedatum | T | R | 86.0 |
| Agaricus bisporus | T | S | 26.3 |
| Agaricus bisporus | T | O | 29.8 |
| Agaricus bisporus | T | W | 36.9 |
| Agaricus bisporus | T | W | 44.0 |
| Agaricus bisporus | T | S | 46.0 |
| Agastache foeniculum | T | S | 70.0 |
| Ageratum conyzoides | T | S | 31.7 |
| Agropyron cristatum | T | R | 86.9 |
| Agropyron repens | T | O | 49.6 |
| Agrostis alba | T | R | 21.9 |
| Agrostis Stolonifera | T | R | 35.8 |
| Alcea rosea | T | S | 35.2 |
| Alchemilla mollis | T | S | 37.9 |
| Allium ampeloprasum | T | O | 48.0 |
| Allium ascalonicum | T | S | 26.2 |
| Allium ascalonicum | T | O | 77.2 |
| Allium cepa | T | O | 92.6 |
| Allium grande | T | R | 60.4 |
| Allium schoenoporasum | T | O | 65.8 |
| Allium schoenoprasum | T | W | 31.0 |
| Allium tuberosum | T | S | 22.8 |
| Allium tuberosum | T | O | 99.7 |
| Althaea officianalis | T | S | 22.8 |
| Althaea officinalis | T | O | 22.1 |
| Amaranthus candathus | T | W | 43.9 |
| Amaranthus gangeticus | T | O | 30.3 |
| Amaranthus gangeticus | T | S | 66.0 |
| Ambrosia artemisiifolia | T | R | 58.7 |
| Amelanchier alnitolia | T | R | 70.5 |
| Amelanchier sanguinea | T | W | 37.3 |
| Ananas comosus | T | W | 23.8 |
| Ananas comosus | T | V | 95.0 |
| Ananas comosus | T | O | 99.6 |
| angelica archangelica | T | S | 30.5 |
| angelica archangelica | T | R | 38.9 |
| Anthemis nobilis | T | O | 41.4 |
| Anthemis nobilis | T | R | 72.8 |
| Anthemis tinctorium | T | S | 27.3 |
| Anthriscus cerefolium | T | W | 35.8 |
| Apium graveolens | T | S | 31.7 |
| Apium graveolens | T | W | 32.4 |
| Apium graveolens | T | R | 56.6 |
| Aralia cordata | T | R | 29.2 |
| Aralia cordata | T | S | 45.0 |
| Arctium minus | T | R | 25.8 |
| Arctostaphylos uva-ursi | T | O | 31.0 |
| Arctostaphylos uva-ursi | T | S | 35.2 |
| Arctostaphylos uva-ursi | T | R | 58.6 |
| Armoracia rusticana | T | W | 24.9 |
| Armoracia rusticana | T | S | 52.9 |
| Aronia melanocarpa | T | W | 40.0 |
| Aronia melanocarpa | T | V | 91.9 |
| Aronia prunifolia | T | W | 100.0 |
| Arrhenatherum elatius | T | R | 22.8 |
| Artemisia draculus | T | S | 74.9 |
| Artemisia dracunculus | T | S | 47.8 |
| Asclepias incarnata | T | R | 20.5 |
| Asctinidia chinensis | T | V | 43.4 |
| Asctinidia chinensis | T | O | 66.4 |
| Asparagus officinalis | T | O | 91.3 |
| Asparagus officiralis | T | R | 23.3 |
| Asparagus officiralis | T | S | 44.7 |
| Aster Linné | T | S | 47.5 |
| Aster sp | T | R | 62.0 |
| Atriplex hortensis | T | R | 54.6 |
| Atropa belladonna | T | R | 20.1 |
| Atropa belladonna | T | S | 51.0 |
| Avena sativa | T | R | 24.8 |
| Avena sativa | T | W | 26.4 |
| Averrhoa carambola | T | W | 23.4 |
| Ayperus esculentus | T | S | 46.2 |
| Beta vulgaris | T | R | 28.2 |
| Beta vulgaris | T | S | 30.4 |
| Beta vulgaris | T | O | 56.8 |
| Beta vulgaris spp. Maritima | T | R | 23.6 |
| Betula glandulosa | T | O | 22.2 |
| Betula glandulosa | T | V | 22.2 |
| Betula glandulosa | T | S | 25.7 |
| Betula glandulosa | T | W | 32.9 |
| Boletus edulis | T | S | 36.2 |
| Boletus edulis | T | O | 90.2 |
| Borago officinalis | T | S | 27.9 |
| Borago officinalis | T | O | 76.1 |
| Brassica cepticepa | T | O | 65.4 |
| Brassica cepticepa | T | S | 71.5 |
| Brassica Chineusis | T | R | 27.1 |
| Brassica juncea | T | O | 51.0 |
| Brassica juncea | T | R | 66.0 |
| Brassica juncea | T | S | 74.1 |
| Brassica Napus | T | S | 22.0 |
| Brassica Napus | T | R | 34.0 |
| Brassica Napus | T | O | 100.0 |
| Brassica nigra | T | S | 26.7 |
| Brassica nigra | T | O | 27.4 |
| Brassica nigra | T | R | 82.5 |
| Brassica oleracea | T | O | 21.2 |
| Brassica oleracea | T | S | 22.1 |
| Brassica oleracea | T | W | 26.2 |
| Brassica oleracea | T | R | 27.2 |
| Brassica oleracea | T | O | 31.3 |
| Brassica oleracea | T | W | 46.5 |
| Brassica oleracea | T | S | 71.2 |
| Brassica oleracea | T | O | 93.5 |
| Brassica rapa | T | R | 25.6 |
| Brassica rapa | T | R | 33.9 |
| Brassica rapa | T | R | 56.0 |
| Brassica rapa | T | S | 69.7 |
| Brassica rapa | T | O | 100.0 |
| Bromus inermis | T | R | 57.3 |
| Campanula rapunculus | T | O | 77.5 |
| Canna edulis | T | O | 75.6 |
| Cantharellus ciparium | T | O | 52.5 |
| Capsella bursa-pastoris | T | O | 35.9 |
| Capsicum annus | T | S | 43.9 |
| Capsicum annuum | T | S | 50.1 |
| Capsicum frutescens | T | S | 28.9 |
| Carica papaya | T | W | 31.1 |
| Carthamus tinctorius | T | R | 37.3 |
| Carum carvi | T | S | 30.1 |
| Castanea spp. | T | W | 21.7 |
| Chaerophyllum bulbosum | T | S | 46.0 |
| Chamaemelum nobile | T | W | 36.8 |
| Chamaemelum nobile | T | W | 48.4 |
| Chelidonium majus | T | O | 46.6 |
| Chenapodium bonus-henricus | T | R | 22.4 |
| Chenopodium bonus-henricus | T | S | 57.6 |
| Chenopodium quinoa | T | V | 35.5 |
| Chenopodium quinoa | T | W | 54.4 |
| Chrysanthemum leucanthemum | T | R | 26.5 |
| Chrysanthemun coronarium (Chp | T | R | 48.4 |
| suey) | | | |
| Chrysanthenum coronarium | T | R | 38.2 |
| Chrysanthenum coronarium | T | S | 63.9 |
| Cicer arietinum | T | S | 20.0 |
| Cichorium endivia | T | S | 25.6 |
| Cichorium endivia crispa | T | O | 38.4 |
| Cichorium intybus | T | S | 30.2 |
| Cimicifuga racemosa | T | S | 33.7 |
| Citrullus colocynthus | T | S | 20.4 |
| Citrullus lanatus | T | O | 68.3 |
| Citrullus lanatus | T | S | 31.9 |
| Citrus limettoides | T | W | 20.4 |
| Citrus limettoides | T | V | 37.5 |
| Citrus limon | T | V | 47.7 |
| Citrus limon | T | O | 72.4 |
| Citrus paradisi | T | W | 23.8 |
| Citrus paradisi | T | V | 33.4 |
| Citrus reticulata | T | V | 20.4 |
| Citrus reticulata | T | V | 20.9 |
| Citrus reticulata | T | W | 26.0 |
| Citrus reticulata | T | S | 40.4 |
| Citrus reticulata | T | O | 50.0 |
| Citrus reticulata | T | O | 79.2 |
| Citrus sinensis | T | W | 25.3 |
| Citrus sinensis | T | V | 59.8 |
| Coix Lacryma-Jobi | T | W | 20.0 |
| Corchorus olitorius | T | S | 38.9 |
| Cornus canadensis | T | S | 35.6 |
| Cosmos sulphureus | T | S | 51.4 |
| Crataegus sp | T | V | 28.0 |
| Crataegus sp | T | R | 60.9 |
| Crataegus submollis | T | O | 25.5 |
| Crithmum maritima | T | S | 50.6 |
| Cryptotaenia canadensis | T | O | 21.2 |
| Cryptotaenia canadensis | T | W | 26.0 |
| Cryptotaenia canadensis | T | V | 40.0 |
| Cucumis anguria | T | S | 38.7 |
| Cucumis anguria | T | O | 46.6 |
| Cucumis melo | T | S | 30.3 |
| Cucumis melo | T | O | 46.2 |
| Cucumis metuliferus | T | W | 32.0 |
| Cucumis sativus Fanfare | T | O | 40.3 |
| Cucurbita maxima | T | S | 23.6 |
| Cucurbita maxima | T | S | 33.1 |
| Cucurbita maxima | T | O | 55.2 |
| Cucurbita moschata | T | S | 20.1 |
| Cucurbita moschata | T | S | 26.7 |
| Cucurbita moschata | T | O | 41.7 |
| Cucurbita pepo | T | S | 41.9 |
| Cucurbita pepo | T | O | 82.9 |
| Curcuma zedoaria | T | S | 100.0 |
| Cydonia oblonga | T | W | 42.9 |
| Cynara scolymus | T | R | 51.6 |
| Cynara scolymus | T | S | 60.9 |
| Dactilis Glomerata | T | R | 25.7 |
| Datura stramonium | T | R | 21.9 |
| Daucus carota | T | R | 25.9 |
| Dioscorea batatas | T | O | 47.6 |
| Dioscorea batatas | T | O | 83.1 |
| Diospiros Kaki | T | W | 34.9 |
| Dirca palustris | T | S | 27.6 |
| Dirca palustris | T | O | 90.4 |
| Dolichus lablab | T | R | 66.4 |
| Dolichus lablab | T | O | 85.3 |
| Dryopteris filix-mas | T | S | 21.9 |
| Dryopteris filix-mas | T | R | 77.9 |
| Echinacea purpurea | T | S | 48.6 |
| Eleusine coracana | T | O | 45.2 |
| Elymus junceus | T | R | 41.0 |
| Erigeron canadensis | T | S | 31.4 |
| Eriobotrya japonica | T | W | 28.3 |
| Eruca vesicaria | T | R | 44.9 |
| Fagopyrum esculentum | T | W | 76.7 |
| Fagopyrum tartaricum | T | W | 42.6 |
| Festuca rubra | T | R | 29.6 |
| Festuca rubra | T | S | 42.9 |
| Foeniculum vulgare | T | V | 22.1 |
| Foericulum vulgare | T | S | 21.6 |
| Foericulum vulgare | T | O | 84.8 |
| Forsythia intermedia | T | O | 70.8 |
| Forsythia x intermedia | T | O | 60.2 |
| Fortunella spp | T | S | 35.7 |
| Fortunella spp | T | W | 50.7 |
| Fortunella spp | T | O | 74.5 |
| Fragaria | T | W | 24.8 |
| Fragaria | T | V | 52.4 |
| Fragaria | T | O | 100.0 |
| Fragaria x ananassa | T | S | 29.3 |
| Galium odoratum | T | R | 26.0 |
| Gaultheria hispidula | T | W | 40.3 |
| Ginkgo biloba | T | V | 27.0 |
| Ginkgo biloba | T | W | 68.9 |
| Glechoma hederacea | T | R | 20.4 |
| Glechoma hederacea | T | S | 30.4 |
| Glycine max | T | O | 26.6 |
| Glycine max | T | R | 47.4 |
| Glycine max | T | S | 82.0 |
| Glycyrrhiza glabra | T | S | 35.4 |
| Glycyrrhiza glabra | T | O | 40.5 |
| Glycyrrhiza glabra | T | W | 100.0 |
| Gossypium herbaceum | T | S | 36.1 |
| Guizotia abyssinica | T | R | 28.9 |
| Guizotia abyssinica | T | S | 40.4 |
| Hamamelis virginiana | T | O | 52.4 |
| Hamamelis virginiana | T | S | 67.5 |
| Hamamelis virginiana | T | R | 84.1 |
| Hedeoma pulegiodes | T | S | 57.4 |
| Helenium hoopesii | T | O | 33.7 |
| Helenium hoopesii | T | S | 49.0 |
| Helianthus annus | T | S | 53.4 |
| Helianthus strumosus | T | R | 20.3 |
| Helianthus strumosus | T | O | 71.7 |
| Helianthus tuberosa | T | W | 22.8 |
| Helianthus tuberosus L. | T | V | 22.6 |
| Helianthus tuberosus L. | T | S | 55.0 |
| Helichrysum angustifolium | T | S | 67.0 |
| Heliotropium arborescens | T | S | 58.9 |
| Helleborus niger | T | S | 31.9 |
| Hibiscus cannabinus | T | S | 48.9 |
| Hordeum vulgare | T | S | 29.2 |
| Humulus lupulus | T | W | 22.4 |
| Humulus lupulus | T | R | 39.1 |
| Humulus lupulus | T | O | 63.1 |
| Humulus lupulus | T | S | 100.0 |
| Hydrastis canadensis | T | S | 20.2 |
| Hydrastis canadensis | T | W | 31.0 |
| Hyoscyamus niger | T | O | 56.8 |
| Hypericum henryi | T | O | 48.8 |
| Hypericum perforatum | T | S | 48.1 |
| Hypericum perforatum | T | O | 63.7 |
| Hypomyces lactiflorum | T | S | 44.8 |
| Hypomyces lactiflorum | T | O | 60.9 |
| Hyssops officinalis | T | W | 22.9 |
| Inula helenium | T | S | 24.6 |
| Juniperus communis | T | S | 33.0 |
| Juniperus communis | T | O | 38.2 |
| Lactuca sativa | T | S | 44.5 |
| Lactuca sativa | T | R | 50.7 |
| Laportea canadensis | T | S | 30.2 |
| Lathyrus Sativus | T | O | 20.4 |
| Lathyrus Sativus | T | R | 52.5 |
| Lathyrus sylvestris | T | W | 27.7 |
| Lathyrus sylvestris | T | O | 36.8 |
| Laurus nobilis | T | S | 52.0 |
| Lavendula angustifolia | T | W | 26.4 |
| Lavendula angustifolia | T | S | 53.2 |
| Lavendula latifolia | T | S | 51.3 |
| Ledum groenlandicum | T | S | 44.4 |
| Lentinus edodes | T | W | 42.1 |
| Lentinus edodes | T | O | 100.0 |
| Lepidium sativum | T | S | 44.2 |
| Levisticum officinale | T | S | 20.8 |
| Levisticum officinale | T | O | 39.4 |
| Linum usitatissimum | T | R | 42.3 |
| Litchi chinensis | T | W | 25.7 |
| Lolium multiflorum | T | S | 20.6 |
| Lolium perenne | T | R | 28.7 |
| Lonicera ramosissima | T | S | 26.3 |
| Lonicera ramosissima | T | O | 40.4 |
| Lonicera ramosissima | T | W | 53.2 |
| Lonicera syringantha | T | W | 95.8 |
| Lotus corniculatus | T | R | 100.0 |
| Lotus tetragonolubus | T | S | 65.4 |
| Lunaria annua | T | O | 55.7 |
| Lunaria annua | T | S | 67.3 |
| Lycopersicon esculentum | T | R | 37.6 |
| Malus | T | W | 31.8 |
| Malus | T | V | 44.4 |
| Malus hupehensis (Pamp.) Rehd. | T | R | 26.3 |
| Malus hupehensis (Pamp.) Rehd. | T | S | 67.0 |
| Malus sp. | T | R | 65.3 |
| Malva moschata | T | S | 41.1 |
| Malva sylvestris | T | S | 36.4 |
| Malva sylvestris | T | O | 47.4 |
| Malva verticillata | T | R | 42.7 |
| Mangifera indica | T | O | 30.5 |
| Manihot esculenta syn. M. utilissima | T | W | 38.3 |
| Manihot esculenta syn. M. utilissima | T | S | 50.4 |
| Manihot esculenta syn. M. utilissima | T | O | 86.5 |
| Melilotus alba | T | R | 30.4 |
| Melilotus officinalis | T | R | 68.1 |
| Melissa officinalis | T | S | 33.7 |
| Melissa officinalis | T | O | 34.7 |
| mentha arvensis | T | R | 53.7 |
| Mentha suaveolens | T | S | 26.8 |
| Menyanthes trifoliata | T | S | 32.8 |
| Miscanthus sinensis Andress | T | R | 22.7 |
| Momordica charantia | T | S | 55.5 |
| Monarda didyma | T | S | 26.8 |
| Monarda fistulosa | T | S | 21.5 |
| Montia perfoliata | T | R | 26.6 |
| Musa paradisiaca | T | W | 29.0 |
| nasturtium officinale | T | S | 35.4 |
| Nepeta cataria | T | W | 26.5 |
| Nepeta cataria | T | O | 27.5 |
| Nepeta cataria | T | S | 41.9 |
| Nephelium longana ou Euphoria | T | W | 43.4 |
| longana | | | |
| Nicotiana rustica | T | O | 26.0 |
| Nicotiana rustica | T | S | 32.7 |
| Nicotiana tabacum | T | S | 25.1 |
| Nicotiana tabacum | T | O | 77.7 |
| Nigella sativa | T | R | 59.3 |
| Nigella sativa | T | R | 100.0 |
| Ocimum Basilicum | T | W | 20.2 |
| Ocimum Basilicum | T | V | 20.2 |
| Ocimum Basilicum | T | S | 32.8 |
| Oenothera biennis linné | T | R | 100.0 |
| Onobrychis viciafolia | T | R | 45.0 |
| Optunia sp. | T | W | 33.4 |
| Origanum marjonara | T | O | 20.5 |
| Origanum vulgare | T | O | 20.8 |
| Origanum vulgare | T | W | 21.6 |
| Oryza sativa | T | W | 42.4 |
| oxyria digyna | T | O | 57.0 |
| oxyria digyna | T | V | 77.9 |
| Panax quinquefolius L. | T | O | 23.5 |
| Panicum miliaceum | T | W | 36.5 |
| Passiflora spp | T | S | 35.8 |
| Passiflora spp | T | V | 38.3 |
| Passiflora spp | T | W | 46.2 |
| Passiflora spp | T | O | 100.0 |
| Pastinaca sativa | T | O | 21.7 |
| Pastinaca sativa | T | R | 38.6 |
| Pastinaca sativa | T | S | 39.2 |
| Persea americana | T | V | 32.5 |
| Persea americana | T | O | 38.6 |
| Petasites Japonicus | T | S | 26.2 |
| Phalaris canariensis | T | O | 80.0 |
| Phaseolus coccineus | T | S | 44.4 |
| Phaseolus coccineus | T | R | 79.1 |
| Phaseolus mungo | T | S | 27.0 |
| Phaseolus mungo | T | O | 37.9 |
| Phaseolus vulgaris | T | R | 20.1 |
| Phaseolus vulgaris | T | S | 51.9 |
| Phaseolus vulgaris | T | O | 61.7 |
| Phlox paniculata | T | S | 22.9 |
| Phlox paniculata | T | O | 44.5 |
| Phoenix dactylifera | T | O | 29.6 |
| Physalis alkekengi | T | R | 32.9 |
| Physalis ixocarpa | T | R | 26.6 |
| Physalis ixocarpa | T | O | 28.3 |
| Physalis pruinosa | T | S | 27.3 |
| Physalis pruinosa | T | R | 47.8 |
| Physalis pruinosa | T | O | 93.1 |
| Physalis sp | T | W | 39.1 |
| Physalis sp | T | V | 60.8 |
| Phytolacca americana | T | S | 41.8 |
| Phytolacca americana | T | O | 100.0 |
| Phytolacca decandra syn. P. | T | O | 85.9 |
| americana | | | |
| Pimpinella anisum | T | S | 20.2 |
| Pimpinella anisum | T | O | 68.4 |
| Pisum sativum | T | W | 20.1 |
| Pisum sativum | T | S | 25.8 |
| Pisum sativum | T | V | 27.0 |
| Pisum sativum | T | O | 51.8 |
| Plantago coronopus | T | R | 21.9 |
| Plantago coronopus | T | O | 48.6 |
| Plantago coronopus | T | S | 66.8 |
| Plantago major | T | S | 35.1 |
| Pleurotus spp | T | W | 25.3 |
| Pleurotus spp | T | S | 59.3 |
| Pleurotus spp | T | O | 85.2 |
| Poa compressa | T | R | 26.2 |
| Poa pratensis | T | O | 21.5 |
| Poa pratensis | T | R | 30.0 |
| Podophyllum peltatum | T | O | 33.9 |
| Podophyllum peltatum | T | S | 50.2 |
| Polygonum aviculare linné | T | R | 31.0 |
| Polygonum pennsylvanicum | T | S | 56.6 |
| Polygonum persicaria | T | S | 20.1 |
| Populus incrassata | T | W | 54.9 |
| Populus Tremula | T | W | 31.0 |
| Populus x petrowskyana | T | W | 100.0 |
| Potentilla anserina | T | S | 22.1 |
| Potentilla anserina | T | O | 41.1 |
| Prunus cerasus | T | V | 30.1 |
| Prunus persica | T | W | 26.6 |
| Prunus persica | T | V | 38.5 |
| Prunus spp | T | S | 24.0 |
| Prunus spp | T | S | 49.1 |
| Psidium guajaba | T | V | 22.5 |
| Psidium guajaba | T | W | 44.3 |
| Psidium guajaba | T | O | 95.4 |
| Psidium spp | T | S | 36.6 |
| Psidium spp | T | W | 47.6 |
| Psidium spp | T | O | 87.6 |
| Pteridium aquilinum | T | R | 22.0 |
| Punica granatum | T | V | 52.1 |
| Pyrus communis | T | V | 39.5 |
| Pyrus pyrifolia | T | W | 33.7 |
| Raphanus raphanistrum | T | O | 24.5 |
| Raphanus raphanistrum | T | S | 44.8 |
| Raphanus raphanistrum | T | S | 46.1 |
| Raphanus sativus | T | V | 25.4 |
| Raphanus sativus | T | R | 32.1 |
| Raphanus sativus | T | W | 38.1 |
| Raphanus sativus | T | S | 63.6 |
| Raphanus sativus | T | O | 93.4 |
| Reseda luteola | T | S | 22.5 |
| Rhamnus frangula | T | S | 34.2 |
| Rhamnus frangula | T | R | 39.5 |
| Rheum officinale | T | S | 100.0 |
| Rheum palmatum | T | W | 20.2 |
| Rheum rhabarbarum | T | S | 33.8 |
| Rianus communis | T | S | 20.9 |
| Ribes nidigrolaria | T | W | 44.5 |
| Ribes nidigrolaria | T | V | 53.1 |
| Ribes nigrum | T | S | 40.7 |
| Ribes nigrum L. | T | W | 50.0 |
| Ribes nigrum L. | T | V | 60.1 |
| Ribes sativam syme | T | W | 47.9 |
| Ribes Sativum | T | R | 48.2 |
| Ribes Silvestre | T | V | 26.3 |
| Ribes Silvestre | T | W | 100.0 |
| Ribes uva-crispa | T | O | 57.5 |
| Rosa rugosa | T | S | 27.8 |
| Rosa rugosa thunb. | T | W | 37.5 |
| Rosa rugosa thunb. | T | V | 45.7 |
| Rosmarinum officinalis | T | R | 44.2 |
| Rosmarinum officinalis | T | W | 65.9 |
| Rubus canadensis | T | S | 45.5 |
| Rubus idaeus | T | W | 31.4 |
| Rubus idaeus | T | V | 57.2 |
| Rubus ideaus | T | S | 28.5 |
| Rubus ideaus | T | O | 38.0 |
| Rubus occidentalis | T | O | 21.4 |
| Rubus occidentalis | T | S | 36.5 |
| Rubus occidentalis | T | R | 60.2 |
| Rumes scutatus | T | O | 84.5 |
| Rumex crispus linné | T | O | 52.5 |
| Rumex crispus linné | T | R | 100.0 |
| Rumex patientia | T | O | 23.1 |
| Rumex patientia | T | S | 65.8 |
| Ruta graveolens | T | S | 37.2 |
| Sabal serrulata syn. Serenoa repens | T | V | 34.4 |
| Sabal serrulata syn. Serenoa repens | T | S | 44.6 |
| Salix purpurea | T | R | 67.8 |
| Salvia (elegens) | T | O | 51.1 |
| Sambucus canadensis | T | S | 44.8 |
| Sambucus canadensis | T | O | 72.4 |
| Sambucus canadensis L. | T | W | 67.8 |
| Sambucus ebulus | T | V | 44.3 |
| Sanguisorba officinalis | T | R | 100.0 |
| Santolina | T | R | 37.9 |
| Satureja montana | T | S | 20.0 |
| Satureja montana | T | O | 21.3 |
| Satureja repandra | T | S | 36.3 |
| Scorzorera hipanica | T | R | 27.1 |
| Scorzorera hipanica | T | S | 31.7 |
| Scuttellaria lateriflora | T | S | 44.3 |
| Secale cereale | T | S | 24.2 |
| Secale cereale | T | W | 31.1 |
| Sechium edule | T | S | 37.8 |
| Sesamum indicum | T | S | 59.2 |
| Setaria italica | T | W | 33.0 |
| Silybum marianum | T | O | 92.4 |
| Sium sisarum | T | O | 32.7 |
| Sium sisarum | T | S | 33.1 |
| Sium sisarum | T | O | 81.3 |
| Solanum melogena | T | O | 21.9 |
| Solanum melogena | T | V | 26.1 |
| Solanum melogena | T | R | 34.0 |
| Solanum melogena | T | S | 67.1 |
| Solanum Tuberosum | T | O | 68.6 |
| Solidago canadensis | T | S | 48.4 |
| Solidago sp | T | R | 31.4 |
| Solidago virgaurea | T | S | 56.2 |
| Sorghum caffrorum | T | O | 23.3 |
| Sorghum dochna bicolor gr technicum | T | W | 20.8 |
| Sorghum dochna Snowdrew | T | S | 21.4 |
| Sorghum dochna Snowdrew | T | O | 27.7 |
| Spinacia oleracea | T | V | 25.0 |
| Spinacia oleracea | T | W | 32.1 |
| Spinacia oleracea | T | S | 47.6 |
| Spinacia oleracea | T | O | 63.1 |
| Stachys affinis | T | R | 31.7 |
| Stachys affinis | T | O | 100.0 |
| Stachys byzantina | T | W | 30.9 |
| Stipa capillata L. | T | R | 20.1 |
| Symphytum officinale | T | S | 24.1 |
| Tanacetum cinerarifolium | T | O | 24.2 |
| Tanacetum cinerarifolium | T | R | 84.4 |
| Tanacetum vulgare | T | R | 25.7 |
| Tanacetum vulgare | T | S | 75.6 |
| Taraxacum officinale (Red ribe) | T | S | 21.1 |
| Tepary | T | R | 56.7 |
| Teucrium chamaedrys L. | T | R | 27.3 |
| Thalpsi arvense | T | S | 61.4 |
| Thymus fragantissumus | T | R | 100.0 |
| Thymus herba-barona | T | W | 22.0 |
| Thymus pseudolanuginosus | T | R | 36.8 |
| Thymus pseudolanuginosus | T | S | 37.1 |
| Thymus serpyllum | T | S | 26.0 |
| Thymus serpyllum | T | W | 42.7 |
| Thymus x citriodorus | T | O | 22.7 |
| Tiarella cordifolia | T | R | 100.0 |
| Tragopogon porrifolius | T | V | 26.8 |
| Tragopogon porrifolius | T | O | 28.4 |
| Tragopogon porrifolius | T | S | 42.1 |
| Tragopogon sp. | T | O | 20.3 |
| Tragopogon sp. | T | S | 32.0 |
| Tragopogon sp. | T | W | 66.3 |
| Trichosanthes kirilowii | T | O | 66.5 |
| Trifolium incarnatum | T | R | 47.9 |
| Trifolium repens | T | R | 81.7 |
| Trigonella foenum graecum | T | S | 39.6 |
| Triticale sp. | T | O | 64.1 |
| Triticum aestivum | T | W | 24.5 |
| Triticum aestivum | T | S | 29.4 |
| Triticum furgidumm | T | S | 35.8 |
| Triticum spelta | T | S | 34.7 |
| Tropaeolum majus | T | O | 90.3 |
| Tropaeolum malus | T | W | 64.4 |
| Tsuga can0adensis | T | O | 21.5 |
| Tsuga can0adensis | T | W | 64.4 |
| Tsuga diversifolia | T | O | 45.9 |
| Tsuga diversifolia | T | W | 100.0 |
| Tsuga F. macrophylla | T | W | 28.1 |
| Typha latifolia L. | T | S | 30.6 |
| Urtica dioica | T | O | 31.4 |
| Urtica dioica | T | R | 36.9 |
| Urtica dioica | T | S | 41.7 |
| Vaccinium angustifolium | T | V | 25.2 |
| Vaccinium angustifolium | T | R | 34.6 |
| Vaccinium angustifolium | T | O | 59.6 |
| Vaccinium angustifolium | T | R | 65.7 |
| Vaccinium macrocarpon | T | O | 30.2 |
| Vaccinium macrocarpon | T | S | 39.0 |
| Vaccinium macrocarpon | T | S | 56.9 |
| Vaccinum macrocarpon | T | V | 39.2 |
| Vaccinum macrocarpon | T | W | 42.3 |
| Veratrum viride | T | O | 20.5 |
| Veratrum viride | T | S | 33.1 |
| Verbascum thapsus | T | S | 43.1 |
| Verbascum thapsus | T | O | 70.2 |
| Veronica officinalis | T | O | 20.5 |
| Viburnum trilobum Marsh. | T | S | 40.6 |
| Vicia faba | T | R | 61.5 |
| Vicia sativa | T | R | 30.1 |
| Vigna angularia | T | R | 32.6 |
| Vigna angularia | T | S | 64.2 |
| Vigna unguiculata | T | R | 32.4 |
| Vigna unguiculata | T | O | 47.4 |
| Vigna unguiculata | T | S | 51.0 |
| Vinca minor | T | S | 21.3 |
| Vitis sp. | T | V | 28.3 |
| Vitis sp. | T | O | 29.4 |
| Vitis sp. | T | S | 45.4 |
| Vitis sp. | T | V | 50.7 |
| Vitis sp. | T | W | 61.6 |
| Vitis sp. | T | R | 100.0 |
| Weigela coracensis | T | W | 35.5 |
| Withania somnifera | T | S | 35.5 |
| Xanthium sibiricum | T | S | 38.6 |
| Xanthium strumarium | T | S | 33.5 |
| Zea mays | T | S | 37.1 |
| Zea mays | T | O | 65.5 |
| Zingiber officinale | T | S | 20.1 |
| Zingiber officinale | T | W | 58.9 |
| Zingiber officinale | T | O | 75.9 |

TABLE 5
Inhibition of HLE by Plant Extracts

| | | | Inhibition |
|---|---|---|---|
| Latin name | Stress | Extract | (%) |
| Achillea millefolium | A | O | 21.9 |
| Achillea millefolium | A | S | 24.5 |
| Aconitum napellus | A | O | 25.8 |
| Adiantum pedatum | A | R | 27.6 |
| Agrimonia eupatoria | A | V | 26.0 |
| Agropyron cristatum | A | R | 21.0 |
| Agropyron repens | A | S | 23.4 |
| Agropyron repens | A | R | 28.2 |
| Agropyron repens | A | S | 39.8 |
| Agrostis Stofonifera | A | O | 38.9 |
| Alchemilla mollis | A | V | 27.9 |
| Alchemilla mollis | A | O | 66.0 |
| Alchemilla mollis | A | R | 100.0 |
| Alchemilla mollis | A | S | 23.5 |
| Alkanna tinctoria | A | S | 26.2 |
| Allium Tuberosum | A | S | 57.9 |
| Aloe vera | A | O | 20.5 |
| Ambrosia artemisiifolia | A | O | 29.1 |
| Amelanchier sanguinea | A | W | 96.5 |
| Amelanchier sanguinea | A | V | 52.4 |
| Anethum graveolens | A | O | 32.1 |
| Anethum graveolens | A | W | 22.8 |
| Angelica archangelica | A | S | 39.2 |
| Anthemis nobilis | A | O | 37.6 |
| Anthemis nobilis | A | S | 26.4 |
| Anthemis tinctoria | A | O | 31.9 |
| Anthemis tinctoria | A | S | 38.4 |
| Apium graveolens | A | S | 49.2 |
| Arctium minus | A | O | 46.4 |
| Arctostaphylos uva-ursi | A | R | 100.0 |
| Aronia melanocarpa | A | O | 21.9 |
| Aronia melanocarpa | A | W | 78.4 |
| Aronia melanocarpa | A | V | 100.0 |
| Aronia melanocarpa | A | R | 29.0 |
| Aronia melanocarpa | A | O | 33.6 |
| Artemisia dracunculus | A | W | 89.2 |
| Ludoviciana | A | O | 33.4 |
| Ludoviciana | A | S | 20.7 |
| Aster sp | A | R | 26.2 |
| Beta vulgaris | A | R | 100.0 |
| Beta vulgaris spp. Maritima | A | R | 92.2 |
| Borago officinalis | A | S | 22.6 |
| Brassica napus | A | S | 68.3 |
| Brassica napus | A | R | 29.5 |
| Brassica nigra | A | S | 32.6 |
| Brassica oleracea | A | O | 22.9 |
| Brassica oleracea | A | V | 20.8 |
| Brassica oleracea | A | R | 22.2 |
| Brassica rapa | A | S | 23.2 |
| Brassica rapa | A | R | 26.9 |
| Bromus inermis | A | O | 34.1 |
| Bromus inermis | A | R | 21.9 |
| Calamintha nepeta | A | O | 35.4 |
| Canna edulis | A | O | 56.4 |
| Canna edulis | A | R | 21.4 |
| Carum carvi | A | O | 24.2 |
| Chaerophyllum bulbosum | A | O | 25.5 |
| chenopodium bonus-henricus | A | R | 24.0 |
| Chenopodium bonus-henricus | A | S | 85.8 |
| Chenopodium quinoa | A | S | 50.4 |
| Chrysanthemum coronarium | A | O | 26.0 |
| Cicer arietinum | A | S | 23.3 |
| Cichorium intybus | A | S | 32.1 |
| Citrullus lanatus | A | R | 26.3 |
| Coix Lacryma-Jobi | A | S | 66.1 |
| Cosmos sulphureus | A | O | 38.8 |
| Cosmos sulphureus | A | S | 20.7 |
| Crataegus sp | A | O | 84.1 |
| Crataegus sp | A | R | 23.6 |
| Crataegus sp | A | S | 21.7 |
| Crataegus submollis | A | S | 34.0 |
| Cryptotaenia canadensis | A | V | 22.1 |
| Cucumis anguria | A | O | 26.2 |
| Cucumis Anguria | A | R | 53.4 |
| Cucumis melo | A | S | 53.6 |
| Cucumis sativus | A | R | 53.3 |
| Curcuma zedoaria | A | O | 24.3 |
| Cymbopogon citratus | A | S | 91.2 |
| Datisca cannabina | A | S | 55.7 |
| Daucus carota | A | R | 100.0 |
| Daucus carota | A | V | 24.7 |
| Daucus carota | A | O | 37.9 |
| Digitalis purpurea | A | S | 34.0 |
| Dirca palustris | A | R | 20.3 |
| Dirca palustris | A | S | 27.9 |
| Dolichos Lablab | A | R | 21.5 |
| Dryopteris filix-mas | A | R | 58.8 |
| Dryopteris filix-mas | A | S | 22.0 |
| Echinacea purpurea | A | O | 38.2 |
| Echinacea purpurea | A | S | 28.1 |
| Eleusine coracana | A | S | 20.7 |
| Erigeron canadensis | A | O | 29.6 |
| Fagopyrum esculentum | A | S | 29.3 |
| Fagopyrum tataricum | A | S | 24.4 |
| Foeniculum vulgare | A | O | 25.1 |
| Fragaria Xananassa | A | O | 22.3 |
| Fragaria Xananassa | A | W | 100.0 |
| Fragaria Xananassa | A | V | 21.4 |
| Fragaria Xananassa | A | S | 29.4 |
| Fragaria Xananassa | A | V | 21.6 |
| Galinsoga ciliata | A | R | 61.6 |
| Galium odoratum | A | R | 21.0 |
| Gaultheria hispidula | A | O | 33.7 |
| Gentiana lutea | A | R | 52.1 |
| Glechoma hederacea | A | O | 21.8 |
| Glycine Max | A | S | 81.3 |
| Glycyrrhiza glabra | A | W | 100.0 |
| Glycyrrhiza glabra | A | S | 63.3 |
| Guizotia abyssinica | A | R | 36.9 |
| Hamamelis virginiana | A | R | 100.0 |
| Helianthus Tuberosus | A | S | 32.1 |
| Heliotropium arborescens | A | R | 22.8 |
| Heliotropium arborescens | A | S | 24.9 |
| Helleborus niger | A | S | 25.6 |
| Hordeum vulgare | A | O | 58.1 |
| Hypericum perforatum | A | S | 24.8 |
| Hyssopus officinalis | A | O | 21.1 |
| Hyssopus officinalis | A | S | 93.6 |
| Lactuca serriola | A | S | 34.3 |
| Laurus nobilis | A | W | 100.0 |
| Lavandula latifolia | A | W | 57.1 |
| Lavandula latifolia | A | O | 43.7 |
| Lavandula latifolia | A | S | 42.2 |
| Leonurus cardiaca | A | R | 100.0 |
| Lepidium sativum | A | O | 100.0 |
| Lolium multiflorum | A | O | 31.0 |
| Lolium perenne | A | O | 20.8 |
| Lolium perenne | A | R | 21.7 |
| Lolium perenne | A | S | 22.1 |
| Malva sylvestris | A | S | 22.9 |
| Matricaria recutita | A | O | 28.5 |
| Melaleuca alternifolia | A | O | 21.9 |
| Melissa officinalis | A | S | 23.4 |
| Mentha piperita | A | O | 31.6 |
| Mentha piperita | A | W | 33.2 |
| Mentha pulegium | A | O | 42.2 |
| Mentha pulegium | A | V | 21.5 |
| Mentha pulegium | A | S | 33.8 |
| Mentha spicata | A | O | 24.3 |
| Oenothera biennis | A | O | 25.2 |
| Oenothera biennis | A | R | 78.8 |
| Origanum majorana | A | V | 37.4 |
| Oxyria digyna | A | V | 28.2 |
| Panicum miliaceum | A | O | 33.3 |
| Peucedanum cervaria | A | R | 23.4 |
| Phalaris arundinacea | A | R | 22.4 |
| Phalaris canariensis | A | O | 27.8 |
| Phaseolus coccineus | A | S | 28.3 |
| Phaseolus mungo | A | R | 37.8 |
| Phaseolus vulgaris | A | O | 24.3 |
| Phaseolus Vulgaris | A | S | 74.3 |
| Phleum pratense | A | R | 27.8 |
| Physalis ixocarpa | A | O | 21.5 |
| Physalis Ixocarpa | A | S | 26.5 |
| Physalis Pruinosa | A | S | 60.2 |
| Phytolacca americana | A | S | 100.0 |
| Plantago coronopus | A | O | 21.1 |
| Plantago coronopus | A | S | 25.7 |
| Plantago major | A | O | 26.0 |
| Plectranthus sp. | A | O | 23.1 |
| Poa pratensis | A | O | 21.7 |
| Polygonum aviculare | A | R | 79.7 |
| Portulaca olevcae | A | O | 34.5 |
| Poterium sanguisorba | A | R | 25.8 |
| Poterium sanguisorba | A | O | 34.6 |
| Poterium sanguisorba | A | W | 31.0 |
| Pteridium aquilinum | A | R | 54.4 |
| Raphanus sativus | A | S | 66.4 |
| Raphanus sativus | A | R | 81.8 |
| Rheum officinale | A | S | 37.9 |
| Ribes nigrum | A | W | 100.0 |
| Ribes nigrum | A | S | 47.6 |
| Ribes nigrum | A | V | 27.5 |
| Ribes rubrum | A | R | 35.4 |
| Ribes Sylvestre | A | W | 100.0 |
| Rosa rugosa | A | W | 95.1 |
| Rosa rugosa | A | R | 24.6 |
| Rosmarinus officinalis | A | R | 58.4 |
| Rubus idaeus | A | W | 27.6 |
| Rubus idaeus | A | S | 33.0 |
| Rubus idaeus | A | R | 27.9 |
| Rubus idaeus | A | O | 37.4 |
| Rumex Acetosa | A | S | 45.2 |
| Rumex crispus | A | O | 26.1 |
| Rumex crispus | A | R | 100.0 |
| Rumex Scutatus | A | V | 43.8 |
| Ruta graveolens | A | O | 28.7 |
| Saccharum officinarum | A | O | 29.6 |
| Saccharum officinarum | A | R | 23.8 |
| Salvia elegans | A | O | 100.0 |
| Salvia officinalis | A | O | 95.7 |
| Salvia officinalis | A | W | 77.9 |
| Salvia officinalis | A | R | 83.7 |
| Salvia officinalis | A | S | 20.5 |
| Salvia sclarea | A | O | 100.0 |
| Salvia sclarea | A | V | 28.6 |
| Santolina chamaecyparissus | A | O | 27.1 |
| Satureja montana | A | W | 23.2 |
| Satureja montana | A | S | 27.7 |
| Scorzonera hispanica | A | R | 60.1 |
| Scutellaria lateriflora | A | S | 45.9 |
| Senecio vulgaris | A | R | 34.0 |
| Sonchus oleraceus | A | O | 29.1 |
| Sorghum dochna | A | O | 21.1 |
| Sorghum dochna | A | V | 24.4 |
| Sorghum durra | A | O | 23.4 |
| Sorghum durra | A | V | 23.6 |
| Spinacia oleracea | A | S | 26.8 |
| Stellaria graminea | A | O | 24.8 |
| Symphytum officinale | A | O | 91.6 |
| Tanacetum cinerariifolium | A | R | 28.3 |
| Tanacetum vulgare | A | O | 46.3 |
| Tanacetum vulgare | A | S | 33.7 |
| Taraxacum officinale | A | W | 26.4 |
| Taraxacum officinale | A | V | 24.0 |
| Taraxacum officinale | A | O | 21.0 |
| Teucrium chamaedrys | A | O | 37.0 |
| Thymus fragantissimus | A | W | 20.2 |
| Thymus herba-barona | A | W | 20.8 |
| Thymus vulgaris | A | R | 77.9 |
| Thymus vulgaris | A | W | 23.6 |
| Thymus × citriodorus | A | W | 21.3 |
| Thymus × citriodorus | A | S | 21.1 |
| Trichosanthes kirilowii | A | O | 23.2 |
| Trigonella foenum graecum | A | S | 32.0 |
| Triticum durum | A | S | 22.0 |
| Triticum turgidum | A | O | 60.0 |
| Triticum spelta | A | S | 47.6 |
| Urtica dioica | A | O | 33.3 |
| Vaccinium augustifolium | A | W | 42.6 |
| Vaccinium Corymbosum | A | W | 22.4 |
| Vaccinium Corymbosum | A | S | 21.6 |
| Vaccinium macrocarpon | A | W | 22.5 |
| Vaccinium macrocarpon | A | S | 54.8 |
| Valerianella locusta | A | O | 49.2 |
| Veronica officinalis | A | O | 43.7 |
| Viburnum trilobum Marsh. | A | W | 75.4 |
| Vitis | A | S | 33.8 |
| Vitis | A | W | 100.0 |
| Vitis | A | O | 21.0 |
| Zea Mays | A | S | 95.2 |
| Achillea millefolium | G | O | 28.8 |
| Achillea millefolium | G | S | 27.3 |
| Aconitum napellus | G | O | 23.1 |
| Aconitum napellus | G | R | 97.7 |
| Acorus calamus | G | S | 20.0 |
| Adiantum pedatum | G | R | 100.0 |
| Agastache foeniculum | G | W | 25.3 |
| Ageratum conyzoides | G | O | 28.5 |
| Agropyron cristatum | G | R | 37.3 |
| Agropyron repens | G | R | 31.4 |
| Alchemilla mollis | G | W | 20.6 |
| Alchemilla mollis | G | O | 56.1 |
| Alchemilla mollis | G | R | 28.1 |
| Alchemilla mollis | G | S | 25.3 |
| Allium cepa | G | O | 20.2 |
| Allium sativum | G | O | 100.0 |
| Allium tuberosum | G | O | 100.0 |
| Althaea officinalis | G | S | 30.8 |
| Amaranthus caudatus | G | S | 22.3 |
| Amelanchier sanguinea | G | W | 88.3 |
| Anethum graveolens | G | O | 26.2 |
| Angelica archangelica | G | S | 43.2 |
| Anthemis nobilis | G | S | 21.7 |
| Arctostaphylos uva-ursi | G | O | 33.1 |
| Arctostaphylos uva-ursi | G | R | 100.0 |
| Arctostaphylos uva-ursi | G | S | 23.4 |
| Armoracia rusticana | G | O | 22.5 |
| Aronia melanocarpa | G | W | 79.0 |
| Aronia melanocarpa | G | V | 100.0 |
| Aronia melanocarpa | G | S | 22.7 |
| Aronia melanocarpa | G | O | 29.6 |
| Artemisia absinthium | G | O | 31.5 |
| Artemisia absinthium | G | V | 24.2 |
| Aster | G | S | 29.2 |
| Beckmannia eruciformis | G | O | 22.7 |
| Beta vulgaris | G | R | 100.0 |
| Betula glandulosa | G | S | 26.7 |
| Borago officinalis | G | O | 25.7 |
| Brassica Napus | G | S | 50.4 |
| Brassica napus | G | R | 48.2 |
| Brassica nigra | G | S | 23.9 |
| Brassica oleracea | G | R | 28.1 |
| Brassica oleracea | G | S | 22.5 |
| Brassica rapa | G | R | 56.4 |
| Calamintha nepeta | G | V | 24.8 |
| Calamintha nepeta | G | O | 38.8 |
| Canna edulis | G | O | 66.3 |
| Capsella bursa-pastoris | G | R | 25.8 |
| Carthamus tinctorius | G | R | 22.2 |
| Chelidonium majus | G | O | 31.6 |
| Chenopodium album | G | S | 21.3 |
| Cichorium endivia subsp. Endivia | G | S | 21.4 |
| Cicer arietinum | G | S | 50.7 |
| Cichorium endivia subsp. Endivia | G | O | 48.5 |
| Cichorium endivia subsp. Endivia | G | S | 27.9 |
| Coix Lacryma-Jobi | G | O | 24.5 |
| Cornus canadensis | G | S | 36.1 |
| Crataegus sp | G | W | 57.8 |
| Cucurbita Pepo | G | R | 23.1 |
| Curcuma zedoaria | G | O | 24.0 |
| Datura metel | G | O | 21.0 |
| Daucus carota | G | O | 32.3 |
| Daucus carrota | G | R | 90.9 |
| Dipsacus sativus | G | O | 32.7 |
| Dirca palustris | G | S | 33.5 |
| Dolichos Lablab | G | R | 32.1 |
| Dryopteris filix-mas | G | R | 80.9 |
| Echinacea purpurea | G | S | 63.0 |
| Elymus junceus | G | R | 25.9 |
| Erigeron canadensis | G | O | 43.0 |
| Erigeron speciosus | G | O | 22.8 |
| Erigeron speciosus | G | S | 24.2 |
| Erysimum perofskianum | G | O | 20.8 |
| Fagopyrum esculentum | G | S | 32.9 |
| Fagopyrum tataricum | G | S | 41.2 |
| Focniculum vulgare | G | V | 25.7 |
| Foeniculum vulgare | G | S | 42.5 |
| Foeniculum Vulgare | G | O | 24.1 |
| Galinsoga ciliata | G | S | 25.0 |
| Galium odoratum | G | R | 89.4 |
| Gaultheria hispidula | G | O | 35.1 |
| Gaultheria hispidula | G | R | 67.2 |
| Gaultheria procumbens | G | S | 74.7 |
| Glycine max | G | R | 24.6 |
| Glycyrrhiza glabra | G | W | 56.8 |
| Glycyrrhiza glabra | G | V | 30.0 |
| Glycyrrhiza glabra | G | R | 92.4 |
| Glycyrrhiza glabra | G | S | 28.6 |
| Hamamelis virginiana | G | R | 100.0 |
| Hamamelis virginiana | G | S | 29.3 |
| Hedeoma pulegioides | G | O | 60.0 |
| Helenium hoopesii | G | O | 37.3 |
| Helenium hoopesii | G | S | 34.7 |
| Helianthus tuberosus | G | V | 21.4 |
| Helichrysum thianschanicum | G | O | 43.0 |
| Helichrysum thianschanicum | G | R | 39.2 |
| Heliotropium arborescens | G | R | 22.8 |
| Heliotropium arborescens | G | S | 39.5 |
| Helleborus niger | G | S | 34.2 |
| Hypericum henryi | G | S | 23.7 |
| Hypericum perforatum | G | S | 23.8 |
| Hyssopus officinalis | G | W | 45.1 |
| Hyssopus officinalis | G | S | 24.2 |
| Inula helenium | G | W | 96.2 |
| Ipomola batatas | G | V | 21.9 |
| Lactuca sativa | G | W | 35.1 |
| Laportea canadensis | G | O | 25.1 |
| Laportea canadensis | G | S | 26.5 |
| Laserpitium latifolium | G | S | 22.1 |
| Lathyrus sativus | G | O | 29.9 |
| Lathyrus sativus | G | W | 27.8 |
| Lathyrus sativus | G | S | 28.1 |
| Laurus nobilis | G | W | 100.0 |
| Lavandula angustifolia | G | O | 65.7 |
| Ledum groenlandicum | G | O | 100.0 |
| Leonorus cardiaca | G | R | 61.3 |
| Lepidium sativum | G | O | 100.0 |
| Levisticum officinale | G | W | 91.4 |
| Lolium perenne | G | O | 37.3 |
| Lotus tetragonolobus | G | S | 21.8 |
| Lupinus polyphyllus | G | O | 42.3 |
| Malus hupehensis | G | S | 25.9 |
| Medicago sativa | G | S | 32.1 |
| Melaleuca alternifolia | G | O | 40.0 |
| Melissa officinalis | G | S | 23.1 |
| Mentha arvensis | G | S | 65.5 |
| Mentha piperita | G | O | 24.2 |
| Mentha piperita | G | S | 23.7 |
| Mentha piperita | G | V | 34.2 |
| Mentha pulegium | G | O | 63.3 |
| Mentha pulegium | G | V | 30.2 |
| Mentha spicata | G | S | 45.9 |
| Monarda didyma | G | S | 47.7 |
| Nepeta cataria | G | R | 100.0 |
| Nicotiana tabacum | G | O | 75.8 |
| Hordeum vulgare subsp. Vulgare | G | O | 33.4 |
| Ocimum basilicum | G | O | 40.1 |
| Ocimum basilicum | G | S | 27.9 |
| Oenothera biennis | G | O | 26.3 |
| Oenothera biennis | G | R | 100.0 |
| Oenothera biennis | G | O | 49.6 |
| Oenothera biennis | G | S | 54.0 |
| Origanum vulgare | G | W | 100.0 |
| Origanum vulgare | G | O | 26.7 |
| Origanum vulgare | G | S | 21.3 |
| Oryza Sativa | G | S | 34.5 |
| Oxalis Deppei Lodd. | G | O | 27.4 |
| Panicum miliaceum | G | O | 25.3 |
| Pastinaca sativa | G | R | 95.0 |
| Petroselinum crispum | G | R | 44.5 |
| Petroselinum crispum | G | S | 26.5 |
| Peucedanum cervaria | G | R | 25.1 |
| Phaseolus coccineus | G | R | 30.9 |
| Phaseolus coccineus | G | O | 27.5 |
| Phaseolus mungo | G | R | 24.3 |
| Phlox paniculata | G | S | 37.9 |
| Physalis pruinosa | G | S | 26.5 |
| Phytolacca americana | G | S | 100.0 |
| Pimpinella anisum | G | S | 23.7 |
| Plantago coronopus | G | O | 25.1 |
| Plantago major | G | O | 25.0 |
| Plantago major | G | R | 20.5 |
| Plantago major | G | S | 23.6 |
| Poa compressa | G | O | 28.5 |
| Poa pratensis | G | O | 37.5 |
| Polygonum aviculare | G | R | 25.4 |
| Polygonum pensylvanicum | G | O | 21.3 |
| Portulaca oleracea | G | O | 28.0 |
| Poterium sanguisorba | G | O | 25.6 |
| Poterium sanguisorba | G | V | 21.9 |
| Prunella vulgaris | G | O | 23.4 |
| Pteridium aquilinum | G | R | 43.1 |
| Reseda odorata | G | O | 46.5 |
| Rhaphanus sativus | G | S | 32.6 |
| Rheum × cultorum | G | S | 20.9 |
| Ribes nidigrolaria | G | W | 29.8 |
| Ribes nidigrolaria | G | V | 53.7 |
| Ribes nigrum | G | V | 20.3 |
| Ribes Silvestre | G | W | 91.6 |
| Ricinus communis | G | S | 46.0 |
| Rosmarinus officinalis | G | R | 60.4 |
| Rubus idaeus | G | W | 28.2 |
| Rubus occidentalis | G | R | 93.6 |
| Rubus occidentalis | G | O | 40.0 |
| Rumex acetosella | G | V | 24.3 |
| Rumex crispus | G | R | 100.0 |
| Rumex patientia | G | O | 32.0 |
| Rumex scutatus | G | V | 28.6 |
| Ruta graveolens | G | S | 23.4 |
| Saccharum officinarum | G | O | 30.2 |
| Salix purpurea | G | S | 24.8 |
| Salvia elegans | G | O | 100.0 |
| Salvia officinalis | G | W | 52.4 |
| Salvia officinalis | G | R | 100.0 |
| Salvia officinalis | G | O | 100.0 |
| Salvia sclarea | G | O | 100.0 |
| Salvia sclarea | G | V | 23.0 |
| Salvia sclarea | G | W | 31.1 |
| Sambucus ebulus | G | O | 52.1 |
| Sambucus ebulus | G | R | 48.6 |
| Sanguisorba officinalis | G | R | 100.0 |
| Santolina chamaecyparissus | G | O | 100.0 |
| Serratula tinctoria | G | S | 56.8 |
| Satureja montana | G | O | 34.1 |
| Scolymus hispanicus | G | R | 37.9 |
| Scutellaria lateriflora | G | S | 54.7 |
| Senecio vulgaris | G | R | 35.3 |
| Solidago sp | G | S | 22.6 |
| Sonchus oleraceus | G | O | 23.7 |
| Sorghum caffrorum | G | V | 27.1 |
| Sorghum dochna | G | S | 40.7 |
| Sorghum dochna | G | O | 21.4 |
| Sorghum sudanense | G | V | 23.3 |
| Sorghum sudanense | G | W | 92.9 |
| Stellaria graminea | G | O | 25.4 |
| Stellaria media | G | O | 30.4 |
| Stellaria media | G | R | 22.0 |
| Tanacetum vulgare | G | O | 57.3 |
| Tanacetum vulgare | G | S | 38.4 |
| Tanacetum vulgare | G | O | 38.2 |
| Tanacetum vulgare | G | W | 26.3 |
| Taraxacum officinale | G | V | 20.0 |
| taraxacum officinale | G | O | 28.0 |
| Thymus fragantissimus | G | R | 79.9 |
| Thymus fragantissimus | G | O | 26.2 |
| Thymus herba-barona | G | W | 20.2 |
| Thymus serpyllum | G | V | 22.2 |
| Triticosecale spp. | G | S | 29.7 |
| Triticum durum | G | S | 37.8 |
| Triticum spelta | G | O | 31.0 |
| Triticum spelta | G | S | 37.9 |
| Typha latifolia | G | S | 27.5 |
| Urtica dioica | G | O | 60.3 |
| Vaccinium corymbosum | G | S | 33.2 |
| Vaccinium angustifolium | G | S | 43.7 |
| Vaccinium macrocarpon | G | W | 57.8 |
| Vaccinium macrocarpon | G | S | 59.9 |
| Valerianella locusta | G | O | 32.1 |
| Veratrum viride | G | O | 22.1 |
| Verbascum thapsus | G | S | 33.8 |
| Viburnum trilobum | G | V | 21.3 |
| Viburnum trilobum | G | W | 73.0 |
| Vicia faba | G | S | 21.2 |
| Vigna unguiculata | G | R | 20.1 |
| Vitis | G | V | 26.0 |
| Vitis | G | W | 66.1 |
| Vitis | G | O | 41.7 |
| Vitis | G | S | 30.7 |
| Xanthium sibiricum | G | O | 22.1 |
| Zea mays | G | S | 20.3 |
| Abies lasiocarpa | T | S | 22.4 |
| Achillea millefolium | T | S | 21.1 |
| Aconitum napellus | T | O | 100.0 |
| Acorus calamus | T | S | 21.0 |
| Ageratum conyzoides | T | O | 20.1 |
| Agrimonia eupatoria | T | W | 59.6 |
| Agropyron cristatum | T | R | 53.4 |
| Agropyron repens | T | S | 22.6 |
| Agrostis alba | T | O | 25.3 |
| Alchemilla mollis | T | W | 88.7 |
| Alchemilla mollis | T | O | 42.6 |
| Alchemilla mollis | T | R | 70.4 |
| Alchemilla mollis | T | S | 31.2 |
| Allium ascalonicum | T | S | 42.9 |
| Allium sativum | T | O | 100.0 |
| Allium tuberosum | T | O | 100.0 |
| Alpinia officinarum | T | O | 21.9 |
| Alpinia officinarum | T | S | 100.0 |
| Amaranthus candatus | T | S | 36.0 |
| Amaranthus gangeticus | T | S | 66.8 |
| Ananas comosus | T | O | 20.3 |
| Ananas comosus | T | W | 23.8 |
| Anethum graveolens | T | O | 35.8 |
| angelica archangelica | T | R | 53.5 |
| Anthemis nobilis | T | O | 45.3 |
| Anthemis tinctorium | T | S | 47.5 |
| Anthriscus cerefolium | T | O | 20.5 |
| Arctium minus | T | O | 54.1 |
| Arctostaphylos uva-ursi | T | O | 28.1 |
| Arctostaphylos uva-ursi | T | R | 100.0 |
| Aronia melanocarpa | T | V | 100.0 |
| Aronia melanocarpa | T | W | 42.7 |
| Aronia prunifolia | T | W | 39.0 |
| Artemisia absinthium | T | O | 25.6 |
| Artemisia dracunulus | T | O | 31.3 |
| Artemisia dracunulus | T | S | 22.3 |
| Aster | T | S | 20.9 |
| Avena sativa | T | S | 100.0 |
| Averrhoa carambola | T | O | 25.8 |
| Beta vulgaris | T | R | 100.0 |
| Beta vulgaris | T | O | 59.3 |
| Beta vulgaris | T | S | 41.4 |
| Betula glandulosa | T | S | 61.8 |
| Boesenbergia rotunda | T | O | 36.9 |
| Boesenbergia rotunda | T | S | 42.5 |
| Boletus edulis | T | S | 43.1 |
| Borago officinalis | T | S | 36.3 |
| Brassica hirta | T | S | 30.2 |
| Brassica juncea | T | R | 41.4 |
| Brassica Napus | T | S | 29.9 |
| Brassica napus | T | R | 22.9 |
| Brassica oleracea | T | R | 25.6 |
| Brassica oleracea | T | V | 27.0 |
| Brassica oleracea | T | R | 26.5 |
| Brassica rapa | T | R | 24.8 |
| Bromus inermis | T | O | 27.8 |
| Canna edulis | T | O | 40.3 |
| Capsicum annuum | T | S | 22.6 |
| Carex morrowii | T | O | 26.0 |
| Carex morrowii | T | R | 49.8 |
| Carya cordiformis | T | S | 28.8 |
| Carya cordiformis | T | O | 21.0 |
| Carya cordiformis | T | W | 88.7 |
| Clematis armandii | T | O | 20.1 |
| Chaerophyllum bulbosum | T | O | 22.8 |
| Chaerophyllum bulbosum | T | S | 24.3 |
| Agaricus bisporatus | T | S | 25.4 |
| Chelidonium majus | T | O | 39.0 |
| Chenopodium bonus-henricus | T | S | 44.3 |
| chrysanthemum coronarium | T | O | 33.4 |
| chrysanthemum coronarium | T | S | 23.9 |
| Cichorium endivia subs. Endivia | T | O | 44.3 |
| Cichorium endivia subs. Endivia | T | S | 20.5 |
| Circium arvense | T | R | 49.7 |
| Citrullus colocynthis | T | R | 37.0 |
| Citrullus colocynthis | T | S | 35.5 |
| Citrus limettoides | T | O | 47.1 |
| Citrus limon | T | S | 26.2 |
| Citrus limon | T | O | 73.9 |
| Citrus sinensis | T | V | 25.2 |
| Coix Lacryma-Jobi | T | O | 32.7 |
| Coix Lacryma-Jobi | T | S | 31.4 |
| Corchorus olitorius | T | O | 24.4 |
| Cornus canadensis | T | S | 41.3 |
| Crataegus sp | T | S | 34.0 |
| Crataegus submollis | T | S | 39.6 |
| Curcuma longa | T | O | 55.3 |
| Curcuma zedoaria | T | O | 24.4 |
| Cydonia oblonga | T | V | 35.2 |
| Cynara scolymus | T | O | 41.2 |
| Cynara scolymus | T | R | 36.8 |
| Dactilis Glomerata | T | O | 31.9 |
| Datura stramonium | T | S | 25.9 |
| Daucus carota | T | R | 92.3 |
| Daucus carota | T | O | 31.0 |
| Dipsacus sativus | T | O | 100.0 |
| Dirca palustris | T | S | 31.4 |
| Dolichos lablab | T | O | 23.1 |
| Dryopteris filix-mas | T | R | 68.2 |
| Echinacea purpurea | T | S | 38.2 |
| Eleusine coracana | T | O | 22.1 |
| Elymus junceus | T | R | 37.9 |
| Erigeron speciosus | T | O | 35.0 |
| Erysimum perofskianum | T | O | 22.6 |
| Erysimum perofskianum | T | S | 23.2 |
| Fagopyrum esculentum | T | S | 24.7 |
| Foeniculum vulgare | T | O | 31.4 |
| Foeniculum vulgare | T | V | 69.1 |
| Foeniculum vulgare | T | S | 38.5 |
| Fragaria × ananassa | T | O | 50.4 |
| Fragaria × ananassa | T | V | 30.2 |
| Fragaria × ananassa | T | S | 28.4 |
| Passiflora spp. | T | O | 30.2 |
| Passiflora spp. | T | V | 59.4 |
| Passiflora spp. | T | S | 24.4 |
| Fucus vesiculosus | T | O | 42.7 |
| Galinsoga ciliata | T | R | 49.3 |
| Gaultheria hispidula | T | W | 36.9 |
| Gentiana macrophylla | T | S | 26.1 |
| Ginkgo biloba | T | V | 27.1 |
| Glycyrrhiza glabra | T | W | 58.1 |
| Glycyrrhiza glabra | T | S | 50.4 |
| Glycyrrhiza glabra | T | R | 25.1 |
| Gossypium herbaceum | T | O | 22.7 |
| Gossypium herbaceum | T | S | 27.3 |
| Guizotia abyssinica | T | S | 38.5 |
| Hamamelis virginiana | T | O | 37.1 |
| Hamamelis virginiana | T | R | 100.0 |
| Hedeoma pulegioides | T | O | 28.5 |
| Hedeoma pulegioides | T | S | 28.2 |
| Helenium hoopesii | T | O | 31.7 |
| Helenium hoopesii | T | S | 56.0 |
| Helianthus tuberosus | T | V | 23.7 |
| Helichrysum thianschanicum | T | O | 38.4 |
| Helichrysum thianschanicum | T | R | 27.0 |
| Helleborus niger | T | S | 32.1 |
| Schizonepeta tenuifolia | T | O | 29.1 |
| Schizonepeta tenuifolia | T | S | 21.1 |
| Hibiscus cannabinus | T | O | 39.9 |
| Hibiscus cannabinus | T | S | 21.1 |
| Humulus lupulus | T | S | 54.8 |
| Humulus lupulus | T | R | 50.5 |
| Hydrastis canadensis | T | O | 20.9 |
| Hypericum henryi | T | O | 32.5 |
| Hypericum perforatum | T | S | 27.9 |
| Hypericum sp | T | W | 55.9 |
| Hypomyces lactifluorum | T | S | 42.7 |
| Iberis amara | T | S | 100.0 |
| Inula helenium | T | S | 30.1 |
| Ipomola batatas | T | V | 27.4 |
| Ipomola batatas | T | S | 44.9 |
| Juniperus communis | T | S | 57.8 |
| Laportea canadensis | T | S | 63.5 |
| Laurus nobilis | T | W | 73.6 |
| Laurus nobilis | T | S | 21.2 |
| Lavandula angustifolia | T | O | 22.7 |
| Lavandula angustifolia | T | S | 25.1 |
| Lavandula latifolia | T | O | 100.0 |
| Lavandula latifolia | T | S | 28.5 |
| Ledum groenlandicum | T | O | 54.3 |
| Lentinus edodes | T | S | 25.7 |
| Leonurus cardiaca | T | R | 24.3 |
| Lepidium sativum | T | O | 100.0 |
| Levisticum officinale | T | R | 41.2 |
| Litchi chinensis | T | S | 100.0 |
| Lolium multiflorum | T | O | 24.0 |
| Lolium perenne | T | O | 27.8 |
| Lonicera ramosissima | T | S | 20.9 |
| Lupinus polyphyllus | T | O | 35.1 |
| Lupinus polyphyllus | T | S | 20.5 |
| Luzula sylvatica | T | R | 22.6 |
| Majorana hortensis | T | V | 20.1 |
| Malus spp. | T | V | 37.8 |
| Malus spp. | T | S | 45.1 |
| Malus hupehensis | T | S | 24.4 |
| Melaleuca alternifolia | T | O | 26.7 |
| Melissa officinalis | T | S | 20.7 |
| mentha arvensis | T | R | 34.0 |
| Mentha piperita | T | S | 60.1 |
| Mentha pulegium | T | V | 24.5 |
| Mentha pulegium | T | W | 24.8 |
| Mentha spicata | T | O | 24.4 |
| Mentha suaveolens | T | S | 28.9 |
| Monarda didyma | T | O | 54.7 |
| Musa paradisiaca | T | O | 21.4 |
| Musa paradisiaca | T | W | 32.8 |
| nasturtium officinale | T | O | 100.0 |
| Nepeta cataria | T | O | 60.1 |
| Nepeta cataria | T | S | 23.4 |
| Nigella sativa | T | S | 23.2 |
| Agaricus bisporatus | T | S | 25.8 |
| Psidium spp. | T | S | 28.3 |
| Pleurotus spp. | T | S | 31.6 |
| Citrus reticulata | T | V | 32.7 |
| Citrus reticulata | T | S | 29.4 |
| Ocimum Basilicum | T | V | 30.7 |
| Ocimum Basilicum | T | W | 30.9 |
| Ocimum Basilicum | T | O | 39.1 |
| Oenothera biennis | T | S | 29.6 |
| Oenothera biennis | T | O | 24.2 |
| Oenothera biennis | T | R | 58.6 |
| Onobrychis viciifolia | T | O | 42.6 |
| Origanum vulgare | T | S | 53.8 |
| Oryza sativa | T | S | 33.3 |
| Oxalis Deppei | T | O | 30.8 |
| Panicum miliaceum | T | S | 21.2 |
| Pastinaca sativa | T | S | 53.9 |
| Pastinaca sativa | T | R | 20.8 |
| Pastinaca sativa | T | O | 26.9 |
| Petroselinum crispum | T | R | 58.2 |
| Phaseolus coccineus | T | S | 27.1 |
| Phaseolus vulgaris | T | W | 37.9 |
| Phaseolus vulgaris | T | O | 22.2 |
| Phaseolus vulgaris | T | S | 23.2 |
| Phlox paniculata | T | S | 21.3 |
| Physalis pruinosa | T | S | 35.2 |
| Phytolacca americana | T | S | 100.0 |
| Plantago coronopus | T | O | 21.2 |
| Plantago coronopus | T | S | 48.2 |
| Poa pratensis | T | O | 50.7 |
| Podophyllum peltatum | T | S | 27.9 |
| Polygonum chinense | T | S | 25.0 |
| Polygonum aviculare | T | O | 26.0 |
| Polygonum aviculare | T | R | 100.0 |
| Polygonum pensylvanicum | T | O | 42.3 |
| Polygonum persicaria | T | O | 28.8 |
| Populus incrassata | T | S | 100.0 |
| Populus Tremula | T | S | 48.5 |
| Populus × petrowskyana | T | S | 44.1 |
| Populus × petrowskyana | T | O | 100.0 |
| Populus × petrowskyana | T | W | 72.0 |
| Portulaca oleracera | T | O | 33.7 |
| Poterium sanguisorba | T | W | 100.0 |
| Prunus spp. | T | S | 39.6 |
| Prunus persica | T | O | 21.4 |
| Prunus persica | T | V | 26.6 |
| Psidium guajava | T | V | 37.7 |
| Psoralea corylifolia | T | S | 51.5 |
| Pteridium aquilinum | T | R | 76.2 |
| Pteridium aquilinum | T | S | 27.9 |
| Punica granatum | T | W | 66.4 |
| Rehmannia glutinosa | T | O | 83.0 |
| Frangula alnus | T | S | 40.7 |
| Raphanus sativus | T | R | 36.5 |
| Raphanus sativus | T | S | 22.4 |
| Reseda luteola | T | S | 23.6 |
| Reseda odorata | T | O | 20.3 |
| Frangula alnus | T | R | 65.3 |
| Rheum officinale | T | O | 100.0 |
| Rheum officinale | T | S | 33.3 |
| Rheum × cultorum | T | S | 34.0 |
| Ricinus communis | T | S | 27.5 |
| Ribes Grossularia | T | W | 24.8 |
| Ribes nidigrolaria | T | W | 24.4 |
| Ribes nigrum | T | S | 50.1 |
| Ribes nigrum | T | V | 23.8 |
| Ribes nigrum | T | W | 64.1 |
| Ribes Sylvestre | T | W | 32.4 |
| Rosa rugosa | T | W | 100.0 |
| Rosmarinus officinalis | T | R | 75.8 |
| Rosmarinus officinalis | T | W | 46.6 |
| Rubus idaeus | T | O | 27.6 |
| Rubus idaeus | T | S | 24.3 |
| Rubus idaeus | T | O | 35.5 |
| Rubus occidentalis | T | R | 93.2 |
| Rubus occidentalis | T | O | 42.1 |
| Rubus occidentalis | T | S | 20.5 |
| Rumex acetosella | T | V | 44.9 |
| Rumex crispus | T | O | 31.3 |
| Rumex crispus | T | R | 100.0 |
| Rumex crispus | T | S | 20.8 |
| Ruta graveolens | T | O | 24.1 |
| Serenoa repens | T | S | 28.5 |
| Salvia officinalis | T | R | 66.5 |
| Salvia officinalis | T | O | 54.0 |
| Salvia officinalis | T | W | 47.2 |
| Sambucus canadensis | T | S | 23.2 |
| Sambucus canadensis | T | O | 35.0 |
| Sambucus canadensis | T | R | 32.6 |
| Sambucus canadensis | T | W | 54.0 |
| Sanguisorba minor | T | W | 50.0 |
| Santolina chamaecyparissus | T | O | 75.8 |
| Santolina chamaecyparissus | T | R | 33.3 |
| Serratula tinctoria | T | S | 36.3 |
| Datura metel | T | O | 36.9 |
| Datura metel | T | S | 21.4 |
| Satureja montana | T | O | 100.0 |
| Satureja montana | T | R | 66.8 |
| Satureja repandra | T | R | 87.4 |
| Scorzorera hispanica | T | R | 42.3 |
| Scorzorera hispanica | T | S | 20.8 |
| Scutellaria lateriflora | T | S | 36.6 |
| Sium sisarum | T | O | 22.1 |
| Solanum melongena | T | O | 22.4 |
| Solidago sp | T | S | 22.6 |
| Sonchus oleraceus | T | R | 41.8 |
| Sorghum caffrorum | T | O | 23.0 |
| Sorghum dochna | T | O | 30.3 |
| Sorghum dochna | T | O | 53.5 |
| Sorghum durra | T | V | 21.6 |
| Sorghum sudanense | T | V | 23.7 |
| Stachys byzantina | T | O | 25.3 |
| Stellaria graminea | T | O | 27.6 |
| Stellaria graminea | T | S | 36.7 |
| Stellaria media | T | O | 22.6 |
| Stipa capillata | T | O | 36.7 |
| Symphytum officinale | T | O | 20.6 |
| Symphytum officinale | T | V | 25.0 |
| Tanacetum cinerariifolium | T | R | 24.9 |
| Tanacetum vulgare | T | O | 46.4 |
| Tanacetum vulgare | T | S | 32.0 |
| Taraxacum officinale | T | O | 63.1 |
| Thlaspi arvense | T | O | 32.5 |
| Thymus fragantissimus | T | R | 36.7 |
| Thymus fragantissimus | T | O | 100.0 |
| Thymus praecox subsp arcticus | T | O | 38.7 |
| Thymus pseudolanuginosus | T | R | 21.5 |
| Thymus vulgaris | T | W | 20.0 |
| Triticosecale spp. | T | O | 26.0 |
| Triticum aestivum | T | O | 20.9 |
| Triticum turgidum | T | O | 49.4 |
| Triticum spelta | T | O | 35.0 |
| Tropaeolum majus | T | S | 23.5 |
| Tsuga diversifolia | T | S | 34.3 |
| Tsuga mertensiana | T | S | 32.8 |
| Typha latifolia | T | S | 36.1 |
| Urtica dioica | T | O | 32.8 |
| Vaccinium angustifolium | T | S | 33.7 |
| Vaccinium macrocarpon | T | V | 24.1 |
| Vaccinium macrocarpon | T | W | 30.3 |
| Vaccinium macrocarpon | T | S | 70.9 |
| Vaccinium macrocarpon | T | O | 57.2 |
| Valeriana officinalis | T | O | 26.0 |
| Valerianella locusta | T | O | 53.7 |
| Verbascum thapsus | T | O | 22.8 |
| Verbascum thapsus | T | S | 25.2 |
| Veronica officinalis | T | O | 29.9 |
| Vitis | T | S | 39.1 |
| Vitis | T | O | 40.0 |
| Vitis | T | W | 23.5 |
| Vitis | T | S | 26.4 |
| Weigela coraeensis | T | S | 20.1 |
| Weigela hortensis | T | S | 25.3 |
| Xanthium sibiricum | T | O | 28.4 |
| Zea mays | T | S | 38.4 |
| Oenothera biennis | A | R | 80.3 |
| Alchemilla mollis | T | R | 96.0 |
| Alchemilla mollis | A | R | 87.2 |
| Symphytum officinale | A | O | 80.2 |
| Fragariax ananassa | A | R | 97.9 |
| Fragariax ananassa | G | R | 93.8 |
| Vaccinium corymbosum | G | R | 58.6 |
| Vaccinium augustifolium | A | R | 71.8 |
| Vaccinium augustifolium | G | R | 53.6 |
| Vitis | A | R | 62.5 |
| Vitis | G | R | 79.4 |
| Petasites japonicus | A | R | 56.5 |
| Petasites japonicus | G | R | 53.0 |
| Nicotiana rustica | G | O | 61.1 |
| Pysalis ixocarpa | A | R | 53.8 |
| Pteridium aquilinum | T | O | 69.2 |
| Pteridium aquilinum | A | R | 66.2 |
| Pteridium aquilinum | G | R | 56.3 |
| Pteridium aquilinum | G | O | 56.2 |
| Matteuccia pensylvanica | T | R | 67.2 |
| Matteuccia pensylvanica | A | R | 59.0 |
| Ocimum tenuiflorum | T | O | 54.8 |
| Carthamus tinctorius | A | R | 50.9 |
| Carthamus tinctorius | G | R | 69.0 |
| Ligustrum vulgare | T | O | 87.0 |
| Ligustrum vulgare | A | O | 76.2 |
| Ligustrum vulgare | G | O | 85.7 |
| Malva verticillata | T | R | 80.1 |
| Malva verticillata | A | R | 82.9 |
| Malva verticillata | G | R | 82.4 |
| Hamamelis virginiana | T | R | 56.1 |
| Arctostaphylos uva-ursi | T | R | 74.8 |
| Arctostaphylos uva-ursi | G | R | 86.0 |
| Vicia faba | T | O | 84.6 |
| Sempervivum tectorum | T | O | 57.3 |
| Sempervivum tectorum | A | O | 74.8 |
| Sempervivum tectorum | G | O | 52.3 |
| Ajuga reptans | T | O | 55.3 |
| Ajuga reptans | A | O | 52.3 |
| Ajuga reptans | G | O | 72.1 |
| Phlox paniculata | T | O | 66.2 |
| Ligularia dentata | A | O | 52.1 |
| Ligularia dentata | G | R | 50.8 |
| Ligularia dentata | G | O | 52.6 |
| Achillea ptarmica | T | O | 50.9 |
| Achillea ptarmica | A | O | 54.3 |
| Achillea ptarmica | G | O | 64.3 |
| Geranium pratense | T | R | 93.4 |
| Geranium pratense | A | R | 98.5 |
| Geranium pratense | G | R | 97.4 |
| Thalictrum aquilegiifolium | T | O | 53.6 |
| Thalictrum aquilegiifolium | G | O | 60.4 |
| Veronica spicata | T | O | 55.9 |
| Veronica spicata | A | O | 59.2 |
| Veronica spicata | G | O | 56.2 |
| Helenium spp. | T | O | 55.7 |
| Salvia sylvestris | T | O | 77.4 |
| Salvia sylvestris | A | O | 66.9 |
| Salvia sylvestris | G | O | 55.0 |
| Salvia regeliana | T | O | 62.6 |
| Crambe cordifolia | G | R | 56.3 |
| Crambe cordifolia | G | O | 56.7 |
| Rudbeckia maxima | G | O | 68.4 |
| Trollius × cultorum | T | R | 97.6 |
| Trollius × cultorum | A | R | 93.2 |
| Trollius × cultorum | G | R | 100.1 |
| Amsonia tabernaemontana | A | R | 53.2 |
| Oenothera fruticosa spp. | T | R | 109.8 |
| Oenothera fruticosa spp. | T | O | 61.3 |
| Oenothera fruticosa spp. | A | R | 97.5 |
| Oenothera fruticosa spp. | G | R | 105.9 |
| Veronica austriaca ssp teucrium | T | O | 68.6 |
| Veronica austriaca ssp teucrium | G | O | 58.1 |
| Coreopsis verticillata | T | R | 55.6 |
| Coreopsis verticillata | G | O | 70.4 |
| Potentilla fruticosa | T | R | 104.8 |
| Potentilla fruticosa | A | R | 99.4 |
| Potentilla fruticosa | G | R | 98.6 |
| Vernonia gigantea | A | R | 50.4 |
| Vernonia gigantea | A | O | 62.3 |
| Vernonia gigantea | G | R | 51.2 |
| Vernonia gigantea | G | O | 50.7 |
| Penstemon digitalis | T | R | 64.5 |
| Penstemon digitalis | A | R | 63.5 |
| Penstemon digitalis | A | O | 57.3 |
| Penstemon digitalis | G | R | 63.4 |
| Penstemon digitalis | G | O | 67.8 |
| Malus spp. | T | R | 56.1 |
| Malus spp. | T | O | 56.7 |
| Malus spp. | A | R | 50.8 |
| Malus spp. | G | R | 51.2 |
| Hosta sieboldiana | G | O | 50.9 |
| Hamamelis mollis | T | R | 99.1 |
| Hamamelis mollis | A | R | 94.1 |
| Hamamelis mollis | G | R | 89.4 |
| Chaenomeles × superba | T | R | 56.2 |
| Chaenomeles × superba | A | R | 71.9 |
| Chaenomeles × superba | G | R | 66.6 |
| Chaenomeles × superba | G | O | 52.0 |
| Centaurea dealbata | T | R | 50.9 |
| Centaurea dealbata | A | R | 74.1 |
| Paeonia spp. | T | R | 79.8 |
| Paeonia spp. | T | O | 58.6 |
| Paeonia spp. | A | R | 79.6 |
| Paeonia spp. | A | O | 58.5 |
| Paeonia spp. | G | R | 82.0 |
| Paeonia spp. | G | O | 60.0 |
| Lysimachia clethroides | T | R | 83.3 |
| Lysimachia clethroides | T | O | 64.3 |
| Lysimachia clethroides | G | R | 85.8 |
| Lysimachia clethroides | G | O | 67.8 |
| Magnolia × loebneri | T | R | 61.4 |
| Iberis sempervirens | T | O | 62.4 |
| Iberis sempervirens | G | O | 63.8 |
| Filipendula vulgaris | T | R | 98.3 |
| Filipendula vulgaris | A | R | 94.5 |
| Filipendula vulgaris | G | R | 96.3 |
| Geranium sanguineum | T | R | 89.4 |
| Geranium sanguineum | T | O | 63.3 |
| Geranium sanguineum | A | R | 82.6 |
| Geranium sanguineum | A | O | 53.2 |
| Garanium sanguineum | G | R | 88.8 |
| Garanium sanguineum | G | O | 57.7 |
| Philadelphus coronarius | A | O | 55.5 |
| paeonia suffruticosa | T | R | 58.9 |
| paeonia suffruticosa | T | O | 52.1 |
| Paeonia suffruticosa | A | R | 73.8 |
| Paeonia suffruticosa | A | O | 52.2 |
| Paeonia suffruticosa | G | R | 58.7 |
| Paeonia suffruticosa | G | O | 50.4 |
| Dahlia spp. | T | R | 77.4 |
| Begonia convolvulacea | T | O | 69.8 |
| Begonia convolvulacea | A | O | 67.5 |
| Begonia convolvulacea | G | O | 72.6 |
| Begonia eminii | T | O | 72.8 |
| Begonia eminii | A | O | 77.2 |
| Begonia eminii | G | O | 75.4 |
| Begonia glabra | T | O | 82.3 |
| Begonia mannii | A | O | 82.5 |
| Begonia mannii | G | O | 72.8 |
| Begonia polygonoides | T | O | 79.0 |
| Begonia polygonoides | A | O | 74.8 |
| Begonia polygonoides | G | O | 73.2 |
| Fushia spp. | T | R | 76.6 |
| Fushia spp. | A | R | 70.7 |
| Fushia spp. | G | R | 76.9 |
| Butomus umbellatus | A | O | 58.8 |
| Onoclea sensibilis | G | O | 54.7 |
| Onoclea sensibilis | G | R | 50.1 |
| Pinus cembra | A | R | 83.2 |
| Pinus cembra | G | R | 76.3 |
| Cornus sericea | T | R | 104.0 |
| Cornus sericea | A | O | 53.4 |
| Cornus sericea | A | R | 91.8 |
| Cornus sericea | G | O | 51.0 |
| Cornus sericea | G | R | 98.5 |
| Hydrangea quercifolia | T | R | 58.1 |
| Solidago caesia | T | R | 60.7 |
| Solidago caesia | A | R | 60.5 |
| Cornus alba | T | R | 98.9 |
| Cornus alba | A | R | 106.7 |
| Cornus alba | G | R | 85.3 |
| Carpinus caroliniana | T | R | 95.4 |
| Carpinus caroliniana | A | R | 86.2 |
| Carpinus caroliniana | G | R | 94.5 |
| Astilbe chinensis | T | R | 54.3 |
| Astilbe chinensis | G | R | 50.3 |
| Symphoricarpos albus | G | R | 52.0 |
| Euphorbia amygdaloides | T | R | 103.8 |
| Euphorbia amygdaloides | A | R | 75.2 |
| Euphorbia amygdaloides | G | R | 71.3 |
| Viburnum plicatum | A | R | 61.0 |
| Viburnum plicatum | G | R | 57.9 |
| Buxus microphylla | T | R | 58.0 |
| Astilboides tabularis | T | R | 104.2 |
| Astilboides tabularis | A | R | 108.1 |
| Astilboides tabularis | G | R | 100.3 |
| Staphylea trifolia | A | R | 63.6 |
| Bergenia × schmidtii | T | R | 100.5 |
| Bergenia × schmidtii | A | R | 113.7 |
| Bergenia × schmidtii | G | R | 99.3 |
| Rodgersia podophylla | T | R | 68.9 |
| Rodgersia podophylla | A | R | 59.4 |
| Rodgersia podophylla | G | R | 56.5 |
| Geranium phaeum | T | R | 92.7 |
| Geranium phaeum | A | R | 84.3 |
| Geranium phaeum | G | R | 101.0 |
| Rubus pubescens | T | R | 71.5 |
| Rubus pubescens | A | R | 76.2 |
| Rubus pubescens | G | R | 82.8 |
| Taxus × media | T | R | 60.1 |
| Taxus × media | A | R | 61.6 |
| Taxus × media | G | R | 52.3 |
| Geranium × cantabrigiense | T | R | 106.1 |
| Geranium × cantabrigiense | A | R | 94.2 |
| Geranium × cantabrigiense | G | R | 95.9 |
| Fuchia magellanica | T | R | 100.2 |
| Fuchia magellanica | A | R | 91.9 |
| Fuchia magellanica | G | R | 102.2 |
| Microbiata decussata | A | R | 51.5 |
| Microbiata decussata | G | R | 51.9 |
| Rhododendron spp. | G | R | 51.2 |
| Stephanandra incisa | T | R | 102.5 |
| Stephanandra incisa | A | R | 104.6 |
| Stephanandra incisa | G | R | 99.1 |
| Corylus maxima | A | R | 50.8 |
| Corylus maxima | G | R | 57.1 |
| Cyperus alternifolius | G | R | 56.2 |
| Soleirolia soleirolii | A | R | 51.2 |
| Soleirolia soleirolii | G | R | 68.0 |
| Strelitzia reginae | T | R | 106.5 |
| Strelitzia reginae | A | R | 94.3 |
| Strelitzia reginae | G | R | 111.7 |
| Hedychium coronarium | T | R | 53.5 |
| Hedychium coronarium | A | R | 86.9 |
| Hedychium coronarium | G | R | 74.6 |
| Strelitzia reginae | T | R | 78.6 |
| Strelitzia reginae | A | R | 78.0 |
| Strelitzia reginae | G | R | 107.3 |
| Symphoricarpos orbiculatus | G | R | 58.7 |
| Rodgersia spp. | A | R | 59.5 |
| Rodgersia spp. | G | R | 59.0 |
| Lamiastrum galeobdolon | T | R | 91.5 |
| Astilbe × arendsii | A | R | 84.5 |
| Clematis alpina | A | R | 54.4 |
| Stewartia pseudocamellia | T | R | 75.5 |
| Stewartia pseudocamellia | A | R | 84.1 |
| Stewartia pseudocamellia | G | R | 81.3 |
| Pinus mugo | T | R | 58.9 |
| Pinus mugo | A | R | 53.7 |
| Pinus mugo | G | R | 61.7 |
| Rubus thibetanus | T | R | 97.6 |
| Rubus thibetanus | A | R | 97.9 |
| Rubus thibetanus | G | R | 95.4 |
| Rubus arcticus | T | R | 89.3 |
| Rubus arcticus | A | R | 85.5 |
| Rubus Phoenicolasius | G | R | 93.2 |
| ribes americanum | T | R | 70.4 |
| Passiflora spp. | T | O | 62.4 |
| Rubus occidentalis | T | R | 70.9 |
| Nicotiana tabacum | G | O | 60.9 |
| Beta vulgaris | T | O | 71.3 |

### Example III

### Exemplary Purification of Inhibitory Activity Found in an Extract

Extracts were separated by HPLC on an Agilent 1100 system (San Fernando, Calif.). Briefly, 100 μL of a crude extract prepared as described in Example I was applied on a C18 reverse-phase column (Purospher RP-18 5 μm, 4.0×125 mm (HP), Agilent, San Fernando, Calif.). Elution of compounds was achieved with a linear gradient of 10-85% acetonitrile. Fractions were collected, evaporated, resuspended in aqueous buffer and then reanalysed for their inhibition activity on specific enzymes as already described. Fractions of interest (demonstrating a biological activity) were then re-isolated at a larger scale for further analysis and characterisation.

### Example IV

### Preparation of Plant Extracts (Method B)

Method B is summarized in general terms in FIG. 5. The method can be divided into two main parts corresponding to preliminary analytical scale extraction and a second larger scale extraction process.

1. Analytical Scale Extraction—Selection of Plants/Extracts

The processed plant materials (leaves, roots, seeds and the like) were obtained by dedicated greenhouse cultivation (with or without physical/chemical stress), from commercial suppliers, or by gathering from non-cultivated natural sources. For each plant used in either analytical scale or large scale extraction, a properly identified and labelled sample was kept in storage in the laboratory.

The extraction protocols for both the preliminary analytical scale and large scale extractions are shown generally in FIG. 6.

The collected dried plant material (2-10 g) was first submitted to solid-liquid extractions to generate crude extract A (mg scale). Two different solvents were tested (ethanol/methanol or ethanol/water mixtures). The extracts were then defatted with hexane to yield hydrbalcoholic or alcoholic extract B and hexane extract C. A partitioning of extract B with ethyl acetate was then performed after dilution with water to yield aqueous extract E and organic extract F.

The extracts were sampled and evaluated for their ability to inhibit one or more target protease and for their ability to affect one or more cellular activity in the skin using the methods described below.

Analysis of the results allows for the selection of plant materials for the large-scale extraction. The selection includes a decision regarding part of the plant and quantity of dried material needed to obtain sufficient mass of extract for pure active compound isolation. The selection also involves a choice of solvent system (aqueous versus alcoholic) and active extract (B, E or F) to be used in further work.

The extracts were also analyzed by Thin Layer Chromatography (TLC) with different reagents specific to classical chemical groups of natural products (terpenes, alkaloids, phenolic acids, polyphenols) to evaluate the increase in concentration achieved by partitioning at each step, and also to remove any materials likely to produce false positive results (fatty acids, chlorophylls) and to provide an indication of which fractionation steps to use in further extractions.

2. Large Scale Extraction—Isolation

For each new specimen, a repeat analytical scale extraction is performed to confirm the biological activity before beginning the large-scale extraction process.

The first step is to release the secondary metabolites from the dried and powdered material by means of an all purpose solvent mixture which is selected based on the results obtained in the analytical scale preparation. This can be done by successive maceration/percolation operations using the same solvent which should dissolve most natural compounds at the same time. The bulk of the inert and insoluble material such as cellulose is then removed by filtration. Conditions of drying and grinding are controlled (temperature of drying less than 45° C., particles size).

The second step is to remove a portion of the unwanted material in a series of liquid-liquid low resolution extractions using solvents of different polarity with the aim of a multi-gram mixture containing all the natural products of interest and to remove the most of the undesired material.

The extraction protocol is illustrated in FIG. 6 and is essentially the same as the procedure for the analytical preparation. The dried and pulverized material (2-3 Kg for large scale) is extracted repeatedly (maceration/percolation) with ethanol/methanol [85:15] v/v (a) or ethanol/water [85:15] v/v (b) mixtures (3×5-10 L) at room temperature for 2×24-48 h, based on the analytical scale results (yield of extraction).

In the case of an alcoholic extraction (a), the combined alcoholic extracts (A) are concentrated under reduced pressure, diluted with water (10-15%) and extracted with hexane (or heptane) to yield hexane extract (C) and hydroalcoholic fraction (B). This is then concentrated and diluted with ethanol (20%) before being extracted with ethyl acetate to yield aqueous (E) and ethyl acetate extracts (F).

In the case of an hydroalcoholic extraction (b), the combined aqueous extracts (A) are extracted with hexane to yield hexane extract (C) and hydroalcoholic fraction (B). The latter is then concentrated until residual water and diluted with ethanol (20%) before extracted with ethyl acetate to yield aqueous (E) and ethyl acetate extracts (F).

All the extracts (A-F) are sampled to verify the process recovery and the aliquots are submitted to a biological evaluation (selective enzymatic inhibition). The results are compared with those obtained on the analytical scale section and the selected positive extract is then concentrated to dryness under reduced pressure.

All the extracts are analyzed by TLC to compare with analytic scale extracts.

### Example V

### Protease Inhibition by Plant Extracts in a Human Skin Model

A cellular model of the skin was used to determine the potential inhibitory effect of aqueous and ethanolic plant extracts prepared as described in Example I in the skin. Human dermal fibroblasts (Cascade Biologics, 5×10⁴/well), type 1 collagen (3 mg/ml, Sigma), and cell culture medium were pipetted into 12 or 24-well untreated Falcon plates and incubated for 1 hour at 37° C., allowing for gel formation. Cell culture medium was then added to the wells and the gels were incubated overnight at 37° C. in a 5% CO₂ controlled atmosphere. The gels were incubated for 5 days, with media changes at days 2 and 4, allowing for fibroblast proliferation, with collagen and protease synthesis and secretion into the gel. On day 5, the media were removed and donor-matched human epidermal keratinocytes (Cascade Biologics, 10⁵ cells/well) in biological medium were gently pipetted onto the gels. The wells were further incubated for 3 days with change of media on day 7, allowing for the establishment of a confluent layer of keratinocytes on the surface of the gel. On day 8, media were removed and culture medium containing the test plant extracts was added to the wells, followed by 6 or 24 hour incubations at 37° C. in a 5% CO₂ controlled atmosphere. The gels were then removed from the wells and extracted with PBS, with 3 freeze-thaw cycles, followed by centrifugation. The proteolytic activity in the supernatants was assayed by means of a fluorometric assay as described above (Example II).

The results are provided in Table 6.

TABLE 6
Inhibition of Proteases in a Human Skin Model

| | | Part of | | | % Inhib. | % Inhib. |
|---|---|---|---|---|---|---|
| Plant | Stress¹ | plant² | Concentration³ | Protease | 6 hr | 24 hr |
| Aconitum napellus | G | L | 2X | MMP-3 | 0 | 31 |
| Acorus calamus | G | L | 2X | MMP-3 | 0 | 9 |
| Agrostis alba | A | L | 1X | MMP-1 | 0 | 0 |
| Alchemilla mollis | A | L | 0.8X | MMP-3 | 55 | 41 |
| Allium cepa | N | Fl | 2X | MMP-2 | 49 | 0 |
| Allium sativum | A | L | 2X | MMP-2 | NA | 10 |
| Allium Tuberosum | A | L | 1X | MMP-3 | 0 | 35 |
| Aloe vera | G | L | 2X | MMP-2 | 0 | 0 |
| Ambrosia artemisiifolia | N | L/St/Fl | 2X | MMP-9 | 11 | 25 |
| Anethum graveolens | A | Fl/L/St | 2X | MMP-2 | 0 | 0 |
| Anethum graveolens | G | L | 1X | MMP-3 | 2 | 31 |
| Anethum graveolens | G | L | 1X | MMP-3 | 0 | 0 |
| Anthemis tinctoria | A | L/St | 2X | MMP-3 | 0 | 35 |
| Aronia melanocarpa | N | L | 2X | MMP-3 | 0 | 38 |
| (Michx.) Ell. | | | | | | |
| Aronia melanocarpa | G | L | 1X | MMP-3 | 0 | 34 |
| (Michx.) Ell. | | | | | | |
| Aronia x prunifolia | N | L/St | 2X | MMP-9 | 0 | 0 |
| Artemisia dracunculus | G | L/St | 2X | MMP-9 | 0 | 0 |
| Artemisia dracunlus | N | L/St/Fr | 2X | MMP-9 | 0 | 0 |
| Avena sativa | N | L | 2X | MMP-2 | 0 | 21 |
| Beta vulgaris | G | L | 2X | MMP-2 | 12 | 10 |
| Beta vulgaris spp. | N | L | 2X | MMP-2 | 0 | 0 |
| Maritima | | | | | | |
| Beta vulgaris subsp. | N | L | 2X | MMP-2 | 0 | 0 |
| Vulgaris | | | | | | |
| Borago officinalis | N | B | 1X | MMP-1 | 16 | 0 |
| Brassica napus | N | L | 0.7X | MMP-9 | 0 | 0 |
| Brassica oleracea | N | L | 2X | MMP-2 | NA | 17 |
| Brassica oleracea | N | L | 2X | MMP-2 | 0 | 0 |
| Brassica oleracea | A | L | 0.7X | MMP-9 | 0 | 14 |
| Brassica oleracea | G | Fl | 1X | MMP-1 | 0 | 0 |
| Brassica oleracea | A | L | 1X | MMP-9 | 9 | 16 |
| Brassica rapa | A | L | 2X | MMP-2 | 16 | 0 |
| Brassica rapa | G | L | 2X | MMP-2 | 11 | 10 |
| Bromus inermis | A | L | 2X | MMP-9 | 0 | 0 |
| Capsicum annuum | G | Fr | 1X | MMP-1 | 0 | 14 |
| Cerastium tomentosum | G | L/St | 2X | MMP-2 | 5 | 40 |
| Chaerophyllum | N | Fl/Fr | 2X | MMP-1 | 0 | 79 |
| bulbosum | | | | | | |
| Chenopodium quinoa | N | L/St | 2X | MMP-9 | 26 | 35 |
| Chichorium endivia | G | L | 2X | MMP-2 | 16 | 23 |
| Circium arvense | G | L/St | 2X | MMP-2 | 0 | 9 |
| Citrullus lanatus | A | L | 0.5X | MMP-9 | 16 | 0 |
| Cornus canadensis | N | L | 2X | MMP-3 | 0 | 44 |
| Cynara cardunculus | G | Fr | 2X | MMP-9 | 4 | 5 |
| subsp. Cardunculus | | | | | | |
| Daucus carota | A | L | 2X | MMP-2 | 0 | 0 |
| Daucus carota | A | L | 2X | MMP-2 | 0 | 0 |
| Daucus carota | G | L | 2X | MMP-2 | 0 | 12 |
| Dioscorea batatas | N | L/Fl/Fr | 2X | MMP-2 | 0 | 0 |
| Dolichos lablab | G | Fl/Fr | 2X | MMP-9 | 14 | 23 |
| Fagopyrum esculentum | G | L | 2X | MMP-1 | 0 | 0 |
| Fagopyrum tataricum | G | L | 1X | MMP-3 | 64 | 38 |
| Foeniculum vulgare | G | Fl | 2X | MMP-2 | 0 | 20 |
| Foeniculum vulgare | N | L | 0.8X | MMP-9 | 0 | 10 |
| Fragaria x ananassa | A | L | 2X | MMP-3 | 0 | 0 |
| Frangula alnus | N | Fr | 2X | MMP-3 | 0 | 44 |
| Galinsoga | N | L/St/Fl | 2X | MMP-2 | 0 | 0 |
| quadriradiata | | | | | | |
| Glycine max | G | Fr | 0.7X | | 0 | 0 |
| Glycyrrhiza glabra | A | L | 2X | MMP-9 | 0 | 0 |
| Glycyrrhiza glabra | G | L/St | 2X | MMP-2 | 0 | 0 |
| Hamamelis virginiana | A | L/St | 2X | MMP-1 | 41 | 37 |
| Helianthus strumosus | G | L | 2X | MMP-2 | 0 | 0 |
| Heliotropium | G | Fl | 2X | MMP-3 | 3 | 40 |
| arborescens | | | | | | |
| Hordeum vulgare | G | L | 1X | MMP-1 | 13 | 0 |
| subsp. Vulgare | | | | | | |
| Hypomyces lactifluorum | N | Fr | 1X | MMP-9 | 12 | 0 |
| Juniperus communis | N | Fr/L/St | 2X | MMP-3 | 10 | 0 |
| Kochia scoparia | N | L/St/Fr | 2X | MMP-1 | 0 | 0 |
| Lactuca sativa | G | L | 2X | MMP-2 | 0 | 0 |
| Lentinus edodes | N | Fr | 2X | MMP-2 | 24 | 15 |
| Lotus corniculatus | A | Fr/L/St | 2X | MMP-9 | 0 | 0 |
| Lotus corniculatus | N | P | 2X | MMP-9 | 0 | 0 |
| Manihot esculenta | N | Fr | 0.5X | MMP-9 | 8 | 0 |
| Matricaria recutita | G | Fl/L/St | 0.5X | MMP-9 | 0 | 0 |
| Melilotus albus | G | L/St | 2X | MMP-9 | 0 | 0 |
| Melissa officinalis | N | L/St | 0.43X | MMP-2 | 0 | 0 |
| Mentha x piperita | N | L/St/Fl | 2X | MMP-2 | 23 | 15 |
| Origanum majorana | A | L/St/Fl | 2X | MMP-3 | 0 | 0 |
| Panax quinquefolius | N | Fr | 2X | MMP-2 | 0 | 0 |
| Pastinaca sativa | A | L | 2X | MMP-2 | 32 | 20 |
| Petroselinum crispum | G | Fl | 2X | MMP-2 | 0 | 9 |
| Phalaris canariensis | G | L/Fl/Fr/St | 2X | MMP-2 | 0 | 0 |
| Phaseolus vulgaris | A | L | 0.5X | MMP-9 | 0 | 0 |
| Phaseolus vulgaris | G | L | 0.5X | MMP-9 | 0 | 0 |
| Physalis philadelphica | A | L | 0.6X | MMP-9 | 26 | 32 |
| Phytolacca decandra | G | Fl. L | 2X | MMP-3 | 0 | 39 |
| Phytolacca decandra | G | Fl/L | 2X | MMP-3 | 0 | 39 |
| syn. P. americana | | | | | | |
| Pimpinella anisum | N | Fr/L/St | 2X | MMP-2 | 0 | 0 |
| Potentilla anserina | N | L | 2X | MMP-3 | 9 | 7 |
| Poterium sanguisorba | G | L/S | 2X | MMP-3 | 0 | 43 |
| Poterium sanguisorba | A | L/S | 2X | MMP-3 | 0 | 33 |
| Pyrus communis | N | Fr | 2X | MMP-2 | 9 | 41 |
| Raphanus raphanistrum | G | L | 0.7X | MMP-9 | 0 | 0 |
| Rheum rhabarbarum | A | L | 2X | MMP-9 | 0 | 36 |
| Ribes nigrum L. | A | Fr | 0.5X | MMP-1 | 0 | 24 |
| Ribes sylvestre | N | L | 2X | MMP-9 | 0 | 27 |
| Ribes sylvestre | G | L/St | 2X | MMP-3 | 0 | 33 |
| Rosmarinus officinalis | A | L/S | 2X | MMP-3 | 0 | 39 |
| Rubus occidentalis | N | Fr | 2X | MMP-9 | 21 | 14 |
| Rumex crispus | A | R | 2X | MMP-9 | 6 | 43 |
| Rumex crispus | G | R | 2X | MMP-9 | 5 | 10 |
| Rumex scutatus | N | L | 0.5X | MMP-9 | 6 | 0 |
| Ruta graveolens | A | L/Fl | 1X | MMP-3 | 69 | 71 |
| Salvia officinalis | A | L/S | 2X | MMP-9 | 0 | 46 |
| Salvia officinalis | G | L/St | 2X | MMP-1 | NA | 20 |
| Salvia officinalis | G | L/St | 2X | MMP-1 | 15 | 0 |
| Sambucus canadensis L. | N | L/Fr | 2X | MMP-2 | 0 | 8 |
| Saponaria officinalis L. | G | L/St | 2X | MMP-2 | 0 | 0 |
| Setaria italica | A | L/Fl | 2X | MMP-2 | 0 | 0 |
| Solanum melongens | N | L | 0.5X | MMP-1 | 0 | 0 |
| Solanum melongens | N | L | 2X | MMP-1 | 13 | 12 |
| Sorghum dochna | N | L | 2X | MMP-2 | 0 | 0 |
| bicolor gr technicum | | | | | | |
| Stellaria media | N | L/St/Fl | 2X | MMP-2 | 0 | 0 |
| Stellaria media | G | L/St/Fl | 2X | MMP-2 | 0 | 0 |
| Tanacetum | G | L | 2X | MMP-9 | 0 | 0 |
| cinerariifolium | | | | | | |
| Taraxacum officinale | N | L | 2X | MMP-2 | 24 | 0 |
| Taraxacum officinale | G | L | 2X | MMP-2 | 0 | 0 |
| Teucrium chamaedrys | A | L/St | 2X | MMP-1 | 25 | 25 |
| Thymus fragantissimus | N | L/S | 2X | MMP-2 | 0 | 0 |
| Thymus fragantissimus | N | L/S | 2X | MMP-2 | 0 | 0 |
| Thymus praecox subsp. | A | R | 1X | MMP-1 | 0 | 0 |
| Arcticus | | | | | | |
| Thymus x citriodorus | G | L/St | 2X | MMP-2 | 0 | 15 |
| Trifolium incarnatum | N | L | 2X | MMP-2 | 0 | 0 |
| Tropaeolum majus | G | Fl | 2X | MMP-2 | 11 | 16 |
| Tropaeolum majus | G | L | 2X | MMP-9 | 0 | 12 |
| Tropaeolum majus | N | L | 0.56X | MMP-9 | 9 | 0 |
| Tsuga diversifolia | N | L/St | 2X | MMP-9 | 0 | 0 |
| Vaccinium | N | Fr | 2X | MMP-9 | 9 | 11 |
| angustifolium | | | | | | |
| Vaccinum angustifolium | G | L/St | 2X | MMP-3 | 32 | 30 |
| Vitia sp. | A | L | 1X | MMP-1 | 13 | 3 |
| Vitia sp. | N | L | 1X | MMP-3 | 0 | 0 |
| x Triticosecale spp. | N | E | 2X | MMP-2 | 7 | 18 |
| Zea mays | G | L | 2X | MMP-2 | 0 | 0 |
| Zea mays | A | L/F | 1X | MMP-2 | 5 | 22 |
| Zea mays | A | L/Fl | 1X | MMP-2 | 0 | 0 |
| Zea mays | G | L | 2X | MMP-2 | 0 | 0 |
| Zea mays | A | L/Fl | 0.5X | MMP-1 | 0 | 0 |
| Zea mays | A | L/Fl | 2X | MMP-2 | 41 | 23 |
| Zea mays | A | L/Fl | 2X | MMP-2 | 0 | 0 |
| Zea mays | A | L/Fl | 2X | MMP-2 | 0 | 12 |
| Zea mays | N | L | 0.5X | MMP-9 | 8 | 24 |
| Zingiber officinale | N | Fr/L/St | 2X | MMP-9 | 0 | 24 |

¹Stress: A: Arachidonic acid; G: Gamma-linolenic acid; N: No stress treatment
²Part of Plant: B: Buds; E: Ears; Fl: Flower; Fr: Fruit; L: Leaf, R: Root; S: Seed; St: Stem
³Original screening dose: 1 X = dose at which an inhibition of 50% was obtained in initial screening.

### Example VI

### Effect of Plant Extracts on Cell Migration

Aqueous and alcoholic plant extracts that inhibit MMP-9, MMP-2 or MMP-1 were prepared as described in Example I and underwent further testing to ascertain that they contain stable, non-cytotoxic molecules that are appropriate for product development. Stability is ascertained by recovery of protease inhibition over time under various conditions, including physiological conditions. Cytotoxicity is ascertained by incubation of the extracts with various cell types, including those indicated below.

In order to test the effect of various plant extracts that are also validated protease inhibitors on cellular migration, a cellular migration assay coupled with a cord formation assay using endothelial cells was conducted. The experimental details are provided below. Concentrations of plant extracts are expressed as a function of the IC₅₀ concentration determined for protease inhibition, which is termed IX. The extracts are, therefore, capable of decreasing the activity of at least one extracellular protease by at least 50% when measured according to one of the assays described herein. The 1× concentration can vary depending on the plant and the solvent used in the preparation of the extract. The average concentration of a IX aqueous extract is about 1.6 mg/ml, whereas the average concentration of a 1× alcoholic extract is about 4 mg/ml. For each extract tested in the assays described below, 4 different concentrations were used (0.31×, 0.62×, 1.25× and 2.5×) in duplicate.

Cell Migration Assays

Migration was assessed using a multi-well system (Falcon 1185, 24-well format), separated by a PET membrane (8 μm pore size) into top and bottom sections. Depending on the cells that are used in the assay, the membrane was coated with 10 μg/ml rat tail collagen and allowed to dry. All solutions used in top sections were prepared in DMEM-0.1% BSA, whereas all solutions used in the bottom sections were DMEM, or other media, containing 10% fetal calf serum.

EGM-2 (700 μl) was added to the bottom chamber as a chemo-attractant. HUVECs (100 μl of 10⁶ cells/ml) and buffer containing the plant extract at the appropriate dilution were added to the upper chamber (duplicate wells of each plant extract at each dilution). After 5 h incubation at 37° C. in a 5% CO₂ atmosphere, the membrane was rinsed with PBS, fixed and stained. The cells on the upper side of the membrane were wiped off, three randomly selected fields were counted on the bottom side.

The percent inhibition of migration is calculated as follows:
[(A−B)/A]×100,
where A is the average number of cells per field in the control well and B is the average number of cells per field in the treated wells.

Cord Formation Assay

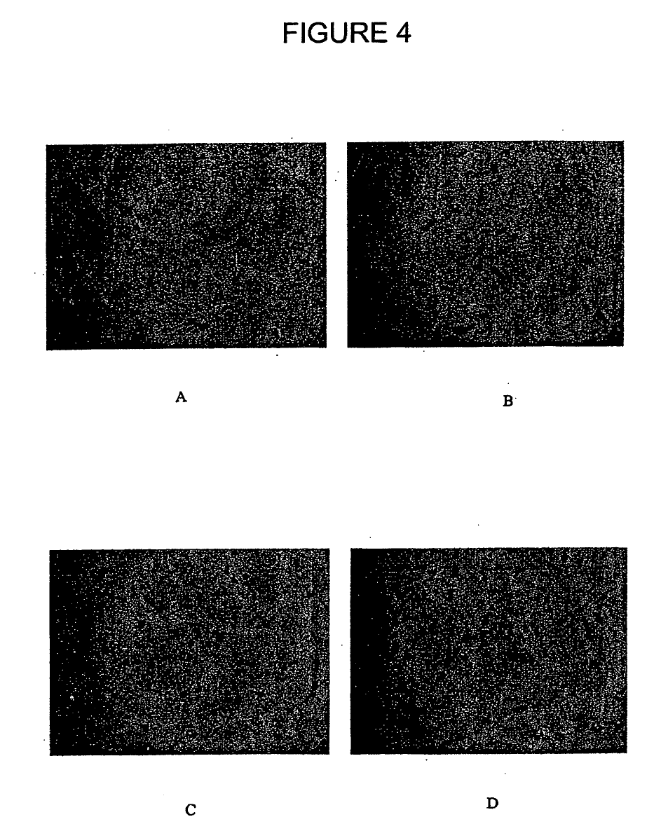

Matrigel (60 μl of 10 mg/ml) was added to a 96-well plate flat bottom plate (Costar 3096) and incubated for 30 minutes at 37° C. in a 5% CO₂ atmosphere. A mixture of HUVECs and plant extract, or positive controls (Fumagillin and GM6001) were added to each well. HUVECs were prepared as suspensions of 2.5×10⁵ cells per ml in EGM-2, then 500 μl of HUVECs preparation was mixed with 500 μl of 2× of the desired dilution of plant extract or control drug and 200 μl were added to each well. Four dilutions of each extract were tested in duplicate. After 18-24 hours at 37° C. in 5% CO₂, the cells had migrated and organized into cords (see FIG. 4, which shows the results using an extract from Rheum rhabarbaram).

The number of cell junctions were counted in 3 randomly selected fields and the inhibition of cord formation is calculated as follows:
[(A−B)/A]×100,
where A is the average number of cell junctions per field in the control well and B is the average number of cell junctions per field in the treated wells.

The results of the above tests are set forth in Table 7.

TABLE 7
Effect of Exemplary Plant Extracts on Endothelial Cell Migration

| | | | Endothelial Cell Migration | | | | | | | |
|---|---|---|---|---|---|---|---|---|---|---|
| | | | Cellular Migration | | | | | | | |
| | | | Assay | | | | Cord Formation Assay | | | |
| | | | % inhibition | | | | % inhibition | | | |
| | | Part of | Concentration³ | | | | Concentration³ | | | |
| Plant | Stress¹ | Plant² | 2.5× | 1.25× | 0.62× | 0.31× | 2.5× | 1.25× | 0.62× | 0.31× |
| Allium cepa | N | L | 19 | 28 | 25 | 36 | 0 | 0 | 0 | 0 |
| Allium sativum | A | L | 16 | 27 | 26 | 34 | 0 | 0 | 0 | 0 |
| Ambrosia artemisiifolia | N | L/St | 100 | 90 | 4 | 0 | 99 | 91 | 61 | 57 |
| Ambrosia artemisiifolia | N | Fl/L/St | 8 | 5 | NA | NA | ND | ND | ND | ND |
| Aronia x prunifolia | N | L/St | 50 | 26 | 20 | 19 | ND | 93 | 75 | 93 |
| Artemisia dracunculus | G | L/St | 81 | 57 | 40 | 30 | 45 | 13 | 22 | 23 |
| Artemisia dracunculus | N | Fl/L/St | 83 | 50 | 41 | 21 | 0 | 6 | 3 | 2 |
| Avena sativa | N | L | 92 | 75 | 34 | 40 | 100 | 8 | 0 | 0 |
| Beta vulgaris | N | L | 30 | 43 | 50 | 47 | 0 | 0 | 0 | 0 |
| Beta vulgaris | A | L | 0 | 0 | 0 | 0 | ND | ND | ND | ND |
| Beta vulgaris | G | L | 100 | 100 | 26 | 50 | ND | ND | ND | ND |
| Brassica napus | N | L | ND | ND | ND | ND | ND | ND | ND | ND |
| Brassica oleracea | N | L | 50 | 29 | 30 | 20 | 35 | 15 | 0 | 4 |
| Brassica oleracea | N | L | 37 | 58 | 23 | 4 | 0 | 0 | 0 | 0 |
| Brassica oleracea | A | L | 65 | 32 | 15 | 21 | 49 | 28 | 27 | 6 |
| Brassica oleracea | A | L | 26 | 17 | 0 | 0 | 0 | 0 | 0 | 0 |
| Brassica rapa | A | L | 0 | 19 | 31 | 23 | 0 | 0 | 0 | 0 |
| Brassica rapa | N | L | 25 | 21 | 14 | 6 | ND | ND | ND | ND |
| Bromus inermis | A | L | 90 | 44 | 36 | 17 | 21 | 14 | 0 | 93 |
| Chenopodium quinoa | N | L/St | 100 | 100 | 44 | 26 | 90 | 85 | 53 | 42 |
| Chenopodium quinoa | N | Fr/L/St | 100 | 100 | 50 | 33 | ND | ND | ND | ND |
| subsp. Quinoa | | | | | | | | | | |
| Chichorium endivia | G | L | 83 | 82 | 15 | 0 | 0 | 0 | 0 | 0 |
| Chichorium endivia | G | L | 48 | 11 | 21 | 16 | ND | ND | ND | ND |
| subsp. Endivia | | | | | | | | | | |
| Citrullus lanatus | A | L | 88 | 35 | 23 | 14 | 21 | 17 | 6 | 0 |
| Daucus carota | A | L | 100 | 63 | 74 | 32 | 92 | 28 | 0 | 0 |
| Daucus carota | A | L | 62 | 10 | 0 | 0 | 53 | 0 | 0 | 0 |
| Daucus carota | G | L | 0 | 0 | 0 | 0 | 86 | 43 | 25 | 36 |
| Dolichos lablab | G | Fl/Fr | 60 | 64 | 68 | 83 | 0 | 0 | 0 | 0 |
| Foeniculum vulgare | N | L | 64 | 47 | 62 | 61 | 69 | 21 | 23 | 11 |
| Foeniculum vulgare | G | L | 46 | 2 | 34 | 45 | ND | ND | ND | ND |
| Glycyrrhiza glabra | A | L | 100 | 56 | 0 | 53 | 0 | 0 | 0 | 0 |
| Glycyrrhiza glabra | G | L/St | 100 | 34 | 41 | 51 | 0 | 0 | 0 | 0 |
| Helianthus strumosus | G | L | 19 | 27 | 2 | 0 | 87 | 68 | 6 | 0 |
| Hypomyces lactifluorum | N | Fr | 46 | 30 | 25 | 20 | 17 | 0 | 0 | 0 |
| Hypomyces lactifluorum | N | Fr | 85 | 59 | 31 | 5 | 77 | 67 | 20 | 11 |
| Lentinus edodes | N | Fr | 40 | 16 | 22 | 14 | 0 | 0 | 0 | 0 |
| Lotus corniculatus | A | Fr | 93 | 83 | 77 | 57 | 9 | 0 | 0 | 0 |
| Lotus corniculatus | N | P | 58 | 11 | 26 | 0 | 0 | 0 | 0 | 0 |
| Lotus corniculatus | A | Fr/L/St | 18 | 8 | NA | NA | ND | ND | ND | ND |
| Lotus corniculatus | A | Fl/L/St | 31 | 35 | NA | NA | ND | ND | ND | ND |
| Lotus corniculatus | N | Fl/L/St | 32 | 36 | NA | NA | ND | ND | ND | ND |
| Manihot esculenta | N | Fr | 33 | 30 | 25 | 26 | 39 | 0 | 0 | 0 |
| Manihot esculenta | N | Fr | 69 | 24 | 22 | 31 | 0 | 7 | 0 | 20 |
| Matricaria recutita | G | Fl/L/St | 55 | 45 | 30 | 24 | 0 | 0 | 0 | 0 |
| Matricaria recutita | G | Fl/L/St | 74 | 6 | 1 | 20 | 34 | 31 | 4 | 0 |
| Melilotus albus | G | L/St | 70 | 15 | 0 | 0 | 0 | 0 | 0 | 0 |
| Melissa officinalis | N | L/St | 7 | 10 | 9 | 7 | ND | ND | ND | ND |
| Phaseolus vulgaris | A | L | 54 | 29 | 10 | 18 | 51 | 17 | 4 | 7 |
| Phaseolus vulgaris | G | L | 82 | 56 | 51 | 41 | 33 | 13 | 25 | 18 |
| Physalis Philadelphica | A | L | 100 | 100 | 100 | 100 | 100 | 72 | 100 | 81 |
| Pimpinella anisum | N | Fr/L/St | 70 | 64 | 65 | 69 | 40 | 51 | 27 | 42 |
| Pisum sativum | N | L/St | 38 | 16 | 13 | 0 | 16 | 24 | 4 | 0 |
| Raphanus raphanistrum | G | L | 88 | 46 | 23 | 23 | 46 | 24 | 0 | 0 |
| Raphanus raphanistrum | N | Fr | ND | ND | ND | ND | ND | ND | ND | ND |
| Rheum x hybridum | A | L | 13 | 0 | NA | NA | ND | ND | ND | ND |
| (=Rheum rhabarbarum) | | | | | | | | | | |
| Ribes sylvestre | N | L | 59 | 49 | 69 | 56 | 96 | 87 | 56 | 26 |
| Rubus occidentalis | N | Fr | 16 | 9 | 0 | 0 | 0 | 0 | 32 | 0 |
| Rumex crispus | G | R | 100 | 86 | 36 | 36 | 95 | 82 | 53 | 48 |
| Rumex crispus | A | R | 100 | 11 | NA | NA | ND | ND | ND | ND |
| Rumex scutatus | N | L | 100 | 20 | 0 | 0 | 70 | 6 | 0 | 0 |
| Setaria italica | A | L/Fl | 93 | 65 | 54 | 30 | 0 | 0 | 0 | 0 |
| Sorghum dochna bicolor | N | L | 32 | 0 | 0 | 0 | 0 | 0 | 0 | 0 |
| gr technicum | | | | | | | | | | |
| Stellaria media | N | Fl/L/St | 33 | 27 | 21 | 28 | 0 | 0 | 0 | 0 |
| Tanacetum | G | L | 18 | 21 | NA | NA | ND | ND | ND | ND |
| cinerariifolium | | | | | | | | | | |
| Taraxacum officinale | N | L | 45 | 11 | 1 | 3 | 5 | 2 | 0 | 2 |
| Taraxacum officinale | G | L | 90 | 40 | 44 | 23 | 0 | 0 | 0 | 0 |
| Thymus fragantissimus | N | L/St | 38 | 15 | 11 | 0 | 0 | 0 | 0 | 22 |
| Thymus x citriodorus | G | L/St | 76 | 12 | 8 | 0 | 32 | 35 | 0 | 0 |
| Trifolium incarnatum | N | L | 47 | 27 | 5 | 10 | 22 | 12 | 24 | 26 |
| Trifolium incarnatum | N | B/L/St | 100 | 100 | 41 | 21 | ND | ND | ND | ND |
| Tropaeolum majus | G | L | 57 | 58 | 49 | 42 | 0 | 0 | 0 | 0 |
| Tropaeolum majus | G | L | 65 | 29 | 18 | 4 | 7 | 0 | 0 | 0 |
| Tsuga canadensis | N | L/St | 68 | 41 | 31 | 31 | ND | 80 | 82 | 64 |
| Tsuga canadensis | N | L/St | 32 | 18 | NA | NA | ND | ND | ND | ND |
| Tsuga diversifolia | N | L/St | 99 | 43 | 18 | 27 | 57 | 8 | 0 | 0 |
| Vaccinium angustifolium | N | Fr | 62 | 7 | 11 | 24 | 59 | 15 | 6 | 0 |
| X Triticosecale spp. | N | E | 80 | 84 | 59 | 49 | 0 | 0 | 0 | 0 |
| Zea mays | G | L | 51 | 27 | 0 | 2 | 6 | 26 | 25 | 30 |
| Zea mays | A | L/Fl | 17 | 0 | 49 | 29 | 0 | 6 | 3 | 2 |
| Zea mays | N | L | 66 | 24 | 14 | 6 | 11 | 0 | 0 | 11 |
| Zingiber officinale | N | Fr | 59 | 38 | 27 | 30 | 0 | 0 | 0 | 0 |
| Zingiber officinale | N | R | 0 | 19 | NA | NA | ND | ND | ND | ND |

¹Stress: A: Arachidonic Acid; G: Gamma-Linolenic Acid; N: No stress treatment
²Part of Plant: B: Buds; Fl: Flower; Fr: Fruit; L: Leaf; P: Pods; R: Root; S: Seed; St: Stem
³Original screening dose: 1 X = dose at which an inhibition of 50% was obtained in initial screening.

### Example VII

### Plant Extracts that Inhibit Human Leukocyte Elastase (HLE)

Plant extracts were prepared as described in Example I and were tested for their ability to inhibit HLE as described in Example II.

Results are presented in Table 8.

TABLE 8
Inhibition of HLE

| Plant | Stress | Part of Plant |
|---|---|---|
| Arctostaphylos uva-ursi | N | L/St |
| Arctostaphylos uva-ursi | N | L/St |
| Beta vulgaris | N | R |
| Cornus sericea | G | L |
| Daucus carota | G | L |
| Euphorbia amygdaloides | G | L/St |
| Galinsoga quadriradiata | A | Fl |
| Gentiana lutea | A | L |
| Geranium sanguineum | N | L/St |
| Oenothera biennis | A | Fl/Fr/L/St |
| Potentilla fruticosa | N | Fl/Fr/L/St |
| Rodgersia spp. | A | L |
| Rubus thibetanus | G | L/St |
| Rumex crispus | A | L/Fr |
| Rumex crispus | G | L |
| Rumex crispus | N | L/Fr |
| Vitia sp. | A | Fr |

¹Stress: A: Arachidonic Acid; G: Gamma-Linolenic Acid; N: No stress treatment
²Part of Plant: Fl: Flower; Fr: Fruit; L: Leaf; R: Root; S: Seed; St: Stem

### Example VIII

### Preparation of Plant Extracts (Method C)

The following protocol was employed to prepare the plant extracts tested in the following Examples (1× to XIV).

For each of the plants, five grams of the dried plant material to be extracted was placed in a beaker and a sufficient amount of solvent was added to allow moderate agitation with a stirring bar. The solvents used in this Example were: butylene glycol (100%), butylene glycol/water (50/50, v/v), butylene glycol/water (20/80, v/v); ethanol (100%), ethanol/water (85/15, v/v), ethanol/water (50/50, v/v); water (100%).

Several different extraction times were employed for each solvent: after mixing for periods of 1 hour, 2 hours, 3 hours, or 4 hours at room temperature, the suspension was centrifuged and filtered through a 0.45 micron paper filter. For the centrifuged and filtered butylene glycol mixtures, the solvent was then evaporated at 120° C. and the residual matter was weighed to determine the yield of extraction at each time point. For the centrifuged and filtered ethanol mixtures, the solvent was removed under reduced pressure at a temperature of less than 45° C. in order to determine the yield of extraction at each time point.

In order to determine the enzymatic and biological properties of the extracts, the 4 hour butylene glycol or ethanol mixtures were assayed without further treatment.

The above protocol is suitable for the preparation of extracts that are to be employed in dermatological formulations. Butylene glycol extracts, for example, can be included directly into formulations intended for topical application. Ethanol extracts may undergo one or more additional steps prior to incorporation into formulations intended for topical application as described in Example XV.

### Example IX

### Protease Inhibition of Plant Extracts Prepared by Method C

Plant extracts prepared as described in Example VIII were tested for their ability to inhibit MMP-1, MMP-2, MMP-3, MMP-9 and/or HLE using the assays described above (Example II).

The results are presented in Table 9.

TABLE 9
Inhibition of Proteases by Plant Extracts Prepared by Method C

| | | | IC50 | |
|---|---|---|---|---|
| Plant Part (treatment) | Extraction Solvent | Enzyme | (μg/mL) | Yield (% wt/wt) |
| | 100% Butylene Glycol | MMP-3 | NA | 1.4 |
| Potentilla anserina L. | 50% Butylene Glycol | MMP-3 | 30.0 | 19.0 |
| Aerial parts (untreated) | 20% Butylene Glycol | MMP-3 | 92.5 | 20.0 |
| | 100% Ethanol | MMP-3 | 28.8 | 19.1 |
| Potentilla anserina L. | 85% Ethanol | MMP-3 | 27.6 | 27.2 |
| Aerial parts (untreated) | 50% Ethanol | MMP-3 | 56.3 | 34.2 |
| | 100% Water | MMP-3 | 58.8 | 25.7 |
| | 100% Butylene Glycol | MMP-3 | 35.9 | 7.7 |
| Rhus typhina L. | 50% Butylene Glycol | MMP-3 | 128.6 | 23.8 |
| Leaf (untreated) | 20% Butylene Glycol | MMP-3 | 27.1 | 22.1 |
| | 100% Ethanol | MMP-3 | 13.3 | 9.9 |
| Rhus typhina L. | 85% Ethanol | MMP-3 | 27.5 | 33.4 |
| Leaf (untreated) | 50% Ethanol | MMP-3 | 38.0 | 38.1 |
| | 100% Water | MMP-3 | 54.8 | 29.0 |
| | 100% Butylene Glycol | MMP-3 | 42.5 | 5.8 |
| Juniperus communis L. | 50% Butylene Glycol | MMP-3 | 46.2 | 14.1 |
| Aerial parts (untreated) | 20% Butylene Glycol | MMP-3 | 37.0 | 12.3 |
| | 100% Ethanol | MMP-3 | 28 | 17.5 |
| Juniperus communis L. | 85% Ethanol | MMP-3 | 52 | 24.0 |
| Aerial parts (untreated) | 50% Ethanol | MMP-3 | 26 | 23.9 |
| | 100% Water | MMP-3 | 136 | 17.1 |
| | 100% Butylene Glycol | MMP-9 | 19.7 | 0.3 |
| Vaccinium | 50% Butylene Glycol | MMP-9 | 58.8 | 1.8 |
| angustifolium Ait. | | | | |
| Press-cake (untreated) | 20% Butylene Glycol | MMP-9 | 110.0 | 1.3 |
| | 100% Ethanol | MMP-9 | 28.4 | 7.3 |
| Vaccinium | 85% Ethanol | MMP-9 | 290.5 | 5.5 |
| angustifolium Ait. | | | | |
| Press-cake (untreated) | 50% Ethanol | MMP-9 | 11.3 | 4.0 |
| | 100% Water | MMP-9 | 11.5 | 2.1 |
| | 100% Butylene Glycol | MMP-9 | 28.1 | 1.2 |
| Tropaeolum majus L. | 50% Butylene Glycol | MMP-9 | 108.1 | 18.3 |
| Aerial parts (G) | 20% Butylene Glycol | MMP-9 | 90.5 | 22.9 |
| | 100% Ethanol | MMP-9 | 48.3 | 5.2 |
| Tropaeolum majus L. | 85% Ethanol | MMP-9 | 69.0 | 20.6 |
| Aerial parts (G) | 50% Ethanol | MMP-9 | 64.0 | 33.9 |
| | 100% Water | MMP-9 | 32.9 | 37.3 |
| | 100% Butylene Glycol | MMP-9 | 93.0 | 2.4 |
| Melilotus alba Medik. | 50% Butylene Glycol | MMP-9 | 30.1 | 13.7 |
| Aerial parts minus main | 20% Butylene Glycol | MMP-9 | 30.4 | 12.6 |
| stem (untreated) | | | | |
| | 100% Ethanol | MMP-9 | 16.7 | 6.9 |
| Melilotus alba Medik. | 85% Ethanol | MMP-9 | 19.4 | 14.8 |
| Aerial parts (untreated) | 50% Ethanol | MMP-9 | 60.3 | 26.5 |
| | 100% Water | MMP-9 | 22.5 | 28.7 |
| | 100% Butylene Glycol | HLE | 11.5 | 1.9 |
| Daucus carota subsp | 50% Butylene Glycol | HLE | 12.2 | 15.2 |
| carota L. | | | | |
| Aerial parts (untreated) | 20% Butylene Glycol | HLE | 68.3 | 16.0 |
| | 100% Ethanol | HLE | 20.2 | 4.7 |
| Daucus carota subsp | 85% Ethanol | HLE | 8.3 | 12.3 |
| carota L. | | | | |
| Aerial parts (untreated) | 50% Ethanol | HLE | 5.8 | 22.6 |
| | 100% Water | HLE | 43.1 | 21.6 |
| | 100% Butylene Glycol | HLE | 0.35 | 0.0 |
| Geranium x cantabrigiense | 50% Butylene Glycol | HLE | 14.10 | 8.3 |
| Leaf (untreated) | 20% Butylene Glycol | HLE | 11.40 | 7.0 |
| | 100% Ethanol | HLE | 0.31 | 5.7 |
| Geranium x cantabrigiense | 85% Ethanol | HLE | 0.27 | 15.9 |
| Leaf (untreated) | 50% Ethanol | HLE | 0.35 | 29.9 |
| | 100% Water | HLE | 0.43 | 21.5 |
| | 100% Butylene Glycol | MMP-9 | 16.5 | 6.6 |
| Chenopodium quinoa | 50% Butylene Glycol | MMP-9 | 5.6 | 10.6 |
| Willd. (Norquin) | | | | |
| Seed (untreated) | 20% Butylene Glycol | MMP-9 | 5.4 | 6.3 |
| | 100% Ethanol | MMP-9 | 20.4 | 7.0 |
| Chenopodium quinoa | 85% Ethanol | MMP-9 | 13.4 | 5.8 |
| Willd. (Norquin) | | | | |
| Seed (untreated) | 50% Ethanol | MMP-9 | 13.8 | 6.8 |
| | 100% Water | MMP-9 | 6.8 | 11.2 |
| | 100% Butylene Glycol | MMP-2 | 0.35 | 0.0 |
| x Triticosecale spp. | 50% Butylene Glycol | MMP-2 | 14.10 | 8.3 |
| Seed (untreated) | 20% Butylene Glycol | MMP-2 | 11.40 | 7.0 |
| | 100% Ethanol | MMP-2 | 11.0 | 2.2 |
| x Triticosecale spp. | 85% Ethanol | MMP-2 | 2.4 | 4.4 |
| Seed (untreated) | 50% Ethanol | MMP-2 | 3.3 | 9.2 |
| | 100% Water | MMP-2 | 3.7 | 10.4 |
| | 100% Butylene Glycol | MMP-9 | 7.5 | 0.8 |
| Chenopodium quinoa | 50% Butylene Glycol | MMP-9 | 98 | 6.1 |
| Willd. (Royal) | | | | |
| Seed (untreated) | 20% Butylene Glycol | MMP-9 | 58.3 | 7.3 |
| | 100% Ethanol | MMP-9 | 16.3 | 7.4 |
| Chenopodium quinoa | 85% Ethanol | MMP-9 | 8.4 | 5.0 |
| Willd. (Royal) | | | | |
| Seed (untreated) | 50% Ethanol | MMP-9 | 19.0 | 5.8 |
| | 100% Water | MMP-9 | 2.8 | 10.8 |
| | 100% Butylene Glycol | MMP2 | 17 | 5.5 |
| Beta vulgaris L. subsp. | 50% Butylene Glycol | MMP2 | 17.8 | 22.8 |
| Vulgaris | | | | |
| Leaf (sandblasted) | 20% Butylene Glycol | MMP2 | 26.1 | 18.8 |
| | 100% Ethanol | MMP2 | 13.5 | 7.8 |
| | 85% Ethanol | MMP2 | 62 | 18.2 |
| Beta vulgaris L. subsp. | 50% Ethanol | MMP2 | 45 | 23.7 |
| Vulgaris | | | | |
| Leaf (sandblasted) | 100% Water | MMP2 | 8.2 | 25.3 |
| | 100% Butylene Glycol | MMP-1 | 40 | 1.9 |
| Zea mays L. | 50% Butylene Glycol | MMP-1 | 25 | 14.1 |
| Leaf (untreated) | 20% Butylene Glycol | MMP-1 | 20 | 14.1 |
| | 100% Ethanol | MMP-1 | 256 | 5.0 |
| | 85% Ethanol | MMP-1 | 338 | 8.0 |
| Zea mays L. | 50% Ethanol | MMP-1 | 405 | 12.8 |
| Leaf (untreated) | 100% Water | MMP-1 | 146 | 14.3 |
| | 100% Butylene Glycol | MMP-9 | 35 | 1.9 |
| Zea mays L. | 50% Butylene Glycol | MMP-9 | 7 | 14.1 |
| Leaf (untreated) | 20% Butylene Glycol | MMP-9 | 7 | 14.1 |
| | 100% Butylene Glycol | MMP-1 | 140 | 3.9 |
| Brassica oleracea L. | 50% Butylene Glycol | MMP-1 | 117 | 20.7 |
| var. italica Plenck | | | | |
| Head (untreated) | 20% Butylene Glycol | MMP-1 | 78 | 23.1 |
| | 100% Ethanol | MMP-1 | 31.5 | 6.2 |
| | 85% Ethanol | MMP-1 | 1465 | 24.7 |
| Brassica oleracea L. | 50% Ethanol | MMP-1 | Negative | 33.5 |
| var. italica Plenck | | | | |
| Head (untreated) | 100% Water | MMP-1 | 105 | 29.0 |
| | 100% Butylene Glycol | MMP-1 | 80 | 1.6 |
| Capsicum annuum L. | 50% Butylene Glycol | MMP-1 | 140 | 23.5 |
| var. annuum | | | | |
| Leaf (untreated) | 20% Butylene Glycol | MMP-1 | 112 | 22.4 |
| | 100% Ethanol | MMP-1 | 323 | 6.6 |
| | 85% Ethanol | MMP-1 | 760 | 22.7 |
| Capsicum annuum L. | 50% Ethanol | MMP-1 | 788 | 36.5 |
| var. annuum | | | | |
| Leaf (untreated) | 100% Water | MMP-1 | 57 | 26.0 |
| | 100% Butylene Glycol | MMP-1 | 35 | 3.8 |
| Solanum melongena L. | 50% Butylene Glycol | MMP-1 | 1100 | 22.2 |
| Leaf (untreated) | 20% Butylene Glycol | MMP-1 | 805 | 24.9 |
| | 100% Ethanol | MMP-1 | 88 | 12.8 |
| | 85% Ethanol | MMP-1 | 960 | 38.3 |
| Solanum melongena L. | 50% Ethanol | MMP-1 | Negative | 37.5 |
| Leaf (untreated) | 100% Water | MMP-1 | 654 | 38.8 |
| | 100% Butylene Glycol | MMP-1 | 23 | 2.9 |
| Pastinaca sativa L. | 50% Butylene Glycol | MMP-1 | 201 | 24.6 |
| Root (untreated) | 20% Butylene Glycol | MMP-1 | 140 | 25.4 |
| | 100% Ethanol | MMP-1 | 53 | 5.8 |
| | 85% Ethanol | MMP-1 | 204 | 19.0 |
| Pastinaca sativa L. | 50% Ethanol | MMP-1 | 365 | 27.1 |
| Root (untreated) | 100% Water | MMP-1 | 459 | 28.4 |

### Example X

### Cytotoxicity Testing of Plant Extracts

This example describes a method of testing the plant extracts for their cytotoxicity and allows non-cytotoxic concentrations of the extracts suitable for further efficacy studies to be selected. Plant extracts were prepared as described in Example VIII.

Normal human skin fibroblasts and keratinocytes (Cascade Biologics, Portland, Oreg.) were tested to evaluate the possible anti-proliferative effect of an extract of the present invention. The latter was done to ascertain that the exposure of cells to a concentration of extract would have no undesirable effect for further cellular assays. The present response was measured in a 96-well plate. Cells were seeded in their media (M106+LSGS for fibroblasts and M154+HKGS for keratinocytes, Cascade Biologics) fibroblasts at 5×10³ cells/100 μl/well and keratinocytes at 8×10³ cells/100 μl well. Plates were incubated for 24 hours at 37° C. in a humidified 5% CO₂ atmosphere. The extracts were obtained and diluted at a concentration 2 mg/ml (2× the final concentration) in both media. Four dilutions were tested for each cell line. Controls were included for each assay, 100 μl of media to reflect the maximum growth and viability of cells and 100 ng/ml of daunorubicin to obtain an 80% cytotoxic effect. All wells were incubated for 72 hours at 37° C. in a humidified 5% CO₂ atmosphere. After incubation, Alamar Blue dye was added to each well¹, fluorescence was read on a Spectrafluor Plus (Tecan, Durham, N.C.). All assays were done in quadruplicate.

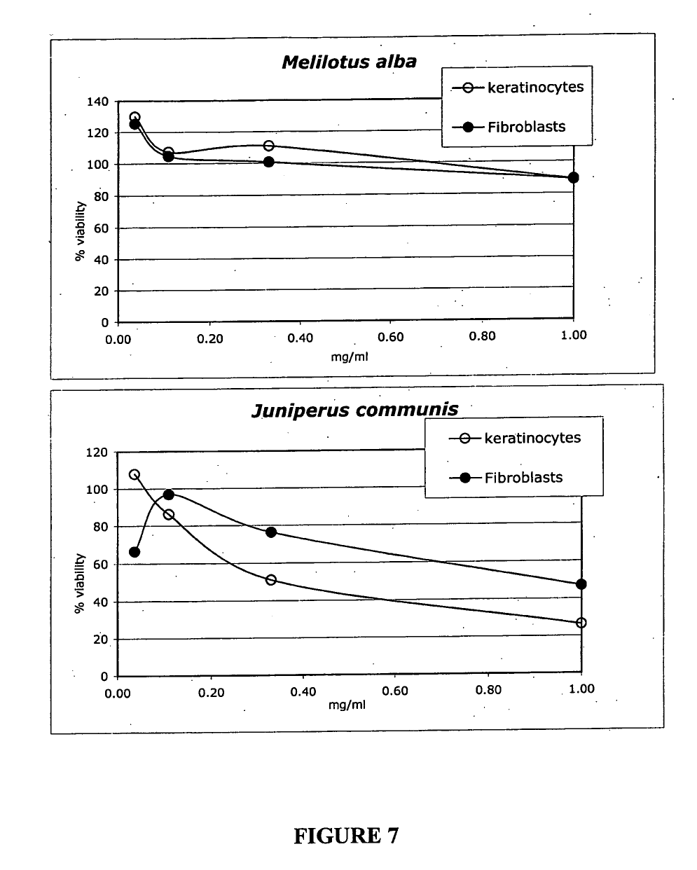

The results are shown in Table 10. FIG. 7 presents the results for the extracts from Melilotus alba and Juniperus communis. The results represent the average of quadruplicate measurements.

TABLE 10
Cytotoxicity of Representative Plant Extracts

| | | | | IC50/100% viability¹ | |
|---|---|---|---|---|---|
| | | Extraction | | (in mg/ml) | |
| Plant | Plant part | Solvent (y/y) | Protease | Keratinocytes | Fibroblasts |
| Potentilla | Aerial parts | BG²/water [50:50] | MMP-3 | 0.12/0.1 | 0.35/0.3 |
| anserina L. | | BG/water [20:80] | MMP-3 | 0.04/0.02 | 0.7/0.3 |
| Rhus typhina L | Leaf | BG/water [50:50] | MMP-3 | <0.03 | 0.5/0.1 |
| | | BG/water [20:80] | MMP-3 | <0.03 | 0.4/0.1 |
| Juniperus | Aerial parts | BG/water [50:50] | MMP-3 | 0.07/0.03 | 0.3/0.03 |
| communis L. | | BG/water [20:80] | MMP-3 | 0.33/0.12 | 1/0.25 |
| Tropaeolum | Aerial parts | BG/water [50:50] | MMP-9 | 100% viable at | 100% viable at |
| majus L. | | | | 0.6 | 0.6 |
| | | BG/water [20:80] | MMP-9 | 100% viable at | 100% viable at |
| | | | | 0.8 | 0.9 |
| Melilotus alba | Aerial parts | BG/water [50:50] | MMP-9 | 100% viable at 1 | 100% viable at 1 |
| Medik. | (minus main | BG/water [20:80] | MMP-9 | 100% viable at 1 | 100% viable at 1 |
| | stem) | | | | |
| Daucus carota | Aerial parts | BG/water [50:50] | HLE | 0.2/0.1 | 0.55/0.3 |
| subsp carota L. | | BG/water [20:80] | HLE | 1/0.3 | 100% viable at |
| | | | | | 0.1 |
| Geranium x catabrigiense | Leaf | BG/water [100:0] | HLE | 0.025/0.017 | 0.025/0.017 |
| | | BG/water [50:50] | HLE | 0.17/0.033 | 0.6/0.33 |
| | | BG/water [20:80] | HLE | 0.1/0.03 | 1/0.6 |
| Beta vulgaris L. | Leaf | BG/water [50:50] | MMP-2 | 100% viable at | 100% viable at 1 |
| subsp. Vulgaris | | | | 0.7 | |
| | | BG/water [20:80] | MMP-2 | 100% viable at 1 | 100% viable at 1 |
| Zea mays L. | Leaf | BG/water [50:50] | MMP-1 | 100% viable at | 100% viable at |
| | | | et-9 | 0.6 | 0.2 |
| | | BG/water [20:80] | MMP-1 | 100% viable at 1 | 100% viable at |
| | | | et-9 | | 0.3 |
| Brassica | Head | BG/water [50:50] | MMP-1 | 100% viable at | 0.55/0.1 |
| oleracea L. var. | | | | 0.7 | |
| italica Plenck | | BG/water [20:80] | MMP-1 | 100% viable at | 0.65/0.3 |
| | | | | 0.8 | |
| Chenopodium | Seed | BG/water [0:100] | MMP-9 | 100% viable at | 100% viable at 1 |
| quinoa Willd. | | | | 0.8 | |
| Triticosecale | Seed | EtOH/water | MMP-2 | 0.48/0.25 | 100% viable at |
| spp. | | [100:0] | | | 0.6 |
| Pastinaca sativa | Root | BG/water [50:50] | MMP-1 | 100% viable at | 100% viable at |
| L. | | | | 0.8 | 0.8 |
| | | BG/water [20:80] | MMP-1 | 100% viable at 1 | 100% viable at |
| | | | | | 0.75 |
| Pastinaca sativa | Root | EtOH/water | MMP-1 | 0.07/0.04 | 0.1/0.07 |
| L. | | [100:0] | | | |
| | | BG/water [100:0] | MMP-1 | 0.11/0.08 | 100% viable at |
| | | | | | 0.12 |
| | | BG/water [20:80] | MMP-1 | 100% viable at | 100% viable at 1 |
| | | | | 0.5 | |
| Brassica | Head | BG/water [100:0] | MMP-1 | 0.04/0.01 | 0.07/0.04 |
| oleracea L. var. | | BG/water [50:50] | MMP-1 | 0.8/0.1 | 100% viable at |
| italica Plenck | | | | | 0.8 |
| | | BG/water [20:80] | MMP-1 | 0.7/0.1 | 100% viable at |
| | | | | | 0.4 |
| Capsicum | Leaf | EtOH/water | MMP-1 | 0.1/0.03 | 0.6/0.35 |
| annuum L. var. | | [20:80] | | | |
| annuum | | BG/water [0:100] | MMP-1 | 0.25/0.05 | 0.7/0.5 |
| Solanum | Leaf | EtOH/water | MMP-1 | 0.07/0.0.4 | 0.07/0.04 |
| melongena | | [100:0] | | | |
| | | BG/water [100:0] | MMP-1 | 0.09/0.035 | 0.12/0.08 |

¹This value represents the concentration at which 100% viability is retained in the tested cell line.
²BG: butylene glycol

### Example XI

### Effect of Plant Extracts on Collagen Production

This following example demonstrates the ability of exemplary plant extracts to stimulate collagen I production in human dermal fibroblast cells. Human dermal fibroblast cells (Cascade Biologics, Portland, Oreg.) were employed in the assay and the ability of the plant extract to stimulate collagen production was measured using the Takara Biomedicals ELISA kit (Takara Mirus Bio, Madison, Wis.), which evaluates the release of the procollagen type I C-peptide (PIP). This free propeptide indicates on a stoichiometric basis the number of collagen molecules synthesised since the PEP peptide is cleaved off the procollagen molecule during the formation of the collagen triple helix. Plant extracts were prepared as described in Example VIII.

Fibroblasts were first grown in a 96-well plate using the complete M106 (M106+LSGS; Cascade Biologics). This media was also used as control. GM6001 (Chemicon, Temecula, CA) was used as positive control at a concentration of 50 μM.

All extracts and controls were diluted in complete M106. Plant extracts were used at the concentration that provided 100% viability of fibroblasts as shown in Table 10. Cells were seeded into 96-well plates at a concentration of 5×10³ cells/well in complete M106 media. Plates were incubated for 72 hours at 37° C. in a humidified 5% CO₂ atmosphere. After incubation, the medium was removed and 200 μl of sample were added to the wells (all in duplicate). Plates were incubated for 48 hours at 37° C. in a humidified 5% CO₂ atmosphere.

The ELISA was performed following the protocol recommended by the manufacturer (Takara Biomedicals). 20 μl of the supernatant from each well were used. Standard buffer and stop solutions were freshly prepared. 100 μl of the antibody-POD conjugate was added into the wells of the pre-coated 96-well plate, then the 20 μl of standard and specimens were added to appropriate wells. The plate was mixed gently, sealed (to limit evaporation) and incubated for 3 hours at 37° C.

After incubation, each well was washed four times with PBS buffer. 100 μl of the substrate solution containing hydrogen peroxide and tetramethylbenzidine (TMBZ) was added to each well and the plate was incubated for 15 minutes. After incubation 100 μl of 1N H₂SO₄ (stop solution) was added to each well. The plate was then gently mixed and the absorbance was read at 450 nm on the Spectrafluor Plus plate reader (Tecan). The reading was taken within 15 minutes of addition of the stop solution. All solutions used were included in the kit except for the PBS and the stop solution.

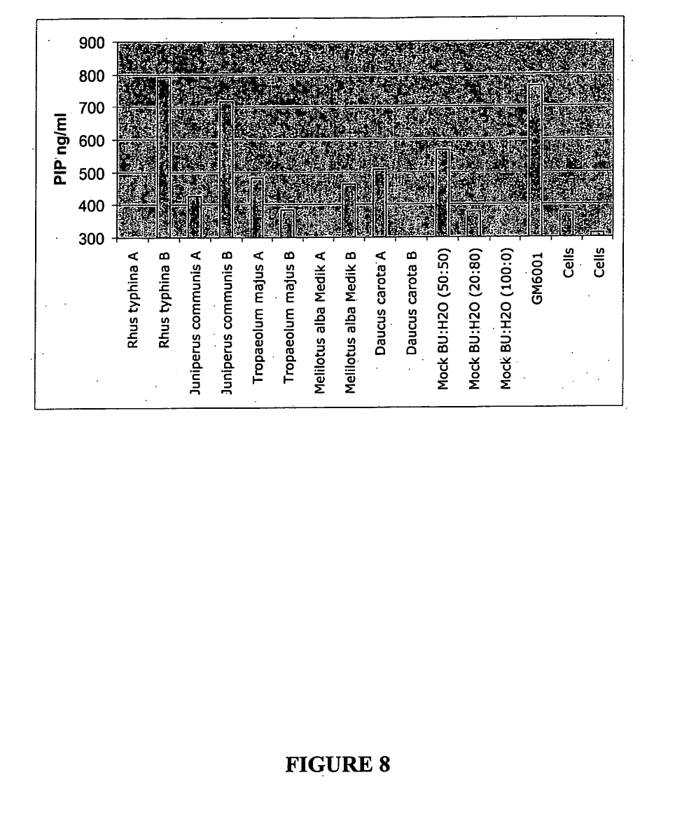

The results are presented in Table 11. FIG. 8 presents results of extracts for various extracts (A: extract using 50:50 v/v butylene glycol:water as solvent; B: extract using 20:80 v/v butylene glycol:water as solvent). The control (Mock BU:H₂O and cells alone) demonstrated the lowest collagen I production compared to the positive control GM6001 at 50 μM.

TABLE 11
Increase in PIP Production Stimulated by Representative Plant
Extracts

| Plant | Plant part | Extraction Solvent (v/v) | PIP/(% increase) |
|---|---|---|---|
| Potentilla anserina | Aerial parts | BG¹/water [50:50] | Negative |
| L. | | BG/water [20:80] | Negative |
| Rhus typhina L | Leaf | BG/water [50:50] | Negative |
| | | BG/water [20:80] | Positive/(+133%) |
| Juniperus | Aerial parts | BG/water [50:50] | Positive/(+25%) |
| communis L. | | BG/water [20:80] | Positive/(+111%) |
| Tropaeolum majus | Aerial parts | BG/water [50:50] | Positive/(+42%) |
| L. | | BG/water [20:80] | Negative |
| Melilotus alba | Aerial parts | BG/water [50:50] | Negative |
| Medik. | (minus main | BG/water [20:80] | Positive/(+36%) |
| | stem) | | |
| Daucus carota | Aerial parts | BG/water [50:50] | Negative |
| subsp carota L. | | BG/water [20:80] | Negative |
| Geranium x catabrigiense | Leaf | BG/water [100:0] | Negative |
| | | BG/water [50:50] | Negative |
| | | BG/water [20:80] | Negative |
| Beta vulgaris L. | Leaf | BG/water [50:50] | Negative |
| subsp. Vulgaris | | BG/water [20:80] | Negative |
| Zea mays L. | Leaf | BG/water [50:50] | Positive/(+17%) |
| | | BG/water [20:80] | Negative |
| Brassica oleracea | Head | BG/water [50:50] | Positive/(+11%) |
| L. var. italica | | BG/water [20:80] | Positive/(+15%) |
| Plenck | | | |
| Chenopodium | Seed | BG/water [0:100] | Positive/(+8%) |
| quinoa Willd. | | | |
| Triticosecale spp. | Seed | EtOH/water [100:0] | Positive/(+21%) |
| Pastinaca sativa L. | Root | BG/water [50:50] | Positive/(+14%) |
| | | BG/water [20:80] | Positive/(+11%) |
| Pastinaca sativa L. | Root | EtOH/water [100:0] | Negative |
| | | BG/water [100:0] | Positive/(+57.5%) |
| | | BG/water [20:80] | Positive/(+72.5%) |
| Brassica oleracea | Head | BG/water [100:0] | Positive/(+360%) |
| L. var. italica | | BG/water [50:50] | Negative |
| Plenck | | BG/water [20:80] | Positive/(+67.9%) |
| Capsicum annuum | Leaf | EtOH/water [20:80] | Positive/(+341%) |
| L. var. annuum | | BG/water [0:100] | Positive/(+306%) |
| Solanum | Leaf | EtOH/water [100:0] | Negative |
| melongena | | BG/water [100:0] | Positive/(+21.3%) |

¹BG: butylene glycol

### Example XII

### Inhibition of Dermal Contraction by Plant Extracts

The following example demonstrates the ability of exemplary extracts prepared as described in Example VIII to inhibit dermal contraction in an in vitro skin model. The skin model comprises human skin fibroblasts imbedded in a collagen I matrix and provides an in vitro representation of dermal contraction resulting from tractional forces generated by fibroblasts. Partial or permanent dermal contraction can play a role in the formation of wrinkles. Thus, extracts capable of inhibiting this type of contraction, have the potential to provide a dermo-decontraction anti-ageing effect in the skin. These extracts also have potential application in wound healing where pathological scarring is observed by excessive contraction.

The ability of exemplary plant extracts to inhibit dermal contraction was evaluated on human skin fibroblasts (Cascade Biologics, Portland, Oreg.). The cells were imbedded in a collagen I matrix to create a derm-like environment. Fibroblasts were grown in complete M106 to 80% confluence. Free-floating fibroblast-populated collagen gels were prepared in 24-well plates. 500 μlof gel contains 2.5 mg/ml of collagen I collagen I (rat tail, BD Biosciences, Bedford, Mass.), M106 5×, NaOH 0.7N; 1×10⁵ cells and fetal bovine serum (FBS) at 20% (Wisent, St-Bruno, QC, Canada). The mix was kept on ice until distribution. The derm-like gels were allowed to polymerize for 1 hour at 37° C. in a humidified 5% CO₂ atmosphere. After incubation, the gels were detached from the wells. Media 106 was used as negative control and GM6001 (Chemicon, Temecula, CA) at a concentration of 50 μM was used as positive control. All extracts were prepared at non-cytotoxic concentration (i.e. the concentration that provided 100% viability of fibroblasts as shown in Table 10) in complete media 106. FBS at a final concentration of 10% was added to each well. The plate was incubated for a maximum of 7 days at 37° C. in a humidified 5% CO₂ atmosphere. All assays were performed in duplicate. Contraction was measured beginning at day 3. Contracting gels were digitally photographed and the gel areas were calculated using ImagePro software.

The results are presented in Table 12. Control gels treated with media alone have the smallest area and represent the contracted control. GM6001 was able to provide limited, but not complete, inhibition of contraction.

TABLE 12
Inhibition of Dermal Contraction by Representative Plant Extracts

| | | | Contraction |
|---|---|---|---|
| | | Extraction Solvent | (% |
| Plant | Plant part | (v/v) | inhibition) |
| Potentilla anserina L. | Aerial parts | BG¹/water [50:50] | 96.5 |
| | | BG/water [20:80] | 94.5 |
| Rhus typhina L | Leaf | BG/water [50:50] | 79.7 |
| | | BG/water [20:80] | 39.2 |
| Juniperus | Aerial parts | BG/water [50:50] | 86.4 |
| communis L. | | BG/water [20:80] | 86.5 |
| Tropaeolum majus L. | Aerial parts | BG/water [50:50] | 44.2 |
| | | BG/water [20:80] | 44.2 |
| Melilotus alba Medik. | Aerial parts | BG/water [50:50] | 62.8 |
| | (minus | BG/water[20:80] | 48.5 |
| | main stem) | | |
| Beta vulgaris L. | Leaf | BG/water [50:50] | 13.3 |
| subsp. Vulgaris | | BG/water [20:80] | 41.6 |
| Zea mays L. | Leaf | BG/water [50:50] | 22.4 |
| | | BG/water [20:80] | 100 |
| Brassica oleracea L. | Head | BG/water [50:50] | 20.2 |
| var. italica Plenck | | BG/water [20:80] | 4.3 |
| Chenopodium quinoa | Seed | BG/water [0:100] | 0 |
| Willd. | | | |
| Triticosecale spp. | Seed | EtOH/water [100:0] | −11 |
| Pastinaca sativa L. | Root | BG/water [50:50] | 15.7 |
| | | BG/water [20:80] | 6.2 |
| Pastinaca sativa L. | Root | EtOH/water [100:0] | 62.4 |
| | | BG/water [100:0] | 25.7 |
| | | BG/water [20:80] | 17.5 |
| Brassica oleracea L. | Head | BG/water [100:0] | 14.7 |
| var. italica Plenck | | BG/water [50:50] | 6.7 |
| | | BG/water [20:80] | 21.2 |
| Capsicum annuum L. | Leaf | EtOH/water [20:80] | 13 |
| var. annuum | | BG/water [0:100] | 37.6 |
| Solanum melongena | Leaf | EtOH/water [100:0] | 33.4 |
| | | BG/water [100:0] | 7.2 |

¹BG: butylene glycol

### Example XIII

### Effect of Plant Extracts on Cytokine Release

The following example demonstrates the non-irritating behaviour of representative plant extracts of the invention prepared as described in Example VIII. The amount of Interleukin-8 (IL-8) released after exposure of keratinocytes to a plant extract, as described below, can be used to quantify any possible irritation reaction to the extract.

IL-8 release was evaluated in human skin keratinocytes (Cascade Biologics, Portland, Oreg.) using the Quantikine hIL-8 ELISA kit (R&D Systems, Minneapolis, Minn.). Keratinocytes were first grown in a 96-well plate using the complete M154 (M154+HKGS from Cascade Biologics). This media was also used as control. Phorbol 12-myristate 13-acetate (PMA) (Sigma-Aldrich Canada, Oakville, Ontario) at a concentration of 2.5 μM was used as a positive control. All tested plant extracts and the controls were diluted in complete M154 at a non-cytotoxic concentration (i.e. the concentration that provided 100% viability of keratinocytes as shown in Table 10). Cells were seeded into 96-well plates at a concentration of 8×10³ cells/well in complete M154 media. Plates were incubated for 48 hours at 37° C. in a humidified 5% CO₂ atmosphere. After incubation, the medium was removed and 200 μl of sample was added to the wells (all in duplicate). Plates were incubated for a further 48 hours at 37° C. in a humidified 5% CO₂ atmosphere.

The ELISA was performed using following the protocol recommended by the manufacturer (R&D Systems). 25 μl of the supernatant from each well was mixed with 25 μl of R5DP 1× diluting buffer. Standards were freshly prepared in R5DP 1×. 100 μl of assay diluent RD1-8 was added to each well of 96-well plate, then the 50 μl of standard and specimens were added to appropriate wells. The plate was mixed gently, sealed (to limit evaporation) and incubated for 2 hours at room temperature (RT°).

After incubation, each well was washed four times with wash buffer. 100 μl of the conjugation solution was added and incubated for 1 hour at RT°. After this incubation, each well was washed four times with wash buffer. 200 μl of substrate solution containing hydrogen peroxide and tetramethylbenzidine (TMBZ) was added to each well and the plate was incubated for 15 minutes. After incubation, 50 μl of 2N H₂SO₄ (stop solution) was added to each well. The plate was then gently mixed and the absorbance read at 450 nm on the Spectrafluor Plus plate reader (Tecan). The reading was taken within 15 minutes following addition of the stop solution. Ali solutions employed were provided in the kit.

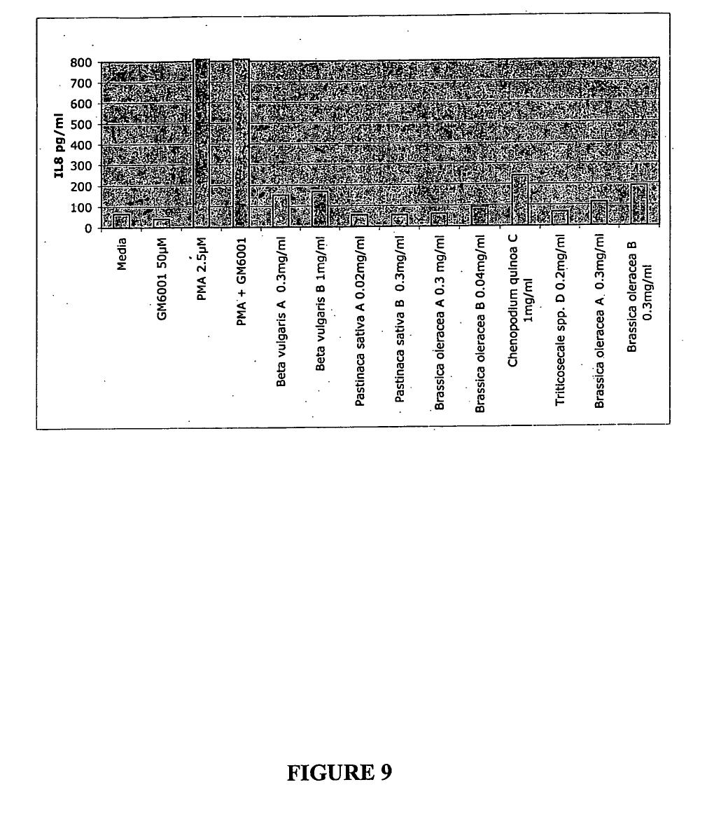

The above-described ELISA evaluates the release of IL-8. A plant extracts that results in a strong release of IL-8 may cause irritation to the skin at the tested concentration. The results are shown in Table 13. FIG. 9 presents results for various extracts (A: extract using 50:50 v/v butylene glycol:water as solvent; B: extract using 20:80 v/v butylene glycol:water as solvent; C: extract using 100% water as solvent; D: extract using 100% ethanol as solvent). The negative control (M154 media) showed the lowest IL-8 release and is considered to represent the minimum IL-8 release. PMA induced a strong inflammatory response and is considered to represent the highest level of IL-8 release. Although some of tested extracts increased in IL-8 release, the increase was small compared to that induced by PMA. Those extracts resulting in a small increase in IL-8 release can be re-assayed at a lower concentration, which will likely result in a relative decrease in the amount of IL-8 released. The evaluation of cytokine release as described above enables a maximum concentration of plant extract for further in vivo studies to be set.

TABLE 13
Effect of Representative Plant Extracts on IL-8 Release

| Plant | Plant part | Extraction Solvent (v/v) | IL-8 (% change)¹ |
|---|---|---|---|
| Potentilla anserina | Aerial parts | BG²/water [50:50] | −83 |
| L. | | BG/water [20:80] | −79 |
| Rhus typhina L | Leaf | BG/water [50:50] | 95 |
| | | BG/water [20:80] | −61 |
| Juniperus | Aerial parts | BG/water [50:50] | −83 |
| communis L. | | BG/water [20:80] | −79 |
| Tropaeolum majus | Aerial parts | BG/water [50:50] | −86 |
| L. | | BG/water [20:80] | −77 |
| Melilotus alba | Aerial parts | BG/water [50:50] | Performed at 2 |
| Medik | (minus main | | concentrations: |
| | stem) | | At 1 mg: 226 |
| | | | At 0.3 mg: 84 |
| | | BG/water [20:80] | −46 |
| Daucus carota | Aerial parts | BG/water [50:50] | −74 |
| subsp carota L. | | BG/water [20:80] | −61 |
| Geranium x catabrigiense | Leaf | BG/water [100:0] | 7 |
| | | BG/water [50:50] | −60 |
| | | BG/water [20:80] | −53 |
| Beta vulgaris L. | Leaf | BG/water [50:50] | 133 |
| subsp. Vulgaris | | BG/water [20:80] | Performed at 2 |
| | | | concentrations: |
| | | | At 1 mg: 158 |
| | | | At 0.3 mg: 54 |
| Zea mays L. | Leaf | BG/water [50:50] | −13 |
| | | BG/water [20:80] | −13 |
| Brassica oleracea | Head | BG/water [50:50] | −7 |
| L. var. italica | | BG/water [20:80] | 41 |
| Plenck | | | |
| Chenopodium | Seed | BG/water [0:100] | Performed at 2 |
| quinoa Willd. | | | concentrations: |
| | | | At 1 mg: 264 |
| | | | At 0.3 mg: 105 |
| Triticosecale spp. | Seed | EtOH/water [100:0] | 3.2 |
| Pastinaca sativa L. | Root | BG/water [50:50] | 72 |
| | | BG/water [20:80] | 190 |
| Pastinaca sativa L. | Root | EtOH/water [100:0] | 36 |
| | | BG/water [100:0] | 103 |
| | | BG/water [20:80] | −67 |
| Brassica oleracea | Head | BG/water [100:0] | −67 |
| L. var. italica | | BG/water [50:50] | Performed at 2 |
| Plenck | | | concentrations: |
| | | | At 1 mg: 201 |
| | | | At 0.3 mg: 159 |
| | | BG/water [20:80] | 13 |
| Capsicum annuum | Leaf | EtOH/water [20:80] | −39 |
| L. var. annuum | | BG/water [0:100] | 54 |
| Solanum | Leaf | EtOH/water [100:0] | −65 |
| melongena | | BG/water [100:0] | 0 |

¹Value indicates the % change relative to the negative control (untreated cells)
²BG: butylene glycol

### Example XIV

### Inhibition of UV-Induced Proteolytic Activity

The following example demonstrates the potential of representative plant extracts to protect the skin from proteolytic damage after sun exposure. Plant extracts were prepared as described in Example VIII.

Keratinocytes were first grown in 24-well plates using the complete M154 (M154+HKGS from Cascade Biologics) at a concentration of 2.5×10⁴ cells/500 μl/well. The plates were incubated 48 hours at 37° C. in a humidified 5% CO₂ atmosphere. The media was removed and the cells were washed 2 times with HBSS. After complete removal of liquid the cells were irradiated with 25 J/cm² of WVA light. After irradiation, test samples were added at 500 μl/well. The media was used as a negative control. GM6001 at a concentration of 50 μM was used as a positive control. All extracts and controls were diluted in complete M154 at a non-cytotoxic concentration (i.e. the concentration that provided 100% viability of keratinocytes as shown in Table 10). Plates were incubated for 24 hours at 37° C. in a humidified 5% CO₂ atmosphere. The supernatant from each well was assayed or kept at −80° C. until use. Supernatants (60 μl) were assayed for their overall proteolytic activity using the MMP2/7 internally quenched peptide (Calbiochem). All: assays were performed in duplicate except for controls, which were performed in quadruplicate.

The results are presented in Table 14.

TABLE 14
Inhibition of UV-Induced Protease Activity by Representative Plant
Extracts

| | | Extraction Solvent | Decrease in |
|---|---|---|---|
| Plant | Plant part | (v/v) | Protease Activity¹ |
| Potentilla anserina L. | Aerial parts | BG²/water [50:50] | 73.5 |
| | | BG/water [20:80] | 0 |
| Rhus typhina L | Leaf | BG/water [50:50] | 28 |
| | | BG/water [20:80] | 16.5 |
| Juniperus communis L. | Aerial parts | BG/water [50:50] | 46.5 |
| | | BG/water [20:80] | 15 |
| Tropaeolum majus L. | Aerial parts | BG/water [50:50] | 0 |
| | | BG/water [20:80] | 0 |
| Melilotus alba Medik. | Aerial parts (minus | BG/water [50:50] | 0 |
| | main stem) | BG/water [20:80] | 0 |
| Daucus carota subsp | Aerial parts | BG/water [50:50] | 7 |
| carota L. | | BG/water [20:80] | 0 |
| Geranium x catabrigiense | Leaf | BG/water [100:0] | 6 |
| | | BG/water [50:50] | 73.5 |
| | | BG/water [20:80] | 42.5 |
| Beta vulgaris L. | Leaf | BG/water [50:50] | 0 |
| subsp. Vulgaris | | BG/water [20:80] | 0 |
| Zea mays L. | Leaf | BG/water [50:50] | 0 |
| | | BG/water [20:80] | 16 |
| Brassica oleracea L. | Head | BG/water [50:50] | 0 |
| var. italica Plenck | | BG/water [20:80] | 0 |
| Chenopodium quinoa | Seed | BG/water [0:100] | n.d. |
| Willd. | | | |
| Triticosecale spp. | Seed | EtOH/water | 22 |
| | | [100:0] | |
| Pastinaca sativa L. | Root | BG/water [50:50] | 0 |
| | | BG/water [20:80] | 0 |
| Pastinaca sativa L. | Root | EtOH/water | 34.5 |
| | | [100:0] | |
| | | BG/water [100:0] | 18 |
| | | BG/water [20:80] | 0 |
| Brassica oleracea L. | Head | BG/water [100:0] | 0 |
| var. italica Plenck | | BG/water [50:50] | 0 |
| | | BG/water [20:80] | 0 |
| Capsicum annuum L. | Leaf | EtOH/water | 0 |
| var. annuum | | [20:80] | |
| | | BG/water [0:100] | 0 |
| Solanum melongena | Leaf | EtOH/water | 0 |
| | | [100:0] | |
| | | BG/water [100:0] | 0 |

¹Decrease in proteolytic activity on MMP 2/7 peptide after UV irradiation relative to untreated control.
²BG: butylene glycol

### Example XV

### Preparation of Ethanolic Plant Extracts for Topical Formulations

As ethanol is not commonly used as a solvent in cosmetic formulations, plant extracts prepared by ethanolic extractions as described in Example VIII can undergo further treatments to prepare them for incorporation into topical formulations. For example, the ethanolic extracts can be de-colourised by treatment with activated charcoal following standard protocols. The ethanol can be removed from extracts, or de-colourised extracts and the reduced extract material resuspended on a solid support or in a liquid solvent that is more acceptable to cosmetic formulators. Thus, the extracts, or de-colourised extracts, can be submitted to an evaporation procedure (for example using a rotary evaporator or soxlet) to remove some, or all, of the ethanol component of the solvent. A dermatologically suitable alcohol, such as a glycol, can be added and the resulting solution incorporated into a carrier suitable for topical application. The activity of the extract may be verified at one or several points in this additional procedure.

The disclosure of all patents, publications, including published patent applications, and database entries referenced in this specification are specifically incorporated by reference in their entirety to the same extent as if each such individual patent, publication, and database entry were specifically and individually indicated to be incorporated by reference.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

1. A dermatological formulation comprising a physiologically acceptable carrier and an effective amount of one or more plant extracts having extracellular protease inhibiting activity, said plant extract derived from any one of the plants listed in Tables 1, 2, 3, 4 and 5 by solvent extraction, and said extracellular protease selected from the group of: matrix metalloprotease-1 (MMP-1), matrix metalloprotease-2 (MMP-2), matrix metalloprotease-3 (MMP-3), matrix metalloprotease-9 (MMP-9) and human leukocyte elastase (HLE), wherein said plant extract modulates one or more cellular activities in skin cells.

2. The dermatological formulation according to claim 1, wherein said one or more cellular activities in skin cells are selected from the group of: attenuating the breakdown of collagen, fibronectin, fibrillin and/or elastin; attenuating endothelial cell migration; increasing collagen production; attenuating UV-induced extracellular protease activity and attenuating tractional forces generated by fibroblasts.

3. The dermatological formulation according to claim 1, wherein said solvent is an aqueous solvent, an alcoholic solvent, or a combination thereof.

4.-9. (canceled)

10. The dermatological formulation according to claim 1, wherein said dermatological formulation is for use in the routine care of the skin, hair and/or nails.

11. The dermatological formulation according to claim 1, wherein said dermatological formulation is for use to improve the health and/or appearance of the skin, hair and/or nails.

12. The dermatological formulation according to claim 1, wherein said dermatological formulation is for use in the treatment or prevention of a dermatological condition.

13. The dermatological formulation according to claim 1, wherein said dermatological formulation is for use to attenuate or prevent skin ageing.

14.-17. (canceled)

18. A process for identifying a plant extract suitable for the preparation of a dermatological formulation, said process comprising the steps of:
(a) generating a plurality of potential extracts by solvent extraction of plant material;
(b) analysing the ability of each of said potential plant extracts to inhibit one or more extracellular protease selected from the group of: matrix metalloprotease-1 (MMP-1), matrix metalloprotease-2 (MMP-2), matrix metalloprotease-3 (MMP-3), matrix metalloprotease-9 (MMP-9) and human leukocyte elastase (HLE);
(c) selecting those potential extracts that are capable of inhibiting the activity of at least one of said extracellular proteases to provide a group of extracts;
(d) analysing each extract in said group of extracts for the ability to modulate one or more cellular activities in skin cells selected from the group of: attenuating the breakdown of collagen, fibronectin, fibrillin and/or elastin; attenuating endothelial cell migration; increasing collagen production; attenuating UV-induced extracellular protease activity and attenuating tractional forces generated by fibroblasts; and
(e) selecting an extract that is capable of modulating one or more of said cellular activities to provide a plant extract suitable for the preparation of a dermatological formulation.

19. The process according to claim 18, wherein said plurality of potential extracts is generated from plant material from a single plant source.

20. The process according to claim 18, wherein said plurality of potential extracts is generated by selecting a group of plants; harvesting plant material from each plant in said selected group of plants; and subjecting said plant material from each plant to a solvent extraction process to provide said plurality of potential extracts.

21. The process according to claim 18, wherein said solvent extraction process employs an alcohol, water, an aqueous buffer, or a combination thereof as solvent.

22. The process according to claim 18, wherein the group of extracts selected in step (c) are capable of inhibiting the activity of at least one of said extracellular proteases by at least 20%.

23. The process according to claim 18, further comprising the steps of subjecting each plant extract in said group of extracts to at least one cytotoxicity, bioavailability or stability test and selecting those extracts that demonstrate physiologically acceptable cytotoxicity, bioavailability and/or stability.

24. The process according to claim 18, wherein said plant material is generated from a plant or group of plants that have been subjected to one or more stress.

25. The dermatological formulation according to claim 1, wherein said plant extract having extracellular protease inhibiting activity is derived by solvent extraction from a plant selected from the group of: Aconitum napellus, Acorus calamus, Alchemilla mollis, Allium cepa, Allium sativum, Allium tuberosum, Ambrosia artemisiifolia, Anethum graveolens, Anthemis tinctoria, Aronia melanocarpa (Michx.) Ell., Arctostaphylos uva-ursi, Aronia×prunifolia, Artemisia dracunculus, Avena sativa, Beta vulgaris, Beta vulgaris L. subsp. Vulgaris, Borago officinalis, Brassica napus, Brassica oleracea, Brassica oleracea L. var. italica Plenck, Brassica rapa, Bromus inermis, Capsicum annuum L. var. annuum, Cerastium tomentosum, Chaerophyllum bulbosum, Chenopodium quinoa, Chenopodium quinoa subsp. Quinoa, Chenopodium quinoa Willd., Chichorium endivia, Chichorium endivia subsp. Endivia, Circium arvense, Citrullus lanatus, Cornus canadensis, Cornus sericea, Cynara cardunculus subsp. Cardunculus, Daucus carota, Daucus carota subsp carota L., Dolichos lablab, Euphorbia amygdaloides, Fagopyrum tataricum, Foeniculum vulgare, Frangula alnus, Galinsoga quadriradiata, Gentiana lutea, Geranium sanguineum, Geranium×cantabrigiense, Glycyrrhiza glabra, Hamamelis virginiana, Helianthus strumosus, Heliotropium arborescens, Hordeum vulgare subsp. Vulgare, Hypomyces lactifluorum, Juniperus communis L., Lentinus edodes, Lotus corniculatus, Manihot esculenta, Matricaria recutita, Melilotus albus, Melilotus alba Medik., Melissa officinalis, Mentha×piperita, Oenothera biennis, Pastinaca sativa L., Petroselinum crispum, Phaseolus vulgaris, Physalis philadelphica, Phytolacca decandra, Phytolacca decandra syn. P. americana, Pimpinella anisum, Pisum sativum, Potentilla anserina L., Potentilla fruticosa, Poterium sanguisorba, Pyrus communis, Raphanus raphanistrum, Rheum×hybridum, Rhus typhina L., Ribes nigrum L., Ribes sylvestre, Rodgersia spp., Rosmarinus officinalis, Rubus occidentalis, Rubus thibetanus, Rumex crispus, Rumex scutatus, Ruta graveolens, Salvia officinalis, Sambucus canadensis L., Setaria italica, Solanum melongena L., Sorghum dochna bicolor gr technicum, Stellaria media, Tanacetum cinerariifolium, Taraxacum officinale, Teucrium chamaedrys, Thymus fragantissimus, Thymus×citriodorus, Trifolium incarnatum, Triticosecale spp., Tropaeolum majus L., Tsuga canadensis, Tsuga diversifolia, Vaccinium angustifolium, Vaccinium angustifolium Ait., Vitia sp., ×Triticosecale spp., Zea mays L. and Zingiber officinale.

26. The dermatological formulation according to claim 1, wherein said plant extract having extracellular protease inhibiting activity is derived by solvent extraction from a plant selected from the group of: Beta vulgaris L., Brassica oleracea L., Capsicum annuum L, Chenopodium quinoa, Daucus carota L., Geranium×cantabrigiense, Juniperus communis L., Melilotus alba, Pastinaca sativa L., Potentilla anserina L., Rhus typhina L., Solanum melongena L., Tropaeolum majus L., Vaccinium angustifolium, ×Triticosecale spp. and Zea mays L.

27. The dermatological formulation according to claim 3, wherein said alcoholic solvent is ethanol or a glycol.

28. The dermatological formulation according to claim 1, wherein said plant is subjected to one or more stress prior to said solvent extraction.

29. A method of preparing a dermatological formulation comprising admixing an effective amount of one or more plant extracts having extracellular protease inhibiting activity with a physiologically acceptable carrier, said plant extract derived from any one of the plants listed in Tables 1, 2, 3, 4 and 5 by solvent extraction, and said extracellular protease selected from the group of: matrix metalloprotease-1 (MMP-1), matrix metalloprotease-2 (MMP-2), matrix metalloprotease-3 (MMP-3), matrix metalloprotease-9 (MMP-9) and human leukocyte elastase (HLE), wherein said plant extract modulates one or more cellular activities in skin cells.
